(19) United States
(12) Patent Application Publication
(10) Pub. No.: US 2018/0163178 A1
(43) Pub. Date: Jun. 14, 2018

(54) AUF1 ENCODING COMPOSITIONS FOR MUSCLE CELL UPTAKE, SATELLITE CELL POPULATIONS, AND SATELLITE CELL MEDIATED MUSCLE GENERATION

(71) Applicants: Robert J. SCHNEIDER, New York, NY (US); Devon M. CHENETTE, New York, NY (US)

(72) Inventors: Robert J. SCHNEIDER, New York, NY (US); Devon M. CHENETTE, New York, NY (US)

(21) Appl. No.: 15/577,851

(22) PCT Filed: May 27, 2016
(86) PCT No.: PCT/US16/34794

§ 371 Date: Nov. 29, 2017

Related U.S. Application Data
(60) Provisional application No. 62/168,476, filed on May 29, 2015.

(51) Int. Cl. C12N 5/077; C07K 14/435; A01K 67/027; C12Q 1/6883; A61P 21/00; C12N 15/86

(52) U.S. Cl. CPC C12N 5/0659; C07K 14/435; A01K 67/0276; C12Q 1/6883; C12N 2015/8536; C12N 15/86; A01K 2217/075; A01K 2227/105; A01K 2267/0306; A61P 21/00

(57) ABSTRACT

The present invention relates to compositions (e.g., AUF1 encoding compositions) for muscle cell uptake, satellite cell populations and compositions containing muscle satellite cell populations, pharmaceutical compositions, methods of producing muscle satellite cell compositions, and methods of causing muscle satellite cell mediated muscle generation.

This application claims priority benefit of U.S. Provisional Patent Application No. 62/168,476, filed May 29, 2015, which is hereby incorporated by reference in its entirety.

This invention was made with government support under grant numbers GM085693, R24OD018339, and T32 13-A0-S1-090476 awarded by the U.S. National Institutes of Health. The government has certain rights in this invention.

### FIELD OF THE INVENTION

The present invention relates to compositions for muscle cell uptake, satellite cell populations and compositions containing muscle satellite cell populations, pharmaceutical compositions, methods of producing muscle satellite cell compositions, and methods of causing muscle satellite cell mediated muscle generation and/or regeneration.

### BACKGROUND OF THE INVENTION

Satellite cells are a population of stem cells located on the basal lamina of myofibers with the capability to regenerate adult skeletal muscle. Once satellite cells are activated in response to injury they rapidly proliferate, recapitulate myogenesis, and fuse together to form fibers (Bernet et al., “p38 MAPK Signaling Underlies a Cell-autonomous Loss of Stem Cell Self-renewal in Skeletal Muscle of Aged Mice,” Nature Medicine 20:265-271 (2014)). Satellite cells must also self-renew and quiesce to prevent their depletion. Satellite cells therefore divide asymmetrically, enabling a small number of stem cells to return to quiescence, in part mediated through interaction with the satellite cell niche. Quiescent satellite cells maintain unique expression of PAX7 while activated satellite cells show expression of myogenic regulatory factors (“MRFs”), starting with expression of myoD and ultimately gaining expression of myogenin prior to terminal differentiation (Seale et al., “A New Look at the Origin, Function, and ‘Stem-cell’ Status of Muscle Satellite Cells,” Develop. Biol. 218:115-124 (2000)). Multiple studies have debated the importance of the PAX7-positive satellite cell population in regeneration due to the robust nature of the myofiber itself and the multiple cell types necessary to achieve complete repair (Brack, “Pax7 is Back,” Skeletal Muscle 4:24 (2014); Gunther et al., “Myf5-positive Satellite Cells Contribute to Pax7-dependent Long-term Maintenance of Adult Muscle Stem Cells,” Cell Stem Cell 13:590-601 (2013); Kudryashova et al., “Satellite Cell Senescence Underlies Myopathy in a Mouse Model of Limb-girdle Muscular Dystrophy 2H,” J. Clin. Invest. 122:1764-1776 (2012); and Lepper et al., “Adult Satellite Cells and Embryonic Muscle Progenitors have Distinct Genetic Requirements,” Nature 460:627-631 (2009)). This debate has resulted in a minimal understanding of satellite cells in myopathic diseases.

Myopathies, which include developmental diseases such as Duchene's muscular dystrophy and late-onset diseases such as limb-girdle muscular dystrophy (“LGMD”), affect the development, function, and aging of skeletal muscle. They can be genetic in etiology or acquired through injury, inflammation, or sarcopenia. Myopathies cause extreme muscle weakness, leaving the patient in pain with limited mobility and dexterity. Current treatments are limited to managing disease through physical therapy and in some cases drug assistance or surgery (Mercuri and Muntoni, “Muscular Dystrophy: New Challenges and Review of the Current Clinical Trials,” Cur. Opin. Ped. 25:701-707 (2013)). Recent studies show satellite cells are an area of high interest and debate for the understanding of late on-set myopathies, like LGMD, and the development of stem cell based therapies. LGMD is a family of adult diagnosed muscular dystrophies with great genetic heterogeneity. Physiologically, patients show reduced muscle mass, limb weakness, and extreme fatigue. Histologically, skeletal muscle fibers show irregular sizes, they contain centralized nuclei suggesting aberrant cell division, and show increased matrix deposits such as collagen (Kudryashova et al., “Satellite Cell Senescence Underlies Myopathy in a Mouse Model of Limb-girdle Muscular Dystrophy 2H,” J. Clin. Invest. 122:1764-1776 (2012)). While satellite cell-based therapies present a novel means to treat this disease, the mechanism of rapid changes in the gene expression of satellite cells are poorly understood.

Many key regulatory mRNAs are controlled through post-transcriptional mechanisms, typically the targeted destabilization of the mRNA, its selective translation, or both (Moore et al., “Physiological Networks and Disease Functions of RNA-Binding Protein AUF1,” Wiley Interdisciplinary Reviews RNA 5:549-564 (2014)). The regulated stability of mRNAs generally comprises those that must respond quickly in abundance to changing stimuli. In fact, almost half of the changes in physiologically rapid inducible gene expression occur at the level of mRNA stability (Cheadle et al., “Control of Gene Expression During T Cell Activation: Alternate Regulation of mRNA Transcription and mRNA Stability,” BMC Genomics 6:75 (2005)). RNA binding proteins (“RBPs”) enable a quick change in gene expression in response to changing external stimuli through regulation of RNA splicing, localization, decay, and translation (Kim et al., “Emerging Roles of RNA and RNA-binding Protein Network in Cancer Cells,” BMB Reports 42:125-130 (2009)). Many of these physiologically potent proteins are encoded by short-lived mRNAs, with half-lives of minutes, where mRNA destabilization is conferred by AU-rich elements (“AREs”) in the 3′ untranslated region (“3′UTR”). A common ARE motif consists of the sequence AUUUA, typically repeated multiple times in the 3′UTR, often contiguously (Moore et al., “Physiological Networks and Disease Functions of RNA-Binding Protein AUF1,” Wiley Interdisciplinary Reviews RNA 5:549-564 (2014)). The ARE is purely a cis-acting element that serves as a binding site for regulatory proteins known as AU-rich binding proteins (“AUBPs”) which bind the ARE with high affinity and control mRNA stability or translation. Several AUBPs have been well studied to date, and all act by recruiting mRNA decay, mRNA stabilizing or translation arrest proteins (Gratacos et al., “The Role of AUF1 in Regulated mRNA Decay,” Wiley Interdisciplinary reviews RNA 1:457-473 (2010)). AUBPs also have different and overlapping target ARE-mRNAs (Garneau et al., “The Highways and Byways of mRNA Decay,” Nat Rev Mol Cell Biol 8:113-126 (2007); Kim et al., “Emerging Roles of RNA And RNA-Binding Protein Network in Cancer Cells,” BMB Reports 42:125-130 (2009)). ARE-mRNAs are thought to encode more than 5% of the protein expressed genome (Gruber et al., “AREsite: A Database for the Comprehensive Investigation of AU-Rich Elements,” Nucleic Acids Res 39:D66-69 (2010)).

AU-rich element RNA-binding protein 1 (“AUF1,” also known as hnRNPD) is an RBP known to target mRNA containing AREs for rapid decay (Zhang et al., “Purification, Characterization, and cDNA Cloning of an AU-rich Element RNA-binding Protein, AUF1,” Mol. Cell. Biol. 13:7652-7665 (1993); Moore et al., “Physiological Networks and Disease Functions of RNA-binding Protein AUF1,” Wiley Interdisciplinary Reviews, RNA 5:549-564 (2014)). AUF1 knockout mice show accelerated aging, including a novel identification of reduced muscle mass (Pont et al., “mRNA Decay Factor AUF1 Maintains Normal Aging, Telomere Maintenance, and Suppression of Senescence by Activation of Telomerase Transcription,” Molecular Cell 47:5-15 (2012)). This observation suggests a possible role of AUF1 in regulating the changing gene network crucial to skeletal muscle maintenance potentially through expression in the satellite cell. However, AUF1's role in such regulation and maintenance has not yet been determined.

The present invention is directed to overcoming deficiencies in the art, particularly as it pertains to treatment of late-onset myopathic diseases.

### SUMMARY OF THE INVENTION

One aspect of the present invention relates to a composition comprising a nucleic acid molecule encoding an AUF1 protein or a functional fragment thereof, and a targeting element which controls muscle satellite cell-specific uptake or expression, where the targeting element is heterologous to the AUF1 gene.

Another aspect of the present invention relates to a composition comprising a muscle satellite cell population, where the cell population comprises a transgene exogenous to the satellite cells and encoding AUF1 protein or a functional fragment thereof.

A further aspect of the present invention relates to a composition comprising a muscle cell population comprising an AUF1 gene encoding AUF1 protein or functional fragment thereof, where expression of the AUF1 gene is controlled by a promoter heterologous to the AUF1 gene.

Yet another aspect of the present invention relates to a pharmaceutical composition comprising (a) one or more of an MMP-9 inhibitor, a Twist1 inhibitor, or a cyclin D1 inhibitor; (b) a targeting element that causes muscle satellite cell-specific uptake or activity of the one or more inhibitors; and (c) a pharmaceutically-acceptable carrier.

Yet another aspect of the present invention relates to a pharmaceutical composition comprising (a) one or more of an IL17 inhibitor, an MMP-8 inhibitor, an IL10 inhibitor, an FGR inhibitor, a TREM1 inhibitor, a CCR2 inhibitor, an ADAM8 inhibitor, or an IL1b inhibitor; (b) a targeting element that causes muscle satellite cell-specific uptake or activity of the one or more inhibitors; and (c) a pharmaceutically-acceptable carrier.

A further aspect of the present invention relates to a method of producing a muscle satellite cell population. This method involves transforming or transfecting Syndecan 4⁺/PAX7⁺ or Syndecan 4⁺/PAX7⁻ muscle satellite cells with a nucleic acid molecule encoding exogenous AUF1 or a functional fragment thereof under conditions effective to express exogenous AUF1 in the muscle satellite cells.

Still another aspect of the present invention relates to a muscle satellite cell population produced by the above method of producing a muscle satellite cell population.

A further aspect of the present invention relates to a method of causing satellite-cell mediated muscle generation in a subject. This method involves selecting a subject in need of satellite-cell mediated muscle generation and administering to the selected subject (i) a composition of the present invention, (ii) a cell population of the present invention, (iii) AUF1 protein, a functional fragment of AUF1 protein, an AUF1 protein mimic, or a combination thereof, or (iv) a combination of (i), (ii), and (iii), under conditions effective to cause satellite-cell mediated muscle generation in the selected subject.

Another aspect of the present invention relates to an in vivo method of producing a muscle satellite cell population expressing exogenous AUF1 or a functional fragment thereof. This method involves transforming or transfecting Syndecan 4⁺/PAX7⁺ or Syndecan 4⁺/PAX7⁻ muscle satellite cells with a nucleic acid molecule encoding exogenous AUF1 or a functional fragment thereof, where when Syndecan 4⁺/PAX7⁺ or Syndecan 4⁺/PAX7⁻ muscle satellite cells are transformed or transfected in an in vitro or an in vivo model with the nucleic acid molecule they express the exogenous AUF1 or the functional fragment thereof.

Another aspect of the present invention relates to a method of treating a subject in need thereof with Syndecan 4⁺/PAX7⁺ or Syndecan 4⁺/PAX7⁻ muscle satellite cells expressing exogenous AUF1. This method involves administering Syndecan 4⁺/PAX7⁺ or Syndecan 4⁺/PAX7⁻ muscle satellite cells transformed or transfected with a nucleic acid molecule encoding exogenous AUF1 or a functional fragment thereof, where the Syndecan 4⁺/PAX7⁺ or Syndecan 4⁺/PAX7⁻ muscle satellite cells express the exogenous AUF1 or the functional fragment thereof in an in vitro or an in vivo model.

The present invention relates to regulating satellite cell fate through the expression of AUF1, ultimately controlling the maintenance of a quiescent population, and linking satellite cell alterations to late on-set myopathies. As AUF1^{-/-} mice age, they show progressive loss of skeletal muscle mass and corresponding muscle weakness starting at 6 months despite developing histologically healthy skeletal muscle. Aging AUF1^{-/-} skeletal muscle shows a phenotype strikingly similar to limb-girdle muscular dystrophy, including reduced myofiber size and increased centralized nuclei. While AUF1 is not expressed in the terminally differentiated myofiber, a significant increase in AUF1 expression in satellite cells following activation was identified. Following injury, AUF1^{-/-} satellite cells show aberrant skeletal muscle repair resulting in the complete loss of the PAX7-positive quiescent population. To understand this phenomenon, RNA-Seq analysis of the AUF1^{-/-} activated satellite cell was completed and resulted in the identification of a significant increase in Matrix Metallopeptidase 9 (“MMP9”) mRNA. MMP9 is a protein involved in the breakdown of extracellular matrix through cleaving multiple collagens. Further studies identified an increased stability of the MMP9 transcript in the absence of AUF1, resulting in an increased secretion of MMP9 from the satellite cell. Using living imaging, MMP9 is seen as being significantly more active in AUF1^{-/-} skeletal muscle following hindlimb injury than in the wild-type (“WT”). Increased MMP9 activity in the uninjured AUF1^{-/-} skeletal muscle is also observed, while none is present in the WT.

The data set forth in the Examples infra shows, inter alia, that in the absence of AUF1 satellite cells enter a “self-sabotaging” program by secreting high levels of MMP9. This increased expression of MMP9 causes (1) the premature activation of satellite cells with aging and (2) the breakdown of the satellite cell niche following traumatic injury. As noted above, satellite cells must also self-renew and quiesce to prevent their depletion. Satellite cells therefore divide asymmetrically, enabling a small number of stem cells to return to quiescence, in part mediated through interaction with the satellite cell niche. The satellite cell niche is loosely defined as the intact laminin-basement membrane structure that provides poorly characterized extrinsic factors crucial for their maintenance. (Bernet et al., “p38 MAPK Signaling Underlies a Cell-Autonomous Loss of Stem Cell Self-Renewal in Skeletal Muscle of Aged Mice,” Nature Medicine 20: 265-271 (2014); Carlson & Conboy, “Loss of Stem Cell Regenerative Capacity Within Aged Niches,” Aging Cell 6:371-382 (2007); Collins et al., “Stem cell function, self-renewal, and behavioral heterogeneity of cells from the adult muscle satellite cell niche,” Cell 122:289-301 (2005); Kuang et al., “Asymmetric Self-Renewal and Commitment of Satellite Stem Cells in Muscle,” Cell 129:999-1010 (2007); Montarras et al., “Direct Isolation of Satellite Cells for Skeletal Muscle Regeneration,” Science 309:2064-2067 (2005); Zammit et al., “Muscle satellite Cells Adopt Divergent Fates: A Mechanism for Self-Renewal?,” J Cell Biol 166:347-357 (2004), each of which is hereby incorporated by reference in its entirety).

The work reported herein shows that AUF1 regulation of MMP9 is crucial to maintaining a satellite cell population, and confirms that MMP9 inhibition in auf1 knockout mice resulted in restoration of muscle wound repair. Furthermore, novel AUF1 targets are identified, and it is explained that late on-set myopathies may have a satellite cell derived origin likely due to the loss or mutation of AUF1. Based on this work, described herein are compositions and methods relating to, inter alia, delivery to satellite cells of (i) functional AUF1 (or a functional fragment of AUF1, or nucleotide molecules encoding such polypeptides); (ii) inhibitors of AUF1 targets described herein (e.g., MMP-9, Twist1, cyclin D1, IL17, MMP-8, IL10, FGR, TREM1, CCR2, ADAMS, and IL1b); or (iii) both (i) and (ii). As described herein, such compositions are of use in both functional AUF1 deficient and functional AUF1 sufficient satellite cells to effect, inter alia, muscle injury repair and/or muscle generation.

### BRIEF DESCRIPTION OF THE DRAWINGS

FIGS. 1A-1E illustrate the results of an initial observation that mice lacking functional AUF1 protein show severe muscle loss with age corresponding to reduced strength. FIG. 1A is a photograph showing a representative image of the hindlimb muscle mass of 6 month old WT and knockout (“KO”) mice. FIG. 1B are photographs showing representative images of 6 month old WT and KO mice, respectively, produced by the Dual Energy X-Ray Absorptiometry (DEXA) Body analyzer. FIG. 1C is a graph showing average whole body skeletal muscle mass calculated from the lean tissue mass DEXA reading normalized to total body mass at different ages in WT and KO mice. FIG. 1D is a graph showing forearm strength measured through strength grip analysis of WT and KO mice. FIG. 1E is a graph showing whole body strength measured through cage flip analysis at different ages in WT and KO mice.

FIGS. 2A-2E relate to the pathology of the AUF1^{-/-} skeletal muscle. Specifically, mice lacking functional AUF1 protein are shown to develop a myopathic phenotype with age due to the premature activation of the satellite cell population. FIG. 2A provides photographs showing hindlimb muscle stained for the perimeter of the muscle bundle by Laminin (green) and the nuclei (DAPI blue) at 4 months of age and 8 months of age in WT and KO mice. FIG. 2B is a graph showing quantification of the centralized nuclei indicating premature activation of satellite cells which are normally localized to the Laminin in the 8 month old KO mice. FIG. 2C is a pair of graphs showing quantification of the Laminin muscle fiber area showing smaller fibers in the 4 month old (top) and 8 month old (bottom) KO mice suggesting muscle loss. FIG. 2D is a pair of graphs showing quantification of the Laminin muscle fiber Minimum Ferret's Diameter, a measurement commonly used in muscle studies that corrects for sectioning errors, showing smaller fibers in the 4 month old (top) and 8 month old (bottom) KO mice suggesting muscle loss. FIG. 2E provides photographs of H&E staining of 8 month old WT and KO mouse skeletal muscle, showing irregular fiber formation and centralized nuclei in the KO mice similar to the diagnostic appearance of LGMD.

FIGS. 3A-3E relate to AUF1 expression in the satellite cell. Satellite cells are the primary cell type in the muscle capable of division, because muscle fibers are unable to grow or divide. AUF1 is shown to be expressed in satellite cells actively involved in skeletal muscle regeneration. FIG. 3A provides photographs of hindlimb muscle from experiments using immunofluorescence analysis for expression of laminin (AF488, green), PAX7 (AF 555, red), AUF1 (AF647, white) and nuclei (DAPI, blue) in uninjured (UI) or 7 day post-injury TA muscle in 4 month old WT mice. TA muscle was injured by BaCl₂ injection. TAs were frozen in OCT, 5 images from 3 sections were analyzed per mouse (scale bar 50 μm). DAPI+2^{nd} is a background control, sections stained with DAPI and secondary antibody only. FIG. 3B shows experimental results demonstrating that AUF1 is expressed in MyoD+ satellite cells. Quantification of AUF1 co-localization to PAX7 in uninjured and 7 days post-injury TA muscle showing AUF1 is expressed in a subset of PAX7+ satellite cells is shown in the graph in the top panel of FIG. 3B. Quantification of AUF1 co-localization with MyoD in cultured myofibers showing AUF1 is expressed in over 50% of MyoD+ satellite cells is shown in the graph in the bottom panel of FIG. 3B. FIG. 3C is a graph showing expression of AUF1 from Sdc4-positive satellite cells sorted 48 hours after injury compared to Sdc4-positive satellite cells sorted from an uninjured hindlimb. FIG. 3D includes photographs showing immunofluorescence analysis for expression of AUF1 (AF488, green), MyoD (AF555, red), and nuclei (DAPI, blue) in myofibers isolated from WT skeletal muscle from 4 month old mice. Ten fibers were analyzed per mouse and three mice were studied (scale bar 50 μm). FIG. 3E is a graph showing quantification of the AUF1 and MyoD co-localization.

FIGS. 4A-4E relate to how the AUF1^{-/-} satellite cell population compares to a healthy WT satellite cell population with respect to repairing injury. Specifically, in the absence of AUF1, satellite cells are shown to be unable to repair skeletal muscle injury resulting in irregular muscle fibers and a loss of the PAX7-positive satellite cell population. FIG. 4A includes photographs showing hindlimb muscle stained for nuclei (DAPI blue), Laminin (green), and PAX7 (red) from the WT or KO mice 7 or 15 days after hindlimb injury by BaCl₂ injection. The DAPI and secondary antibody panel are a control showing that in the KO mouse muscle satellite cells are unable to form proper laminin fibers and, therefore, exhaust and deplete the population. FIG. 4B is a pair of graphs showing quantification of the 15 days post-injury laminin fiber area and Minimum Ferret's Diameter showing significantly smaller fibers in the KO mice and significantly larger fibers in the WT mice suggesting a loss of muscle mass. FIG. 4C is a graph showing quantification of the PAX7-positive cells showing minimal PAX7 expansion 7 days post-injury and complete PAX7 depletion 15 days post-injury in the KO mice. FIG. 4D is a graph showing the number of satellite cells able to be isolated through Sdc4 selection in the hindlimb at 6 months of age in WT and KO mice. FIG. 4E is a pair of photographs showing fibers isolated from the hindlimb muscle of WT and KO mice stained for nuclei (DAPI blue) and PAX7 (green) showing complete loss of PAX7 following satellite cell activation in the KO mice.

FIGS. 5A-5C relate to how myogenesis is altered in the absence of AUF1. Specifically, in the absence of AUF1, satellite cells are shown to rapidly proliferate without differentiation. FIG. 5A includes photographs showing cultured hindlimb muscle lysate from WT and KO mice stained for nuclei (DAPI blue), MyoD (red), the late muscle differentiation factor Myogenin (green), and the division identifier EDU (white) showing significantly more dividing cells with no multi-nucleated myofibers in the KO mice population. FIG. 5B includes photographs showing fibers isolated from the hindlimb muscle of WT and KO mice stained for nuclei (DAPI blue), MyoD (green), and Myogenin (red) showing significantly more cells dividing in the KO fibers. FIG. 5C is a graph showing quantification of nuclei from the WT and KO mouse fibers showing a constant cell division in the KO mouse fibers despite expression of late differentiation factors.

FIGS. 6A-6B show results from experiments conducted to test whether the proliferating satellite cell phenotype can be rescued with the addition of AUF1. Specifically, ex vivo addition of AUF1 p40, p42, or p45 to KO mouse fibers is shown to rescue the proliferating phenotype. FIG. 6A shows photographs of fibers isolated from WT or KO mice hindlimb muscle treated with either AUF1 p37, p40, p42, or p45 stained for AUF1 (red). FIG. 6B is a graph showing quantification of nuclei showing hyper-proliferation in the KO mice with an empty vector or the addition of just p37.

FIGS. 7A-7E relate to the analysis of transcript levels in auf1^{-/-} satellite cells as compared to wild tyle. FIG. 7A is a heat map of RNA-Seq analysis from sorted WT and KO satellite cells. Three mice per genotype were studied. Ninety-one genes were differentially expressed in KO satellite cells with the majority showing increased expression (red). FIG. 7B is an IPA characterization of top cellular function and disease pathways for satellite cell ARE-mRNAs dysregulated in the absence of AUF1 expression. Numbers represent P-value×10⁻⁵. FIG. 7C is a heat map of Affymetrix data from whole hindlimb skeletal muscle. Whole hindlimb skeletal muscle from WT and KO 6 month old mice was surgically removed and RNA was extracted from the fibers according to manufacturer's instructions (TRIzol). Muscle was isolated from 5 mice per genotype. RNA was cleaned using RNeasy Mini Elute Kit (Qiagen) and analyzed on Affymetrix chips. Twelve genes were differentially expressed, significantly fewer than in satellite cells. Upregulated genes are shown in red and downregulated genes are shown in green. FIG. 7D is a table showing those mRNAs altered in abundance in satellite cells as determined by RNA-Seq analysis. Ninety-one genes were identified, indicated by the gene abbreviated name, and the log 2 fold change from RNA-Seq analysis was reported. Transcripts containing probable ARE sequences in the 3′UTR are marked with an asterisk (*). Transcripts containing at least two ARE pentames are marked with two asterisks (**). Transcripts decreased in abundance are indicated by a minus sign. FIG. 7E is a table summarizing the known AUF1 target mRNAs identified as altered in AUF1^{-/-} satellite cells. Genes significantly altered in the AUF1^{-/-} satellite cells detected by RNA-Seq analysis were subject to in silico characterization for known AUF1 association. Four genes were identified. Shown are the gene abbreviated name, number of predicted AUF1-targeted ARE motifs in the mRNA 3′UTR, the fold change extrapolated from the log 2 change calculated from RNA-Seq analysis, and whether the gene has been linked to skeletal muscle regeneration (Y=Yes, N=No). Genes decreased in abundance are indicated by a minus sign.

FIGS. 8A-8C show experimental results demonstrating that MMP9 is significantly more active in the auf1^{-/-} skeletal muscle following injury. FIG. 8A shows Bioluminescence (IVIS) images of representative 4 month old mice treated with MMPSense for 48 h to assess MMP9 activity 24 h following TA BaCl₂ injury of left hind limb, compared to an uninjured control (right hind limb). Three mice per genotype were studied. FIG. 8B shows IVIS images of representative WT (left) and KO (right) excised TA muscles treated with MMP-Sense for 48 h to assess MMP9 activity 24 h after injury. FIG. 8C is a graph showing quantification of MMP-Sense IVIS images in WT and KO injured TA muscles 24 h post-injury. *P<0.05, unpaired t-test. Independent confirmation of the AUF1 temporal expression profile was obtained using the murine myoblast C2C12 cell line. C2C12 cells can mimic the post-activated satellite cell state initiating at the progenitor myoblast level (Ho, et al., “PEDF-Derived Peptide Promotes Skeletal Muscle Regeneration Through its Mitogenic Effect on Muscle Progenitor Cells,” Am J Physiol Cell Physiol 309(3):C159-68 (2015); Silva et al., “Inhibition of stat3 Activation Suppresses Caspase-3 and the Ubiquitin-Proteasome System, Leading to Preservation of Muscle Mass in Cancer Cachexia,” J Biol Chem 290:11177-11187 (2015), each of which is hereby incorporated by reference in its entirety).

FIGS. 9A-9C relate to whether AUF1 can be studied in a murine tissue culture model of myogenesis known as C2C12 cells. In particular, FIGS. 9A-C show that differentiation is delayed when AUF1 is partially silenced in C2C12 cells. FIG. 9A shows protein expression in C2C12 cells following myogenesis, showing AUF1 expression throughout differentiation by no AUF1 expression once myofibers are formed corresponding to expression of the known AUF1 target Cyclin D1. FIG. 9B shows that using an siAUF1 construct, AUF1 can effectively be silenced in the C2C12 cells. FIG. 9C is a pair of photographs providing representative images of the C2C12 cell population 24 hours after differentiation showing myotube formation in the non-silenced cells while no myotubes are present in the si-AUF1 cells.

FIGS. 10A-10G relate to whether MMP9 is more active in C2C12 cells treated with siAUF1. MMP9 is shown to be significantly more active when AUF1 is partially silenced in the C2C12 cells. FIG. 10A is a graph showing mRNA levels of AUF1 and MMP9 from cultured C2C12 cells treated with vehicle (black) or siAUF1 (grey). Two siAUF1 targeting sequences were used. mRNA levels were normalized to GapDH. Each experiment was performed in triplicate. *P<0.05, **P<0.005, unpaired t-test. FIG. 10B is a graph showing relative MMP9 mRNA decay rate in cultured C2C12 cells treated with control (black) or siAUF1-1 (grey). Cells were collected post-actinomycin D treatment and RNA isolated per manufacturer instructions (TRIzol). Partial decay curve is shown. Inset: immunoblot of AUF1 levels post-silencing. *P<0.001, unpaired t-test. FIG. 10C is a graph of experimental results demonstrating that AUF1 promotes the destabilization of MMP9 through ARE-rich regions in the 3′UTR. The longest ARE-repeat (˜200 kB) was cloned behind the luciferase region of a pzeo-luc vector. This plasmid was transient transfected in untreated (C2C12) or siAUF1 treated (siAUF1) C2C12 cells for 48 hours. Luciferase activity was measured using a Dual Luciferase Report Assay (Promega). (**P<0.005, unpaired t-test). FIG. 10D is a graph showing RNA-immunoprecipitation of IgG or AUF1 analyzed for MMP9 association showing increased MMP9 in the AUF1 IP from C2C12 cells without si-AUF1 treatment. FIG. 10E shows protein levels of secreted MMP9 from C2C12 cells with or without siAUF1 treatment. FIG. 10F is a graph showing ELISA measuring MMP9 activity of C2C12 cells with or without siAUF1 treatment. FIG. 10G shows RNA-Immunoprecipitation of IgG (black) or endogenous AUF1 (grey) in C2C12 cells analyzed for MMP9 and ITGB1 mRNA levels.

FIGS. 11A-11D show results demonstrating that inhibition of MMP9 activity in auf1^{-/-} mice restores maintenance of the PAX7⁺ satellite cell population. FIG. 11A shows IVIS images of 4 month old mice treated with MMP-Sense with (right, KO+SB-3CT) or without (left, KO) SB-3CT for 48 h to assess MMP9 activity 24 h after TA BaCl₂ injury (left hind limb) compared to an uninjured TA (right hind limb). Three mice per treatment were studied. FIG. 11B is a graph showing quantification of MMP-Sense IVIS imaging in KO and KO+SB-3CT injured TA muscles 24 h post-injury. **P<0.005, unpaired t-test. FIG. 11C includes images showing immunofluorescence for the expression of laminin (AF488, green), PAX7 (AF555, red), and nuclei (DAPI, blue) in 7 days post-injury skeletal muscle in 4 month old KO and KO+SB-3CT mice. TA muscle was injured through BaCl₂ injection. TA muscles were frozen in OCT, 5 images from 3 sections were analyzed per mouse (scale bar 50 μm). FIG. 11D is a graph showing quantification of PAX7 expression in KO and KO+SB-3CT mice in 7 days post-injury skeletal muscle. *P<0.05, unpaired t-test.

FIG. 12 is a schematic illustration showing that loss or mutation of AUF1 results in a “self-sabotaging” satellite cell phenotype, in which cells are unable to be maintained in aging or during injury. Specifically, FIG. 12 shows how AUF1^{-/-} satellite cells are altered in both aging and injury ultimately resulting in a myopathic phenotype due to increased active MMP9.

FIG. 13 is a schematic illustration showing exemplary ex vivo and in vivo therapeutic routes of the present invention.

FIGS. 14A-14E provide evidence that other genes are altered in the siAUF1 C2C12 population during terminal differentiation. Specifically, Twist1, the stem-maintenance transcription factor, is altered in the absence of AUF1 during C2C12 myogenesis. FIG. 14A is a graph showing RNA levels of AUF1, Myogenin, Nascent Myogenin (Unaltered by RNA-binding proteins), Twist1, and MYF6 (a control differentiation factor) in differentiating C2C12 cells with or without siAUF1 treatment. FIG. 14B is a graph showing RNA stability levels of Twist1 in differentiating C2C12 cells with or without siAUF1 treatment. FIG. 14C is a graph showing RNA-immunoprecipitation of IgG or AUF1 analyzed for Twist1 association. FIG. 14D includes photographs showing protein levels of Myosin (identifying differentiation), GapDH, and Twist1 in differentiating C2C12 cells with or without siAUf1 treatment. FIG. 14E is a schematic illustration showing the effect of increased Twist1 expression on myogenesis.

FIG. 15 is a schematic illustration showing function of AUF1 in activation and differentiation of satellite cells.

### DETAILED DESCRIPTION OF THE INVENTION

A first aspect of the present invention relates to a composition comprising a nucleic acid molecule encoding an AUF1 protein or a functional fragment thereof, and a targeting element which controls muscle satellite cell-specific uptake or expression, where the targeting element is heterologous to the AUF1 gene.

As used herein the terms “satellite cell,” “satellite stem cell,” “muscle satellite cell,” and the like are used interchangeably to refer to cells located on the basal lamina of myofibers having the capability to regenerate adult skeletal muscle.

AUF1 is encoded by a single copy gene comprised of 10 exons on chromosome 4 (4q21), and is expressed as a family of four protein isoforms generated by alternative pre-mRNA splicing of exons 2 and 7 (Zucconi and Wilson, “Modulation of Neoplastic Gene Regulatory Pathways by the RNA-binding Factor AUF1,” Front. Biosci. 16:2307-2325 (2013); Wagner et al., “Localization and Physical Mapping of Genes Encoding the A+U-rich Element RNA-binding Protein AUF1 to Human Chromosomes 4 and X,” Genomics 34:219-222 (1996); and Wagner et al., “Structure and Genomic Organization of the Human AUF1 Gene: Alternative Pre-RNA Splicing Generates Four Protein Isoforms,” Genomics 48:195-202 (1998), which are hereby incorporated by reference in their entirety). The AUF1 protein isoforms include p37^{AUF1}, p40^{AUF1}, p42^{AUF1}, and p45^{AUF1} (Zucconi and Wilson, “Modulation of Neoplastic Gene Regulatory Pathways by the RNA-binding Factor AUF1,” Front. Biosci. 16:2307-2325 (2013), which is hereby incorporated by reference in its entirety). Each of these four isoforms include two centrally-positioned, tandemly arranged RNA recognition motifs (“RRMs”) which mediate RNA binding (DeMaria et al., “Structural Determinants in AUF 1 Required for High Affinity Binding to A+U-rich Elements,” J. Biol. Chem. 272:27635-27643 (1997), which is hereby incorporated by reference in its entirety).

The general organization of an RRM is a β-α-β-β-α-β RNA binding platform of anti-parallel β-sheets backed by the a-helices (Zucconi and Wilson, “Modulation of Neoplastic Gene Regulatory Pathways by the RNA-binding Factor AUF1,” Front. Biosci. 16:2307-2325 (2013); Nagai et al., “The RNP Domain: A Sequence-specific RNA-binding Domain Involved in Processing and Transport of RNA,” Trends Biochem. Sci. 20:235-240 (1995), which are hereby incorporated by reference in their entirety). Structures of individual AUF1 RRM domains resolved by NMR are largely consistent with this overall tertiary fold (Zucconi and Wilson, “Modulation of Neoplastic Gene Regulatory Pathways by the RNA-binding Factor AUF1,” Front. Biosci. 16:2307-2325 (2013); Nagata et al., “Structure and Interactions with RNA of the N-terminal UUAG-specific RNA-binding Domain of hnRNP D0,” J. Mol. Biol. 287:221-237 (1999); and Katahira et al., “Structure of the C-terminal RNA-binding Domain of hnRNP D0 (AUF1), Its Interactions with RNA and DNA, and Change in Backbone Dynamics Upon Complex Formation with DNA,” J. Mol. Biol. 311:973-988 (2001), which are hereby incorporated by reference in their entirety).

The term “fragment” or “portion” when used herein with respect to a given polypeptide sequence (e.g., AUF1), refers to a contiguous stretch of amino acids of the given polypeptide's sequence that is shorter than the given polypeptide's full-length sequence. A fragment of a polypeptide may be defined by its first position and its final position, in which the first and final positions each correspond to a position in the sequence of the given full-length polypeptide. The sequence position corresponding to the first position is situated N-terminal to the sequence position corresponding to the final position. The sequence of the fragment or portion is the contiguous amino acid sequence or stretch of amino acids in the given polypeptide that begins at the sequence position corresponding to the first position and ends at the sequence position corresponding to the final position. Functional or active fragments are fragments that retain functional characteristics, e.g., of the native sequence or other reference sequence. Typically, active fragments are fragments that retain substantially the same activity as the wild-type protein. A fragment may, for example, contain a functionally important domain, such as a domain that is important for receptor or ligand binding.

Accordingly, in certain embodiments, functional fragments of AUF1 as described herein include at least one RRM domain. In certain embodiments, functional fragments of AUF1 as described herein include two RRM domains.

AUF1 or functional fragments thereof as described herein may be derived from a mammalian AUF1. In one embodiment, the AUF1 or functional fragment thereof is a human AUF1 or functional fragment thereof. In another embodiment, the AUF1 or functional fragment thereof is a murine AUF1 or a functional fragment thereof. The AUF1 protein according to embodiments described herein may include one or more of the AUF1 isoforms p37^{AUF1}, p40^{AUF1}, p42^{AUF1}, and p45^{AUF1}. The GenBank accession numbers corresponding to the nucleotide and amino acid sequences of each isoform is found in Table 1 below, each of which are hereby incorporated by reference in their entirety.

TABLE 1
GenBank Accession Numbers of AUF1 Sequences

| | Human | | Mouse | |
|---|---|---|---|---|
| Isoform | Nucleotide | Amino Acid | Nucleotide | Amino Acid |
| p37^{AUF1} | NM_ | NP_ | NM_ | NP_ |
| | 001003810.1 | 001003810.1 | 001077267.2 | 001070735.1 |
| | (SEQ ID NO: 1) | (SEQ ID | (SEQ ID | (SEQ ID |
| | | NO: 2) | NO: 3) | NO: 4) |
| p40^{AUF1} | NM_002138.3 | NP_002129.2 | NM_007516.3 | NP_031542.2 |
| | (SEQ ID NO: 5) | (SEQ ID | (SEQ ID | (SEQ ID |
| | | NO: 6) | NO: 7) | NO: 8) |
| p42^{AUF1} | NM_031369.2 | NP_112737.1 | NM_ | NP_ |
| | (SEQ ID NO: 9) | (SEQ ID NO: | 001077266.2 | 001070734.1 |
| | | 10) | (SEQ ID NO: | (SEQ ID NO: |
| | | | 11) | 12) |
| p45^{AUF1} | NM_031370.2 | NP_112738.1 | NM_ | NP_ |
| | (SEQ ID NO: | (SEQ ID NO: | 001077265.2 | 001070733.1 |
| | 13) | 14) | (SEQ ID NO: | (SEQ ID NO: |
| | | | 15) | 16) |

It is noted that the sequences described herein may be described with reference to accession numbers that include, e.g., a coding sequence or protein sequence with or without additional sequence elements or portions (e.g., leader sequences, tags, immature portions, regulatory regions, etc.). Thus, as will be understood, reference herein to such sequence accession numbers or corresponding sequence identification numbers refers to either the sequence fully described therein or some portion thereof (e.g., that portion encoding a protein or polypeptide of interest in the invention (e.g., AUF1 or a functional fragment thereof); the mature protein sequence that is described within a longer amino acid sequence; a regulatory region of interest (e.g., promoter sequence or regulatory element) disclosed within a longer sequence described herein; etc). Likewise, variants and isoforms of accession numbers and corresponding sequence identification numbers described herein are also contemplated.

Accordingly, in certain embodiments, the AUF1 protein referred to herein has an amino acid sequence as set forth in Table 1, or is functional fragment thereof. In one embodiment, the functional fragment as referred to herein includes an amino acid sequence that has at least 80%, at least 85%, at least 90%, at least 95%, at least 97%, or at least 99% amino acid sequence identity to an amino acid sequence identified in Table 1.

As noted above, compositions according to the present invention may include a nucleic acid molecule encoding AUF1 protein or a functional fragment thereof. Such nucleic acid molecules include those having a nucleotide sequence set forth in Table 1, or portions thereof that encode a functional fragment of an AUF1 protein as described supra.

As described in more detail below, compositions according to the present invention are useful in gene therapy, which includes both ex vivo and in vivo techniques. Thus, host cells can be genetically engineered ex vivo with a nucleic acid molecule (or polynucleotide), with the engineered cells then being provided to a patient to be treated. Delivery of the active agent of a composition described herein in vivo may involve a process that effectively introduces a molecule of interest (e.g., AUF1 protein or a functional fragment thereof) into the cells or tissue being treated. In the case of polypeptide-based active agents, this can be carried out directly or, alternatively, by transfecting transcriptionally active DNA into living cells such that the active polypeptide coding sequence is expressed and the polypeptide is produced by cellular machinery. Transcriptionally active DNA may be delivered into the cells or tissue, e.g., muscle, being treated using transfection methods including, but not limited to, electroporation, microinjection, calcium phosphate coprecipitation, DEAE dextran facilitated transfection, cationic liposomes, and retroviruses. In certain embodiments, the DNA to be transfected is cloned into a vector.

Alternatively, cells can be engineered in vivo by administration of the polynucleotide using techniques known in the art. For example, by direct injection of a “naked” polynucleotide (Feigner et al., “Gene Therapeutics,” Nature 349:351-352 (1991); U.S. Pat. No. 5,679,647; Wolff et al., “The Mechanism of Naked DNA Uptake and Expression,” Adv Genet. 54:3-20 (2005), which are hereby incorporated by reference in their entirety) or a polynucleotide formulated in a composition with one or more other targeting elements which facilitate uptake of the polynucleotide by a cell.

Targeting elements include, without limitation, agents such as saponins or cationic polyamides (see, e.g., U.S. Pat. Nos. 5,739,118 and 5,837,533, which are hereby incorporated by reference in their entirety); microparticles, microcapsules, liposomes, or other vesicles; lipids; cell-surface receptors; transfecting agents; peptides (e.g., one known to enter the nucleus); or ligands (such as one subject to receptor-mediated endocytosis). Suitable means for using such targeting elements include, without limitation: microparticle bombardment; coating the polynucleotide with lipids, cell-surface receptors, or transfecting agents; encapsulation of the polynucleotide in liposomes, microparticles, or microcapsules; administration of the polynucleotide linked to a peptide which is known to enter the nucleus; or administration of the polynucleotide linked to a ligand subject to receptor-mediated endocytosis (see, e.g., Wu et al., “Receptor-Mediated in vitro Gene Transformation by a Soluble DNA Carrier System,” J. Biol. Chem. 262:4429-4432 (1987), which is hereby incorporated by reference in its entirety), which can be used to target cell types specifically expressing the receptors. Alternatively, a polynucleotide-ligand complex can be formed allowing the polynucleotide to be targeted for cell specific uptake and expression in vivo by targeting a specific receptor (see, e.g., PCT Application Publication Nos. WO 92/06180, WO 92/22635, WO 92/203167, WO 93/14188, and WO 93/20221, which are hereby incorporated by reference in their entirety).

Accordingly, as noted above, compositions according to the present invention may also include a targeting element which controls satellite cell-specific uptake or expression. Combinations of targeting elements are also contemplated.

In certain embodiments, the targeting element is a satellite cell-specific promoter (e.g., Pax7 promoter, MyoD promoter, myogenin promoter), which drives cell-specific expression. Although the Pax7 promoter, MyoD promoter, myogenin promoter are described, as will be understood, any satellite cell-specific promotor may be used in accordance with the present invention. The targeting element may also be a satellite cell surface protein binding partner (e.g., a binding partner of the satellite cell surface protein Syndecan 4). Such binding partners include, for example and without limitation, antibodies (or binding fragments thereof), aptamers, receptors for cell-surface proteins, and ligands for cell-surface proteins. In certain embodiments, compositions described herein are contained within a vesicle and the vesicle contains the binding partner on its surface. As will be understood, such vesicles include synthetic and naturally occurring cell-derived vesicles, (e.g., liposomes, nanocapsules, microparticles, microspheres, lipid particles, vesicles, and the like). Lee et al., “Exosomes and Microvesicles: Extracellular Vesicles for Genetic Information Transfer and Gene Therapy,” Hum. Mol. Genet. 21 (R1): R125-R134 (2012), which is hereby incorporated by reference in its entirety.

Also encompassed are expression systems comprising nucleic acid molecules described herein. Generally, the use of recombinant expression systems involves inserting a nucleic acid molecule encoding the amino acid sequence of a desired peptide into an expression system to which the molecule is heterologous (i.e., not native or not normally present). One or more desired nucleic acid molecules encoding a peptide described herein (e.g., AUF1) may be inserted into the vector. When multiple nucleic acid molecules are inserted, the multiple nucleic acid molecules may encode the same or different peptides. The heterologous nucleic acid molecule is inserted into the expression system or vector in proper sense (5′→3′) orientation relative to the promoter and any other 5′ regulatory molecules, and correct reading frame.

The preparation of the nucleic acid constructs can be carried out using standard cloning procedures well known in the art as described by Joseph Sambrook et al., MOLECULAR CLONING: A LABORATORY MANUAL (Cold Springs Harbor 2012), which is hereby incorporated by reference in its entirety. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in a suitable host cell.

A nucleic acid molecule encoding an AUF1 protein or functional fragment thereof, a heterologous targeting element (e.g., promoter molecule of choice) including, without limitation, enhancers, and leader sequences; a suitable 3′ regulatory region to allow transcription in the host or a certain medium, and any additional desired components, such as reporter or marker genes, are cloned into the vector of choice using standard cloning procedures in the art, such as described in Joseph Sambrook et al., MOLECULAR CLONING: A LABORATORY MANUAL (Cold Springs Harbor 2012); Frederick M. Ausubel, SHORT PROTOCOLS IN MOLECULAR BIOLOGY (Wiley 2002); and U.S. Pat. No. 4,237,224 to Cohen and Boyer, which are hereby incorporated by reference in their entirety.

A variety of genetic signals and processing events that control many levels of gene expression (e.g., DNA transcription and messenger RNA (“mRNA”) translation) can be incorporated into the nucleic acid construct to maximize protein production. For the purposes of expressing a cloned nucleic acid sequence encoding a desired protein, it is advantageous to use strong promoters to obtain a high level of transcription. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited to, lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR. In one embodiment, the composition described herein may include a muscle satellite cell specific promoter (e.g., a Pax7, a MyoD, or a myogenin promoter and/or enhancer). (GenBank Accession No. AJ130875.1 (SEQ ID NO:61, nt 1-3245), Homo sapiens PAX-7 Gene Promoter Region and Exon 1, Partial; Murmann et al., “Cloning and Characterization of the Human Pax7 Promoter,” Biol Chem 381(4):331-5 (2000); Riuzzi et al., “RAGE Signaling Deficiency in Rhabdomyosarcoma Cells Causes Upregulation of PAX7 and Uncontrolled Proliferation,” J. Cell Science 127:1699-1711 (2014); GenBank Accession No. U21227.1, Human myoD Gene, Core Enhancer Sequence; Goldhamer et al., “Embryonic Activation of the myoD Gene is Regulated by a Highly Conserved Distal Control Element,” Development 121(3):637-49 (1995); Accession No. NC_000011.10 (MyoD); and Accession No. NC_000001.11 (Myogenin), each of which is hereby incorporated by reference in its entirety). For instance, a MyoD or myogenin promoter or conserved control or regulatory element may be identified from Accession No. NC_000011.10 (MyoD) and Accession No. NC_000001.11 (Myogenin), each of which is hereby incorporated by reference in its entirety.

There are other specific initiation signals required for efficient gene transcription and translation in prokaryotic cells that can be included in the nucleic acid construct to maximize protein production. Depending on the vector system and host utilized, any number of suitable transcription and/or translation elements, including constitutive, inducible, and repressible promoters, as well as minimal 5′ promoter elements, enhancers or leader sequences may be used.

In one embodiment, the expression vector can be a viral-based vector. Examples of viral-based vectors include, but are not limited to, those derived from replication deficient retrovirus, lentivirus, adenovirus, and adeno-associated virus. Retrovirus vectors and adeno-associated virus vectors are currently the recombinant gene delivery system of choice for the transfer of exogenous genes in vivo, particularly into humans. These vectors provide efficient delivery of genes into cells, and the transferred polynucleotides are stably integrated into the chromosomal DNA of the host.

The polynucleotide is usually incorporated into the vector under the control of a suitable promoter that allows for expression of the encoded polypeptide in vivo, as described above. Suitable promoters which may be employed include, but are not limited to, adenoviral promoters, such as the adenoviral major late promoter, the E1A promoter, the major late promoter (MLP) and associated leader sequences or the E3 promoter; the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTR, the histone, pot III, and pectin promoters; B 19 parvovirus promoter; the SV40 promoter; and human growth hormone promoters. The promoter also may be the native promoter for the gene of interest. The selection of a suitable promoter will be dependent on the vector, the host cell and the encoded protein and is considered to be within the ordinary skills of a worker in the art.

The development of specialized cell lines (termed “packaging cells”) which produce only replication-defective retroviruses has increased the utility of retroviruses for gene therapy, and defective retroviruses are well characterized for use in gene transfer for gene therapy purposes. Thus, a recombinant retrovirus can be constructed in that part of the retroviral coding sequence (gag, pot, env) that has been replaced by the subject polynucleotide and renders the retrovirus replication defective. The replication defective retrovirus is then packaged into virions that can be used to infect a target cell through the use of a helper virus by standard techniques. Protocols for producing recombinant retroviruses and for infecting cells in vitro or in vivo with such viruses can be found in CURRENT PROTOCOLS IN MOLECULAR BIOLOGY (Frederick M. Ausubel et al. eds., 1989), which is hereby incorporated by reference in its entirety, and other standard laboratory manuals.

Furthermore, it has been shown that it is possible to limit the infection spectrum of retroviruses and consequently of retroviral-based vectors by modifying the viral packaging proteins on the surface of the viral particle (see, e.g., PCT Publication Nos. WO93/25234 and WO94/06920, which are hereby incorporated by reference in their entirety). For instance, strategies for the modification of the infection spectrum of retroviral vectors include: coupling antibodies specific for cell surface antigens to the viral env protein (Roux et al., PNAS 86:9079-9083 (1989); Julan et al., J. Gen. Virol. 73:3251-3255 (1992); and Goud et al., Virology 163:251-254 (1983), which are hereby incorporated by reference in their entirety); or coupling cell surface receptor ligands to the viral env proteins (Neda et al., J. Biol. Chem. 266: 14143-14146 (1991), which is hereby incorporated by reference in its entirety). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the polynucleotides contained in the vector.

Another viral vector useful in gene therapy techniques is an adenovirus-derived vector. The genome of an adenovirus can be manipulated such that it encodes and expresses a gene product of interest but is inactivated in terms of its ability to replicate in a normal lytic viral life cycle. See, e.g., Berliner et al., BioTechniques 6:616 (1988); Rosenfeld et al., Science 252:431-434 (1991); and Rosenfeld et al., Cell 68:143-155 (1992), which are hereby incorporated by reference in their entirety. Suitable adenoviral vectors derived from the adenovirus strain Ad type 5 dl 324 or other strains of adenovirus (e.g., Adz, Ad3, Adz, etc.) are well known to those skilled in the art. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Another viral vector useful in gene therapy techniques is an adeno-associated viral vector. These delivery vehicles can be constructed and used to deliver a nucleic acid molecule to cells, as described in Shi et al., “Therapeutic Expression of an Anti-Death Receptor-5 Single-Chain Fixed Variable Region Prevents Tumor Growth in Mice,” Cancer Res. 66:11946-53 (2006); Fukuchi et al., “Anti-Aβ Single-Chain Antibody Delivery via Adeno-Associated Virus for Treatment of Alzheimer's Disease,” Neurobiol. Dis. 23:502-511 (2006); Chatterjee et al., “Dual-Target Inhibition of HIV-1 In Vitro by Means of an Adeno-Associated Virus Antisense Vector,” Science 258:1485-1488 (1992); Ponnazhagan et al., “Suppression of Human Alpha-globin Gene Expression Mediated by the Recombinant Adeno-associated Virus 2-based Antisense Vectors,” J. Exp. Med. 179:733-738 (1994), which are hereby incorporated by reference in their entirety. In vivo use of these vehicles is described in Flotte et al., “Stable In Vivo Expression of the Cystic Fibrosis Transmembrane Conductance Regulator With an Adeno-Associated Virus Vector,” Proc. Nat'l. Acad. Sci. 90:10613-10617 (1993), which is hereby incorporated by reference in its entirety.

In certain embodiments, the adenoviral vectors for use in accordance with the present invention are deleted for all or parts of the viral E2 and E3 genes, but retain as much as 80% of the adenoviral genetic material (see, e.g., Jones et al., Cell 16:683(1979); Berliner et al., BioTechniques 6:616 (1988); and Graham et al., in Methods in Molecular Biology, E. J. Murray, Ed. (Humane, Clifton, N.J., 1991) vol. 7. pp. 109-127, which are hereby incorporated by reference in their entirety). Generation and propagation of replication-defective human adenovirus vectors requires a unique helper cell line. Helper cell lines may be derived from human cells such as human embryonic kidney cells, muscle cells, hematopoetic cells, or other human embryonic mesenchymal or epithelial cells. Alternatively, the helper cells may be derived from the cells of other mammalian species that are permissive for human adenovirus, i.e., that provide, in bans, a sequence necessary to allow for replication of a replication-deficient virus. Such cells include, for example, 293 cells, Vero cells, or other monkey embryonic mesenchymal or epithelial cells.

The present invention also contemplates the intracellular introduction of the polynucleotide (i.e., encoding AUF1 protein or a functional fragment thereof) and subsequent incorporation within host cell DNA for expression by homologous recombination using techniques described above or by use of genome editing or alteration. Such techniques for targeted genomic insertion involve, for example, inducing a double stranded DNA break precisely at one or more targeted genetic loci followed by integration of a chosen transgene or nucleic acid molecule (or construct) during repair. Such techniques or systems include, for example, zinc finger nucleases (“ZFN”) (Urnov et al., “Genome Editing with Engineered Zinc Finger Nucleases,” Nat Rev Genet. 11:636-646 (2010), which is hereby incorporated by reference in its entirety), transcription activator-like effector nucleases (“TALEN”) (Joung and Sander, “TALENs: A Widely Applicable Technology for Targeted Genome Editing,” Nat Rev Mol Cell Biol. 14: 49-55 (2013), which is hereby incorporated by reference in its entirety), clustered regularly interspaced short palindromic repeat (“CRISPR”)-associated endonucleases (e.g., CRISPR/CRISPR-associated (“Cas”) 9 systems) (Wiedenheft et al., “RNA-Guided Genetic Silencing Systems in Bacteria and Archaea,” Nat 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety).

Another aspect of the present invention relates to a composition comprising a muscle satellite cell population, where the cell population comprises a transgene exogenous to the satellite cells and encoding AUF1 protein or a functional fragment thereof.

A further aspect of the present invention relates to a composition comprising a muscle cell population comprising an AUF1 gene encoding AUF1 protein or functional fragment thereof, where expression of the AUF1 gene is controlled by a promoter heterologous to the AUF1 gene. In one embodiment, the cell population expresses the AUF1 protein or functional fragment thereof. Such a muscle cell population may be a satellite cell population.

Satellite cells express various markers during culture, such as Syndecan 4 and/or PAX7, comprising quiescent and/or early-activation satellite cell states. In one embodiment, the cells of compositions described herein are Syndecan 4⁺/PAX7⁺. In another embodiment, the cells of compositions described herein are Syndecan 4⁺/PAX7⁻.

A further aspect of the present invention relates to a method of producing a muscle satellite cell population. This method involves transforming or transfecting Syndecan 4⁺/PAX7⁺ or Syndecan 4⁺/PAX7⁻ muscle satellite cells with a nucleic acid molecule encoding exogenous AUF1 or a functional fragment thereof under conditions effective to express exogenous AUF1 in the muscle satellite cells. Prior to the transformation or transfection, the Syndecan 4⁺/PAX7⁺ or Syndecan 4⁺/PAX7⁻ muscle satellite cells may be functional AUF1 deficient. Prior to the transformation or transfection, the Syndecan 4⁺/PAX7⁺ or Syndecan 4⁺/PAX7⁻ muscle satellite cells may be functional AUF1 sufficient.

Still another aspect of the present invention relates to a muscle satellite cell population produced by the method of producing a muscle satellite cell population of the present invention described herein.

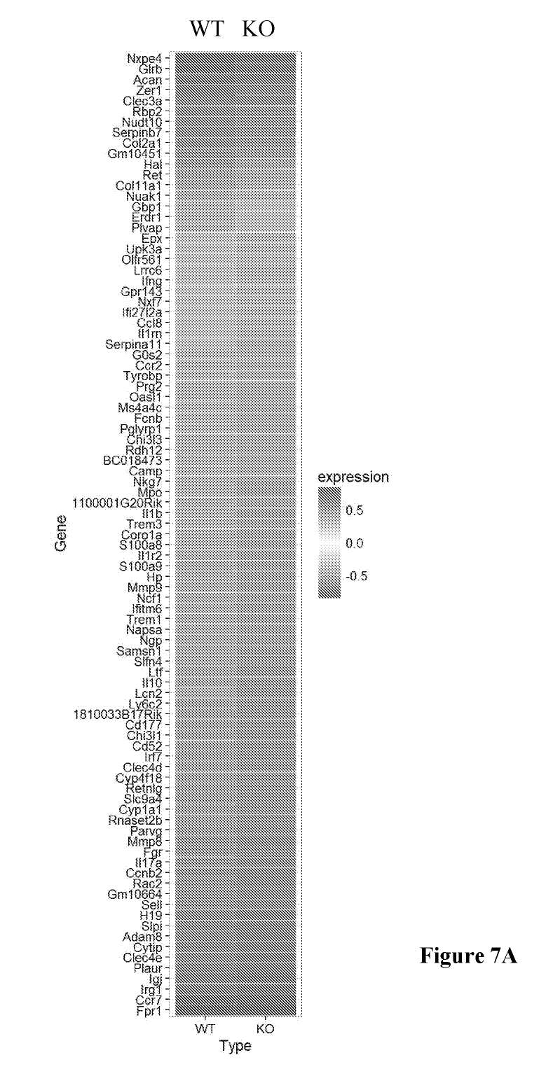

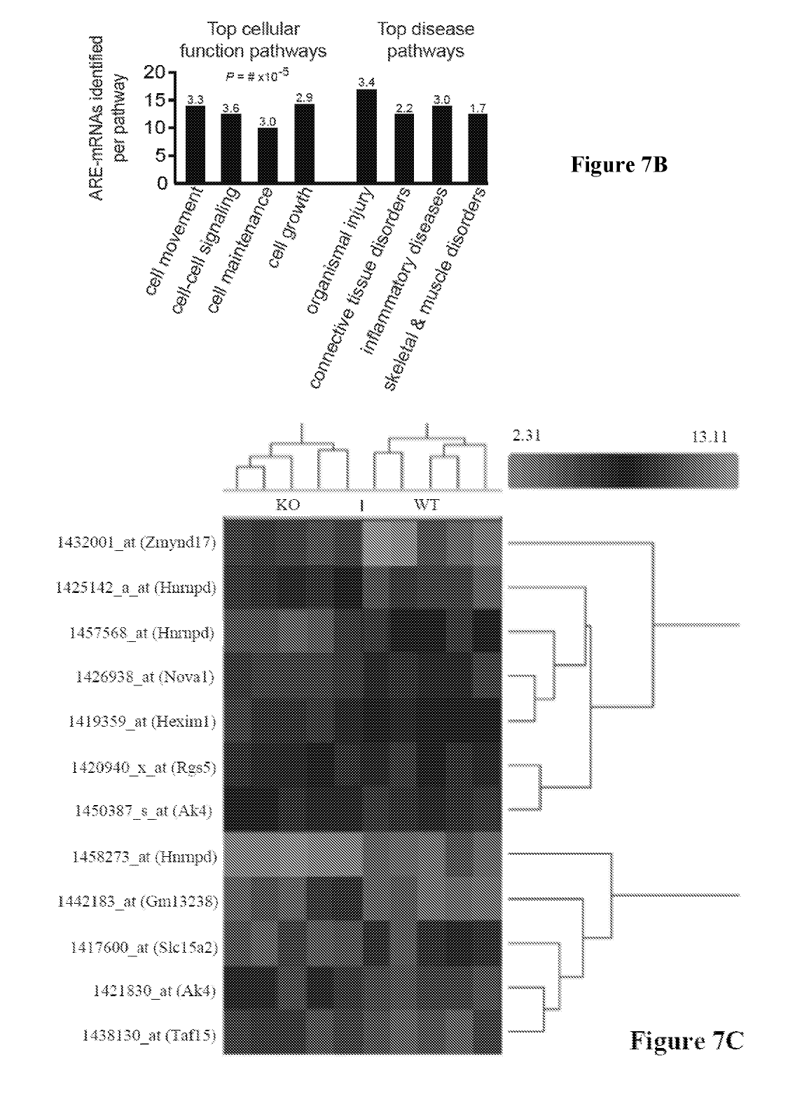

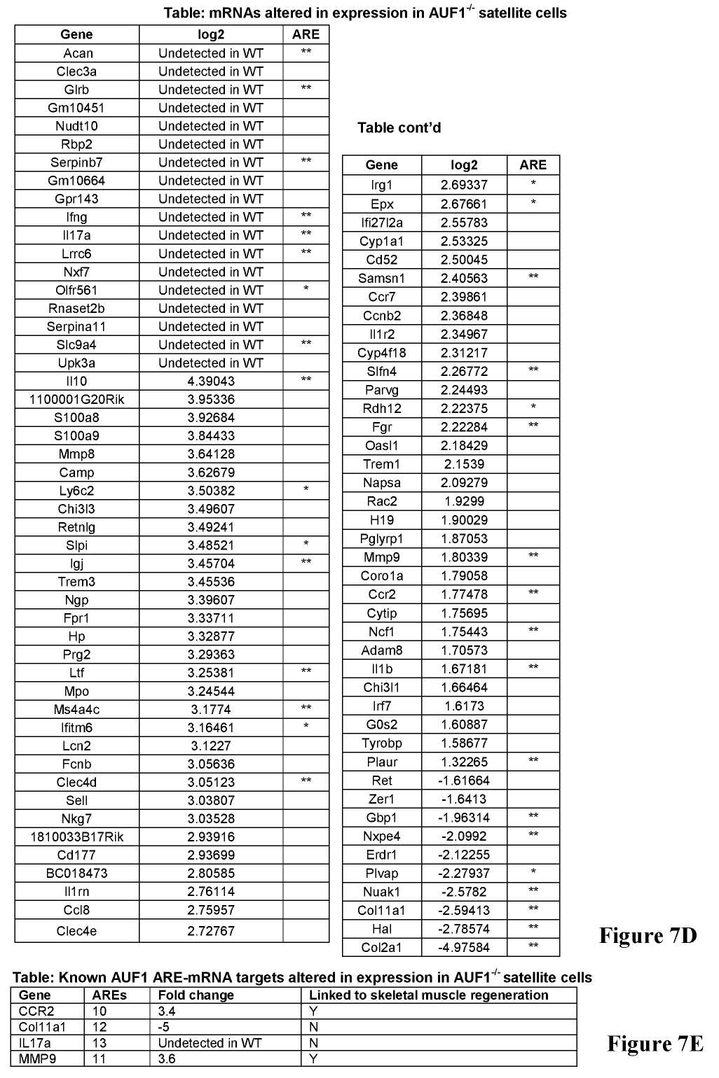

Compositions according to the present invention may include one or more inhibitors of genes and expression products of genes (and variants or isoforms thereof) identified as increased in abundance in the Tables found in FIGS. 7D and 7E (referred to herein as target genes or targets). Compositions according to the present invention may include one or more of an MMP-9 inhibitor, a Twist1 inhibitor, or a cyclin D1 inhibitor. Compositions may include one or more of an IL17 inhibitor, and MMP-8 inhibitor, an IL10 inhibitor, an FGR inhibitor, a TREM1 inhibitor, a CCR2 inhibitor, an ADAM8 inhibitor, or an IL1b inhibitor. Exemplary target inhibitors include, but are not limited to, inhibitors of target expression, antagonists which bind a target or a target's receptor (e.g., an antibody, a polypeptide, a dominant negative variant of a target, a mutant of a natural target receptor, a small molecular weight organic molecule, and a competitive inhibitor of receptor binding), and substances which inhibit one or more target functions without binding thereto (e.g., an anti-idiotypic antibody). The inhibitor may be, for example, a nucleic acid molecule, a polypeptide, an antibody, or a small molecule.

As will be appreciated, inhibitors described herein may be based on the nucleotide sequence of the target or target gene, which will be readily identifiable. Such sequences may be of mammalian origin (e.g., human or murine). For instance, human and mouse amino acid and nucleotide sequence accession numbers (GenBank or NCBI Reference Sequence (“NCBI Ref. Seq.”) corresponding to MMP-9, Twist1, cyclin D1, IL17, MMP-8, IL10, FGR, TREM1, CCR2, ADAM8, and IL1b are found in Table 2 and are each is hereby incorporated by reference in its entirety:

TABLE 2

| | Human | | Mouse | |
|---|---|---|---|---|
| Target | Nucleotide | Amino Acid | Nucleotide | Amino Acid |
| MMP-9 | NCBI Ref. Seq.: | NCBI Ref. Seq.: | NCBI Ref. Seq.: | NCBI Ref. Seq. |
| | NG_011468.1 | NP_004985.2 | AY902320.1 | AAX90605.1 |
| | (SEQ ID NO: 17) | (SEQ ID NO: 18) | (SEQ ID NO: 39) | (SEQ ID NO: 40) |
| Twist1 | NCBI Ref. Seq.: | NCBI Ref. Seq. | NCBI Ref. Seq.:: | NCBI Ref. Seq.: |
| | NG_008114.1 | NP_000465.1 | NM_011658.2 | NP_035788.1 |
| | (SEQ ID NO: 19) | (SEQ ID NO: 20) | (SEQ ID NO: 41) | (SEQ ID NO: 42) |
| Cyclin | NCBI Ref. Seq.: | NCBI Ref. Seq.: | NCBI Ref. Seq.: | NCBI Ref. Seq.: |
| D1 | NG_007375.1 | NP_444284.1 | NM_007631.2 | NP_031657.1 |
| | (SEQ ID NO: 21) | (SEQ ID NO: 22) | (SEQ ID NO: 43) | (SEQ ID NO: 44) |
| IL17 | NCBI Ref. Seq.: | NCBI Ref. Seq.: | NCBI Ref. Seq.: | NCBI Ref. Seq.: |
| | NG_033021.1 | NP_002181.1 | NM_010552.3 | NP_034682.1 |
| | (SEQ ID NO: 23) | (SEQ ID NO: 24) | (SEQ ID NO: 45) | (SEQ ID NO: 46) |
| MMP-8 | NCBI Ref. Seq.: | NCBI Ref. Seq.: | NCBI Ref. Seq.: | NCBI Ref. Seq.: |
| | NG_012101.1 | NP_002415.1 | NM_008611.4 | NP_032637.3 |
| | (SEQ ID NO: 25) | (SEQ ID NO: 26) | (SEQ ID NO: 47) | (SEQ ID NO: 48) |
| IL10 | NCBI Ref. Seq.: | NCBI Ref Seq.: | GenBank: M84340.1 | GenBank: |
| | NG_012088.1 | NP_000563.1 | (SEQ ID NO: 49) | AAA39275.1 |
| | (SEQ ID NO: 27) | (SEQ ID NO: 28) | | (SEQ ID NO: 50) |
| FGR | NCBI Ref. Seq.: | NCBI Ref. Seq.: | NCBI Ref. Seq.: | NCBI Ref. Seq.: |
| | NC_000001.11 | NP_005239.1 | NM_010208.4 | NP_034338.3 |
| | (SEQ ID NO: 29) | (SEQ ID NO: 30) | (SEQ ID NO: 51) | (SEQ ID NO: 52) |
| TREM1 | NCBI Ref. Seq.: | NCBI Ref. Seq.: | NCBI Ref. Seq.: | NCBI Ref. Seq.: |
| | NG_029525.2 | NP_061113.1 | NM_021406.5 | NP_067381.1 |
| | (SEQ ID NO: 31) | (SEQ ID NO: 32) | (SEQ ID NO: 53) | (SEQ ID NO: 54) |
| CCR2 | NCBI Ref. Seq.: | NCBI Ref. Seq.: | NCBI Ref. Seq.: | NCBI Ref. Seq.: |
| | NG_021428.1 | NP_001116513.2 | NM_009915.2 | NP_034045.1 |
| | (SEQ ID NO: 33) | (SEQ ID NO: 34) | (SEQ ID NO: 55) | (SEQ ID NO: 56) |
| ADAM8 | NCBI Ref. Seq.: | NCBI Ref. Seq.: | NCBI Ref. Seq.: | NCBI Ref. Seq.: |
| | NC_000010.11 | NP_001100.3 | NM_007403.3 | NP_031429.1 |
| | (SEQ ID NO: 35) | (SEQ ID NO: 36) | (SEQ ID NO: 57) | (SEQ ID NO: 58) |
| IL1b | NCBI Ref. Seq.: | NCBI Ref. Seq.: | NCBI Ref. Seq.: | NCBI Ref. Seq.: |
| | NG_ 008851.1 | NP_000567.1 | NM_008361.4 | NP_032387.1 |
| | (SEQ ID NO: 37) | (SEQ ID NO: 38) | (SEQ ID NO: 59) | (SEQ ID NO: 60) |

As will be understood, inhibitors of variants and isoforms of the above-noted exemplary sequences are also encompassed. In one embodiment, such variants and isoforms include nucleotide or amino acid sequence that have at least 80%, at least 85%, at least 90%, at least 95%, at least 97%, or at least 99% sequence identity to a sequence identified in Table 2.

The inhibitor may be a nucleic acid molecule effective in silencing expression of one or more target genes. In one embodiment, the inhibitor is a nucleic acid molecule effective in silencing expression of MMP-9, Twist 1, cyclin D1, Il17, MMP-8, IL10, FGR, TREM1, CCR2, ADAM8, or IL1b (e.g., via RNAi). The inhibitor may silence expression of one or more of MMP-9, Twist1, or cyclin D1. The inhibitor may silence expression of one or more of IL17, MMP-8, IL10, FGR, TREM1, CCR2, ADAM8, or IL1b.

RNA interference (“RNAi”) is mediated by siRNA. The siRNA comprises an RNA strand (the antisense strand) having a region which is less than 30 nucleotides in length, generally 19-24 nucleotides in length, and is substantially complementary to at least part of an mRNA transcript of the target gene(s). Various assays are known in the art to test siRNA for its ability to mediate RNAi (see, e.g., Elbashir et al., Methods 26:199-213 (2002), which is hereby incorporated by reference in its entirety). Use of double-stranded ribonucleic acid (dsRNA) molecules for inhibiting the expression of the target gene is also contemplated. The dsRNA comprises at least two sequences that are complementary to each other. The dsRNA comprises a sense strand comprising a first sequence and an antisense strand comprising a second sequence. The antisense strand comprises a nucleotide sequence which is substantially complementary to at least part of an mRNA encoding target gene. The region of complementarity may be less than 30 nucleotides in length. In one embodiment, the region of complementarity is 19-24 nucleotides in length. It will be understood that any other RNA inducing agent may be used, including shRNA, miRNA or an RNAi-inducing vector whose presence within a cell results in production of an siRNA or shRNA targeted to a transcript. Such siRNA or shRNA comprises a portion of RNA that is complementary to a region of the target transcript. Essentially, the RNAi-inducing agent or RNAi molecule downregulates expression of the targeted protein via RNA interference.

Accordingly, the nucleic acid molecule may encode an antisense form of at least a portion of a nucleic acid molecule that encodes a target. The nucleic acid molecule may also be an antisense form of a least a portion of a nucleic acid molecule that encodes a target. The nucleic acid molecule may also include a first segment encoding the target and a second segment that is an antisense form of the first segment, as well as an optional linker between the first and second segments. The nucleic acid molecule inhibitor may be included in a nucleic acid construct for delivery, as described above.

In another embodiment, gene alteration or editing using an endonuclease system is used for target inhibition. Such techniques or systems include, for example, zinc finger nucleases (“ZFNs”) (Urnov et al., “Genome Editing with Engineered Zinc Finger Nucleases,” Nat. Rev. Genet. 11: 636-646 (2010), which is hereby incorporated by reference in its entirety), transcription activator-like effector nucleases (“TALENs”) (Joung & Sander, “TALENs: A Widely Applicable Technology for Targeted Genome Editing,” Nat. Rev. Mol. Cell Biol. 14: 49-55 (2013), which is hereby incorporated by reference in its entirety), clustered regularly interspaced short palindromic repeat (“CRISPR”)-associated endonucleases (e.g., CRISPR/CRISPR-associated (“Cas”) 9 systems) (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety).

Accordingly, in one embodiment, the nucleic acid molecule encodes an endonuclease for targeted alteration of genes encoding a target (e.g., MMP-9, Twist1, cyclin D1, IL17, MMP-8, IL10, FGR, TREM1, CCR2, ADAM8, or IL1b). In one embodiment, the nucleic acid molecule encodes an endonuclease for targeted alteration of genes encoding MMP-9, Twist1, cyclin D1, or a combination thereof. The nucleic acid molecule may encode an endonuclease for targeted alteration of the gene encoding IL17, MMP-8, IL10, FGR, TREM1, CCR2, ADAM8, or IL1b. The endonuclease may be a ZFN, TALEN, or CRISPR-associated endonuclease.

Nucleic acid aptamers that specifically bind to a target are also useful as inhibitors in accordance with the present invention. Nucleic acid aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

In yet another embodiment, the inhibitor is a polypeptide. In a more specific embodiment, the inhibitor is an antibody.

As used herein, the term “antibody” is meant to include intact immunoglobulins derived from natural sources or from recombinant sources, as well as immunoreactive portions (i.e. antigen binding portions) of intact immunoglobulins. Antibodies may exist in a variety of forms including, for example, polyclonal antibodies, monoclonal antibodies, intracellular antibodies, antibody fragments (e.g. Fv, Fab and F(ab)2), single chain antibodies (scFv), single-domain antibodies, chimeric antibodies, and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988); Bird et al, “Single-Chain Antigen-Binding Proteins,” Science 242:423-426 (1988), which are hereby incorporated by reference in their entirety).

Single chain antibodies lack some or all of the constant domains of the whole antibodies from which they are derived. Therefore, they can overcome some of the problems associated with the use of whole antibodies (i.e., free of certain undesired interactions between heavy-chain constant regions and other biological molecules). Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Single-domain antibodies (sdAb; nanobody) are antibody fragments consisting of a single monomeric variable antibody domain (˜12-15 kDa). The sdAb are derived from the variable domain of a heavy chain (V_{H}) or the variable domain of a light chain (V_{L}). sdAbs can be naturally produced, i.e., by immunization of dromedaries, camels, llamas, alpacas, or sharks (Ghahroudi et al., “Selection and Identification of Single Domain Antibody Fragments from Camel Heavy-Chain Antibodies,” FEBS Letters 414(3): 521-526 (1997), which is hereby incorporated by reference in its entirety). Alternatively, the antibody can be produced in microorganisms or derived from conventional whole antibodies (Harmsen et al., “Properties, Production, and Applications of Camelid Single-Domain Antibody Fragments,” Appl. Microbiol. Biotechnology 77:13-22 (2007); Holt et al., “Domain Antibodies: Proteins for Therapy,” Trends Biotech. 21(11): 484-490 (2003), which are hereby incorporated by reference in their entirety).

Fab (Fragment, antigen binding) refers to the fragments of the antibody consisting of the VL, CL, VH, and CH1 domains. Those generated following papain digestion simply are referred to as Fab and do not retain the heavy chain hinge region. Following pepsin digestion, various Fabs retaining the heavy chain hinge are generated. Those fragments with the interchain disulfide bonds intact are referred to as F(ab′)2, while a single Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises CH2 and CH3 domains. The Fc of an IgA or an IgM antibody further comprises a CH4 domain. The Fc is associated with Fc receptor binding, activation of complement mediated cytotoxicity and antibody-dependent cellular-cytotoxicity (ADCC). For antibodies such as IgA and IgM, which are complexes of multiple IgG-like proteins, complex formation requires Fc constant domains.

Methods for monoclonal antibody production may be carried out using techniques well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest (i.e., target protein) either in vivo or in vitro. The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur. J. Immunol. 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

Monoclonal antibodies or antibody fragments can also be isolated from antibody phage libraries generated using the techniques described in McCafferty et al., “Phage Antibodies: Filamentous Phage Displaying Antibody Variable Domains,” Nature 348:552-554 (1990), which is hereby incorporated by reference in its entirety. Clackson et al., “Making Antibody Fragments using Phage Display Libraries,” Nature 352:624-628 (1991); and Marks et al., “By-Passing Immunization. Human Antibodies from V-Gene Libraries Displayed on Phage,” J. Mol. Biol. 222:581-597 (1991), which are hereby incorporated by reference in their entirety, describe the isolation of murine and human antibodies, respectively, using phage libraries. Subsequent publications describe the production of high affinity (nM range) human antibodies by chain shuffling (Marks et al., BioTechnology 10:779-783 (1992), which is hereby incorporated by reference in its entirety), as well as combinatorial infection and in vivo recombination as a strategy for constructing very large phage libraries (Waterhouse et al., Nuc. Acids. Res. 21:2265-2266 (1993), which is hereby incorporated by reference in its entirety). Thus, these techniques are viable alternatives to traditional monoclonal antibody hybridoma techniques for isolation of monoclonal antibodies.

Alternatively, monoclonal antibodies can be made using recombinant DNA methods as described in U.S. Pat. No. 4,816,567 to Cabilly et al, which is hereby incorporated by reference in its entirety. The polynucleotides encoding a monoclonal antibody are isolated from mature B-cells or hybridoma cells, for example, by RT-PCR using oligonucleotide primers that specifically amplify the genes encoding the heavy and light chains of the antibody. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, generate monoclonal antibodies.

The polynucleotide(s) encoding a monoclonal antibody can further be modified using recombinant DNA technology to generate alternative antibodies. For example, the constant domains of the light and heavy chains of a mouse monoclonal antibody can be substituted for those regions of a human antibody to generate a chimeric antibody. Alternatively, the constant domains of the light and heavy chains of a mouse monoclonal antibody can be substituted for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity and affinity of a monoclonal antibody.

“Humanized” forms of non-human (e.g., rodent) antibodies are chimeric antibodies that contain minimal sequences derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. See Jones et al., Nature 321:522-525 (1986); Riechmann et al., Nature 332:323-329 (1988); and Presta, Curr. Op. Struct. Biol. 2:593-596 (1992), which are hereby incorporated by reference in their entirety.

Methods for humanizing non-human antibodies have been described in the art. As an alternative to humanization, human antibodies can be generated. See, e.g., Jakobovits et al., Proc. Natl. Acad. Sci. USA 90:2551 (1993); Jakobovits et al., Nature 362:255-258 (1993); U.S. Pat. No. 5,545,806 to Lonberg et al, U.S. Pat. No. 5,569,825 to Lonberg et al, and U.S. Pat. No. 5,545,807 to Surani et al; McCafferty et al., Nature 348:552-553 (1990), which are hereby incorporated by reference in their entirety.

In addition to whole antibodies, the present invention encompasses binding portions of such antibodies. Such binding portions include the monovalent Fab fragments, Fv fragments (e.g., single-chain antibody, scFv), single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES:PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

Bispecific antibodies are antibodies that have binding specificities for at least two different epitopes. Exemplary bispecific antibodies may bind to two different epitopes of a single target (e.g., MMP-9, Twist1, cyclin D1, IL17, MMP-8, IL10, FGR, TREM1, CCR2, ADAM8, or IL1b) or of two different targets. In one embodiment, the inhibitor is a bispecific antibody for a satellite cell marker and a target. In another embodiment, the bispecific antibody binds to Pax7 and a target (e.g., MMP-9, Twist1, cyclin D1, IL17, MMP-8, IL10, FGR, TREM1, CCR2, ADAM8, or IL1b). In one embodiment, the bispecific antibody binds to Pax7 and MMP-9.

Techniques for making bispecific antibodies are common in the art (Brennan et al., “Preparation of Bispecific Antibodies by Chemical Recombination of Monoclonal Immunoglobulin G1 Fragments,” Science 229:81-3 (1985); Suresh et al, “Bispecific Monoclonal Antibodies From Hybrid Hybridomas,” Methods in Enzymol. 121:210-28 (1986); Traunecker et al., “Bispecific Single Chain Molecules (Janusins) Target Cytotoxic Lymphocytes on HIV Infected Cells,” EMBO J. 10:3655-3659 (1991); Shalaby et al., “Development of Humanized Bispecific Antibodies Reactive with Cytotoxic Lymphocytes and Tumor Cells Overexpressing the HER2 Protooncogene,” J. Exp. Med. 175:217-225 (1992); Kostelny et al, “Formation of a Bispecific Antibody by the Use of Leucine Zippers,” J. Immunol. 148: 1547-1553 (1992); Gruber et al., “Efficient Tumor Cell Lysis Mediated by a Bispecific Single Chain Antibody Expressed in Escherichia coli,” J. Immunol. 152:5368-74 (1994); and U.S. Pat. No. 5,731,168 to Carter et al., which are hereby incorporated by reference in their entirety). Generally, bispecific antibodies are secreted by triomas (i.e., lymphoma cells fuse to a hybridoma) and hybrid hybridomas. The supernatants of triomas and hybrid hybridomas can be assayed for bispecific antibody production using a suitable assay (e.g., ELISA), and bispecific antibodies can be purified using conventional methods. These antibodies can then be humanized according to methods known in the art.

Target inhibitors of the present invention also include inhibitory peptides. Suitable inhibitory peptides of the present invention include short peptides based on the sequence of the target that exhibit inhibition of target binding to receptors or complexes and direct biological antagonist activity. The amino acid sequence of targets from which inhibitory peptides are derived are known and include those described in Table 2 above. Such inhibitory peptides may be chemically synthesized using known peptide synthesis methodology or may be prepared and purified using recombinant technology. Such peptides are usually at least about 4 amino acids in length, but can be anywhere from 4 to 100 amino acids in length.

MMP9 inhibitors are also known in the art. Suitable examples may include, without limitation, PCK 1345, which is a synthetic peptide small molecule inhibitor of PSP94 (a regulator of MMP9) and is a Phase II prostate cancer drug of Ambrilia Biopharma (see U.S. Patent Application Publication No. 2005/0026833 and Hu et al., “Matrix Metalloproteinase Inhibitors as Therapy for Inflammatory and Vascular Diseases,” Nature Reviews Drug Discovery 6:480-498 (2007), which are hereby incorporated by reference in their entirety); Apratastat, which is a synthetic peptide small molecule inhibitor of MMP1, MMP9, MMP13, and TNF in Phase II clinical trials for rheumatoid arthritis for Amgen/Wyeth (see PCT Publication No. WO 2007/107663 and Hu et al., “Matrix Metalloproteinase Inhibitors as Therapy for Inflammatory and Vascular Diseases,” Nature Reviews Drug Discovery 6:480-498 (2007), which are hereby incorporated by reference in their entirety); doxycycline (see U.S. Pat. No. 5,045,538; U.S. Patent Application Publication No. 2012/0107284; Wang et al., “Doxycycline Inhibits Leukemic Cell Migration via Inhibition of Matrix Metalloproteinase and Phosphorylation of Focal Adhesion Kinase,” Mol. Med. Rep. (May 2015); Doroszko et al., “Effects of MMP-9 Inhibition by Doxycycline on Proteome of Lungs in High Tidal Volume Mechanical Ventilation-induced Acute Lung Injury,” Proteome Science 8:3 (2010); Lindeman et al., “Clinical Trial of Doxycycline for Matrix Metalloproteinase-9 Inhibition in Patients with an Abdominal Aneurysm,” Circulation 119:2209-2216 (2009); Kim et al., “Doxycycline Inhibits TGF-Beta1-induced MMP9-via Smad and MAPK Pathways in Human Corneal Epithelial Cells,” IOVS 46:840-848 (2005), which are hereby incorporated by reference in their entirety); AZD 1236, which is a synthetic peptide small molecule inhibitor of MMP9 and MMP12 (AstraZeneca) (see Chaturvedi and Kaczmarek, “MMP9 Inhibition: A Therapeutic Strategy in Ischemic Stroke,” Mol. Neurobiol. 49:563-573 (2014), which is hereby incorporated by reference in its entirety); TIMP1 in vivo gene transfer, which is a potent genetic inhibitor of MMP (see Jayasankar et al., “Cardiac Transplantation and Surgery for Congestive Heart Failure,” Circulation 110:II-180-II-186 (2004) (direct injection of replication deficient adenovirus for TIMP1), which is hereby incorporated by reference in its entirety); atorvastatin, which is an HMG coA reductase inhibitor (Pfizer) (see Mohebbi et al., “Effects of Atorvastatin on Plasma Matrix Metalloproteinase 9 Concentrations After Glial Tumor Resection; A Randomized, Double Blind, Placebo Controlled Trial,” DARU 22:10 (2014); Xu et al., “Atorvastatin Lowers Plasma Matrix Metalloproteinase 9 in Patients with Acute Coronary Syndrome,” Clinical Chemistry 50:750-753 (2004); Ballard et al., “Increases in Creatine Kinase with Atorvastatin Treatment are not Associated with Decrease in Muscle Performance,” Atherosclerosis 230:121-124 (2013); Cloutier et al., “Atorvastatin is Beneficial for Muscle Reinnervation after Complete Sciatic Nerve Section in Rats,” J. Plastic Surg. Hand Surg. 47:446-450 (2013); Parker et al., “Effects of Statins on Skeletal Muscle Function,” Circulation 127:96-103 (2013); Rosenbaum et al., “Discontinuation of Statin Therapy Due to Muscular Side Effects: A Survey in Real Life,” Nutr. Metab. Cardiovasc. Dis. 23:871-875 (2013), which are hereby incorporated by reference in their entirety); melatonin (see Rudar et al., “Melatonin Inhibits Matrix Metalloproteinase 9 Activity by Binding to its Active Site, J. Pineal. Res. (2013); Jang et al., “Melatonin Reduced the Elevated Matrix Metalloproteinase 9 Level in a Rat Photothrombotic Stroke Model,” J. Neurol. Sci. (2012); Mishra et al., “Downregulation of Matrix Metalloproteinase 9 by Melatonin During Prevention of Alcohol Induced Liver Injury in Mice,” Biochimie (2011); Swarnakar et al., “Matrix Metalloproteinase 9 Activity and Expression is Reduced by Melatonin During Prevention of Ethanol-induced Gastric Ulcer in Mice,” J. Pineal. Res. (2007); Stratos et al., “Melatonin Restores Muscle Regeneration and Enhances Muscle Function After Crush Injury in Rats,” J. Pineal. Res. (2012); Hibaoui et al., “Melatonin Improves Muscle Function of the Dystrophic Mdx5Cv Mouse, a Model for Duchenne Muscular Dystrophy,” J. Pineal. Res. (2011); Teodoro et al., “Melatonin Prevents Mitochondria Dysfunction and Insulin Resistance in Rat Skeletal Muscle,” J. Pineal. Res. (2014); Kim et al., “Melatonin Induced Autophagy is Associated with Degradation of MyoD Protein in C2C12 Myoblast Cells,” J. Pineal. Res. (2012), which are hereby incorporated by reference in their entirety); SB-3CT, which is a synthetic small molecule inhibitor of MMP9 (see Jia et al., “MMP9 Inhibitor SB-3CT Attenuates Behavioral Impairments and Hippocampal Loss After Traumatic Brain Injury in Rat,” J. Neurotrama. (2014); Gu et al., “A Highly Specific Inhibitor of Matrix Metalloproteinase 9 Rescues Laminin from Proteolysis and Neurons from Apoptosis in Transient Focal Cerebral Ischemia,” J. Neurosci. (2005), which are hereby incorporated by reference in their entirety); BMS-275291, which is a small molecule inhibitor of MMP2 and MMP9 (Bristol Myers Squibb) (see Poulaki et al., “BMS-275291. Bristol Myers Squibb,” Curr. Opinion Investig. Drugs (2002); Leighl et al., “Randomized Phase III Study of Matrix Metalloproteinase Inhibitor BMS-275291 in Combination with Paclitaxel and Carboplatin in Advanced Non-small Cell Lung Cancer: National Cancer Institute of Canada-clinical Trials Group Study BR18,” J. Clin. Oncol. (2005); Miller et al., “A Randomized Phase II Feasibility Trial of BMS-275291 in Patients with Early Stage Breast Cancer,” Clin. Cancer Res. (2004); Rizvi et al., “A Phase I Study of Oral BMS-275291, A Novel Nonhydroxamate Sheddase-sparing Matrix Metalloproteinase Inhibitor, in Patients with Advanced or Metastatic Cancer,” Clinc. Cancer Research (2004); Lockhart et al., “Reduction of Wound Angiogenesis in Patients Treated with BMS-275291 a Broad Spectrum Matrix Metalloproteinase Inhibitor,” Clin. Cancer Res. (2003); Naglich et al., “Inhibition of Angiogenesis and Metastasis in Two Murine Models by the Matrix Metalloproteinase Inhibitor, BMS-275291,” Cancer Res. (2001); Brinker et al., “Phase ½ Trial of BMS-275291 in Patients with Human Immunodeficiency Virus Related Kaposi Sarcoma: A Multicenter Trial of the AIDS Malignancy Consortium,” Cancer (2008), which are hereby incorporated by reference in their entirety); batimastat, which is a small molecule inhibitor of MMP1, MMP2, MMP3, MMP7, and MMP9 (British Biotech) (see Kumar et al., “Matrix Metalloproteinase Inhibitor Batimastat Alleviates Pathology and Improves Skeletal Muscle Function in Dystrophin Deficient Mdx Mice,” Am. J. Pathol. (2010); Giavazzi et al., “Batimastat, A Synthetic Inhibitor of Matrix Metalloproteinases, Potentiates the Antitumor Activity of Cisplatin in Ovarian Carcinoma Xenografts,” Clinic. Cancer Res. (1998), which are hereby incorporated by reference in their entirety); marimastat, which is small molecule inhibitor of MMP1, MMP2, MMP7, and MMP9 (British Biotech) (see Sparano et al., “Randomized Phase III Trial of Marimastat Versus Placebo in Patients with Metastatic Breast Cancer Who have Responding or Stable Disease After First-line Chemotherapy: Eastern Corporative Oncology Group Trial E2196,” J. Clinc. Oncol. (2004), which is hereby incorporated by reference in its entirety); COL-3, which is modified tetracycline (Collagenex); prinomastat agouron (see Bissett et al., “Phase III Clinical Study of Matrix Metalloproteinase Inhibitor Prinomastat in Non-small-cell Lung Cancer,” J. Clin. Oncol. (2005), which is hereby incorporated by reference in its entirety); and GS-5745, which is a monoclonal antibody (Gilead) (see Marshall et al., “Selective Allosteric Inhibition of MMP9 is Efficacious in Preclinical Models of Ulcerative Colitis and Colorectal Cancer,” PLOS (2015), which is hereby incorporated by reference in its entirety).

Cyclin D is a known therapeutic target in cancer (Musgrove et al., “Cyclin D as a Therapeutic Target in Cancer,” Nature Rev. (2011), which is hereby incorporated by reference in its entirety, and cyclin D inhibitors are known in the art. Suitable examples may include, without limitation, BAY1000394, a CDK4/cyclinD1 inhibitor (Bayer, Phase I advance malignancy) (see Seimeister et al., “BAY1000394, A Novel Cyclin Dependent Kinase Inhibitor, with Potent Antitumor Activity in Mono and in Combination Treatment upon Oral Application,” Mol. Cancer Ther. (2012), which is hereby incorporated by reference in its entirety); PD0332991/Palboiclib, a CDK4/cyclinD1 inhibitor (Pfizer) in multiple phase I/II cancer (see Saab et al., “Pharmacologic Inhibition of Cyclin Dependent Kinase 4/6 Activity Arrests Proliferation in Myoblasts and Rhabdomyosarcoma-derived Cells,” Mol. Cancer Ther. (2006); Finn et al., “PD0332991, A Selective Cyclin D Kinase 4/6 Inhibitor, Preferentially Inhibits Proliferation of Luminal Estrogen Receptor Positive Human Breast Cancer Cell Lines In Vitro,” Breast Cancer Res. (2009), which is hereby incorporated by reference in its entirety); R547, which is a CDK4/cyclinD1 inhibitor (Hoffma-Roche, Phase I advance solid tumors) (see Depinto et al., “In Vitro and In Vivo Activity of R547: A Potent and Selective Cyclin Dependent Kinase Inhibitor Currently in Phase I Clinical Trials,” Mol. Cancer Ther. (2006), which is hereby incorporated by reference in its entirety); RGB-286638, which is a CDK4/6/cyclinD1 inhibitor (GPC Biotech/Agennix Phase I hematological malignancies) (see van der Biessen et al., “Phase I Study of RGB-286638, a Novel, Multitargeted Cyclin Dependent Kinase Inhibitor in Patients with Solid Tumors,” Clin. Cancer Res. (2014), which is hereby incorporated by reference in its entirety); nanoparticles-in-microsphere oral system (NiMOS) silencing cyclin D1 (see Kriegel et al., “Dual TNF-Alpha/Cyclin D1 Gene Silencing with an Oral Polymeric Microparticle System as a Novel Strategy for the Treatment of Inflammatory Bowel Disease,” Clin. Transl. Gastroenterol. 2:e2 (2011), which is hereby incorporated by reference in its entirety); and abemaciclib, which is a CDK4 and CDK6 inhibitor (Lilly).

Exemplary IL17 inhibitors include, but are not limited to, a dominant negative variant of an IL17 (e.g., PCT/US2010/052194, which is hereby incorporated by reference in its entirety), a polypeptide (e.g., as described in US Patent Publication No. 2013/0005659, which is hereby incorporated by reference in its entirety), or an antibody (e.g., as described in US Patent Application Publication Nos. 2012/0107325 or 2012/0129219, each of which is hereby incorporated by reference in its entirety), antibody fragment, or antibody variant, for example, a domain antibody, a bispecific antibody that has at least one site that can specifically bind to an IL17 or IL17R, a diabody, or other structure comprising CDRs derived from an antibody in non-antibody scaffolding. Additional, non-limiting examples of IL17 inhibitors include ixekizumab, secukinumab, RG4936, RG4934, RG7624, and SCH-900117. The inhibitor may also bind to an IL17 receptor, e.g., brodalumab. (See IL17 direct and indirect inhibitors described in Truchetet et al., “IL-17 in the Rheumatologist's Line of Sight,” BioMed Research Int'l Volume 2013 (2013), which is hereby incorporated by reference in its entirety).

Exemplary TWIST1 inhibitors include, but are not limited to, modified poly(amidoamine) dendrimer-siRNA (PAMAM-siRNA) complexes (e.g., as described in Finlay et al., “RNA-Based TWIST1 Inhibition via Dendrimer Complex to Reduce Breast Cancer Cell Metastasis,” Biomed Res Int 2015:382745 (2015), which is hereby incorporated by reference in its entirety); miR-720 (Li et al., “miR-720 Inhibits Tumor Invasion and Migration in Breast Cancer by Targeting TWIST1,” Carcinogenesis 35(2):469-78 (2014), which is hereby incorporated by reference in its entirety); shTWIST1-1 and shTWIST1-2 (Burns et al., “Inhibition of TWIST1 Leads to Activation of Oncogene-Induced Senescence in Oncogene Driven Non-Small Cell Lung Cancer,” Mol Cancer Res 11(4):329-338 (2013), which is hereby incorporated by reference in its entirety); Tamoxifen (Ma et al., “Tamoxifen Inhibits ER-Negative Breast Cancer Cell Invasion and Metastasis by Accelerating Twist1 Degradation,” Int J Biol Sci 11(5):618-628 (2015), which is hereby incorporated by reference in its entirety); Sirtuin SIRT6 (Han et al., “Sirtuin SIRT6 Suppresses Cell Proliferation Through Inhibition of Twist1 Expression in Non-Small Cell Lung Cancer,” Int J Clin Exp Pathol 7(8):4774-81 (2014), which is hereby incorporated by reference in its entirety); miR-300 (Yu et al., “miR-300 Inhibits Epithelial to Mesenchymal Transition and Metastasis by Targeting Twist in Human Epithelial Cancer,” Mol Cancer 12:121 (2014), which is hereby incorporated by reference in its entirety); and Moscatilin (Pai et al., “Moscatilin Inhibits Migration and Metastasis of Human Breast Cancer MDA-MB-232 Cells Through Inhibition of Akt and Twist Signaling Pathway,” J Mol Med (Berl) 91(3):347-56 (2013), which is hereby incorporated by reference in its entirety).

Exemplary MMP-8 inhibitors include, but are not limited to, hydroxyamate-based inhibitors, synthetic inhibitors such as batimastat; BB-1101; CGS-27023-A (MMI270B); COL-3 (metastat; CMT-3); doxycycline; FN-439 (p-aminobenzoyl-Gly-Pro-D-Leu-D-Ala-NHOH, MMP-Inh-1); GM6001 (ilomastat); marimastat (BB-2516; Cl₅H₂₉N₃O₅); ONO-4817 (C₂₂H₂₈N₂O₆); Ro 28-2653; and antibody-based inhibitors (Vandenbroucke et al., “Is There New Hope for Therapeutic Matrix Metalloproteinase Inhibition,” Nat Rev Drug Disc 13:904-927 (2014), which is hereby incorporated by reference in its entirety).

Exemplary IL10 inhibitors include, but are not limited to, antibodies, antagonists, antisense nucleic acid molecules, and ribozymes, as described in, e.g., U.S. Patent Application Publication No. 20050025769, which is hereby incorporated by reference in its entirety. Examples also include IFN-gamma; Rituximab (Alas et al., “Inhibition of Interleukin 10 by Rituximab Results in Down-Regulation of Bcl-2 and Sensitization of B-cell Non-Hodgkin's Lymphoma to Apoptosis,” Clin Cancer Res 7:709 (2001), which is hereby incorporated by reference in its entirety); 15d-PGD2 (Kim et al., “Inhibition of IL-10-induced STAT3 activation by 15-deoxyΔ12,14-prostaglandin J2,” Rheumatology 44(8):983-988, which is hereby incorporated by reference in its entirety); and AS101 (ammonium trichloro(dioxoethylene-o-o′)tellurate) (Kalechman et al., “Inhibition of Interleukin-10 by the Immunomodulator AS101 Reduces Mesangial Cell Proliferation in Experimental Mesangioproliferative Glomerulonephritis,” JBC 279(23):24724-24732 (2004), which is hereby incorporated by reference in its entirety).

Exemplary FGR inhibitors include, but are not limited to, dasatinib (Montero et al., “Inhibition of Src Family Kinases and Receptor Tyrosine Kinases by Dasatinib: Possible Combinations in Solid Tumors,” Clin Cancer Res 17:5546 (2011), which is hereby incorporated by reference in its entirety).

Exemplary triggering receptor expressed on myeloid cells 1 (“TREM-1”) inhibitors include, but are not limited to, antibodies, fusion proteins, and/or inhibitory peptides or proteins (e.g., soluble forms of TREM receptors, LP17, LR12, TLT-1) (U.S. Patent Application Publication No. 20080247955; Piccio et al., “Identification of Soluble TREM-2 in the Cerebrospinal Fluid and its Association with Multiple Sclerosis and CNS Inflammation,” Eur J Immunol 37:1290-301 (2007); U.S. Patent Application Publication Nos. 20090081199, 20030165875, and 20060246082; Murakami et al., “Intervention of an Inflammation Amplifier, Triggering Receptor Expressed on Myeloid Cells 1, for Treatment of Autoimmune Arthritis,” Arthritis Rheum 60:1615-23 (2009); Gibot et al., “Effects of the TREM 1 Pathway Modulation During Hemorrhagic Shock in Rats,” Shock 32:633-7 (2009); and Derive et al., Attenuation of Responses to Endotoxin by the Triggering Receptor Expressed on Myeloid Cells-1 Inhibitor LR12 in Nonhuman Primate,” Anesthesiology 120:935-942 (2014), each of which is hereby incorporated by reference in its entirety); and inhibitory peptide variants that act on, e.g., the TREM/DAP-12 signaling complex (U.S. Patent Application Publication No. 20140154291, which is hereby incorporated by reference in its entirety).

Exemplary CCR2 inhibitors include, but are not limited to the chemokine receptor 2 (CCR2) inhibitors as described in, for example, U.S. patent and patent application Publication Nos.: U.S. Pat. Nos. 9,320,735; 7,799,824; 8,067,415; 2007/0197590; 2006/0069123; 2006/0058289; and 2007/0037794, each of which is hereby incorporated by reference its entirety. Exemplary inhibitors of CCR2 also include Maraviroc; cenicriviroc; CD192; CCX872; CCX140; CKR-2B; 2-thioimidazoles; 2-((Isopropylaminocarbonyl)amino)-N-(2-((cis-2-((4-(methylthio)benzoyl)amino)cyclohexyl)amino)-2-oxoethyl)-5-(trifluoromethyl)-benzamide; vicriviroc; SCH351125; TAK779; Teijin; and RS-504393 (Kothandan et al., “Structural Insights from Binding Poses of CCR2 and CCR5 with Clinically Important Antagonists: A Combined In Silico Study,” Plos ONE 7(3): e32864 (2012), which is hereby incorporated by reference in its entirety); the small molecule CCR2 antagonists (e.g., RS-504393, Benzimidazoles, SB-380732, AZD-6942, 3-Aminopyrrolidines, and INCB-003284), as described in Higgins et al., “Small Molecule CCR2 Antagonists,” CHEMOKINE BIOLOGY—BASIC RESEARCH AND CLINICAL APPLICATION, Vol. II, p. 1145-123 (2007), which is hereby incorporated by reference in its entirety; resveratrol (Cullen et al., “Resveratrol inhibits expression and binding activity of the monocyte chemotactic protein-1 receptor, CCR2, on THP-1 monocytes,” Atherosclerosis 195(1):e125-33 (2007), which is hereby incorporated by reference in its entirety; GSK1344386B (Olzinski et al., “Pharmacological inhibition of C—C chemokine receptor 2 decreases macrophage infiltration in the aortic root of the human C—C chemokine receptor 2/apolipoprotein E−/− mouse,” Arterioscler Thromb Vasc Biol 30(2):253-9 (2010), which is hereby incorporated by reference in its entirety; the CCR2 antagonist identified by CAS 445479-97-0, which is hereby incorporated by reference in its entirety; INCB3344 (Shin et al., “Pharmacological Characterization of INCB3344, a Small Molecule Antagonist of Human CCR2,” Biochem Biophys Res Com 387(2): 251-55 (2009), which is hereby incorporated in its entirety); and the cis-3,4-disubstituted piperidines described in Cherney et al., “Synthesis and evaluation of cis-3,4-disubstituted piperidines as potent CC chemokine receptor 2 (CCR2) antagonists,” Bioorg Med Chem Lett. 18:5063-5065 (2008), which is hereby incorporated by reference in its entirety.

Exemplary ADAM8 inhibitors include, but are not limited to, the inhibitory amino acid sequences of U.S. Pat. No. 9,156,914, which is hereby incorporated by reference in its entirety; BK-1361 (Schlomann et al., “ADAM8 as a Drug Target in Pancreatic Cancer,” Nat Commun 28(6):6175 (2015), which is hereby incorporated by reference in its entirety); the zinc chelator 1,10-phenanthroline (Amour et al., “The Enzymatic Activity of ADAM8 and ADAMS is not regulated by TIMPs,” FEBS Letters 524:154-158 (2002), which is hereby incorporated by reference in its entirety); and the cyclic peptides of WO 2009047523, which is hereby incorporated by reference in its entirety.

Exemplary IL1b inhibitors include, but are not limited to anakinra, canakinumab, rilonacept, gevokizumab, IL-1 traps, and antibodies (U.S. Patent Application Publication No. 20160120941 and U.S. Pat. Nos. 6,927,044; 6,472,179; 7,459,426; 8,414,876; 7,361,350; 8,114,394; 7,820,154 and 7,632,490, each of which is hereby incorporated by reference in its entirety).

Yet another aspect of the present invention relates to a pharmaceutical composition comprising (a) one or more target inhibitors; (b) a targeting element that causes muscle satellite cell-specific uptake or activity of the one or more inhibitors; and (c) a pharmaceutically-acceptable carrier. One embodiment relates to a pharmaceutical composition comprising (a) one or more inhibitors of MMP-9, Twist1, cyclin D1, IL17, MMP-8, IL10, FGR, TREM1, CCR2, ADAM8, or IL1b; (b) a targeting element that causes muscle satellite cell-specific uptake or activity of the one or more inhibitors; and (c) a pharmaceutically-acceptable carrier. In one embodiment, the pharmaceutical composition includes one or more inhibitors of MMP-9, Twist1, or cyclin D1. The pharmaceutical composition may include one or more inhibitors of IL17, MMP-8, IL10, FGR, TREM1, CCR2, ADAM8, or IL1b.

Further, the present invention also relates to a pharmaceutical composition comprising a combination of: (a) one or more target inhibitors; (b) a targeting element that causes muscle satellite cell-specific uptake or activity of the one or more inhibitors; (c) a pharmaceutically-acceptable carrier; and (d) an AUF1 protein, a functional fragment of AUF1 protein, an AUF1 protein mimic, or a combination thereof (or a nucleotide sequence encoding (d), as described herein). The one or more inhibitors may be of MMP-9, Twist1, cyclin D1, IL17, MMP-8, IL10, FGR, TREM1, CCR2, ADAM8, or IL1b. In one embodiment, the pharmaceutical composition includes one or more inhibitors of MMP-9, Twist1, or cyclin D1. The pharmaceutical composition may include one or more inhibitors of IL17, MMP-8, IL10, FGR, TREM1, CCR2, ADAM8, or IL1b.

Compositions as described herein, including pharmaceutical compositions may include one or more carriers (e.g., a buffer or buffer solution).

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. In one embodiment, the pharmaceutically acceptable carrier is a buffer solution.

The term “pharmaceutically acceptable” means it is, within the scope of sound medical judgment, suitable for use in contact with the cells of humans and lower animals without undue toxicity, irritation, allergic response, and the like, and is commensurate with a reasonable benefit/risk ratio.

In one embodiment, the pharmaceutical composition includes an organotropic targeting agent. In one embodiment, the targeting agent is covalently linked to a protein or polypeptide as descried herein via a linker that is cleaved under physiological conditions.

Proteins or polypeptides according to the present invention may also be modified using one or more additional or alternative strategies for prolonging in vivo half-life. One such strategy involves the generation of D-peptide chimeric proteins, which consist of unnatural amino acids that are not cleaved by endogenous proteases. Alternatively, the proteins may be fused to a protein partner that confers a longer half-life to the protein upon in vivo administration. Suitable fusion partners include, without limitation, immunoglobulins (e.g., the Fc portion of an IgG), human serum albumin (HAS) (linked directly or by addition of the albumin binding domain of streptococcal protein G), fetuin, or a fragment of any of these. The proteins may also be fused to a macromolecule other than protein that confers a longer half-life to the protein upon in vivo administration. Suitable macromolecules include, without limitation, polyethylene glycols (PEGs). Methods of conjugating proteins or peptides to polymers to enhance stability for therapeutic administration are described in U.S. Pat. No. 5,681,811 to Ekwuribe, which is hereby incorporated by reference in its entirety. Nucleic acid conjugates are described in U.S. Pat. No. 6,528,631 to Cook et al., U.S. Pat. No. 6,335,434 to Guzaev et al., U.S. Pat. No. 6,235,886 to Manoharan et al., U.S. Pat. No. 6,153,737 to Manoharan et al., U.S. Pat. No. 5,214,136 to Lin et al., or U.S. Pat. No. 5,138,045 to Cook et al., which are hereby incorporated by reference in their entirety.

The pharmaceutical composition according to the present invention can be formulated for administration orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes. The formulations may conveniently be presented in unit dosage form and may be prepared by any of the methods well known in the art of pharmacy.

Compositions according to the present invention may further include and may be delivered via a solid, gel or semi-solid growth support (e.g., agar, a polymer scaffold, matrix, or other construct). For example, the compositions according to the present invention may further include or be delivered via a tissue scaffold.

A further aspect of the present invention relates to a method of causing satellite-cell mediated muscle generation in a subject. This method involves selecting a subject in need of satellite-cell mediated muscle generation and administering to the selected subject (i) a composition of the present invention, (ii) a cell population of the present invention, (iii) AUF1 protein, a functional fragment of AUF1 protein, an AUF1 protein mimic, or a combination thereof, or (iv) a combination of (i), (ii), and (iii), under conditions effective to cause satellite-cell mediated muscle generation in the selected subject. In one embodiment, the administering is carried out by injection of (i), (ii), (iii), or (iv) into the muscle.

AUF1 protein, functional fragments of AUF1 protein, an AUF1 protein mimic, or a combination thereof may be generated according to techniques known in the art.

Proteins or polypeptides according to the present invention may be prepared for use in accordance with the present invention using standard methods of synthesis known in the art, including solid phase peptide synthesis (Fmoc or Boc strategies) or solution phase peptide synthesis. Alternatively, they may be prepared using recombinant expression systems. For instance, a nucleic acid molecule encoding the protein or polypeptide may be provided for recombinant expression of the protein or polypeptide. Further, purified proteins may be obtained by several methods readily known in the art, including ion exchange chromatography, hydrophobic interaction chromatography, affinity chromatography, gel filtration, and reverse phase chromatography. The protein is preferably produced in purified form (preferably at least about 80% or 85% pure, more preferably at least about 90% or 95% pure) by conventional techniques. Depending on whether the recombinant host cell is made to secrete the protein into growth medium (see U.S. Pat. No. 6,596,509 to Bauer et al., which is hereby incorporated by reference in its entirety), the protein can be isolated and purified by centrifugation (to separate cellular components from supernatant containing the secreted protein) followed by sequential ammonium sulfate precipitation of the supernatant. The fraction containing the protein is subjected to gel filtration in an appropriately sized dextran or polyacrylamide column to separate the protein of interest from other proteins. If necessary, the protein fraction may be further purified by HPLC.

The compositions and methods described herein are also useful in any application where satellite-cell mediated muscle generation is desired. This includes generation of muscle for various therapeutic applications. In particular, the compositions and methods described herein are useful for promoting tissue formation, regeneration, repair, or maintenance of tissue in a subject. The tissue may be muscle and, in some embodiments, the muscle is skeletal muscle.

Therapeutic applications include administering a composition to a subject in need of regeneration of lost or damaged muscle tissue, for example, after muscle injury, or in the treatment or management of diseases and conditions affecting muscle. In some embodiments, the disease or condition affecting muscle may include a wasting disease (e.g., cachexia), muscular attenuation or atrophy (e.g., sarcopenia), ICU-induced weakness, prolonged disuse (e.g., coma, paralysis), surgery-induced weakness (e.g., following joint replacement), or a muscle degenerative disease (e.g., muscular dystrophies or other myopathies).

In some embodiments, compositions and methods described herein are employed where there is a need or desire to increase the proportion of resident stem cells, or committed precursor cells, in a muscle tissue, for example, to replace damaged or defective tissue, or to prevent muscle atrophy or loss of muscle mass, in particular, in relation to diseases and disorders such as muscular dystrophy, neuromuscular and neurodegenerative diseases, muscle wasting diseases and conditions, atrophy, cardiovascular disease, stroke, heart failure, myocardial infarction, cancer, HIV infection, AIDS, and the like.

Methods according to the present invention include selecting a subject in need of satellite-cell mediated muscle generation. The subject may have, be suspected of having, or be at risk of having muscle injury, degeneration, or atrophy. The muscle injury may be disease related or non-disease related. The muscle injury, in some embodiments, is the result of functional AUF1 deficiency. The muscle injury, in some embodiments, is a myopathy or muscle disorder that is mediated by functional AUF1 deficiency in the muscle tissue. It will be understood that functional AUF1 deficiency includes a decreased level of functional AUF1 in muscle tissue as compared to a normal or control muscle tissue. Likewise, methods of producing muscle satellite cell populations described herein may involve transforming or transfecting functional AUF1 deficient cells or functional AUF1 sufficient cells.

The subject may be a mammal. In one embodiment, the subject is a human. In another embodiment, the subject is a rodent.

The subject may exhibit or be at risk of exhibiting muscle degeneration or muscle wasting. The muscle degeneration or muscle wasting may be caused in whole or in part by a disease, for example AIDS, cancer, a muscular degenerative disease, or a combination thereof.

Muscle degeneration or injury may be due to a myopathy or muscle disorder. The myopathy or muscle disorder may be a muscular dystrophy. The myopathy or muscle disorder may also be a late-onset or adult-onset myopathy or muscle disorder. Such disorders include Limb-Girdle Muscular Dystrophy (LGMD). LGMD includes, for example, bethlem myopathy (collagen 6 mutation; dominant); calpainopathy (calpain mutations; recessive; LGMD2A); desmin myopathy (desmin mutation; dominant; a form of myofibrillar myopathy; LGMD1E); dysferlinopathy (dysferlin mutations; recessive; LGMD2B); myofibrillar myopathy (mutations in desmin, alpha-B crystallin, myotilin, ZASP, filamin C, BAG3 or SEPN1 genes; all dominant except desmin type, which can be dominant or recessive); sarcoglycanopathies (sarcoglycan mutation; recessive; LGMD2C, LGMD2D, LGMD2E, LGMD2F); and ZASP-related myopathy (ZASP mutation; dominant; a form of myofibrillar myopathy).

In an alternative embodiment, the promotion of muscle cell formation can be for increasing muscle mass in a subject.

The compositions and methods described herein may be used in combination with other known treatments or standards of care for given diseases, injury, or conditions. For example, in the context of muscular dystrophy, a composition of the invention for promoting muscle satellite cell expansion can be administered in conjunction with such compounds as CT-1, pregnisone, or myostatin. The treatments (and any combination treatments provided herein) may be administered together, separately or sequentially.

The inventive work reported here identifies a novel animal model of LGMD, which enables the elucidation of the mechanism by which satellite cells are able to pre-maturely exit quiescence in the absence of AUF1. This indicates a crucial role for AUF1 in promoting regeneration and maintaining the satellite cell population through controlling the expression of MMP9, among other targets. This knowledge presents a route to improve stem cell therapies for skeletal muscle regeneration.

Satellite cells can be isolated through fluorescent-activated cell sorting (FACS) with their unique surface marker, Sdc4, and excluding endothelial markers CD45 and Sca1. Such a population can be verified through the expression of the PAX7 transcription factor, exclusively expressed in satellite cells.

Verification of treatment compositions can be carried out based on in vitro and/or in vivo models. Thus, another aspect of the present invention relates to an in vivo method of producing a muscle satellite cell population expressing exogenous AUF1 or a functional fragment thereof. This method involves transforming or transfecting Syndecan 4⁺/PAX7⁺ or Syndecan 4⁺/PAX7⁻ muscle satellite cells with a nucleic acid molecule encoding exogenous AUF1 or a functional fragment thereof, where when Syndecan 4⁺/PAX7⁺ or Syndecan 4⁺/PAX7⁻ muscle satellite cells are transformed or transfected in an in vitro or an in vivo model with the nucleic acid molecule they express the exogenous AUF1 or the functional fragment thereof.

Another aspect of the present invention relates to a method of treating a subject in need thereof with Syndecan 4⁺/PAX7⁺ or Syndecan 4⁺/PAX7⁻ muscle satellite cells expressing exogenous AUF1. This method involves administering Syndecan 4⁺/PAX7⁺ or Syndecan 4⁺/PAX7⁻ muscle satellite cells transformed or transfected with a nucleic acid molecule encoding exogenous AUF1 or a functional fragment thereof, where the Syndecan 4⁺/PAX7⁺ or Syndecan 4⁺/PAX7⁻ muscle satellite cells express the exogenous AUF1 or the functional fragment thereof in an in vitro or an in vivo model.

Following purification, satellite cells have been used in skeletal muscle stem cell therapies; however, with limited implantation success. The reason for this limited success is due to a lack of understanding of how satellite cells differentiate and return to quiescence, ultimately creating fully functional skeletal muscle. Most satellite cell transplants are re-introduced to the muscle with limited alterations. With the novel understanding of the role of AUF1 in the satellite cell disclosed here, it is proposed that increased expression of AUF1 in sorted satellite cells, combined with silencing of MMP9, would result in a novel cell population that is primed to repair skeletal muscle injury. Furthermore, because satellite cells express the unique transcription factor PAX7, it is possible to create a viral system that can be directly exposed to the skeletal muscle but only active in early stage satellite cells. Once these implanted cells begin to differentiate and lose PAX7 expression, the virus cDNA will be turned off. Ultimately this creates a novel cell population primed for repair.

### EXAMPLES

The following examples are provided to illustrate embodiments of the present invention but they are by no means intended to limit its scope.

### Example 1—The mRNA Binding Protein AUF1 Controls the Regenerative Potential of Activated Skeletal Muscle Stem Cells

The family of RBPs has emerged as orchestrators of complex molecular pathways. AUF1 is primarily implicated in promoting the degradation of mRNA targets. In this example, it is shown that AUF1 is a regulator of the regenerative potential of activated skeletal muscle stem cells, known as satellite cells, by associating to and promoting the decay of critical AU-rich mRNAs. See also, Exhibit B attached hereto.

### Materials and Methods for Examples 1 and 2

### Generating AUF1^{-/-} Mice

All AUF1^{-/-} mice and WT mice are of the 129-background F3 and F4 generation breed from AUF1 heterozygous mice. Ages varied from 6-12 months and are specified for each procedure.

### Statistical Analysis

Student's t-test was used when applicable to determine significance. Significant values are considered p<0.05 and noted by as asterisk (*).

### Dual Energy X-Ray Absorptiometry (DEXA)

The Lunar Pixi DEXA was used to record lean tissue mass. It does so by using low energy x-rays which are absorbed by the bone and lean tissues at different rates, enabling a reading of mass. Male and female mice 6 months old were weighed for total body mass and scanned for lean body mass. A ratio of lean body mass to total body was used. 5 mice per genotype were scanned in triplicate and averaged with the standard deviation.

### Cage Flip

Male and female mice were placed on top of a grid for 30 seconds to acclimate before being inverted for up to 60 seconds. The time they let go of the grid is recorded. Mice were divided into the following month age groups: 6, 7-9, 10-12. 5 mice per genotype per age group were tested and averaged with the standard deviation.

### BaCl₂ Hindlimb Injury

Male and female mice 4-6 months of age were injected by 20 uL 1.2% BaCl₂ in saline directly to the left TA muscle. Right TA muscle was left uninjured. Mice were monitored and sacrificed by protocol for 1-30 days post-injection. 2 mice per genotype per time point were studied.

### Immunofluorescence

Male and female mice 4-6 months of age had their TA removed and preserved in OCT. Skeletal muscle samples were prepared as previously described (Bernet et al., “p38 MAPK Signaling Underlies a Cell-autonomous Loss of Stem Cell Self-renewal in Skeletal Muscle of Aged Mice,” Nature Medicine 20:265-271 (2014), which is hereby incorporated by reference in its entirety). Samples were post-fixed in 4% paraformaldehyde and blocked in 3% BSA in TBS-T. Primary antibodies were incubated at 4° C. overnight. Alexa Fluor donkey 488, 555, and 647 secondary antibodies were used at 1:500 and incubated for 1 hour at room temperature. Slides were sealed with Vectashield with DAPI. The following antibody dilutions were used: rat antibody to Laminin (Sigma, L0663, 1:250), mouse antibody to PAX7 (Santa Cruz Biotechnology, SC-81648, 1:500), goat antibody to hnRNPD (Santa Cruz Biotechnology, SC-22368, 1:250).

### Microscopy, Image Processing, and Analysis

Images were acquired using a Zeiss LSM 700 confocal microscope, primarily with the 20× lens. Images were processed and scored using ImageJ64. If needed, color balance was adjusted linearly for the entire image and all images in experimental set. All images were quantified based on field of view. At least 5 images per experimental animal and at least 2 animals per genotype were used for all experiments.

### Fiber Preparation

Fibers were harvested from the hindlimb muscles of 4-6 months of age male and female mice and maintained in culture for 72 hours prior to 4% PFA fixation for immunofluorescence.

### In Vivo MMP9 Activity

WT and KO male and female mice 4 months of age were given an IP injection with PerkinElmer MMPSense 750 solution 24 h prior to injury and the time of BaCl₂ injection, 24 h prior to imaging. Animals were imaged using IVIS L-III. Three mice per genotype were analyzed, then means and standard deviations calculated. Data were analyzed with an unpaired t-test.

### SB-3CT Treatment

KO male and female mice 4 months of age were given an IP injection with 25 mg/kg SB-3CT (Sigma-Aldrich) every 24 h, starting 24 h prior to BaCl₂ injury with MMPSense injection. Three mice per treatment were analyzed, then means and standard deviations calculated. Data were analyzed with an unpaired t-test.

Results

AUF1^{-/-} Mice Increasingly Lose Muscle Mass and Strength with Age

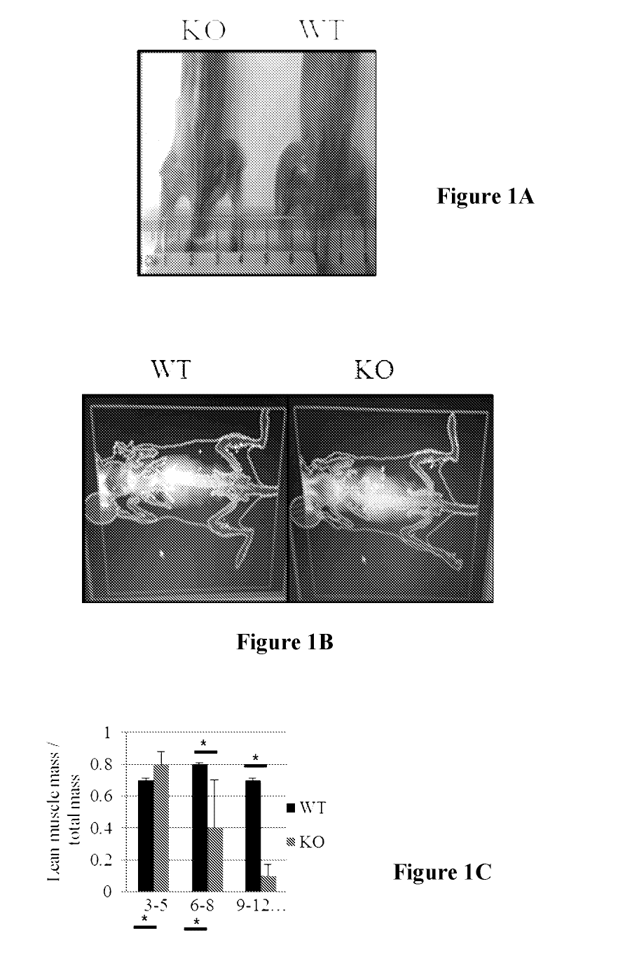

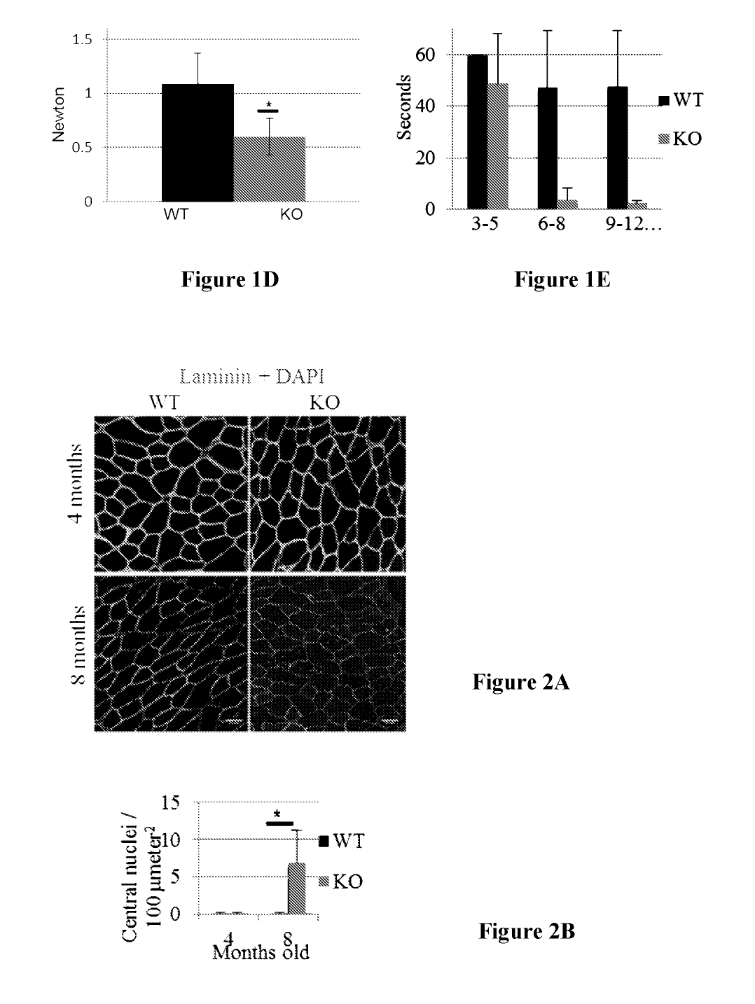

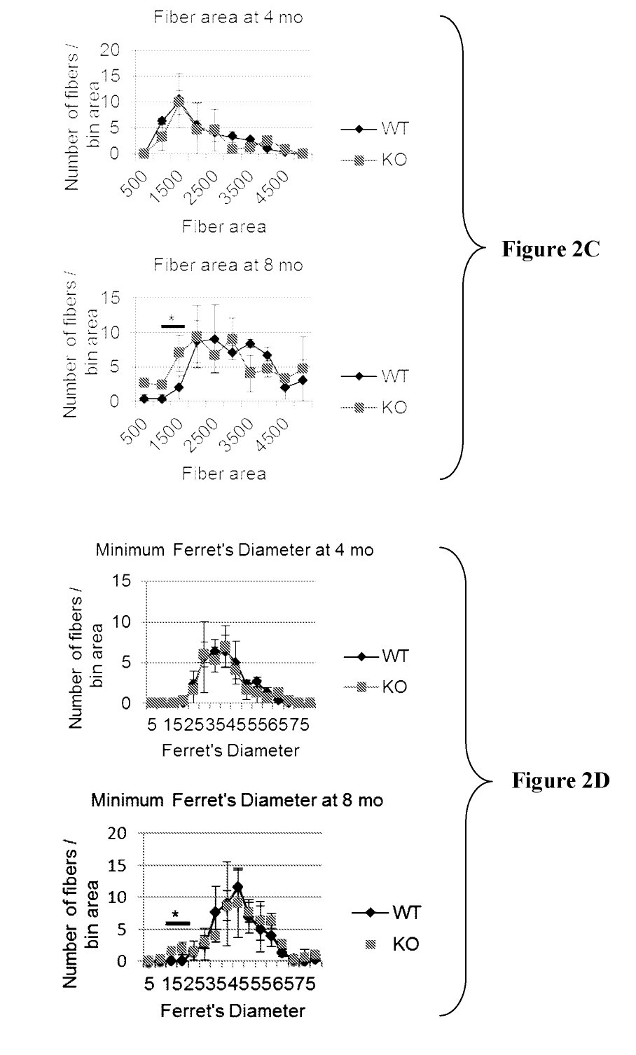

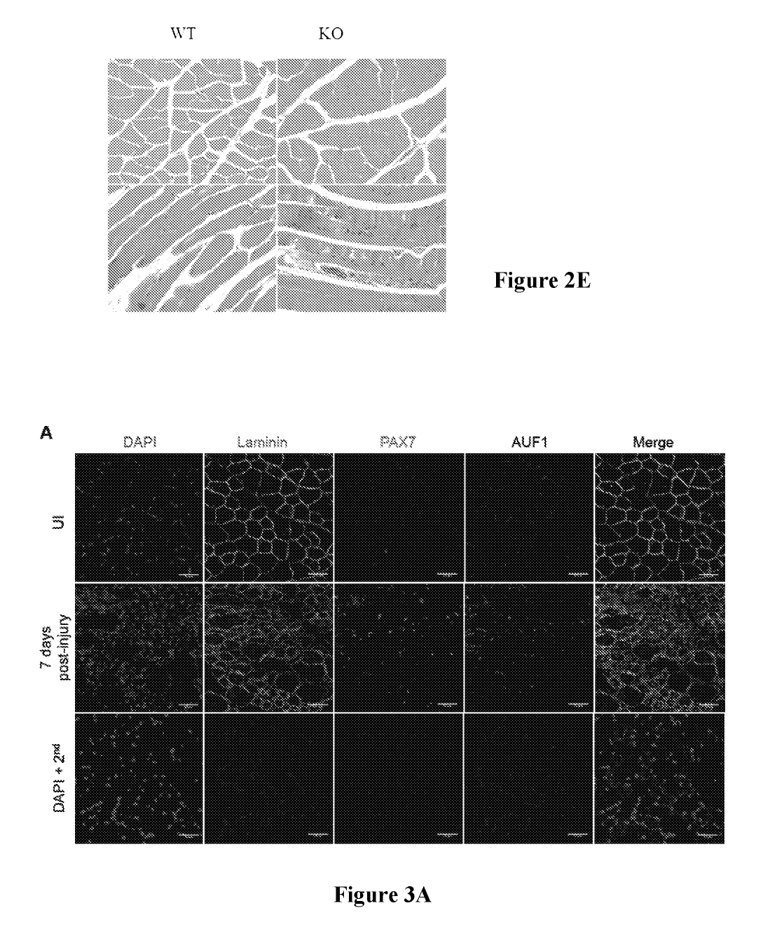

In AUF1^{-/-} mice, a profound loss of skeletal muscle mass and muscle weakness that worsens with age was observed (FIGS. 1A-1E). FIGS. 1A-1E illustrate the results of an initial observation that mice lacking functional AUF1 protein show severe muscle loss with age corresponding to reduced strength. FIG. 1A are photographs showing representative images of the hindlimb muscle mass of 6 month old WT and KO mice. FIG. 1B are photographs showing representative images of 6 month old WT and KO mice produced by the DEXA Body analyzer. FIG. 1C is a graph showing average whole body skeletal muscle mass calculated from the lean tissue mass DEXA reading normalized to total body mass at different ages in WT and KO mice. FIG. 1D is a graph showing forearm strength measured through strength grip analysis of WT and KO mice. FIG. 1E is a graph showing whole body strength measured through cage flip analysis at different ages in WT and KO mice. This phenotype is strikingly similar to limb girdle muscular dystrophy (LGMD) (FIGS. 2A-2E).

FIGS. 2A-2E relate to the pathology of the AUF1^{-/-} skeletal muscle. Specifically, mice lacking functional AUF1 protein are shown to develop a myopathic phenotype with age due to the premature activation of the satellite cell population. FIG. 2A provides photographs showing hindlimb muscle stained for the perimeter of the muscle bundle by Laminin (green) and the nuclei (DAPI blue) at 4 months of age and 8 months of age in WT and KO mice. FIG. 2B is a graph showing quantification of the centralized nuclei, indicating premature activation of satellite cells which are normally localized to the Laminin in the 8 month old KO mice. Increase in centrally located nuclei within muscle fibers is indicative of ongoing satellite cell regeneration efforts and a phenotypic hallmark of myopathic disease (Wicklund & Kissel, “The Limb-Girdle Muscular Dystrophies,” Neurol Clin 32:729-749, ix (2014), which is hereby incorporated by reference in its entirety). FIG. 2C is a pair of graphs showing quantification of the Laminin muscle fiber area showing smaller fibers in the 4 month old and 8 month old KO mice, suggesting muscle loss. FIG. 2D is a pair of graphs showing quantification of the Laminin muscle fiber Minimum Ferret's Diameter, a measurement commonly used in muscle studies that corrects for sectioning errors, showing smaller fibers in the 4 month old and 8 month old KO mice suggesting muscle loss. FIG. 2E provides photographs of H&E staining of 8 month old WT and KO mouse skeletal muscle showing irregular fiber formation and centralized nuclei in the KO mice similar to the diagnostic appearance of LGMD. In fact, a mutation in a family cohort affected with LGMD was association-mapped to the same chromosomal location as human AUF1.

### AUF1 is Expressed in Activated Satellite Cells

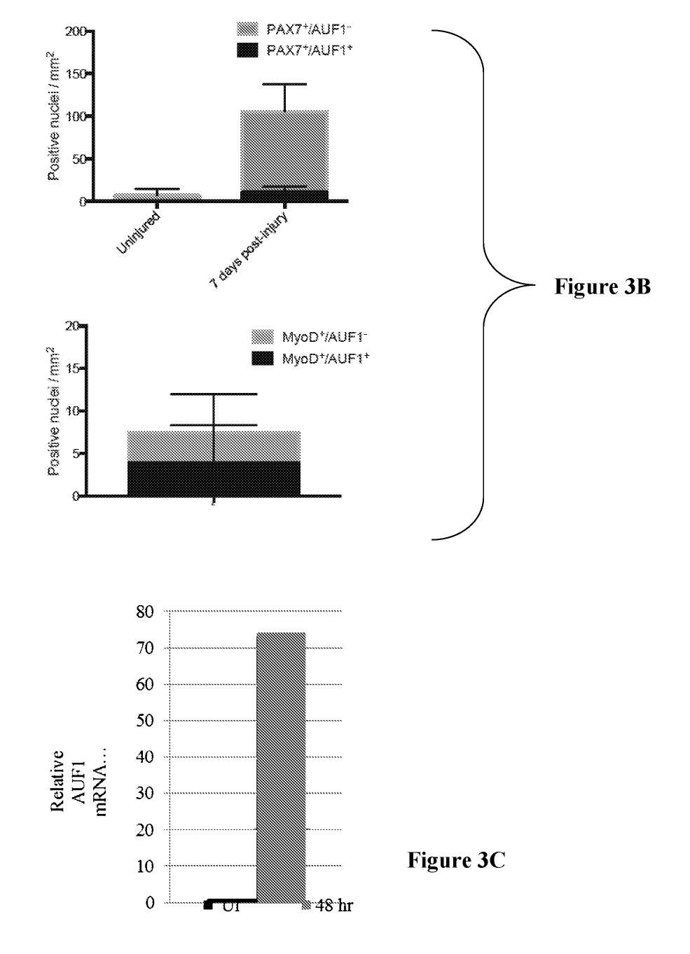

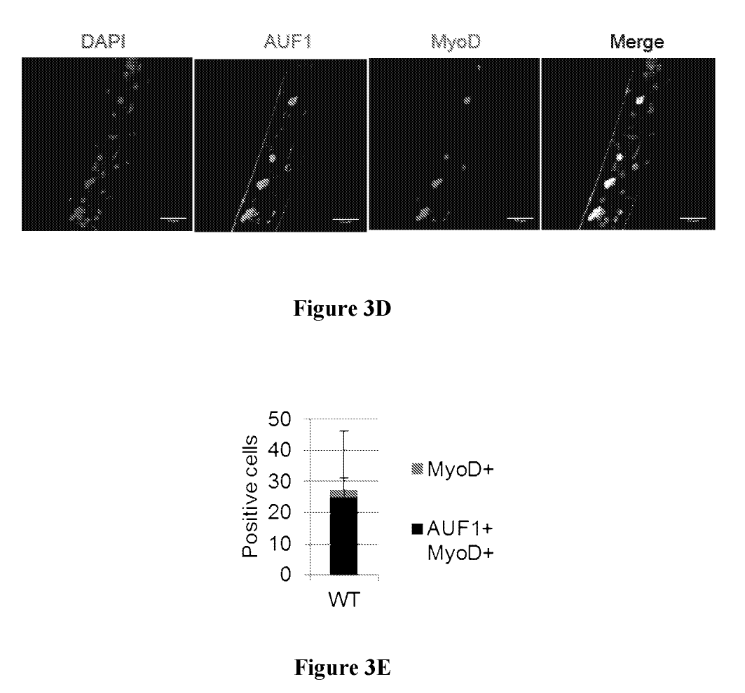

Studies have shown that AUF1 is expressed at extremely low or negligible levels in skeletal muscle fibers (Lu et al., “Tissue Distribution of AU-Rich mRNA-Binding Proteins Involved in Regulation of mRNA Decay,” The Journal of Biological Chemistry 279:12974-12979 (2004), which is hereby incorporated by reference in its entirety) (FIG. 3A, 3D). AUF1 expression was therefore screened using immunofluorescence specifically in the quiescent and activated satellite cell population in vivo following injury, and in vitro on isolated skeletal muscle fibers. Quiescent satellite cells are identified by expression of PAX7 and Syndecan-4 (Sdc4), while activated satellite cells additionally gain expression of myogenic regulatory factors (“MRFs”), such as MyoD (Cornelison, et al. “Single-Cell Analysis of Regulatory Gene Expression in Quiescent and Activated Mouse Skeletal Muscle Satellite Cells,” Dev Biol 191:270-283 (1997); Seale et al., “A New Look at the Origin, Function, and “Stem-Cell” Status of Muscle Satellite Cells,” Dev Biol 218:115-124 (2000), each of which is hereby incorporated by reference in its entirety).

Using a mouse skeletal muscle injury time course model, it was found that AUF1 levels increase in satellite cells by 24 hours post-injury activation (FIGS. 3A-3E). FIGS. 3A-E relate to AUF1 expression in the satellite cell. Satellite cells are the primary cell type in the muscle capable of division, because muscle fibers are unable to grow or divide. AUF1 is shown to be expressed in satellite cells actively involved in skeletal muscle regeneration. FIG. 3A provides photographs of hindlimb muscle stained for nuclei (DAPI blue), Laminin (green), the quiescent and early activated satellite cell marker PAX7 (red), and AUF1 (white) in an uninjured state or 7 days post-injury with the DAPI and secondary antibody control panel showing that AUF1 is expressed in the PAX7-positive cells following injury. FIG. 3B shows experimental results demonstrating that AUF1 is expressed in MyoD+ satellite cells. Quantification of AUF1 co-localization to PAX7 in uninjured and 7 days post-injury TA muscle showing AUF1 is expressed in a subset of PAX7+ satellite cells is shown in the graph in the top panel of FIG. 3B. Quantification of AUF1 co-localization with MyoD in cultured myofibers showing AUF1 is expressed in over 50% of MyoD+ satellite cells is shown in the graph in the bottom panel of FIG. 3B. FIG. 3C is a graph showing expression of AUF1 from Sdc4-positive satellite cells sorted 48 hours after injury compared to Sdc4-positive satellite cells sorted from an uninjured hindlimb. There was little or no detectable AUF1 expression in quiescent satellite cells prior to muscle injury. However, AUF1 was co-expressed in ˜25% of the activated PAX7+ satellite cells 7 days post-injury (FIG. 3A). In both the uninjured and the 5 days post-injury skeletal muscle, AUF1 expression was not observed in the skeletal muscle fibers (FIG. 3A). AUF1 is therefore specifically expressed in a subset of activated satellite cells.

To further validate restriction of AUF1 expression to activated satellite cells, skeletal muscle fibers were isolated, cultured, and screened for AUF1 co-expression with MyoD, an early time point MRF. The isolation of muscle fibers activates associated satellite cells that attempt to repair the sensed “wound” by differentiation. FIG. 3D are photographs showing fibers isolated from the hindlimb muscle stained for nuclei (DAPI blue), AUF1 (green), and the early muscle determination factor MyoD (red) showing that AUF1 is expressed in the MyoD-positive cells. FIG. 3E is a graph showing quantification of the AUF1 and MyoD co-localization. At 72 hours of culture, AUF1 was strongly co-expressed in >50% of the MyoD+ satellite cells (FIG. 3D). Of note, AUF1 distribution was found to be nuclear and cytoplasmic, indicative of increased cytoplasmic ARE-mRNA decay function. AUF1 has been shown to shuttle between the nucleus and the cytoplasm; the cytoplasm being where it promotes ARE-mRNA decay. At steady-state AUF1 is primarily nuclear with export to the cytoplasm occurring as a result of specific mRNA association for decay (Moore et al., “Physiological Networks and Disease Functions of RNA-Binding Protein AUF1,” Wiley Interdisciplinary Reviews RNA 5:549-564 (2014); Sarkar et al., “Nuclear Import and Export Functions in the Different Isoforms of the AUF1/Heterogeneous Nuclear Ribonucleoprotein Protein Family,” The Journal of Biological Chemistry 278:20700-20707 (2003); Suzuki et al., “Two Separate Regions Essential for Nuclear Import of the hNRNP D Nucleocytoplasmic Shuttling Sequence,” FEBS J272:3975-3987 (2005); Yoon et al., “AUF1 Promotes let-7b Loading on Argonaute 2,” Genes & Development 29:1599-1604 (2015); He et al., “14-3-3sigma is a p37 AUF1-Binding Protein that Facilitates AUF1 Transport and AU-Rich mRNA Decay,” The EMBO Journal 25:3823-3831 (2006), each of which is hereby incorporated by reference in its entirety). Collectively, these data demonstrate that AUF1 is only expressed in activated satellite cells in skeletal muscle, and not in muscle fibers.

### Auf1^{-/-} Satellite Cells are Unable to Self-Renew Once Activated

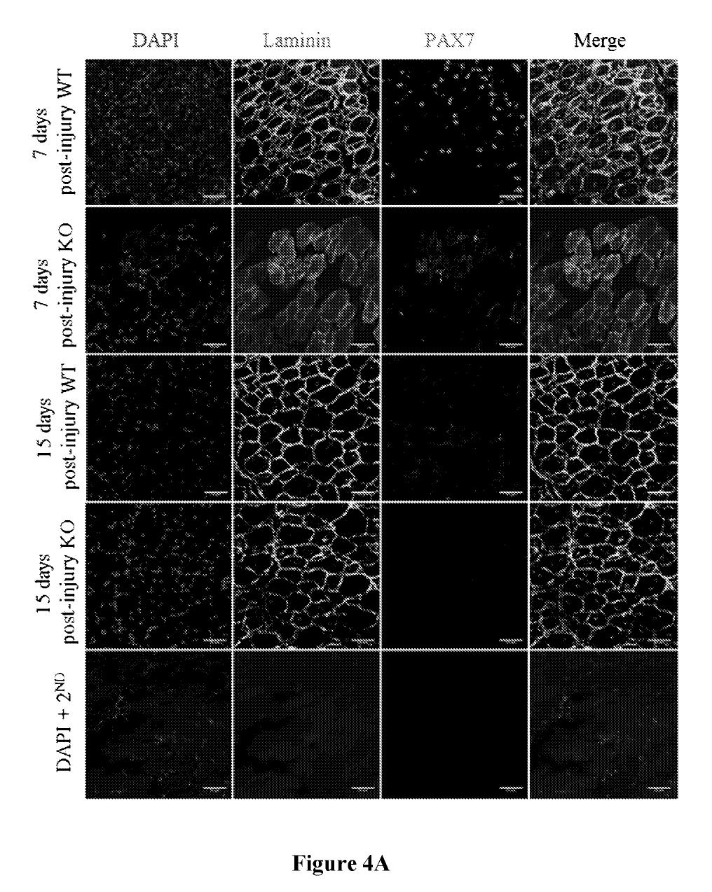

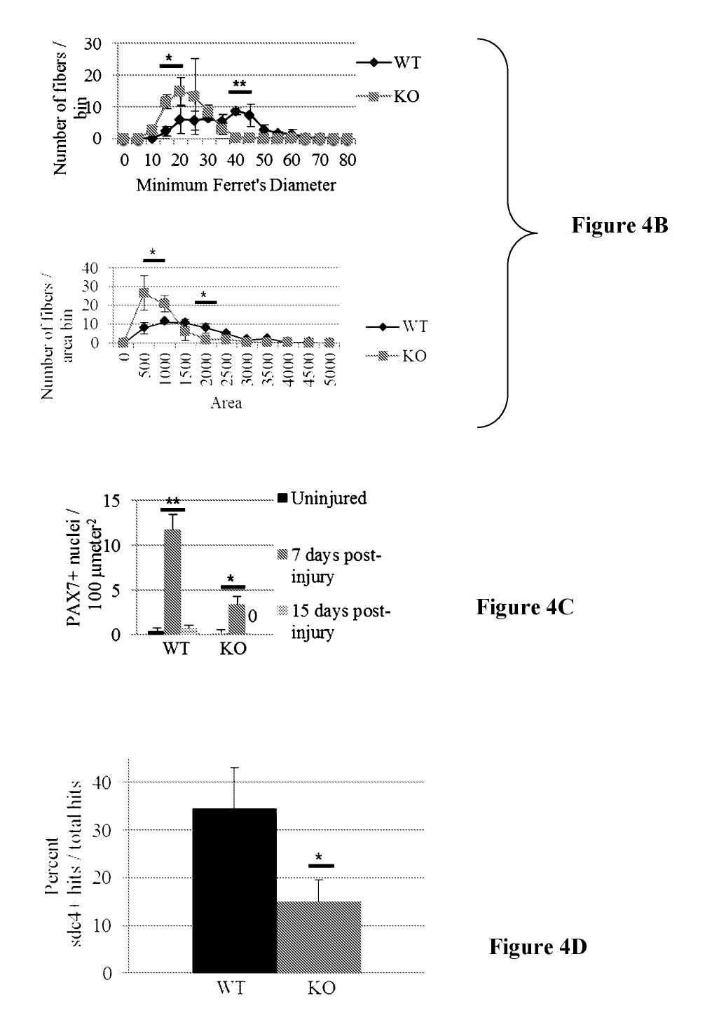

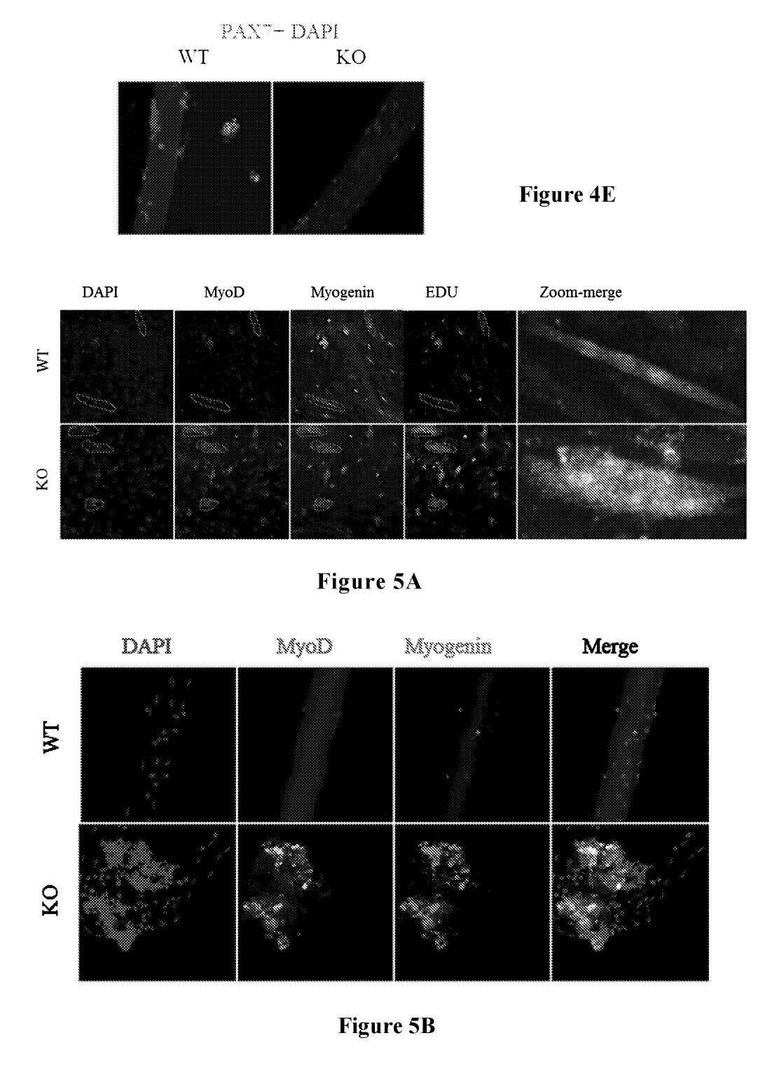

With this knowledge, the rate of skeletal muscle regeneration between WT and AUF1^{-/-} mice following hind limb injury was compared (FIGS. 4A-4E). FIGS. 4A-E relate to how the AUF1^{-/-} satellite cell population compares to a healthy WT satellite cell population with respect to repairing injury. Specifically, in the absence of AUF1, satellite cells are shown to be unable to repair skeletal muscle injury resulting in irregular muscle fibers and a loss of the PAX7-positive satellite cell population. FIG. 4A are photographs showing hindlimb muscle stained for nuclei (DAPI blue), Laminin (green), and PAX7 (red) from the WT or KO mice 7 or 15 days after hindlimb injury by BaCl₂ injection. The DAPI and secondary antibody panel are a control showing that in the KO mouse muscle satellite cells are unable to form proper laminin fibers and, therefore, exhaust and deplete the population. FIG. 4B is a pair of graphs showing quantification of the 15 days post-injury laminin fiber area and Minimum Ferret's Diameter showing significantly smaller fibers in the KO mice and significantly larger fibers in the WT mice suggesting a loss of muscle mass. FIG. 4C is a graph showing quantification of the PAX7-positive cells showing minimal PAX7 expansion 7 days post-injury and complete PAX7 depletion 15 days post-injury in the KO mice. FIG. 4D is a graph showing the number of satellite cells able to be isolated through Sdc4 selection in the hindlimb at 6 months of age in WT and KO mice. FIG. 4E is a pair of photographs showing fibers isolated from the hindlimb muscle of WT and KO mice stained for nuclei (DAPI blue) and PAX7 (green) showing complete loss of PAX7 following satellite cell activation in the KO mice. While the WT mice show significant repair within 15 days, the AUF1^{-/-} skeletal muscle shows almost no regeneration. AUF1 expression is therefore crucial for maintenance of both the satellite cell niche and the PAX7⁺ stem cell population. In the absence of AUF1, following muscle injury, satellite cells are unable to significantly expand and self-renew following activation.

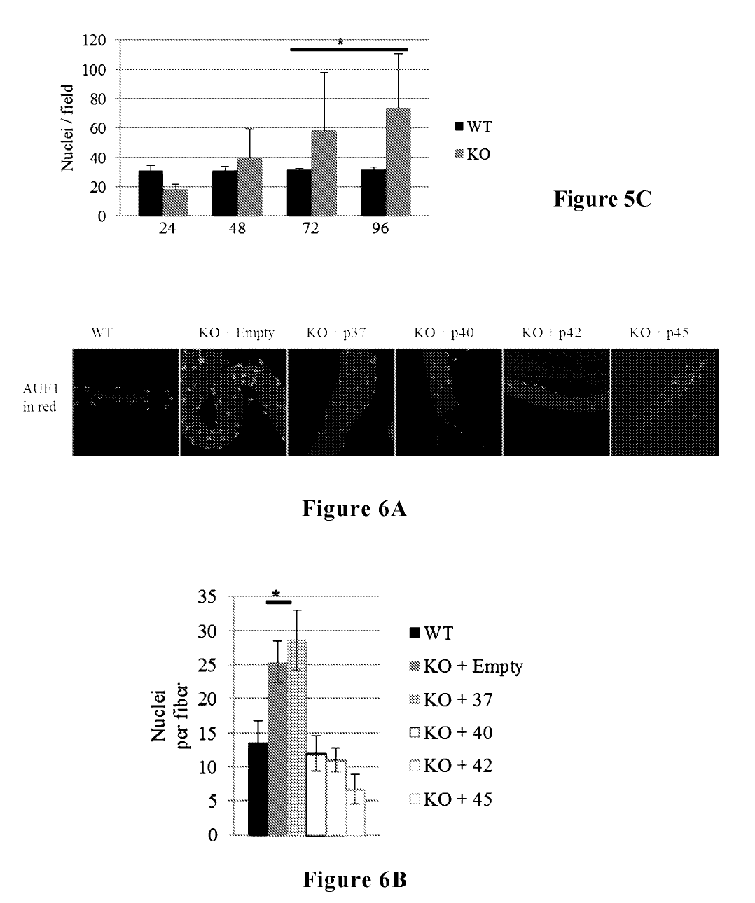

Mouse primary explant skeletal muscle fiber culture studies show that AUF1^{-/-} stem cells are activated following injury but unable to express the late stage myogenic regulatory factor, myogenin (FIGS. 5A-5C). FIGS. 5A-5C relate to how myogenesis is altered in the absence of AUF1. Specifically, in the absence of AUF1, satellite cells are shown to rapidly proliferate without differentiation. FIG. 5A are photographs showing cultured hindlimb muscle lysate from WT and KO mice stained for nuclei (DAPI blue), MyoD (red), the late muscle differentiation factor Myogenin (green), and the division identifier EDU (white) showing significantly more dividing cells with no multi-nucleated myofibers in the KO mice population. FIG. 5B are photographs showing fibers isolated from the hindlimb muscle of WT and KO mice stained for nuclei (DAPI blue), MyoD (green), and Myogenin (red) showing significantly more cells dividing in the KO fibers. FIG. 5C is a graph showing quantification of nuclei from the WT and KO mouse fibers showing a constant cell division in the KO mouse fibers despite expression of late differentiation factors. Without expression of myogenin, satellite cells remain in an activated myoblast-like state and are unable to differentiate. This suggests that in the absence of AUF1 following severe trauma or repeat injury, there is depletion of the quiescent stem cell population and increased loss of skeletal muscle.

Levels of Pax7 expression, an early stage satellite cell marker that functions in the maintenance of the quiescent population, were tested to confirm this phenotype. A complete loss of Pax7 expression in AUF1^{-/-} satellite cells following injury activation was observed (FIGS. 4A-4E). This confirms that in AUF1^{-/-} satellite cells there is a depletion of the satellite cell population following injury.

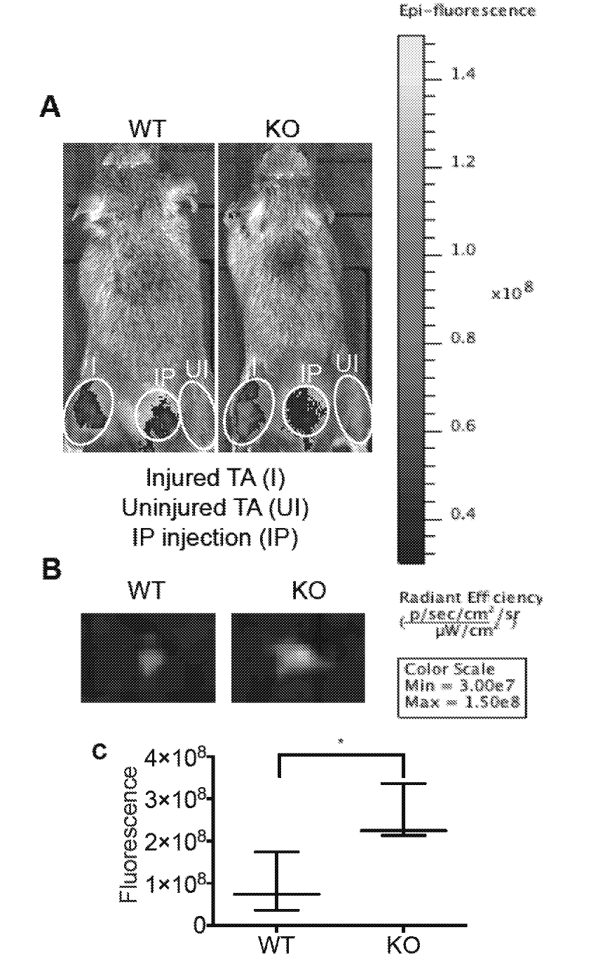

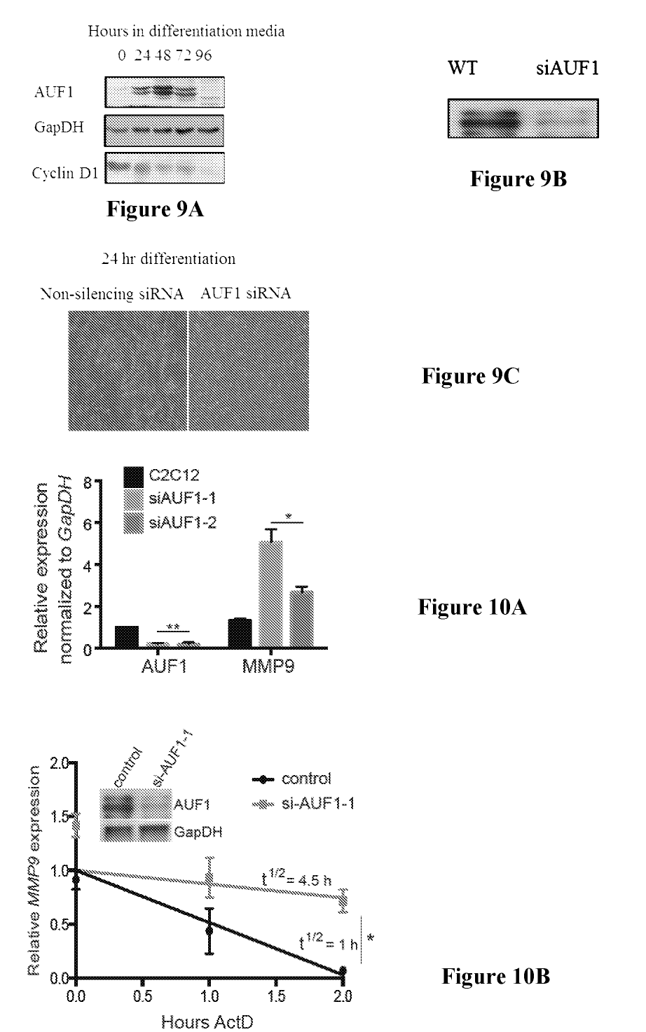

To understand the molecular role of AUF1 in the determination of satellite cell fate, studies using C2C12 cells, an established mouse myoblast cell line were performed (FIGS. 9A-9C). When AUF1 is partially silenced, significantly delayed myogenesis was observed due to reduced expression of Myogenin, complementing the observation made in the primary muscle fiber mouse explant studies. FIGS. 9A-C show that differentiation is delayed when AUF1 is partially silenced in C2C12 cells. FIG. 9A shows protein expression in C2C12 cells following myogenesis showing AUF1 expression throughout differentiation by no AUF1 expression once myofibers are formed corresponding to expression of the known AUF1 target Cyclin D1. FIG. 9B shows that using an siAUF1 construct, AUF1 can effectively be silenced in the C2C12 cells. FIG. 9C are photographs providing representative images of the C2C12 cell population 24 hours after differentiation showing myotube formation in the non-silenced cells while no myotubes are present in the si-AUF1 cells. The expression of nascent Myogenin is also reduced with partial AUF1 silencing; for this reason, the expression of myogenin regulating transcription factors was examined.

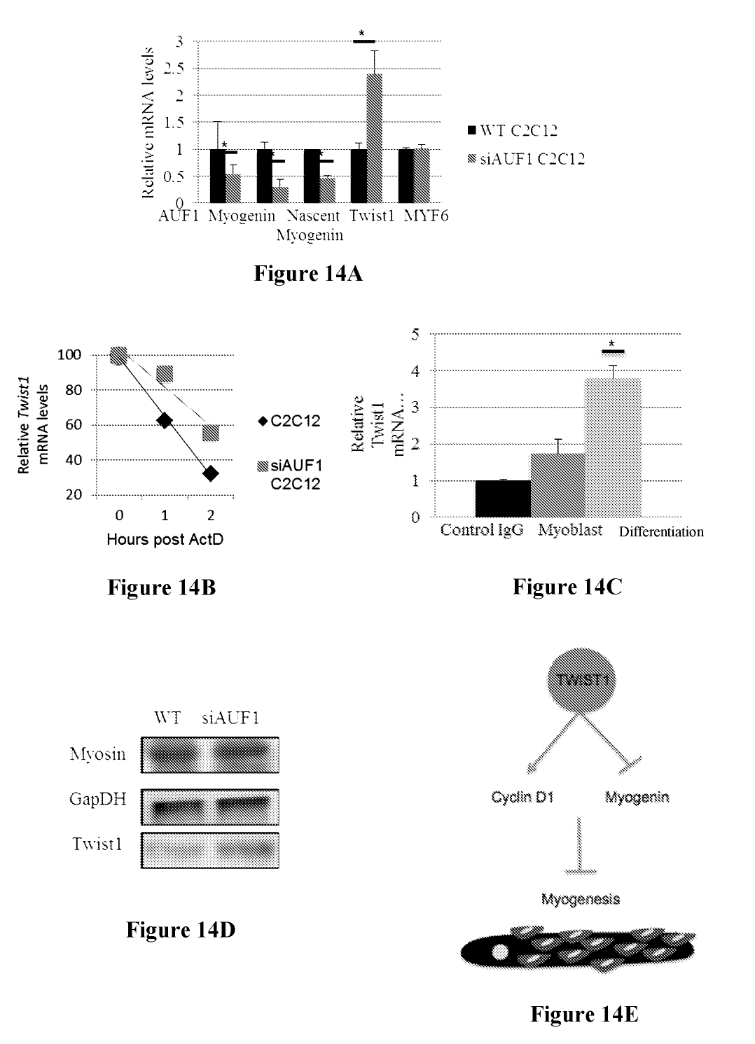

It is shown that when AUF1 is partially silenced there is a 2.5 fold increase in expression of Twist1, an inhibitor of myogenesis that directly represses Myogenin transcription (FIGS. 14A-14E). Specifically, Twist1, the stem-maintenance transcription factor, is altered in the absence of AUF1 during C2C12 myogenesis. FIG. 14A is a graph showing RNA levels of AUF1, Myogenin, Nascent Myogenin (Unaltered by RNA-binding proteins), Twist1, and MYF6 (a control differentiation factor) in differentiating C2C12 cells with or without siAUF1 treatment. FIG. 14B is a graph showing RNA stability levels of Twist1 in differentiating C2C12 cells with or without siAUF1 treatment. FIG. 14C is a graph showing RNA-immunoprecipitation of IgG or AUF1 analyzed for Twist1 association. FIG. 14D are photographs showing protein levels of Myosin (identifying differentiation), GapDH, and Twist1 in differentiating C2C12 cells with or without siAUf1 treatment.

Twist1 is encoded by an mRNA enriched in 3′UTR AU-rich motifs, potential AUF1 binding sites. Using RNA immuno-precipitation a direct interaction between AUF1 and Twist1 mRNA was identified during C2C12 cell differentiation (FIG. 14E).

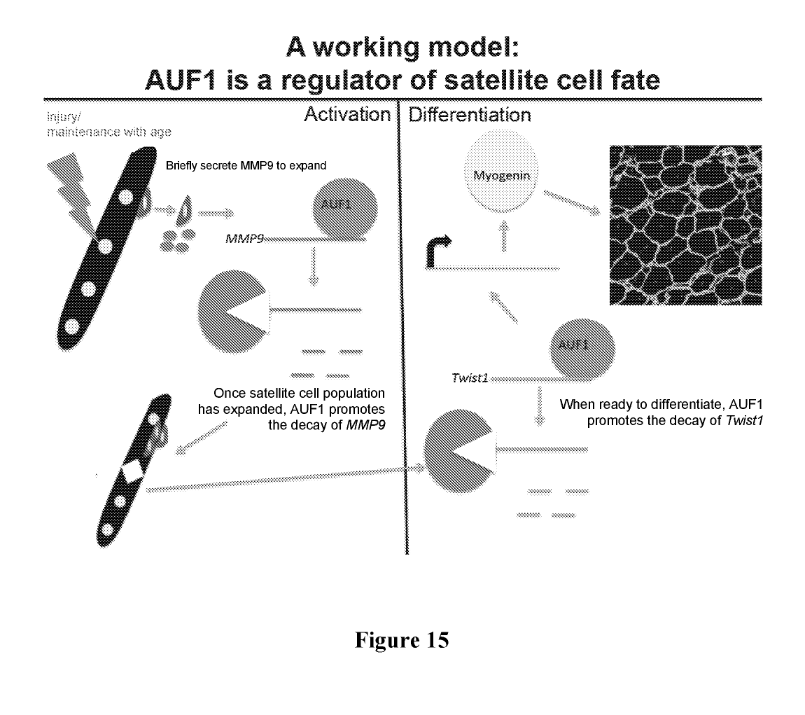

This suggests that AUF1 mediated decay of Twist1 mRNA is crucial for the ability of activated muscle (stem) satellite cells to express Myogenin and complete regeneration. Without Myogenin expression the satellite cell population maintains a “stem-like” phenotype and depletes the quiescent population. These data demonstrate the importance of the RNA binding protein and mRNA decay factor AUF1 as a fundamental regulator of stem cell fate, and implicate loss or mutation of AUF1 in the development of LGMD through reduced skeletal muscle stem cell regeneration (FIG. 15).

### Example 2—Enhanced AUF1 Expression Combined with Inhibition of MMP9 in the Satellite Cell Population of Skeletal Muscle Results in a Modified Cell Type which is Optimal for Regeneration, Identifying a Novel Target and Mechanism of Stem Cell Therapy

Rapid repair of skeletal muscle injury by satellite cells involves tightly regulated but poorly understood gene expression changes. Due to this limited knowledge, the importance of satellite cells is currently debated in the fields of myopathies and regenerative medicines. The work described in this example addresses this debate through studying the progressive loss of skeletal muscle mass and muscle weakness in mice lacking AUF1.

As noted in the previous example, in the aging AUF1^{-/-} skeletal muscle, satellite cells become prematurely activated. This results in a late on-set myopathic phenotype similar to LGMD. Following hindlimb injury in the absence of AUF1, satellite cells show a reduced rate of regeneration and are unable to return to quiescence. Taken together, this suggests a role of AUF1 in maintain a quiescent satellite cell population (FIGS. 6A-B). FIGS. 6A-B pertain to whether the proliferating satellite cell phenotype can be rescued with the addition of AUF1. Specifically, ex vivo addition of AUF1 p40, p42, or p45 to KO mouse fibers is shown to rescue the proliferating phenotype. FIG. 6A shows photographs of fibers isolated from WT or KO mice hindlimb muscle treated with either AUF1 p37, p40, p42, or p45 stained for AUF1 (red). FIG. 6B is a graph showing quantification of nuclei showing hyper-proliferation in the KO mice with an empty vector or the addition of just p37.

In particular, to identify mRNA targets of AUF1, an RNA-Seq analysis was performed, which identified 91 genes that were altered in the absence of AUF1 (FIGS. 7A-7B). FIG. 7A is a heat map of 91 genes altered in Sdc4-positive sorted satellite cells from the KO mouse hindlimb muscle compared to the WT mouse, identifying an increase in MMP9 levels. More specifically, since the primary function of AUF1 is to target ARE-mRNAs for rapid decay, identification of mRNAs with altered abundance in sorted satellite cells from auf1 KO mice compared to WT was examined. Genome-wide, satellite cell-specific RNA-Sequencing (RNA-seq) mRNA expression analysis was conducted. Satellite cells were isolated from auf1 WT and auf1^{-/-} KO mouse whole hind limb skeletal muscle from 4-6 month old animals by fluorescence-activated cell sorting (FACS), gating on cells positive for satellite cell marker Sdc4 and negative for endothelial cell markers. Ninety-one mRNAs were altered in abundance in auf1 KO compared to WT satellite cells, with ˜75% (˜70 mRNAs) showing >2-fold increased or decreased abundance. Of these, 34/70, or almost half, were mRNAs containing 3′UTRs with putative AUF1/AUBP-binding AREs based on the ARE-motif AUUUA, typically with at least two contiguous AUUUA sequences required for AUF1 binding. (Moore et al., “Physiological Networks and Disease Functions of RNA-Binding Protein AUF1,” Wiley Interdisciplinary Reviews RNA 5:549-564 (2014)), which is hereby incorporated by reference in its entirety). Additionally, the majority of the ARE-mRNAs were increased in abundance, supporting the role of AUF1 in promoting ARE-mRNA decay in the stem cell population (FIG. 7A, Table in FIG. 7D). Interestingly, 18 mRNAs were increased in abundance only in auf1 KO satellite cells and were not detectable in the WT, of which 8 contain 3′UTR multiple AREs, including established targets of AUF1 such as IL17 (Han et al., “Interleukin-17 Enhances Immunosuppression by Mesenchymal Stem Cells,” Cell Death Differ 21:1758-1768 (2014),which is hereby incorporated by reference in its entirety). Other established ARE-mRNA targets of AUF1 increased in abundance in auf1 KO satellite cells compared to WT, and include IL10 (Sarkar et al., “AUF1 Isoform-Specific Regulation of Anti-Inflammatory IL10 Expression in Monocytes,” J Interferon Cytokine Res 28:679-691 (2008), which is hereby incorporated by reference in its entirety) MMP9 (Liu et al., “AUF-1 Mediates Inhibition by Nitric Oxide of Lipopolysaccharide-Induced Matrix Metalloproteinase-9 Expression in Cultured Astrocytes,” J Neurosci Res 84:360-369 (2006), which is hereby incorporated by reference in its entirety), GBP1 and SAMSN1 (Sarkar et al., “RNA-Binding Protein AUF1 Regulates Lipopolysaccharide-Induced IL10 Expression by Activating Ikappab Kinase Complex in Monocytes,” Mol Cell Biol 31:602-615 (2011), which is hereby incorporated by reference in its entirety) and IL1beta (Pont et al., “mRNA Decay Factor AUF1 Maintains Normal Aging, Telomere Maintenance, and Suppression of Senescence by Activation of Telomerase Transcription,” Molecular Cell 47:5-15 (2012), which is hereby incorporated by reference in its entirety).

In silco analysis was next performed to identify favorable AUF1-regulated ARE-mRNAs, focusing on mRNAs upregulated in the auf1 KO satellite cells consistent with the primary function of AUF1 in mediating ARE-mRNA decay. mRNAs with at least one canonical ARE motif (AUUUA) in the 3′-UTR were identified using ARESite (Table in FIG. 7D, identified by *). These mRNAs were further prioritized as AUF1-prefered targets based on established AUF1 preference for at least two ARE pentamers, often adjacent (Gratacos et al., “The Role of AUF1 in Regulated mRNA Decay,” Wiley Interdisciplinary reviews RNA 1:457-473 (2010)), which is hereby incorporated by reference in its entirety). (Table in FIG. 7D, identified by **). The prioritized gene list was subjected to Ingenuity Pathway Analysis (IPA) to determine functional clusters. IPA assigns gene lists to experimentally authenticated biochemical and molecular networks.

IPA analysis revealed that upregulated mRNAs were enriched for functions including cell movement, cell-to-cell signaling, cell maintenance and cell growth (FIG. 7B). These pathways provide crucial signaling for the proper activation, differentiation, and self-renewal of stem cells in adult tissue. Notably, the upregulated MMP9 transcript was identified in most of these cellular function pathways. The importance of the genes identified by IPA analysis were characterized by established function in skeletal muscle regeneration. Four ARE-mRNAs were identified (Table in FIG. 7E) with two (IL17, MMP9) having been previously shown to bind AUF1 in other cell types (Han et al., “Interleukin-17 Enhances Immunosuppression by Mesenchymal Stem Cells,” Cell Death Differ 21:1758-1768 (2014), which is hereby incorporated by reference in its entirety).

MMP9 has a central importance in muscle regeneration and wound repair (Webster et al., “Intravital Imaging Reveals Ghost Fibers as Architectural Units Guiding Myogenic Progenitors During Regeneration,” Cell Stem Cell (2015); Gu et al., “A Highly Specific Inhibitor of Matrix Metalloproteinase-9 Rescues Laminin from Proteolysis and Neurons from Apoptosis in Transient Focal Cerebral Ischemia,” J Neurosci 25:6401-6408 (2005); Hindi et al., “Matrix Metalloproteinase-9 Inhibition Improves Proliferation and Engraftment of Myogenic Cells in Dystrophic Muscle of Mdx Mice,” PLoS One 8:e72121 (2013); Murase et al., “Matrix Metalloproteinase-9 Regulates Survival of Neurons in Newborn Hippocampus,” JBC 287:12184-12194 (2012),which are hereby incorporated by reference in their entirety) and was found in most of the relevant pathways analyses conducted and herein described. MMP9 is a matrix metallopeptidase that degrades extracellular matrix (ECM) proteins, including skeletal muscle laminin, a component of the satellite cell niche (Gu et al., “A Highly Specific Inhibitor of Matrix Metalloproteinase-9 Rescues Laminin from Proteolysis and Neurons from Apoptosis in Transient Focal Cerebral Ischemia,” J Neurosci 25:6401-6408 (2005); Hindi et al., “Matrix Metalloproteinase-9 Inhibition Improves Proliferation and Engraftment of Myogenic Cells in Dystrophic Muscle of Mdx Mice,” PLoS One 8:e72121 (2013); Murase et al., “Matrix Metalloproteinase-9 Regulates Survival of Neurons in Newborn Hippocampus,” JBC 287:12184-12194 (2012),which are hereby incorporated by reference in their entirety). While controlled remodeling of the ECM is required for skeletal muscle regeneration, excessive and/or continuous post-wounding MMP9 activity would be predicted to deregulate satellite cell function and impair stem cell regenerative capacity through chronic degradation of the surrounding matrix (Webster et al., “Intravital Imaging Reveals Ghost Fibers as Architectural Units Guiding Myogenic Progenitors During Regeneration,” Cell Stem Cell (2015); Shiba et al., “Differential Roles of MMP-9 In Early and Late Stages Of Dystrophic Muscles in a Mouse Model of Duchenne Muscular Dystrophy,” Biochim Biophys Acta 1852:2170-2182 (2015), each of which is hereby incorporated by reference in its entirety). Accordingly, inhibition of MMP9 has been shown to improve skeletal muscle repair in certain models of muscular dystrophy (Hindi et al., “Matrix Metalloproteinase-9 Inhibition Improves Proliferation and Engraftment of Myogenic Cells in Dystrophic Muscle of Mdx Mice,” PLoS One 8:e72121 (2013); Li, et al., “Matrix Metalloproteinase-9 Inhibition Ameliorates Pathogenesis and Improves Skeletal Muscle Regeneration in Muscular Dystrophy,” Hum Mol Genet 18:2584-2598 (2009)); Shiba et al., “Differential Roles of MMP-9 In Early and Late Stages Of Dystrophic Muscles in a Mouse Model of Duchenne Muscular Dystrophy,” Biochim Biophys Acta 1852:2170-2182 (2015), each of which is hereby incorporated by reference in its entirety). Moreover, the extensive pathological effects of muscle wounding in auf1 KO mice are consistent with the predicted phenotype of increased MMP activity.

However, importantly, the source of MMP9 expression during muscle wound repair and its pathological relevance when overexpressed have not been studied and/or determined before the present studies described herein. It was therefore first confirmed that changes in MMP9 and other mRNAs identified by genome-wide satellite cell RNA-seq analysis are in fact satellite cell autonomous. To do so, a genome-wide gene expression analysis of mRNAs in the WT and auf1 KO mouse skeletal muscle fibers taken from 4-6 month old animals (FIG. 7C) was conducted. MMP9 mRNA was undetectable in both WT and KO skeletal muscle fibers, indicating that MMP9 expression is solely satellite cell-autonomous, and the source of MMP9 overexpression in auf1 KO mice.

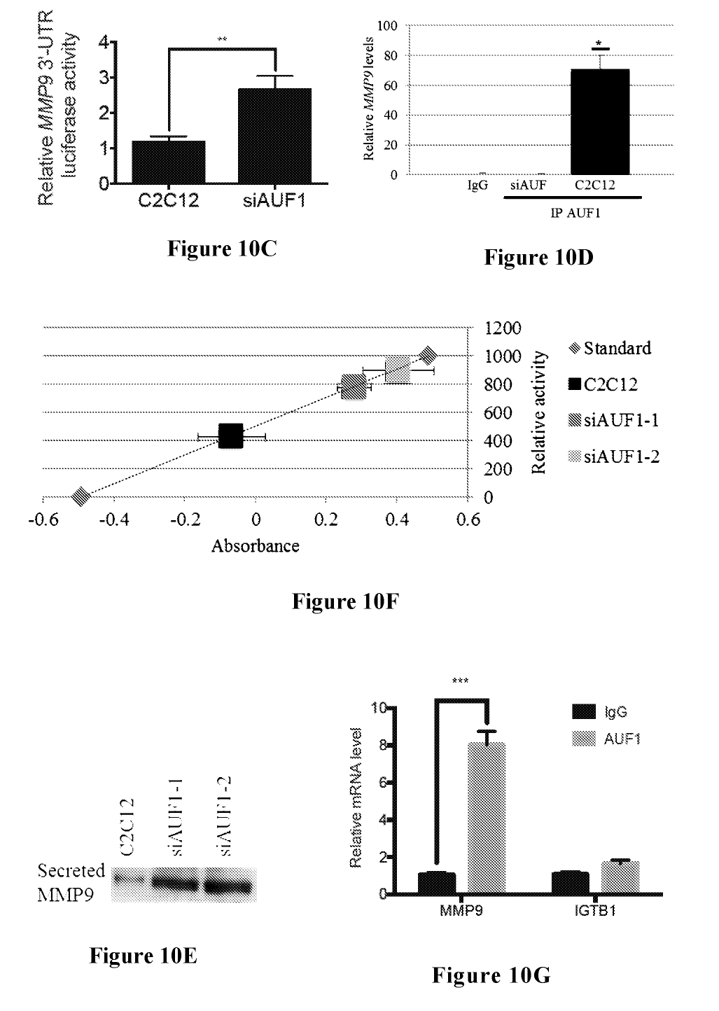

Based on the observed phenotype, focus was placed on increased expression of the matrix protease MMP9. It is demonstrated here (infra) that in the absence of AUF1, MMP9 mRNA has an increased stability and, therefore, increased expression with subsequent activation (FIGS. 8A-8C, FIGS. 10A-10G). This increased expression of MMP9 causes (1) the premature activation of satellite cells with aging and (2) the breakdown of the satellite cell niche following traumatic injury.

FIGS. 10A-10G relate to whether MMP9 is more active in C2C12 cells treated with siAUF1. Verification that AUF1 promotes MMP9 mRNA degradation was obtained in C2C12 myoblast cells, since it is not feasible to study mRNA decay rates in the animal satellite cell population. MMP9 is shown to be significantly more active when AUF1 is partially silenced in the C2C12 cells. Silencing of AUF1 by two different siRNAs (˜80%) increased MMP9 mRNA levels by ˜4 fold (FIG. 10A), consistent with that identified in the RNA-Seq data from satellite cells. MMP9 mRNA relative half-life, determined by addition of actinomycin D to block transcription, and qRT-PCR quantitation was increased from 1 h in vehicle treated controls to 4.5 h in C2C12 cells treated with siAUF1 (˜80% silenced) (FIG. 10B). To confirm that this destabilization is the result of AUF1 interaction with the ARE repeats in the 3′UTR, the longest ARE-rich region (˜200 kB) was cloned behind a luciferase reporter (pzeo-luc). This construct was transfected into untreated or siAUF1 treated C2C12 cells. Cells treated with siAUF1 showed significantly increased luciferase activity, validating the role of AUF1 in promoting MMP9 instability (FIG. 10C). FIG. 10D is a graph showing RNA-immunoprecipitation of IgG or AUF1 analyzed for MMP9 association showing increased MMP9 in the AUF1 IP from C2C12 cells without si-AUF1 treatment. FIG. 10E shows protein levels of secreted MMP9 from C2C12 cells with or without siAUF1 treatment. FIG. 10F is a graph showing ELISA measuring MMP9 activity of C2C12 cells with or without siAUF1 treatment. Additionally, MMP9 mRNA was found strongly bound to immunoprecipitated AUF1 from WT C2C12 cells (FIG. 10G). A known AUF1 target mRNA, Integrinβ-1 (ITGB1), that was not altered in the satellite cell RNA-Sequencing data was used as a control. ITGB1 did not associate with AUF1 in the C2C12 cells, validating the interaction with MMP9 (FIG. 10G). FIG. 10G shows RNA-Immunoprecipitation of IgG (black) or endogenous AUF1 (grey) in C2C12 cells analyzed for MMP9 and ITGB1 mRNA levels.

### MMP9 Inhibition Rescues Depletion of Auf1^{-/-} Satellite Cells Following Injury

FIGS. 8A-C relate to whether MMP9, a protein involved in the break-down of extracellular matrix and healthy tissue, is more active in the AUF1^{-/-} hindlimb following injury. In particular, MMP9 is shown to be significantly more active in the absence of AUF1 in both the injured and uninjured hindlimb.

It was determined in vivo in live animals whether loss of AUF1-targeted decay of the MMP9 ARE-mRNA is in large part responsible for the post-injury muscle regeneration defect, validating that this phenotype is caused by overexpression from auf1 KO satellite cells. Live animal imaging was used to visualize MMP9 activity in auf1 WT and auf1^{-/-} KO TA skeletal muscle at 24 h post-injury in 4 month-old mice. This corresponds to a time point at which satellite cells are activated in the absence of an immune infiltrate (Dumont et al., “Intrinsic and Extrinsic Mechanisms Regulating Satellite Cell Function,” Development 142:1572-1581 (2015), which is hereby incorporated by reference in its entirety). Mice were injected intraperitoneally (IP, abdominal cavity) with an optically silent collagen matrix analog designed for selective MMP9 cleavage starting 24 hours prior to injury. Once cleaved, the matrix releases a fluorophore localized to the site of MMP9 activity. MMP9 activity at the site of repeated needle IP injections is expected. Following BaCl₂ TA muscle injury, MMP9 was strongly (>3-fold) more active in the injured TA skeletal muscle of auf1 KO mice compared to WT mice (FIG. 8A). No MMP9 activity was evident in the uninjured right hind limb control in both the WT and auf1 KO mice (FIG. 8A). Surgical excision of the injured TA muscle from WT and auf1 KO mice followed by bioluminescence imaging (FIGS. 8B and 8C) confirmed that there is an average 3-fold increase in continuous MMP9 activity in auf1 KO mice compared to the WT mice. These data indicate that activated auf1 KO satellite cells secrete continuous and increased levels of MMP9 following muscle injury, which is likely exacerbated as the satellite cell population expands.

Inhibition of MMP9 Activity in Auf1^{-/-} Mice Restores Maintenance of the PAX7⁺ Satellite Cell Population

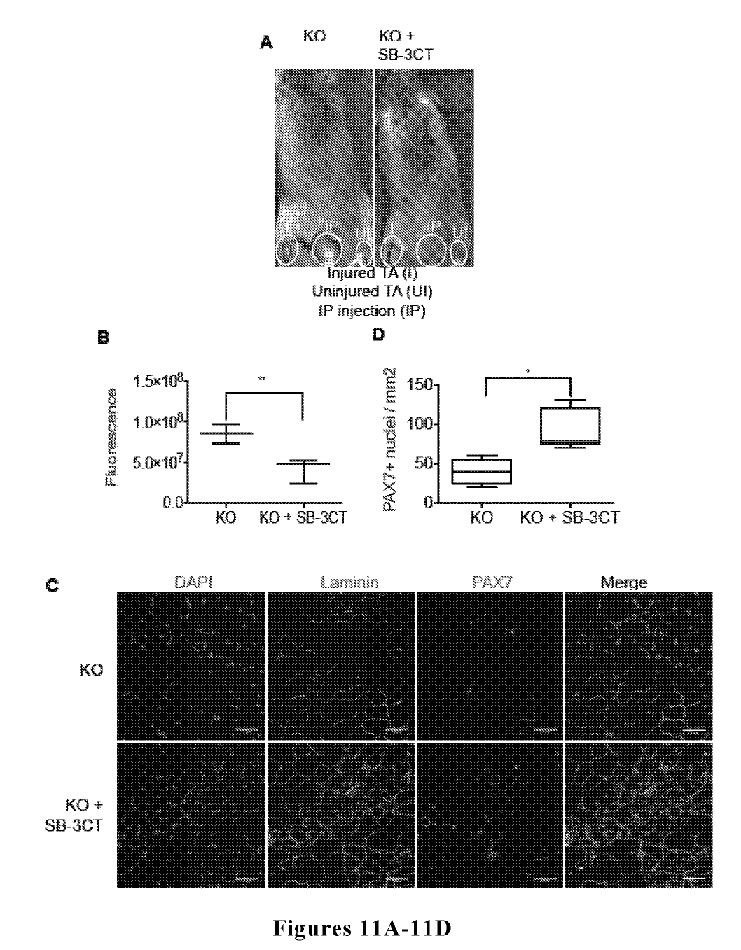

It was next determined whether the increased expression and activity of MMP9 is responsible for the auf1 KO injury phenotype observed, particularly the severe loss of laminin and depletion of the satellite cell population. Chronically increased MMP9 activity may promote excessive ECM damage and subsequent disruption of the satellite cell niche, ultimately inhibiting satellite cell return to PAX7+ quiescence by interrupting crucial cell-niche crosstalk. To test this, a MMP9 small molecule irreversible inhibitor, SB-3CT (Jia et al., “MMP-9 Inhibitor SB-3CT Attenuates Behavioral Impairments and Hippocampal Loss After Traumatic Brain Injury In Rat,” J Neurotrauma 31:1225-1234 (2014); Sassoli et al., “Defining the Role of Mesenchymal Stromal Cells on the Regulation of Matrix Metalloproteinases in Skeletal Muscle Cells,”. Exp Cell Res 323:297-313 (2014), each of which is hereby incorporated by reference in its entirety), was administered through IP injection to auf1 KO mice in conjunction with BaCl2-mediated TA injury. SB-3CT blocks MMP9 activity through an irreversible covalent interaction (Jia et al., “MMP-9 Inhibitor SB-3CT Attenuates Behavioral Impairments and Hippocampal Loss After Traumatic Brain Injury In Rat,” J Neurotrauma 31:1225-1234 (2014); Sassoli et al., “Defining the Role of Mesenchymal Stromal Cells on the Regulation of Matrix Metalloproteinases in Skeletal Muscle Cells,” Exp Cell Res 323:297-313 (2014), each of which is hereby incorporated by reference in its entirety). Mice were treated with 10 mg/kg SB-3CT daily starting 24 hours prior to injury in combination with MMP9-specific collagen matrix injections (Cai et al., “Hypoxia-Controlled Matrix Metalloproteinase-9 Hyperexpression Promotes Behavioral Recovery after Ischemia,” Neurosci Bull 31:550-560 (2015), which is hereby incorporated by reference in its entirety). Auf1 KO mice treated with SB-3CT showed near complete extinction of MMP9 activity at the site of IP injection and significantly reduced MMP9 activity in the injured TA muscle (FIG. 11A). Bioluminescence analysis demonstrated a >5-fold reduction in MMP9 activity in SB-3CT treated mice post-injury (FIG. 11B). The scale used to quantitate fluorescence is shown in FIG. 8.

The reduction in MMP9 activity by SB-3CT treatment in injured auf1 KO mice resulted in restoration of muscle wound repair. Laminin expression was strongly increased and near-normal muscle fibers were evident in injured, SB-3CT treated auf1 KO animals, consistent with repair of the satellite cell niche (FIG. 11C). Furthermore, the PAX7+ satellite cell population underwent significant increased expansion 7 days post-injury only in MMP9 inhibited (SB-3CT treated) auf1 KO mice (FIG. 11C). Specifically, a ˜4-fold increase was found in the PAX7+ satellite cell population with SB-3CT treatment following injury (FIG. 11D). These data demonstrate that the severe myopathic pathology of auf1 KO mice following skeletal muscle injury is due to loss of AUF1 targeted ARE-mRNA decay, resulting in increased and constitutive muscle tissue remodeling through elevated MMP9 activity and subsequent loss of stem cell maintenance. These findings further identify the source of late onset myopathy observed in aging auf1 KO mice—the accelerated depletion of the satellite cell population and increased degradation of laminin due to loss of AUF1-mediated regulation of ARE-mRNA decay. In both phenotypes, the source of increased MMP9 is the activated auf1 KO satellite cell, itself causing loss of self-renewal, making auf1−/− satellite cells act in a self-sabotaging manner.

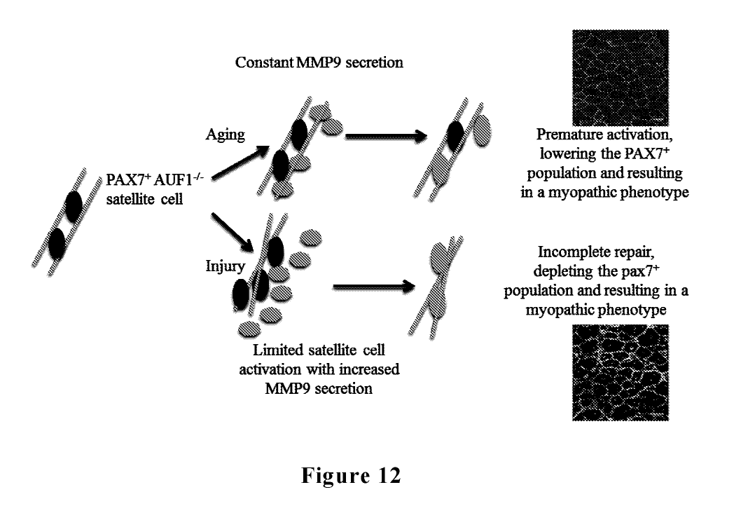

The work described here shows AUF1 regulation of MMP9 is crucial to maintaining a satellite cell population (FIG. 12). Furthermore, novel AUF1 targets are identified, indicating that late on-set myopathies have a satellite cell derived origin due to the loss or mutation of AUF1.

### Discussion of Examples 1 and 2

The targeted decay of ARE-mRNAs by AUBPs has emerged as a major regulator of many complex physiological pathways and a source of disease when it goes awry (Moore et al., “Physiological Networks and Disease Functions of RNA-Binding Protein AUF1,” Wiley Interdisciplinary Reviews RNA 5:549-564 (2014), which is hereby incorporated by reference in its entirety). AUBPs have multiple poorly understood roles in orchestrating the process of myogenesis, whether during development or regeneration following wound repair. Studies indicate that the complex and temporally ordered process of muscle regeneration, including the regulation, differentiation and restoration of satellite cells in this process, involves a tightly regulated AUBP network (Dormoy-Raclet et al., “HuR and miR-1192 Regulate Myogenesis by Modulating the Translation of HMGB1 mRNA,” Nat Commun 4:2388 (2013); Figueroa et al., “Role Of Hur In Skeletal Myogenesis Through Coordinate Regulation of Muscle Differentiation Genes,” Mol Cell Biol 23:4991-5004 (2003); Hausburg et al., “Post-Transcriptional Regulation of Satellite Cell Quiescence by TTP-Mediated mRNA Decay,” Elife 4:e03390 (2015); Legnini et al., “A Feedforward Regulatory Loop Between HuR and the Long Noncoding RNA Linc-MD1 Controls Early Phases of Myogenesis,” Molecular Cell 53:506-514 (2014); Panda et al., “RNA-Binding Protein AUF1 Promotes Myogenesis by Regulating MEF2C Expression Levels,” Mol Cell Biol 34:3106-3119 (2014); Singh et al., “Rbfox2-Coordinated Alternative Splicing of Mef2d and Rock2 Controls Myoblast Fusion During Myogenesis,” Molecular Cell 55:592-603 (2014), each of which is hereby incorporated by reference in its entirety). The individual AUBP molecular activities and coordination of their respective functions are very poorly understood, particularly in the context of stem cell mediated regeneration. Here we focused on the role of AUF1 in satellite cell mediated skeletal muscle repair, demonstrating that in the absence of functional AUF1, certain ARE-mRNAs in satellite cells are increased in abundance, disrupting satellite cell differentiation and self-renewal following wounding. The elevated expression of active MMP9, encoded by an AUF1 targeted ARE-mRNA, was found to uncontrollably degrade the surrounding skeletal muscle ECM, including laminin and the satellite cell niche, generating a myopathic phenotype similar to a variety of late onset human myopathic diseases.

The finding that auf1^{-/-} mice show accelerated skeletal muscle wasting with aging is likely a result of increased satellite cell-secreted MMP9 activity following accumulative minor wounds over time. These findings demonstrate that continuous MMP9 activity damages the laminin and ECM structures, disrupting the quiescent satellite cell niche. This results in a relentless cycle of destructive degradation and repair established by an MMP9-driven muscle wounding response, which pre-maturely activates and depletes yet more satellite cells. Activated satellite cells then fuse to existing myofibers, as indicated by the increase in centrally located nuclei in 8 month old auf1^{-/-} mice. Consequently, the loss of functional AUF1 specifically in satellite cells leads to a late onset myopathy, with no phenotype present at a young age. The chronic and increased expression of MMP9 in the absence of AUF1-mediated ARE-mRNA decay is therefore clearly a major driver of age-related and post-injury myopathy. Importantly, the disruption of the satellite cell niche by increased and unregulated MMP activity in auf1^{-/-} mice leads to the partial depletion of the quiescent PAX7⁺ satellite cell population, culminating in the development of a late onset myopathy observed in aging and following muscle injury.

Additional ARE-mRNAs other than MMP9 were identified in the satellite cell RNA-seq analysis and likely contribute to determination of satellite cell fate and the regulation of skeletal muscle integrity and regeneration. However, it is clear that AUF1 regulation of MMP9 ARE-mRNA decay defines a primary controlling step. In this regard, the ability to not only restore laminin expression, and therefore muscle regeneration, but also increase expansion of auf1^{-/-} PAX7⁺ satellite cells by treatment with the MMP9 inhibitor SB-3CT underscores the important function of AUF1-mediated decay of a single ARE-mRNA (MMP9). This further validates the importance of AUF1-regulated ARE-mRNA decay in the activation and self-renewal of satellite cells, mediated through their interaction with the niche. Future studies will be directed to understanding the role of AUF1 in later stages of muscle regeneration, including expansion, differentiation and fusion of the satellite cell. Accordingly, MEF2C, a late stage MRF and AUF1 mRNA target (Panda et al., “RNA-Binding Protein AUF1 Promotes Myogenesis by Regulating MEF2C Expression Levels,” Mol Cell Biol 34:3106-3119 (2014), which is hereby incorporated by reference in its entirety), was not identified in our RNA-seq analysis, presumably due to the time point in regeneration at which auf1^{-/-} satellite cells were selected and sorted for this study.

The chronic and increased expression of MMP9 in the absence of AUF1-mediated ARE-mRNA decay is therefore clearly a major driver of age-related and post-injury myopathy. Importantly, the disruption of the satellite cell niche by increased MMP9 activity in auf1^{-/-} mice leads to the partial depletion of the quiescent PAX7⁺ satellite cell population, culminating in the development of a late onset myopathy observed in aging and following muscle injury.

This work addresses the importance of post-transcriptional control in the coordinated process of tissue regeneration. Studies could prove extremely beneficial to further understand the multiple roles of the different AUBPs in coordinating myogenesis and muscle regeneration. Clearly, AUF1 functions at different temporal points in the process of myogenesis, shown by work in C2C12 cells (Panda et al., “RNA-Binding Protein AUF1 Promotes Myogenesis by Regulating MEF2C Expression Levels,” Mol Cell Biol 34:3106-3119 (2014), which is hereby incorporated by reference in its entirety) and here. HuR, another AUBP that often opposes AUF1 action and stabilizes ARE-mRNAs (Figueroa et al., “Role Of Hur In Skeletal Myogenesis Through Coordinate Regulation of Muscle Differentiation Genes,” Mol Cell Biol 23:4991-5004 (2003), which is hereby incorporated by reference in its entirety), increases dramatically in satellite cells in the very early stages of activation (Legnini et al., “A Feedforward Regulatory Loop Between HuR and the Long Noncoding RNA Linc-MD1 Controls Early Phases of Myogenesis,” Molecular Cell 53:506-514 (2014), which is hereby incorporated by reference in its entirety), at a time before the rise in AUF1 expression. HuR promotes the stability of certain MRFs such as myogenin and MyoD. (Figueroa et al., “Role Of Hur In Skeletal Myogenesis Through Coordinate Regulation of Muscle Differentiation Genes,” Mol Cell Biol 23:4991-5004 (2003), which is hereby incorporated by reference in its entirety). HuR was also recently shown to stabilize the non-coding RNA line-MD1, with high expression in the earliest stages of myogenesis (Legnini et al., “A Feedforward Regulatory Loop Between HuR and the Long Noncoding RNA Linc-MD1 Controls Early Phases of Myogenesis,” Molecular Cell 53:506-514 (2014), which is hereby incorporated by reference in its entirety), and the mRNA hmgb1 following injury. HMGB1 promotes a motility program involved as an early activator of the skeletal muscle repair response. (Dormoy-Raclet et al., “HuR and miR-1192 Regulate Myogenesis by Modulating the Translation of HMGB1 mRNA,” Nat Commun 4:2388 (2013), which is hereby incorporated by reference in its entirety). Yet another AUBP, TTP, which is also an ARE-mRNA decay mediator, is highly expressed in only quiescent satellite cells, when AUF1 is not expressed. Furthermore, TTP shows immediate inactivation following injury when AUF1 expression increases dramatically. (Hausburg et al., “Post-Transcriptional Regulation of Satellite Cell Quiescence by TTP-Mediated mRNA Decay,” Elife 4:e03390 (2015), which is hereby incorporated by reference in its entirety). In the quiescent satellite cell, TTP mediates the rapid decay of the MyoD mRNA, preventing expansion of the satellite cell population. Previous studies have shown that AUF1 and TTP tend to show mutually exclusive expression or activity (Moore et al., “Physiological Networks and Disease Functions of RNA-Binding Protein AUF1,” Wiley Interdisciplinary Reviews RNA 5:549-564 (2014), which is hereby incorporated by reference in its entirety), consistent with these findings and our data that AUF1 is only expressed following satellite cell activation.

Reported data lead to the possibility that loss or mutation of AUF1 is related to the development of LGMD, a late onset human myopathy. Multiple family cohorts with LGMD type 1G have a mutation in the 4q21 locus which contains the auf1 gene, and one family was shown to have a mutation in HNRNPDL, a poorly described AUF1 homolog (Starling et al., “A New Form of Autosomal Dominant Limb-Girdle Muscular Dystrophy (LGMD1G) with Progressive Fingers and Toes Flexion Limitation Maps to Chromosome 4p21,” European J. Hum Gen 12:1033-1040 (2004); Vieira et al, “A Defect in the RNA-Processing Protein HNRPDL Causes Limb-Girdle Muscular Dystrophy 1G (LGMD1G),” Hum Mol Genet 23:4103-4110 (2014), each of which is hereby incorporated by reference in its entirety). The age of onset for LGMD type 1G ranges from 30-47 years with no childhood history of myopathy. (Starling et al., “A New Form of Autosomal Dominant Limb-Girdle Muscular Dystrophy (LGMD1G) with Progressive Fingers and Toes Flexion Limitation Maps to Chromosome 4p21,” European J. Hum Gen 12:1033-1040 (2004); Vieira et al, “A Defect in the RNA-Processing Protein HNRPDL Causes Limb-Girdle Muscular Dystrophy 1G (LGMD1G),” Hum Mol Genet 23:4103-4110 (2014), each of which is hereby incorporated by reference in its entirety). As clinically described, LGMD disease shows a similar relative age of onset and histological representation as identified in the auf1^{-/-} mouse.

This work is the first to identify a myopathy of true satellite cell origin in an animal model and places the AUBP mRNA decay protein AUF1 as a key regulator of adult stem cell fate. These findings have important clinical implications. While healthy skeletal muscle can develop in the absence of functional AUF1, the satellite cell population is clearly altered and, once activated, is quickly depleted. Activated auf1^{-/-} satellite cells secrete elevated levels of MMP9 that continuously breaks down the ECM and niche, causing premature satellite cell activation, satellite cell depletion, and subsequent development of myopathy with age. Consequently, a combination of MMP9 inhibition and potential AUF1-medated satellite cell therapy has a role in regenerative medicine for chronic and acute adult myopathies.

### Prophetic Example 3—Therapeutic Approach

Isolated Satellite Cells

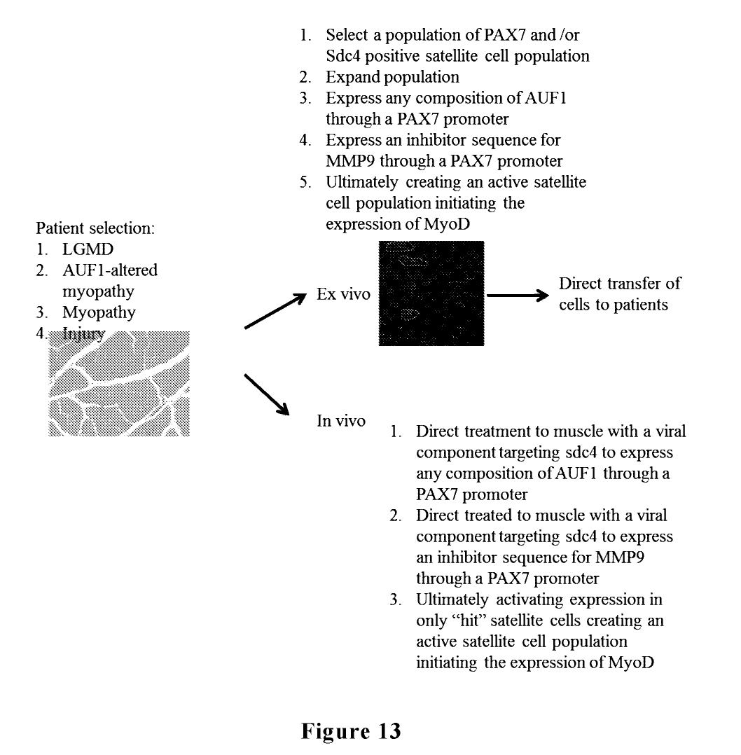

Satellite cells will be isolated from patient or donor biopsies using a Sdc4+CD45-Sca1-FACS model. These cells will be treated with a virus construct to overexpress the four isoforms of AUF1, or any of the four AUF1 isoforms or combinations thereof, and a virus construct to silence MMP9. Both will be under the promoter of PAX7, making their expression limited to the active satellite cell. Treated cells will then be re-implanted into myopathic tissue or site of muscle injury (FIG. 13).

Direct Skeletal Muscle Virus Injection

A mix of virus constructs to overexpress the four isoforms of AUF1, or any of the four AUF1 isoforms or combinations thereof, and virus constructs to silence MMP9 would be directly injected to the site of myopathy of muscle injury. Both will be under the promoter of PAX7, making their expression limited to satellite cells but shut off once cells enter differentiation.

### Prophetic Example 4—Validating Satellite Cell-Mediated Regenerative Therapy

Validating the efficacy of a satellite cell-mediated skeletal muscle regenerative therapy can be accomplished in a murine model experiment. Male 4 month old C57BL/6J mice, or a comparable non-transgenic inbred strain, is divided into two cohorts: source of satellite cells and subject for therapy validation.

On experiment day 1, the therapy validation cohort will receive injury to one tibialis anterior muscle, leaving the contralateral muscle as an uninjured control. Injury would be induced by injection of 20 μL of sterile 1.2% BaCl₂ saline solution while mice are temporarily anesthetized by isoflurane.

On experiment day 2, the source of satellite cell cohort will be sacrificed and both hindlimbs will be removed for complete skeletal muscle isolation and digestion. This digested skeletal muscle will be stained for fluorescence-activated cell sorting with the following channel markers (1) Sdc4 (2) CD45, Sca-1. Cells that are positive for channel 1 and negative for channel 2 will be selected and cultured in appropriate conditions (Bernet et al., “p38 MAPK Signaling Underlies a Cell-autonomous Loss of Stem Cell Self-renewal in Skeletal Muscle of Aged Mice,” Nature Medicine 20:265-271 (2014), which is hereby incorporated by reference in its entirety). Once in culture, these cells will be treated by any method claimed for increased expression of AUF1 and silencing of and combination of MMP9, Twist1, or Cyclin D1.

Following treatment, the satellite cell population will be injected into the injured TA of mice. Injured and uninjured TAs will be removed and frozen in OCT at 7 and 14 days post-injury (Gunther et al., “Myf5-positive Satellite Cells Contribute to Pax7-dependent Long-term Maintenance of Adult Muscle Stem Cells,” Cell Stem Cell 13:590-601 (2013), which is hereby incorporated by reference in its entirety).

Regeneration will be validated through immunofluorescence. Samples will be post-fixed in 4% paraformaldehyde and blocked in 3% BSA in TBS-T (Lepper et al., “Adult Satellite Cells and Embryonic Muscle Progenitors have Distinct Genetic Requirements,” Nature 460:627-631 (2009), which is hereby incorporated by reference in its entirety). The following primary antibodies will be incubated at 4° C. overnight: Laminin to identify skeletal muscle fiber regeneration, PAX7 to identify the satellite cell population, and AUF1 to identify increased AUF1 expression specifically in the satellite cell. Additional staining would be completed for any genes that are silenced. Alexa Fluor 488, 555, and 647 secondary antibodies will be used at 1:500 and incubated for 1 hour at room temperature. Slides will be sealed with Vectashield with DAPI.

Images will be acquired through confocal microscopy. To address satellite cell specificity, images will be analyzed for co-localized expression of PAX7 and AUF1 and/or any combination of MMP9, Twist1, and Cyclin D1. To address regeneration, images will be analyzed for laminin fiber development and size.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A composition comprising:
a nucleic acid molecule encoding an AUF1 protein or a functional fragment thereof, and
a targeting element which controls muscle satellite cell-specific uptake or expression, wherein the targeting element is heterologous to the AUF1 gene.

2. The composition according to claim 1 further comprising:
a buffer solution.

3. The composition according to claim 1, wherein the composition comprises a plasmid comprising the nucleic acid molecule.

4. The composition according to claim 1, wherein the nucleic acid molecule comprises the targeting element.

5. The composition according to claim 4, wherein the targeting element is a muscle satellite cell-specific promoter.

6. The composition according to claim 5, wherein the promoter is a Pax7 promoter, MyoD promoter, or a myogenin promoter.

7. The composition according to claim 1, wherein the targeting element is a binding partner for a muscle satellite cell surface protein.

8. The composition according to claim 7, wherein the composition is contained within a vesicle and the vesicle contains the binding partner on its surface.

9. The composition according to claim 7 or 8, wherein the satellite cell surface protein is Syndecan4.

10. The composition according to claim 1, wherein the composition comprises a viral vector comprising the nucleic acid molecule.

11. The composition according to claim 10, wherein the viral vector is a lentivirus, adenovirus, or adeno-associated virus vector.

12. The composition according to claim 11, wherein the viral vector is an adeno-associated virus vector.

13. The composition according to any of claims 1-12 further comprising:
one or more of an MMP-9 inhibitor, a Twist1 inhibitor, or a cyclin D1 inhibitor.

14. A composition comprising:
a muscle satellite cell population, wherein the cell population comprises a transgene exogenous to the satellite cells and encoding AUF1 protein or a functional fragment thereof.

15. A composition comprising:
a muscle cell population comprising an AUF1 gene encoding AUF1 protein or functional fragment thereof, wherein expression of the AUF1 gene is controlled by a promoter heterologous to the AUF1 gene.

16. The composition according to claim 14 or 15, in which the cell population expresses the AUF1 protein or functional fragment thereof.

17. The composition according to claim 14 or 15, wherein the cell population is Syndecan 4⁺/PAX7⁺.

18. The composition according to claim 13, wherein the cell population is Syndecan 4+/PAX7⁻.

19. The composition according to any of claims 1-13 or the cell population according to any of claims 14-18 further comprising:
one or more of an MMP-9 inhibitor, a Twist1 inhibitor, or a cyclin D1 inhibitor.

20. A pharmaceutical composition comprising:
(a) one or more of an MMP-9 inhibitor, a Twist1 inhibitor, or a cyclin D1 inhibitor;
(b) a targeting element that causes muscle satellite cell-specific uptake or activity of the one or more inhibitors; and
(c) a pharmaceutically-acceptable carrier.

21. The pharmaceutical composition according to claim 20 further comprising:
an AUF1 protein, a functional fragment of AUF1 protein, an AUF1 protein mimic, or a combination thereof.

22. A method of producing a muscle satellite cell population comprising:
transforming or transfecting Syndecan 4⁺/PAX7⁺ or Syndecan 4⁺/PAX7⁻ muscle satellite cells with a nucleic acid molecule encoding exogenous AUF1 or a functional fragment thereof under conditions effective to express exogenous AUF1 in the muscle satellite cells.

23. The method according to claim 22, wherein the method is carried out ex vivo.

24. The method according to claim 23 further comprising:
culturing the muscle satellite cells ex vivo under conditions effective to express exogenous AUF1.

25. The method according to claim 22, wherein the method is carried out in vivo.

26. The method according to claim 22, wherein the Syndecan 4⁺/PAX7⁺ or Syndecan 4⁺/PAX7⁻ muscle satellite cells are AUF1 deficient.

27. The method according to claim 22, wherein the Syndecan 4⁺/PAX7⁺ or Syndecan 4⁺/PAX7⁻ muscle satellite cells are transformed with the nucleic acid molecule encoding exogenous AUF1 or functional fragment thereof.

28. The method according to claim 22, wherein the Syndecan 4⁺/PAX7⁺ or Syndecan 4⁺/PAX7⁻ muscle satellite cells are transfected with the nucleic acid molecule encoding exogenous AUF1 or functional fragment thereof.

29. The method according to claim 22, wherein the Syndecan 4⁺/PAX7⁺ or Syndecan 4⁺/PAX7⁻ muscle satellite cells are AUF1 sufficient.

30. The method according to claim 22 further comprising:
contacting the cell population with one or more of an MMP-9 inhibitor, a Twist1 inhibitor, or a cyclin D1 inhibitor.

31. A muscle satellite cell population produced by the method according to claim 22.

32. A method of causing satellite-cell mediated muscle generation in a subject, the method comprising:
selecting a subject in need of satellite-cell mediated muscle generation and
administering to the selected subject
(i) the composition according to any one of claim 1-21 or 30,
(ii) the cell population according to claim 31,
(iii) AUF1 protein, a functional fragment of AUF1 protein, an AUF1 protein mimic, or a combination thereof, or
(iv) a combination of (i), (ii), and (iii),
under conditions effective to cause satellite-cell mediated muscle generation in the selected subject.

33. The method according to claim 32, wherein the subject has a muscle injury and said administering is carried under conditions effective to treat the muscle injury by causing satellite-cell mediated muscle regeneration.

34. The method according to claim 32, wherein said administering is carried out by injection of (i), (ii), (iii), or (iv) into muscle in the selected subject.

35. The method according to claim 33, wherein the muscle injury is a myopathy or muscle disorder mediated by functional AUF1 deficiency.

36. The method according to claim 33, wherein the muscle injury is a myopathy or muscle disorder not mediated by functional AUF1 deficiency.

37. The method according to claim 33, wherein the muscle injury is an adult-onset myopathy or muscle disorder.

38. The method according to claim 37, wherein the adult-onset myopathy or muscle disorder is a Limb-Girdle Muscular Dystrophy (LGMD).

39. The method according to claim 33 further comprising:
administering to the selected subject one or more of an MMP9 inhibitor, a Twist1 inhibitor, or a cyclin D1 inhibitor.

40. The compositions, cell populations, or methods according to any of claim 1-19 or 21-39, wherein the AUF1 protein is one or more of p37^{AUF1}, p40^{AUF1}, p42^{AUF1}, or p45^{AUF1}.

41. The compositions, cell populations, or methods according to any of claim 1-19 or 21-39, wherein the AUF1 protein is p37^{AUF1}.

42. The compositions, cell populations, or methods according to any of claim 1-19 or 21-39, wherein the AUF1 protein is p40^{AUF1}.

43. The compositions, cell populations, or methods according to any of claim 1-19 or 21-39, wherein the AUF1 protein is p42^{AUF1}.

44. The compositions, cell populations, or methods according to any of claim 1-19 or 21-39, wherein the AUF1 protein is p45^{AUF1}.

45. The composition according to claim 13, 19, 20, or 21 or the method according to claim 30 or 39, wherein the inhibitor is a nucleic acid molecule, a polypeptide, or a small molecule.

46. The composition according to claim 45, wherein polypeptide is an antibody.

47. The composition according to claim 46, wherein the antibody is a bispecific Pax7/MMP-9 antibody.

48. The composition according to claim 45, wherein the inhibitor is a nucleic acid molecule effective in silencing expression of MMP-9, Twist1, cyclin D1, or a combination thereof.

49. The composition according to claim 48, wherein the nucleic acid molecule encodes an endonuclease for targeted alteration of gene(s) encoding MMP-9, Twist1, cyclin D1, or a combination thereof.

50. The composition according to claim 49, wherein the endonuclease is a ZFN, TALEN, or CRISPR-associated endonuclease.

51. The composition according to 45, wherein the nucleic acid molecule encodes an antisense form of at least a portion of a nucleic acid molecule encoding MMP-9, Twist1, or cyclin D1.

52. The composition according to 45, wherein the nucleic acid molecule comprises an antisense form of at least a portion of a nucleic acid molecule encoding MMP-9, Twist1, or cyclin D1.

53. The composition according to 45, wherein the nucleic acid molecule comprises a first segment encoding MMP-9, Twist1, or cyclin D1 and a second segment in an antisense form of the first segment.

54. An in vivo method of producing a muscle satellite cell population expressing exogenous AUF1 or a functional fragment thereof, the method comprising:
transforming or transfecting Syndecan 4⁺/PAX7⁺ or Syndecan 4⁺/PAX7⁻ muscle satellite cells with a nucleic acid molecule encoding exogenous AUF1 or a functional fragment thereof, wherein when Syndecan 4⁺/PAX7⁺ or Syndecan 4⁺/PAX7⁻ muscle satellite cells are transformed or transfected in an in vitro or an in vivo model with the nucleic acid molecule they express the exogenous AUF1 or the functional fragment thereof.

55. The method according to claim 54, wherein the in vivo muscle satellite cell population causes muscle satellite cell regeneration, and wherein said regeneration occurs in an in vitro or in vivo model.

56. A method of treating a subject in need thereof with Syndecan 4⁺/PAX7⁺ or Syndecan 4⁺/PAX7⁻ muscle satellite cells expressing exogenous AUF1 comprising:
administering Syndecan 4⁺/PAX7⁺ or Syndecan 4⁺/PAX7⁻ muscle satellite cells transformed or transfected with a nucleic acid molecule encoding exogenous AUF1 or a functional fragment thereof, wherein the Syndecan 4⁺/PAX7⁺ or Syndecan 4⁺/PAX7⁻ muscle satellite cells express the exogenous AUF1 or the functional fragment thereof in an in vitro or an in vivo model.

57. The method according to claim 56, wherein said administering is effective to cause satellite-cell mediated muscle regeneration in the subject, and wherein said regeneration occurs in an in vitro or in vivo model.

58. The composition according to any of claims 1-13, the pharmaceutical composition according to any of claims 20-21, or the cell population according to any of claims 14-19 further comprising:
one or more of an IL17 inhibitor, an MMP-8 inhibitor, an IL10 inhibitor, an FGR inhibitor, a TREM1 inhibitor, a CCR2 inhibitor, an ADAM8 inhibitor, or an IL1b inhibitor.

59. The method according to claim 22, 30, 33, or 39 further comprising:
contacting the cell population with one or more of an IL17 inhibitor, an MMP-8 inhibitor, an IL10 inhibitor, an FGR inhibitor, a TREM1 inhibitor, a CCR2 inhibitor, an ADAM8 inhibitor, or an IL1b inhibitor.

60. A pharmaceutical composition comprising:
(a) one or more of an IL17 inhibitor, an MMP-8 inhibitor, an IL10 inhibitor, an FGR inhibitor, a TREM1 inhibitor, a CCR2 inhibitor, an ADAM8 inhibitor, or an IL1b inhibitor;
(b) a targeting element that causes muscle satellite cell-specific uptake or activity of the one or more inhibitors; and
(c) a pharmaceutically-acceptable carrier.

61. The pharmaceutical composition according to claim 60 further comprising:
an AUF1 protein, a functional fragment of AUF1 protein, an AUF1 protein mimic, or a combination thereof.

62. The composition or cell population according to claim 58, the pharmaceutical compositions according to claim 58, 60, or 61, or the method according to claim 59, wherein the inhibitor is a nucleic acid molecule, a polypeptide, or a small molecule.


SEQUENCE LISTING (61 sequences)

SEQ ID NO: 1 - DNA, 2053 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 2 - protein, 287 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 3 - DNA, 6607 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 4 - protein, 287 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 5 - DNA, 2110 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 6 - protein, 306 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 7 - DNA, 6664 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 8 - protein, 306 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 9 - DNA, 2200 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 10 - protein, 336 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 11 - DNA, 6754 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 12 - protein, 336 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 13 - DNA, 2257 bp, Homo sapiens
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 14 - protein, 355 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 15 - DNA, 6811 bp, Mus musculus
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 16 - protein, 355 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 17 - DNA, 14654 bp, Homo sapiens
ggccaggagtttaagactagcctgggcaacgtggcgaaaccctgtctctacaaaaaaacaaaaattagctgagcttgatggagcacacctgtagtcccagatactcagaaggctgaggtcgaaggatggcttgaacccagaggcggaggctgcagtgagcctagatcacatcattgcactccagcctgggtgacagagtgagaccctgactcaaaaaaaaaaccccaaaaaacaaaaaaacctccacttgactgttctctgccctaccagctacctcactcttattttccttttttatctccaacttttctttcctacagatttcagccaaataactcacatctgttccctctgttggttcctaaacataactcctccagcctggcttctgggcaatcacccctccctgcccctagactgctgcccccatggacttcctttttgcgagagccagtgacctgatcttagcacgggattgggagtcagaaggcctaggcagctggtcactcaccagctgtgtgaccgtgagcaacttaaagcacttctctgagccatgggtatgaacgttaatttgcaaagttgagatgagagcattagagtaatagaatcataatagctaaactgtgtgtgtgtgtgtgtgtgtacctgctaagagccaggcactgtgctgagaatttgccatgtataatcacatttaatccccactacaacacctccaaatgaggtctgcttctttccccactttacaaatgagaaaagggaggcacagagaggtaaagtgatttgcctaaagtcccacagcccataaatgataggtagtagaatatgaacccaggaggtctgattccaaaatttatgccctatggcttccttactgacggtgctcagtacagataagtgggaggtgtgacttggtgttaatagggactctaagagtcagtgtgggaattgccattcccctgaagctagggatggcaggtgtgtttgtgggttgggggtggggtacttgagtgatgctctcccctgaggcaatgtcttgcaggatacacacctttctacttcatctcagatttcagtgtctcctccacatcctgccagagcctctctggcctggctgcccctcccctctctggcaggtttgggagaaagaggcttggagttcccccatcctagatagcgggacagattcctcatctcgtgaggctgaccctgatctgggggcctgttattaataatggcctgcagggctgtgtcatccagctagtgattcaggactccagggtcagcaccctccccatcctgatggagctacctcagtgaggggcttagagtctaagctatgactctacgtgagggtatatgctgttctctgcctgcactcccagggtcttggctaaactctattgtggtattaaaaagagaggctgggggaagtggggaaggggatgtggtaaccagaaccacttcctgccactgcagtgttgaaagccaagctctgtccaagttccccttgatgggggttggggagccaggaggtaaaggaggaagttgatgggagttgggggggatgttaaaaaaaggaggattcttatttggattgactccaggaatcaagatctgggtttcctggctgctctctccctgctcccatttggtaaaaaacagcggctgtcctgaaggtggtcctaattagcatagcagtgaacacttattatcatgtgccaggcactttatatttgctaacactttgagccttacaaccaccccatgaaggaagcactattattactccaattttacagatggggaaactgaggcactgaaatgttgtgtcagttgcccttgtcacacagcaagtcagtggcagagctgagatttgaacttaggcagcctggctccagagtgcaggctcttgctctgtgaccactctgagcctcagtttcctcattgcatgatgagggtaattggaatacataaatccactttcctttcttgatgcctgattctctggtaattaattattcattcaatttctccccatgctgaactctcagatccatgaacaatgagacaaaggctatcttggtcactacttgtattcccaattcttagcataatgattggcctagaaaaactcaactagaatgtgttgagtaaatggtctttaaaaggtctgaagatcatcaacaatggtttgagtatctgataaaagttttagaccctttacatagaaaaatcaacatataccagtagctgggggtgagtctgcccctcccactcacccagggtacatttggcaatgtttggtaacatttctggttgtcacaactggcagggggaggtgctactggcatccagtgggtaggggtcagggacgctgctaaacatcctacaatgcacacgacagccccaagtgccaatagtgctaagtttgagctatgtgacatacatacatagatatgtattgtatgtcatgtttggtaccattgatggcaagtggggggtagttaacggattttcctaaaagcccgtgtctgccacccagatgaagatcctccagattggaagatctgttcctctaaactcatattactctatgacctcacattcccgagtctagaatctagttcctcctgggtcccctgtcctagcctctgattccctaaatgcctgggcctcctctcttaccttcagtgggtagcaggggaccagaggagaagcatccaggttttatgtgctgcccctgtcaccgcatccacctacccacttctatacctgggtcatcacagttccctgtaaatggtaataaagatgaaaagcttcagagccaggcagttctgggcttgaacactagttctgtggattaactcgctctgtgatcacaggcaaattccttaactctctgagccttagtttccccctctgaaaacaggagggatactcattaaacttaccttacaggtggtgaggatgaaacgagaggcttatagagaacttattacggtgcttgacacagtaaatctcaaaaaatgcattattattattatggttcagaggtaaagtgacttgcccaaggtcacatagctggaaaatggcagagccgggatggaaatccaggacttcgtgactgcaaagcagatgttcattggttagtgaactttagaacttcaacttttctgtaaaggaagttaattatctccatctcacagtctcatttattagataagcatataaaatgcctggcacatagtaggccctttaaatacagcttattgggccgggcgccatggctcatgcccgtaatcctagcactttgggaggccaggtgggcagatcacttgagtcagaagttcgaaaccagcctggtcaacgtagtgaaaccccatctctactaaaaatacaaaaaatttagccaggcgtggtggcgcacgcctataataccagctactcgggaggctgaggcaggagaattgcttgaacccgggaggcagatgttgcagtgagccgagatcacgccactgcactccagcctgggtgacagagtgatactacaccccccaaaaataaaataaaataaataaatacaactttttgagttgttagcaggtttttcccaaatagggctttgaagaaggtgaatatagaccctgcccgatgccggctggctaggaagaaaggagtgagggaggctgctggtgtgggaggcttgggagggaggcttggcataagtgtgataattggggctggagatttggctgcatggagcagggctggagaactgaaagggctcctatagattattttcccccatatcctgccccaatttgcagttgaagaatcctaagctgacaaaggggaaggcatttactccaggttacactgcagcttagagcccaataacctggtttggtgattccaagttagaatcatggtcttttggcagggtctcgctctgttgcccaggctggagtgcagtgacataatcatggctcactgtatccttgaccttctttctgggctcaagcaatcctcccacctcggcctcccaaagtgctaagattacaggaatgagccaccatacctggccctgaatcttgggtcttggccttagtaattaaaaccaatcaccaccatccgttgcggacttacaacctacagtgttctaaacattttatatgtttgatctcatttaatcctcacatcaatttagggacaaagagccccccaccccccgtttttttttttacagctgaggaaacacttcaaagtggtaagacatttgcccgaggtcctgaaggaagagagtaaagccatgtctgctgttttctagaggctgctactgtcccctttactgccctgaagattcagcctgcggaagacagggggttgccccagtggaattccccagccttgcctagcagagcccattccttccgcccccagatgaagcagggagaggaagctgagtcaaagaaggctgtcagggagggaaaaagaggacagagcctggagtgtggggaggggtttggggaggatatctgacctgggagggggtgttgcaaaaggccaaggatgggccagggggatcattagtttcagaaagaagtctcagggagtcttccatcactttcccttggctgaccactggaggctttcagaccaagggatgggggatccctccagcttcatccccctccctccctttcatacagttcccacaagctctgcagtttgcaaaaccctacccctcccctgagggcctgcggtttcctgcgggtctggggtcttgcctgacttggcagtggagactgcgggcagtggagagaggaggaggtggtgtaagccctttctcatgctggtgctgccacacacacacacacacacacacacacacacacacacacacacacaccctgacccctgagtcagcacttgcctgtcaaggaggggtggggtcacaggagcgcctccttaaagcccccacaacagcagctgcagtcagacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggtgggcaaacacctagtctagagttggggagggctgtccgtgagggtgttgagtgtcccagagaggatgcagggcctcagaggagatgctttaggggtgtgttggtggtgatgggcgtatctgaagaacagaggtgtccagggttaggcagtggggggtcttgtggaggctttgagcagtgatggccagaaatgggcaatggggctttcctaggtgggaaatgggaaatggtttggggtgggggaggcattggagggttctggggtaagcataggctgggagtgaacaggggcaaaccttatgcagctgtggggtagaaatgggctagaggcatccaggggtgagaaggagctgaggatgtctaaggaggggagatccctgggtggtcagaaagcactggtgtctggaaagcatttaatgctttattaaatgttagtccctgctgggcatgacggctcacacttgtaatcccagcactttgggaggctgaggtggtaggatcgctgaagctcaggagtttgagcccagcctaggcaacatagtaagatcctgtctctacaaaaaaattaaagaaatagccaggcacagtgatgtgcacctgtagttccagctatgcagaaggctgagatgggaggatcgcttgagtccaggaggtccaggctgcagtgggctgataccgtctctccgaaaaagaaaaagaaaaaagactccctccatgagtgtctggagggagtcctttggccccagctgggcagagaaaggggtcagagatctggcatgtgtgtgtcccttcatccacaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggtgagccggggccgtgggggcagcggggtggggcggggaggccaggtctggctcttgggccagcggtgaacatgtcctgtcttggacgcgtccctgggtttcactatttaatgtgtggcccctggggagtgtccccacctctgagcctctgtttctccttcagggaaatggctcttgcaatccaagtcctcctgccagggccattgtgagggtctaagtagacaaaaaaaaaaaaaaaaaaaacagtctggaagcaatttatagatgagagcgtggacggcagagagcattgtgtatgttgaagtctctgcgatatggggtgtccctgctgccccgctccagcctttcacttctgacctccttcctctggctcttacgctacaggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcgggtgagaacgtgaggagggaaaatccaagagacctgggcggggtcagggaagggaggaccacggagagcgtggaggcagcagtggccccggcttcctcttgcctgcccgcgctgccctggcttatacggcccctcctgccagacagtgcacagggccagggcgccaggctgggagagcttcgcgcaggcgggatttcagcccgcacttatttcggagcccttgccttgggcagcgcacaatctgcgcagcagtactcggctaaccctcttcctctcgacctgtttcttcagagcacggagacgggtatcccttcgacgggaaggacgggctcctggcacacgcctttcctcctggccccggcattcagggagacgcccatttcgacgatgacgagttgtggtccctgggcaagggcgtcggtgagattctgagtcctcctggcccctgattcccttcattctctcccactcatcacccgccgccctaactccggtcccccctcctcctgcagtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagtgagtgagggggctcgccgagggctgggggcgcccaccacccttgatggtcctgggttctaattccagctctgccactagtgctgtgtggcctgcaattcaccctcccgcactctgggcccaattttctcatctgagaaatgatgagagatgggatgaactgcagaccatccatgggtcaaagaacaggacacacttgggggttataatgtgctgtctccgccttctccccctttcccacatcctcctcgccccaggactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgaggtacctccaccctgtctaccaggttcagccccgccctctcatcatgtattggcccccaaaacgcggctcttccctcccatcagtttgtctttccactctcattggtcctcaggacgaccgtgactccgcccacctacaccacatttccaccactatccctgacttccaatggccccgccccagccactaaggttcggccttttctgcccagctggccgcctcttccttggtctggtgtcccaggcaccgcccacgggtctagcctcttctcaggagtgctctacagcgccccctaggccaccaagattgtttagctccctgtcgggtcggcccctgactccttattggactcatccatctggctcatccaaggccttgggtctctccagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggtaggcgtggtcccgcggctccggggctggggttcccggcagtggtggtggtggggtggccagggctgggggctcggcccggcgctcacgtctcaggctccctctccctccaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtgaggcaggggcagggatgggaggaggaggggaaagggcgtggctgtgccacagtaccaaagaattgggggttggggatcgggggaggaacggggcgtgcaggagaggtgggacctcaacgtctgtctggaagcagagcctgggcccagtcgctgccatgtcagtgcttagaggtggtgataaagagactctagagagagataggtgtgacttcaaaagccagtctactctgggcatggtggctcacgcctctaatcccagggctttgggagacccaaggcgggaggattgcttaagcccaggagttccagaccagcctcggcaacatagccagactcccatctctacaaaaaataaatgagcaaggcgtgaaggcacatgtctgtagtcctagctactctggaggctgaggtgggaggatctcttgagcccaggagttcgaggctgtagtgagctatgattgcaccactgcattccatcctgggccatagaggatgtcgcttaaaacgaaaaagaagaagaagaaagtcctgtggtttgggaagggaggctgagtgaggaggggcctgtgtgccagaggaggcttcactgagaagcttaggggagcagatgttctaggggtacagaggtatgcaggaataggaagagtctcaccccgtgtctctttttaggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggtgaggaggcggggttgtgtggatgcgggagggggctttgcggaggggctgcccgtcccttcccgcccactggccctgtgtccaaggcttagagcccgtcctttccctcctcgctttctcaggaagtactggcgattctctgagggcagggggagccggccgcagggccccttccttatcgccgacaagtggcccgcgctgccccgcaagctggactcggtctttgaggagcggctctccaagaagcttttcttcttctctggttagttacctactttccctcccccgcccggtcaatccccatcagtcaaggaggctcaagagaccatcgataacccacgaaacgtcttgtgcgttttagaaaaatacgccccctggcggacgcagtttagcaaacgtaggggcggctgagtttctgccccctcctctccacgccctcgcgtcgctctacccagcgcctctgcccctgggttgcagggactgcgggcacgcgggctaggaaaggcctcgccggaatctccctcctcgcgttctaggagtacgtgctccctctgcgcccccaaaccgacgtgaccctcctcccctgcagggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggtgagcgccgccgcggccgccggcagggggagcccgggcgccgtcggtccgtccgctagccggctcagcacctgtctcctccgcgcctgcccgcaggttcgacgtgaaggcgcagatggtggatccccggagcgccagcgaggtggaccggatgttccccggggtgcctttggacacgcacgacgtcttccagtaccgaggtgagggctgaggaggatcccttcgtgagacaccacactaagctcctcttagtgagtggtcaaattctgagcgaggaagaaaaagcccttggaaatggaaacaaatgccccagcacagacaagatcccagcagaggcagaggccttctccaggtcatttaggaagtcagggatgcaaccaagaccaggacccagatttcctgcctccccggctggaagctctttctccttcagtacaggacggcaggtggtttgtatggaagctcagttattagacaacagtcatcaagtgccgataatgtgccaggcactgtgctacagggagagataagacaattcacagctctgtgactttgggcaagtcactgcttctctactcttcgagcctcagtttccccatctgtaatatggggactatagctggaattacacttgacttccctttcttaccagtcacatccaaacagttgacaaggtgaacaagatttcctgccaccaaaatctttttcgagtctgtcattttttttgccatcttctttataaacaccccagcccaaaccatactggctgtccaggacctttaacaaattccatgagattagagagggggtaggagtgaagggcaatggtcttgggagtgaccccagatgaattccaaggtcaaagaaattaagaggatctgacactccacccccgtgttctcatctcttcccactcctcctgttatttactctgctccacccacactggctgctctttgaacagatcaaggtcattcctagcttacagcctttgtgccagttgttccctctgtctggaaagcttcccctccagattgtcactgggccatcccactgtcttccttcaggtttcagtgctaaggccattgcttcaatgaggccttctttgatgcttattatctatttacttgtttttattttctccatagctttctatattttctttttttttcttttttctttttttttttttttgagatggagtcttgctctgtcgcccaggctggagtgcagtggcacgatctcagctcactgcaacctccgcctcccgggttcaagcgattctcctgcctcagcctcccaagtagctgggattacaggtgcctgccaccacgcttggctaattttttgtattttttagtagagacggggtttcaccatcttggccaggctggtcttgaactcctgacctcgtgatccacccgcctcagcctcccaaagtgctgggattacaggcatgagccaccgcacccagccgctttctatattttcaaaaccaatctcatttatttatgtgtttgcttaattgtctcttgcctcactagagtgtaagcaccaagataattgagatcatgcctgcattttttctgcttatccccagtatcttgaacaaagcacatagtagatgctcagtaaatgatgaatgaacagatttgttcaatgaatgagcgttgaatgaattgttctgagcattaagatagttggtctattcatttgttaattcattcacaaaatgtgtatggtgtacctactgtgtgctaggctctgtggcagtgctttgggcactgaggtctgtgccctccagcatctcacagaacctcacagcatctcacaggttggggggatggaggtgatatgtgaaaaccttagaaagttctagaaatggcagaagagatggttgtcaagatcttgttcctatttctgtatatgtgggagaattagaatcactcctcttatgcctgcctgtctcctgcagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttagaagcagactttatttatatatgtatgcacgtatgtatgcatgtatgtatttaactgatagagtgcaaaaaaaaaaaaaaaaaggaaaaacaaataactgatagagtgctttctacgtgccagaaagtgttctaggccgggcacggtagctcactcctagcactttgggaggccgaggcaggcggatcacgaggtcaggagattgagaccaccctggctaacacgatgaaaccctgtctctactaaaaaaaaaatagaaaaaattagccgggcgtggtggcgggcgcctgtagtcccagctacttgggaggctgaggcaggagaatggcttgaacctgggaggtggagcttgcagtgagccgagatcacgccactgcactccagcctgggaggtggagcttgcagtgagccgagatcacgccactgcactccagcctgggtgactgagcaagactccgtctcaaaaaaaaaaaaaatagtgttctaggcactttgtaaatgttaacatattcaatcattcctgttaggaaagtatgatgtgattatttctattttacagtcaaggaaatgatcaacctgtttattcattcatcaaacatttattgagcccctacatggagccaggccctgtactgggcaatggggatagagaaatgagttagactttagaatgcataagattccccttggaacttgcttaaaatgcaactccaggctccacctccagagagtctgacctatttacaagggtgattctatggctggtggcggtgggatcacacttggggagtgtgcagtgcatgatcttttattctacaataatggtgtgtgtgtgtgcacatgtgtgtgtgtctgtgttgtggttgaggtccaggaacgtttctcagtcaagatgacatctgaaccggaactgaatcagaaaggatgaacgagcttcttcctgtgcaagggacaatcttcttacagggtaatttaccaagaaccactaaacctaaaaatgtaactactttctgaccctcacatgtgtgcacaagacaacacatagaaggatgttgattgcagcattgtttgttagtgaaaaactggcaataacctagctgcccaggagtaagtcagtggataaataaaatatggatcatatacctgacgttagtagctatccacggtacagcactatgtgccaggcactgttctatgtgcattttgtgtgacaaaacaaccagaggagataggtactcctattactttgtatgtactattattactgccatcaccaatcccttgttttacagataaggaaactaaggcacagagagttatacaactcacccaaagtctcatagctagtgagtgataagagtgggatttgaatgtgggttcttggtgcaagtgcacgaagagaaaaagaagagaaaaagcagtgggtgtctggagaacttcaaatggctcagaactccagcatggggtagtagcaggtggctaagagggaatggagaggattccagaatggttaagatagcagtgctagtttgggctagatgagagaagtcctgaatgccaagctgaatatgaatttgaccttacaggttagcaaagggacttgactgaaagattggggcccgtgagtggagatgatgggtttgatcagggcccacgtgggaatttaagtaggagtccagaaaactgccctcctgagcttcctctttcatctgtttcttgctcaaatgacccactcccacttggcgtacaaacattccccgccatggcttgtttacaatctgtcttaacccctgcctcccactagatactagtctatttctctaccttcctatccctattcacaataaaacttcccaaaagcatggtctctactcactgtctccacttcctcaattcccattctctcctcagccatttccagcctggctcccaccccactacaccaccaaagtcaccctcttcaaagccatttatgaccttcttgtagccagatgtgacag

SEQ ID NO: 18 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 19 - DNA, 9205 bp, Homo sapiens
aataatttattatcctaaaatttgcattaaaatataagtggaaaactataaaacatagttttcatatagttttcaatgtaaagacaacccaaatgcctgaagtagaaattcaaactgttaatggcattttgaaaaatcttttagaaaaaaaaaataaactgtttttcattgccaaacatggaattaagtaaaattcaaagtcaccaagagttttctacaaaattatgtcaacgtaaaataaacattttcccttaactacattagtatatttagtatattttctttatgttctaagaaagtacaaattataaaatcttcaaaagtttgaatgctaactcatgtttttttaattaataaaaatggcataaatcaaatagacatctccaaatgatattcattaattgtgtgttatttttgctgttttgttagctttcctcttctctgagcttctttctaaacaaagttatctgggaaattgtgtcctcttgtggtgactggcaaaaacaacatgttttctgcagaaagaaccaaaccaaacaatagcagcaggacataaggcttcaggaaaacccatttgtgaaaaacggaaatcccttccagtgaagaaaggtgaaagttatgagtaatcagaaagagaaattgaattgaaaaaacatagaatgtaaatcattccatttatatttaaattactggtcatcggatgattaaaaagaagacacataatgctatgtagaaagactgagaccatgataatagttcctatgaataactatttatttcttgcattctttcatcagttattccagcaactctttgatacaagtgaagtgagtgtgttttccaacaaggttaatgggcagctaagacaccaactctccagcctgcacaacccctgaaaaaaaaaatgaaaaaaaaaaatgctcttcaagactccatacttttccatattctgatcctgcactttctacaggttagacctatgtctcctcatcccatccttaagctgagttcagaatgtcagctattttaaagattaacatttatttgaaaatttcacttgcggtactgttcgcaccctaatttattgtgttttttactttttcgttttatttctggattgaaaacttcacattttataactttagtttgttttttttacttttaaagatattcagatagaaaaatgtagaatcagttgagtttatgaatcaaatcatcatttaaagagaaagagaatgagtttttatatagggaaaggtgttcaaacatcattaaggctaccagggtcatgaaagaagaataagcttttacacagttattgtgttgacatacatgactttatattccaccttactaaaattctctcatttaaacagcagtccctctggagtgttcaaagcaagatgaaatctaagatatacaacctcatctgatattcactcaattgcagcctttctaattttcactcccttccgtgcgaatgctcctgtgcactatttatgcaattcagataaattatttctcagaactcggagccaacaaacaggtgataggccatctaaggtctgaggagaaagaagagtaggaacagattgaggtactcaccttttatgcaagcccaaactccagaatttaccagttggctaaccattctatagtgtgtgtttcttttccgtatctaaaagtatttataattggttgttgctaagagaatgtcacagcatattcaaactgccaggatacttaatacttattgtatatgttcaatgtgagatttctaactctgttacttttaacagtattttttgtttcaatttttaacacagcacacattcctcaggcaggattgcaaacatgccaagtttgcagtttgaaaacaaagatcgttttgaactatccaagtacaaaagaaattttttttttcagttaaatttttttggggaggactttactttagaaggaggcaaaatgactgaattccagttaatctagcattcccaaagaaggtagtccccactgacatacttccagttaggctaacttcagcctcaaaagaaaatttctaagattgttttcatgatctcaagattgggattttagaaagaagctttcaaatttaaactaaacattgcagaagttgcaattattgttgcatatataagagaatccagaattatacctgacctgtcaacaagtaaaaagggagccatccttgcaaatgtggaaaagaaaattgttaggtaaaatgttagataagaaagagctgtatatgagcagactggactcgtcagccagggcgtgttgcagtttgcaaagcagggagtatctagctccagccattttttgagaggtacctcctggactacttttgctccaaaagtaacattcaagccccacttaagaactcaaaagtcgtctcatcctccactccccggaacagttcaagagccatttcttcttagaactagtcatcaattgtgactgtggctgaaagagtccatgggcaggatggttctggttaggtgttcattttgaattcccctctcagtattctggcatcagagcgtcgcctgagccttgcggggatccctgtacccaatggccaggagctcttcatcagctagaagtttagtgccgggaaaagggctgggctcacttgtttaagaaccagtctttgagactgatgactttgcaagatggactggctaatgacccctggctgctgcgctgctgtggacttggtttctcctctagcttgtccgctcccctccccttcccaaattccccttggtcaggtaagatttcctttacactttacccacactttcctgtcttacttatccgtggccacaaaggaaagagtccaatcattcgatctctttatttatttttgagaaaagagaaaaaaagaacaaaacaaaaataagatttatctttaaaaaaatagctgaagtggaaaaggtttcgagatttctgcagccacgttctaaataagaattgcagaatactgtaaattcagatttacaaaaagaacacttggtggagagtggggcagaatttctgccgcattctctaagcgcttccaagagataaaatcctgtagcggaagatgcaaacgcaagggtgcaggggtgactgttttgagaactgctagagtgctactgaaattaagtggaggtcaagtcgaatctgattttcagacaattttacagtaaggcagcggctcactaaacaggccagttgacaagctgtagtcactttctgagtatttctgtaaaaatggtaagggatcaactctgcaatttgtccctcccatgaaagcacagtcttgtttacacctcgctggagaaataacactcgccctcacttctcccaaaaagctgaacccttcagtcggcccaagcagctccacaccctgaggtttccaagaccaaagctgcgagtctcagcagggaacagccacgtggcctgcctgcgcctcgcctgggctcttgccttcagcttgagatatctgcagccgcgaaccttgctccagcccagaaaggggcgctttgctcaattaattgttcccgccggcgagtccgtactgagaagcccatgagcggaccttatgtgcagggtactccagcgcggtgcacaaaactcgtcgcccccaaacgctgcccccaccccaacactgtgtactgactccagctttttactttgccatgtaagggatggacctgaaacggttattttacctcaattcatttcaaaaaggaaacaagtatggcattgcaaaagatgggcttcttatccaaggcgacttcctttctggttcaccaactttgctgcttccagtttgccaggatctacattaacaccctctttggggctcttcgttttaacttacagacagaaatgcttaaaatgttagcgtatccaagcatttggaattggggctcacgaagcctaattgtccactggatgccctagatagtgggggctggggcggggggggtctcagagcgggcagcccctatgtctaggcgctatcaaattcccacttcactctcttacaagctggcctttcaaggtcacaatgcggagcctaatttgggggtggggatgaaatggccacagggtctctcccttgggttggcattgccagctgttagggccgcagcaaaggcgctgcgctgcccccctctggctctgctgcctttcccatggactgggtttccttccaccgaagagtgaacttctgcctctttcgagcaccttccgaggcgtagtcctttggatgttggggagcgtcagactgggtcgttgtagaggggaaaggagggcccagaagggcgagagagcaggccgggacgcaaatcctcagcccccgcggcgcggccacgtcttcagaaacgcccaggacctccgggctgggccgccgcggtttggcctttggaactccaaggggttcgtctacctgaccattgggtgggctccgcggttgacacttttcttggcatgcccccccaccccgcgccacaccacccccccagccccagcaatcccaaatcggccccacggacctagagggctcttgggcgagatgagacatcacccactgtgtagaagctgttgccattgctgctgtcacagccactccggatggggctgccaccgcggccaggacagtctcctccgaccgcttcctgggctgcgctagggttcgggggcgctgcccgcacgctccggcggggaaggaaatcgccccgcgcccgccggaggaaggcgacggggagggaagggggagggcggctaggaggcgggtggaggggccggccgcccgggccaggtcgtttttgaatggtttgggaggacgaattgttagaccccgaggaagggaggtgggacgggggagggggactggaaagcggaaactttcctataaaacttcgaaaagtccctcctcctcacgtcaggccaatgacactgctgcccccaaactttccgcctgcacggaggtataagagcctccaagtctgcagctctcgcccaactcccagacacctcgcgggctctgcagcaccggcaccgtttccaggaggcctggcggggtgtgcgtccagccgttgggcgctttctttttggacctcggggccatccacaccgtcccctccccctcccgcctccctccccgcctcccccgcgcgccctccccgcggaggtccctcccgtccgtcctcctgctctctcctccgcgggccgcatcgcccgggccggcgccgcgcgcgggggaagctggcgggctgaggcgccccgctcttctcctctgccccgggcccgcgaggccacgcgtcgccgctcgagagatgatgcaggacgtgtccagctcgccagtctcgccggccgacgacagcctgagcaacagcgaggaagagccagaccggcagcagccgccgagcggcaagcgcgggggacgcaagcggcgcagcagcaggcgcagcgcgggcggcggcgcggggcccggcggagccgcgggtgggggcgtcggaggcggcgacgagccgggcagcccggcccagggcaagcgcggcaagaagtctgcgggctgtggcggcggcggcggcgcgggcggcggcggcggcagcagcagcggcggcgggagtccgcagtcttacgaggagctgcagacgcagcgggtcatggccaacgtgcgggagcgccagcgcacccagtcgctgaacgaggcgttcgccgcgctgcggaagatcatccccacgctgccctcggacaagctgagcaagattcagaccctcaagctggcggccaggtacatcgacttcctctaccaggtcctccagagcgacgagctggactccaagatggcaagctgcagctatgtggctcacgagcggctcagctacgccttctcggtctggaggatggagggggcctggtccatgtccgcgtcccactagcaggcggagccccccaccccctcagcagggccggagacctaggtaaggaccgcgccgctgcaccccttcgcctctcaggtggcagacggcaggccggccaggccgcggttcccagtccacctcgatttcctcccctctcccactctccgctcagccttcccacctcacttggcaccgttgcctcgcgcccccagcgtccccggaaggccggtctgaccccgctagggagagcagtctccagggggatgcgccctggtgaggggtgtgtgtgcgcgtgagtgtgcgtgacaggaggggagacagagacacccagggtcacgggtaaggaccgttttgtcagcgccaccctttctttcggctttcaatttttgttctccttaaaacaaatgttttaaaacaaattccacctcctcctcctttccacccacccacttcctcttgcccttgggctgaaatccttccaggttgttcagcttaatttctcagtggtggtgataagaacagtgctcactagtcttagaaaacagccgcagagacctaaacaataaccgactcccccccccccctctgggtttttgcagatgtcattgtttccagagaaggagaaaatggacagtctagagactctggagctggataactaaaaataaaaatatatgccaaagattttcttggaaattagaagagcaaaatccaaattcaaagaaacagggcgtggggcgcacttttaaaagagaaagcgagacaggcccgtggacagtgattcccagacgggcagcggcaccatcctcacacctctgcattctgatagaagtctgaacagttgtttgtgttttttttttttttttttttgacgaagaatgtttttatttttatttttttcatgcatgcattctcaagaggtcgtgccaatcagccactgaaaggaaaggcatcactatggactttctctattttaaaatggtaacaatcagaggaactataagaacacctttagaaataaaaatactgggatcaaactggcctgcaaaaccatagtcagttaattctttttttcatccttcctctgaggggaaaaacaaaaaaaaacttaaaatacaaaaaacaacattctatttatttattgaggacccatggtaaaatgcaaatagatccggtgtctaaatgcattcatatttttatgattgttttgtaaatatctttgtatatttttctgcaataaataaatataaaaaatttagagaaccttagagtttggtctatatttttaaaactaaagattaagttggtggtaaatacctgcttgtttaattctagaggcacccaggagggagggggcactaatataaacaaagcaatgaaaaactcaaataaagcagctactgacaggcacaagcatgttattttaaaagacagctttattattattccagtttggtattcagagggcttagtagcatctcttcatctcttactgtctccaaacagcaaaaacttaacaaatttgctattcacctcctttttattttgaggtgatagttgttaaaacagttaaatgcaaataaggagtataaaacctgcactattctcttattatgtactttttgtcatgctcttcactctttgtgaagattcctaaataacaggttcttttcagtgaaaacatatatagtgttatgaaaattatctatacacatatagaaatgaccagaaatgcatagaaaataaagtgtaaatcagcagaaaagagaaaattccatttgtaatttgcttaactcttctctcatgaatgaatatgacgtttagtgtcatgtttgctaaaaagaaaagttaatatatcgctatattgacgctatttgggaagcatcattctttttttatgtgtaaagaatataaggctgtggaataatatacaaaaatgaagatcctgaactttcagtatccttggtaactggatataatcacagttggaataatcacagttttaaatactacagctatgcagttactagtcagttcattagcgttatattttatccagaattacacagaatttttccctggtgagatcatacatacacacacacacaatctgaagttaagtaaaacaataatgggcaaacgggggtgtttgattttttatttcattctgattatttgtgggccaggaacatactggcttttataattaacattattagaaatagtgattttgtatcagttcttagcttttacatccgatttactgcatccaagtgtaactggggaagctttcaaactggaaaatcattttggtgcctgtttcaaacaatgactaataagtgtatgtatccaaacagatcgtttttgcttagctcaggtcaaagttattttattgcctgaaggtttttgttttcttcttcacacagcaccatagaatgctgacataagaatattatgaatttattgttaatatttcagactatattttacaataaggagtctgcagcattgacttgtattaaaaggttagtgaaagcttggagaagtattattatttttttattttgatagtcctttagcaaataacagaaagggaaggcgattatgtgttggtgtatttgttgggggtgtgtgtgtgttgtgtgtaaggcaggttagaaatgtaaggagggtgccctaggaggatgagggaaagcattaggttggtaattcttttttgtatgtatcatccaaccacctccattttctaaattattcatggggagtctccttgttcactagcttggtctgactatttgatatttcagatttcttaaggtacaccaggatggagctggccatgactgctgagaaggtaattgttatcttaactctggatataagaactagatgtgtcaaagaaaggtagctctccatttgcctttcttttatttctttctttttttaatgtgttttgctttttctacttttttcataaggggaaacactttggagaaaagcaaatgccatatttatagtaatattaaatactaatctgaaggtaatgtttccccccagattttcagagaaataaatgaatgaaaaaggtggtaattttattttgaatgaggcgactgcacaatgaaaaatattaactactggaatgcagtttccagcttgcagattctaatccctacccctgtatacacaagacataaatcgtgaaattcttttttagaatagaaatgaagccacctgtttcttttcctccaccattatatctt

SEQ ID NO: 20 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 21 - DNA, 20370 bp, Homo sapiens
atcatgccctgaacctcatgagcccaggcaggcatcctgttggttaaggtcggcctcagggtccagggatgcactagattctttatcttaaagtgaccctcaagaaaaaaaaaataaaatgtagctataggcagtttctggacgttttcctatttgttaagcaccttcttgctccgagctttccagccacagtgcactggacgccatcggaaggctgcagctgctatttggtgctttaccaccgctgagaaggtggaatggtcacggcccggttctgtcactttcttcagccggacagtcgccttattacggattccagtagggccgagcacactccttcctgcccgtctttacagatgaacatgctatcgctccagcagtacaacccgccttattacgtaaaaaagcagcccctatctacccgcagggaaggagtatgttcggcaccacagctggactggtgctcgagttaagcgtcctgggaggtccgcatgcgctccggaaccgtaatgcgcgctttttctaagccttacggtaaacgcggacgcagggcaaccacgtggcggtggaaccgaggcccggcgggaatgcgcggaatgcgcggcgcggcctcgcgcggttcccgagccacggcccagggtccggcggcgcgcgctctcgcctcctcccctcacctctcccagccgcaccccggccctggccctgccgcccagaactcgctgggcaagtcgtgccccgcgtgaacacacagaaggggcttggggaccgagcgcggcccatcagtccctcagaccctgaggacccagaattccctaaggggtccgaatccgagtcctgcccccagcccttaaggcacgggctccagggaccccaggggaagggcgcggggcattaggtacgcaacccgtttccccgcacctggaaaaaaactccctttccctcccctcccctgcttgttgagtgtccggataaccagaactctaaggcgccccgtaataacgaccccgctgtccctccacccacccccaagtgccaaagcgagggatggaagcgctttcaagcgttccaagggcattgaggagcgagctggagaggcgcggggatgcggggtcctccccgcagtcttccggaaagggcgggggagggcgcggcaagttccggagtggggcatgccgtgggagcccacgagggcctcagcgcggatcctccgccggaaaaccggctcccgcgagccgccgccgcaggtttcctaggccccgcgagtcccgcagcgaagccctgcgtctccgtccgacgcgggggtctgctcagcctcgggtgggccgcggccaggcctgactgcgggggagagggccgaacgtgacctccgaggtcacccccagccagctttctctcctgtggtcggaagtggttttcttctcgatctgggcgcctactccccaccacttggtctgagaggggctggggccggaaggccagggaatctctggtggatttgggggttcatattgctcagggtaccagccgatgcgttttgaggggcgggagtcgaggaattagaatcgcctttaaccctcaagagttgcgccttcagcctcgggatcccagatgcgtcgttggagccagggccgcccccctacctgttgggtttgcgttttaactccagcgcacaccttgccggcagccctcggagctaggggaggggtctcgtttccccgcagcccgccggacagacgactggggcacgggaggggcggtggcagggtggtctgtgtgtggctgaaactaattgatctggagcggaaacgcacgtctgcggttggggcgatggggggggcggtgcggctgtccatgtgccgagcgtgtggctgtctcgggtgggcactggggccggagttcgccccggcccacctcgcagttttggggcgcctgggatcggcgctacgtaagcgaagcagagctgccatagcacgtgggccgccacgcgcaccccaaaagcaagcagtgtggggggaaggggagctcgagcgccttcggagcccaggggccggctttcggaagcgttttcccgggcgacttaagggcttaacaatggaaaactcgcggagcctgagccaagtcctttcaagtcgccgccaggtatgcggctgcaggtgaccccacctgggtgcgcccgcccgccagccgccctggtggaaaagcgggtgcgggaggtcgctggcgaaaggtcgggactggtccctgcaccacccgcccccaacccaagccccgagccccgcggcgcgcagccgcgctgagtcccggggtctgcgtcgcggcgcgccggttcctgaatgaacgcgctcccttcccccgcctgaatgaaggttcccacagccagggacggtggcgaacacgcgcctgcagcggaattcgctttctcctgaccgaccatccgcccaggccgcggtcaccggggcgggggccagggggcgaggaaagcgtgaaggtgatttcagttaattttggattttctttcaaacaacgtggttaccctcccgactgggccacttgccctttgtctccaaatggtcaccaagaaataagaacagagcactttaaatgagcccagaatccgcagttcctgcttcgtggtgggttttaagaagacagtgtaaagtaaaactgcaaccgaaaagttttttaaagttgcttttctctttggaaaaaataaaatcaaaatgctttctctgcgcttcttgaagcaatgaccctcaaaagcccagaggtattggccccctcgggggacccgggggccgccaagcagggttcccccaggtgggggctgggcagctggcgctccccgccgggccccaaattccagcgccgggccccaaattccagcgcctcccccgcgggttcctggacggctctttacgctcgctaaccgggcttgcaattttgcgctcgtccctgagccgggaaatcaacgaagttcctagtcgagatctgcccggtccgcctagtaacagcgccgcgcccccattggctcatgctaattccagtttcctctgtcttgcgcccgggatgggggggtgaagctccctcctggacccagagccggttgtgccggagtgggcgagcctctttatgccctgctgcccctagccgacttcggcccgcttcgcgcctcgggctgggccagggcgcacgcggggctcggggcccctcgccccacgggatgggagaggccgggtgatagctccgggccccataaatcatccaggcggccgccgggtcgggattttatgaatgaaaaagcagctgggccgcccttgtgcgcgggctgatgctctgaggcttggctatgcgggggccaacgcgattgtgggtgctcggggagtgggggggggcacgaccgtaggtgctccctgctggggcaacccatcgctccccatgcggaatccgggggtaattacccccccaggacccggaatattagtaatcctaattcccggcgggggagggggcgcgggaggaattcaccctgaaaggtgggggtggggggggtcgcatcttgctgtgagcaccctggcgaaggggagagggctttttctatcagttttctttgagcttttactgttaagagggtacggtggtttgatgacactgaactatattcaaaaggaagtaaatgaacagttttcttaatttggggcaggtactgtaaaaataaaaacaaaagttaagacagtaaaatgtccttttattttttaatgcaccaaagagacagaacctgtaattttaaaaactgtgtattttaatttacatctgcttaagtttgcgataatattggggaccctctcatgtaaccacgaacacctatcgattttgctaaaaatcagatcagtacactcgtttgtttaattgataattgttctgaattatgccggctcctgccagccccctcacgctcacgaattcagtcccagggcaaattctaaaggtgaagggacgtctacacccccaacaaaaccaattaggaaccttcggtggtcttgtcccaggcagaggggactaatatttccagcaatttaatttcttttttaattaaaaaaaatgagtcagaatggagatcactgtttctcagctttccattcagaggtgtgtttctcccggttaaattgccggcacgggaagggagggggtgcagttggggacccccgcaaggaccgactggtcaaggtaggaaggcagcccgaagagtctccaggctagaaggacaagatgaaggaaatgctggccaccatcttgggctgctgctggaattttcgggcatttattttattttattttttgagcgagcgcatgctaagctgaaatccctttaacttttagggttacccccttgggcatttgcaacgacgcccctgtgcgccggaatgaaacttgcacaggggttgtgtgcccggtcctccccgtccttgcatgctaaattagttcttgcaatttacacgtgttaatgaaaatgaaagaagatgcagtcgctgagattctttggccgtctgtccgcccgtgggtgccctcgtggcgttcttggaaatgcgcccattctgccggcttggatatggggtgtcgccgcgccccagtcaccccttctcgtggtctccccaggctgcgtgtggcctgccggccttcctagttgtcccctactgcagagccacctccacctcaccccctaaatcccgggggacccactcgaggcggacggggccccctgcacccctcttccctggcggggagaaaggctgcagcggggcgatttgcatttctatgaaaaccggactacaggggcaactccgccgcagggcaggcgcggcgcctcagggatggcttttgggctctgcccctcgctgctcccggcgtttggcgcccgcgccccctccccctgcgcccgcccccgcccccctcccgctcccattctctgccgggctttgatctttgcttaacaacagtaacgtcacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtgcggggcttcgggcggctctcttaagacttccctgcaacttgttgcccagacccacgtttctttgctactcacccccctcccttctctcccgctagaactttgaagtttgccgtggtgtttctagggatccgtattttcaaaataaaaattgcgggtattttctgaaggaggaaggggtgggggtgggggtgctagaagtagcgtttcgtgggaggggagaagggggtccgggaggggtgccttcgggagaagccagtgccaggggcaccccaatgggcccgagggtgcgggctggcaggctgggtgcgctttgtgtcccccgcctgcgccccagcccggctgcgcctcagcggccgggagccgccaactccggggggagggggcatagatttgatttttaaattaatatccatggacacgtatgcaagggccgctcgtgccagtattatgcgccatctttgctcttttattgcaaagcaaaagtgtttattaataattgggggcagggtgggggcggggagcggccgccgggcgctggggccgcagctaagggccgcgcggctgccgggagcccgcgggaggggcgcagggacgcggcatgggtagttttggggggacgccgctagggaagggggggcctttgttcaagcagcgagtcccggggcgccccgaacgggcagcctgggccggagagcacggcgagctgcaaggtcgcgtggcccccaagacgccagggcttgatccccgtctgcagggatatcggcttggaggaccttctccgagcgagccgggggcctgggagcacattttcagaccttcggtgggcgcctgaggggcccgcaagtattttaaaataatttttgaaagtgcggcgtggtgcccttgcgagagggaaacgccgcccgcgcccagggggaagggggggccccggagtttgaattcctggggctccccccggagcctgtaacgaactcccaacccccggcctgggtaaagggtcgcccgagggtcattttcagggtttttttatgcacttagttatttttttaatatttttaaatattttttgaaaagatgacgtctggggaaatgcggcgcggcggcctgggacgccacctttgtgtctcgcaggcgcggcgcccaaccccgcggcccgttccgcggccccgcaccccagttggtgtcgacccccagtcagagggaccacggagctccagggcgggccagggtcccgggggccggcagcccgcgccgccgcgcacgccgcccagctgtgcccgctcccgcccccaccgtgccagcctcgcggggactttccctttcagtttcggggagggtgggtactggggacgcgcgggggagggggcgcatcacgggaagctcctgccgcccccagccccgacccctcggcgccctccagacctggcggccctgccaagcgcgatggggggtgcgggggcgtgcgggggggcggcgcgacctggcggcggcggtcacgggccccgtgcctccgtaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctggtaaccactggaccccgccgccccccgccccccgcgagccgcacgcaggaccacggggccggggaaggtgcaggcggtggcggccggcccgcctctgacatatctgctcctccgagggagggcggccccgccgccgggcgtccctgtccggggagcgggcgggatcctagccgccctcgtcccgccgccctgtgtgcgcttgcctgcgactcccaccgcgttcgcgccccgcggtgtggccgaaaagtgggcggcgcgcgccctccagcggctgcacgaggagcgccgcgctcggcgctgagcctccagttccaggtggtgggaggtctttttgtttccacttgcagagtcttttcacgcggcgggcgccttttctgttttgatctgggattgcgtgttgccccagctcccttgagtccccagcattcgccagccctcccctccaacatccaggaccgcacgagacgcaggggccagtgctctgagccggaggtgcggcgtggcccggcccccgtgctgccggcttccccgcgcccccgggctggcccgcacctcccctgatggccgctcaccctgtgttcgcagcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacaggtagggcaggcccggcagcccccggcctccccttgagagccggctccttaggtgaccctggccggcttcttgctctccacctgggtgctgtctgggaagatgtccccagaccccctcctgcgctggagagcgctcttccagctctggtgagcagaggccctggattgtttgtcgcgctggatggagggagatttgctccctcacggccaccatgcagtaccttgggcattggtgtggacggctcagcctgcctgtgtcccgttactctggcctcgtccttcaggccaggcagcctgtggccactccatgctgaaaggggtttaccttggccacagggccgcctcctttctccacccacctccagcccttcttgtgtccttaaggagcctgagctgcagaggccccctcctggcctctcccaggctgggccacctgccagaggcgcctccaggggcggggagagctgtcggcctgcctgcaccacgtgctctgggcagccgagtgcaggggtgtccagcagaggagctcggctgcctgaggccctgccaggggtgccggcagccagccgggctcagctgagccctgagggggcgcttcagagcactctcagcttgggccgccaccgtgggcagcagaagcacccagtcctcacttcccctggcatggccccagaggcccctccctgacatggccttggccccagaacccagtggggacagactcgcacatacacagggtgccgcctcctgctgtccccagccctgcctctgacccccctgtgaccgcctccttccctggcccaggaggcctggttaccttcatgggggagcatggccccatcccacccagctctgctgtggcccacctttggtcaagcctcagttgtcacatctgtttgggggctcactctgggtgacctaggccacaaggcccacggggcatcaaagaggcagtagcatcttctcccctccccagagggcagagccccccaagcctacttcagagctcccttctgacaccggtagcccgcagccggtattccagaatgggttctggtttaggcgtgaggcctcccccacctcctccacctgcttggggcatgaacccctcccccacgtttccaagcgagtccccaaggtgggcagatgaagatgccaaggatgtcgaccagtctggatgggtctggggtgggggggcatgcggcagacagggaggcattctctggctggtgctcctcagaggagagaggcctccggagactccagacagccttttatggagctgaaagtggcttcagagaaatgcaaagtttcctggagagaacgtggggcgtggttcttgcacagcctccctacagggtggctccagcagtggagctcccctcccaggacccctgggtgctagtgggaggcagtgggcaggtgcagattctcgtccttcccactactgcacaccctttgtctgcgaaggcgcccccagcggtgggtgaaggaggagggacacttggggacccagctgtgcacgtgctctcagtgactgtggagtccactccagggtgggtcccgagggaggggcaggagaccaggggacccacccctgcaaagtgctccgggtcctgacccgtggccaccccatggaacgtaactgagcagccagtgccttgttcctgctggacatctgtggagacaagagtgacttacggctgcttaaagtcagaaacaggttgaaggaggtggaggcgtgggaaagagtctaggaaggtgtttttgccctccacgtggcaaaggttacatttaaaggtgatgctgggtgttctccctgcactaggcattcctggccccaggtccccagcaggtgtgcacatgctgcatacactcacgcatgggggtttcagggcaggtgcgcccttggctccgtgggaggccaggtgaggaacgtccagtgccaaggagcttccgggacagctgtcacttccctttacaaccaggcagcggatagggtcaaatcctggagctttggtgtctaattctgggtggctcctaatctaagcacagacagcaccacacactggggtgggggcacgagcttctgaaacaacgtggccccagtgactccacgctgtgtgtgcccctggagacgggggggtgcacaaggtgcggagccagctagaacctgtcgctccctgcagaagcggtttctgtgtgcggttctgatttgcctcaatgagaaggttttcattcatggctcccggctctcagactgggtggaactgctcccatttaaaggggaaaagaggtggctcggctcgttaaggatttctttttctaagttgttacggcgcccagcagccggctttgtctccccttcagggtggctgcctttcttcccggcccctcgccggcggccctctctttaacaaggccgaagttgtttattctctcgggatgaagtctcggatgggccgccacacccctggcggcccgtgggggcccctctccctttgtgcctgggtcggctcccattcagctcccccgaccccccttgttcccgggcgctcagtggcgcgagatgaggcgatggggccgacaaagatgccacactcatccctgccgacgtccggctcccagcccagggcccctggttcctgtgcagaattcctcgtgggtgtgacaaaaggctgcccccaggctccgctggggtgggggccaggccaagaggcacatcccacactggcccacctgtccacggtaggcgcatgactgccctgaggaggggaggccggcattccccgccacaaaccaggacgtaattggtggcagggctctctgtggaaagagccagtctgctgtttgtctaggaggtcagtcacagaggccccgagacgcccactactgcagcctggcaggcggatgagcccagtatctggcagtgaccagagggagttttgtgcagaccacaaaggctgatgggccgccctagattggtgtccctcttggaagtgggcccagatgtgcgggacagtccccaggaagccccaggtgagggcactggtgccctcttgggaaagctgctccctcctggggcccggctcccggcccagtcctccaggggtgtcccatggtgactggtgctaggaaccccacacctcttcccttacttgggaagtcactggaattgttgggctacatcagacggcccagaaaagtgtttttgtcatcggccagaaataggagagttgtgagtagagggcccgggtggagttggggtgtacttggtctgtgctctgaaggtcactgtgacagtcatggtcccatggtaaggggcatgggttgctggaagagctcttccttcccgagtgagccaagccgggctctcctggcgccagggcctgagccgcagccacaccacagccgccctgaaggctgccggccagggcttacccctcaagggacacggaatggcttcatcagtaccctgcagccccgtggcctggcccgggtggaggcctaggcttcagccatgcgatgtcccttcagaatatgacttgtctgcaatccctgctgctggggggtggcaggtacttggggtgagggttagggtcatagaagcgacatctctacgtcctcatatttgcgtcatctaattttgtttttgtgaatacgtgataacattcacaaggctcaagatgctaaaaggatgagaaggcagtgatgtccccatcacctgtcctgtgtcttcccgtggctttctctttccttggttatgtttgagtcaacagtggggctgacgttccaggagggtccgtgggccaggctcttgctctccgagtgcccagggatggctggaggctgaggagggcctggatgtggagcctcagataccgagtgcttcccttcaggccgggccgcttgctcagagccagcacacagggatgcccggatcacgggggccctgagagggtcccctgctcacagcctccttccctctctccttctgcctcagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggtaagtgagggtgatgtcccaggcagccttgccggggcttacagggggagacacctagtgccacggaaatgccgaggctggtgccaaggcccccaagggtgacaaggttggggctggggctgggcccctcggaccccaggccacagactgacagggcaccggcttcttccactgctcctagaacttactgactggctgggaggtcctcacagccttctcacgtcccctggggcttccaggagccgtagagtttctgggcgaagcgtccgggacggaggccccaggcggccccagccaatggtctgtgtggtgatggtgtgtggggttaggcccaggcgagctttgtttgggccacaatgtgcgtggccaataaatagatgcttgaaaagggctcctgtgaggtccgagacaccggacaacgggcggatagagacagccttgttgtttacggcctctttgagaggctgctgctgttaaaccctgggatgactgtgtctttcttcttaaaaatgccattgttttattcccgagtcttttcttaaagaaagaattaaaatgacaatcaaaagggtttgtggcatttaccaaattagaccagagaggtggccgggtcagccgccggccccgcggtgtgtgagggagtgaccgcctgaccccagcttggggctgggtgggcctgcaagacccgttttggctctggcctgggccgcctcttggtggtctgccctcgagcctcccggggactccgcacgggtctcagcagatgctatctagggtccacctgcctgtcccctgcctagtggtgcctctgtcccggggacactgggagtagcggctgcccagcccatgtgtgtctcggaagaggaagaagcttttttgccgtgggacaccgaagttggcaggggcctcccttctgtgttctcggccatggcctcccttgcaccctgccccgtgttatcctttgggggtggtgaggtgtcctcacccgctgtagggtggaggccagcagcccgcagctctctcaggaaaatggctcagaaacaccatcgaggcctccagaagcccagcaaagagaaagcccctccatcaaaatgaaactcgcgtctgcacttttcatttcgaactccacgccctgagtgaaaaccgcttccccgccaggggtgactgccctgggatgttgctgtcttcgggcagttgtgggaagttgggcgctggcccttatttgagtagagaccatcttaactagattggaggcacacgtctcacagctgacagacacacggggtgaagttacccgaggcggagtccactctgcctgatcagctagtgaccaacgtagctgagcccagactcagaaaaaccgtccacagcagaggcccctgcattttctagggcgtgttctagaattttctttggtgggtggaatgtccatctgtgcaaatcgggtgcgcagtgccacacaccagtgacttttcgcggaggagcgtgctgcctttttggagcttctggctgtgggagaacagctttgtccaccggggtagccttgcaggcagctgtggggccagaggaatgaaggaaggtcctggagtctagctgcatgtgtgaccctggagtgggtcatgggcgagggacgggccgcaggtgaagaatccctggatggagctgccaggcccctggggctgagaattgaagctggctggtgttttaggttgaacgtcaggagtcttgtatctcaccccaggcctctggcctcagtttccccatctgtacagtgggactgtttgtgcagccagcccggccagcttcatttgccatgatgagaatttatctgaggggcgggagaggaaagccctccctataaaggtacaggcgctaaaatgtcgtgacctcagtggtccacctaaaagtcgttctggcctgggtcatcgcctgtcgtgctatgcctttgtccagccccttctggttgggagttaagtggcacctgtgcggcacgtggtggggctgtggcccagccctgctccttgtggaaggtctgtttcctgggctgcctagagacttggcttgaagccctagcgtggcttcctggcagttgggacacacacagccccaacacatggagccggttctccatccagaagcccccgggcagtaagcagccacttcaggctgcgtgggacttgcccgtggtggagcctaggagaggcccctggctgggcgtggcgttccagatttcacggctgctctttcccactgacagtgtggtgtggacgctgccaagggagtctggagccccagagggtggaggtgcaggacttccaggagcgtccgtcgcactccacccgagggcgagcacctcagtggccgcagtgggtggatgcatgctgtgccaggctgatggctggccccggggcacaggcctgagcgggagaggatggaggggagggatcaatggtccaggtccccctggccacccagcattcatcctcagtcatgcacggcccaaggcttcgacagccattgatcatggaaggccaggttcacctcaagggctgccacatggagaggttaagtctgaaaaggctgaaaaggcagggttcaaagggcctcctgtccagatcagatggcactgaattccccagggagctggcacggccagtgggaacaggcggtgaaggcgctgttggacatggggacgggcagggggtgtgcagggtgggcgggcaagcatctggtgtcttgtggctccagagaccaggtgggaggtggaggcatttggtcctgagtgtcctgacaggtgatggcagctcccacatctcgctcaggttcagaggaggcagcatgggccgagggacagtttttggcttagtcttgctcttataaaggcttccgggtcatggcacctgggaaggggccctcgctgcaggccccttctaaggaccccctcttcccacctctccccaccctctctctctcaggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccagcgtggtgcctgcctctttgcttcctgggctggccgtgagccagggacgcgtgtcctggtgccctagaaccagggcagggtggcaggcttggcggatgtgggaggccgcagcctgtcctgtgcgctgtgggaagttcagcagcatcctgacctccatccccgggatgacagtcacgccacccgccgtgacaaccaagaatgtctcctgacactgccacatccccgggggtggggacagaatccagccaggagcaggcacacccctcccaactgggaggaagccctcagcacaggtgtgtgaggtgggaggcggtgtcctgtccccgggaggctccagagaataatttgcaggctgcctggctgggtgagcccacctccaaccacgcgagacaacagctccggcctgggtgacgtgagcggtgcccattgatggggaacatcttccccctcttccttgccccaccagtttgtcttcccgggttatttgcagataggaaaataaataaagccggcattcgttaaccctcttctggcgcaaactgctgtttgctctggatgaatcatggtcctttggcgacgccaggctccgggagagcaaagcaccgtgtcagggccatgatccggggtggcctttcactgggatcgtggggacctggaggccgccttataggacacccatgacgcccacctctggatttcaggtgcacgtgactggacttaacttcaaaccccagggtggaggcaggtagtgggagtgccctgggaaggtgtcctcggaccttggtcactgctcctgaacccatctgtgaggctggtttgtcctcatcccaagctaagtggaagctcaggtcccaagccaccgatgggtgctacttgtcagctgcaggttgaatctccgtggcctttatgaagcacctgctgtctacccttcctgccttgtagagcactcctcccagggctcaacagtggggccggggtggtcggtgtgttggctccacaggcgcctgccctgggaggaaggtggggtgtggagggaaacgcttggcccctgtaggtctccaccagcctctcccctgagggtgggggctccgggagccttcctcgagggagtcctatattgagtgggtgggggagcctgcaaggtgcccctgacaggtcacatcagaaagagctcaagggacagtcggagccagaggtgacactggtggccactcgggtggctcacaaggcccagctcctccttgctcctgggcaaattactctgaaggcagggaccaggtctgcaccattgcggctctccagttccaggcaatggccaggtcctgtgtcagggctggggtcctagggaagccatgtccccacccccggcctgcagctgggtttacattcatcccccgagagcacatgggtgtagcaggaggcctgtgcagagagctccgaccatcgcacagggcacctttggttgtttcacggagcaggcaagggagccatcggatcctgttaggtttgagcaaggatgtggggaagaagctggagagccactttgccatgcagggagaggagcacatgggtctagggatctactttagtgtttggaaggttttttaagatgaaagagggatgtgtaggctgataggtctggcagagccaaaaggcagcgacatgtctactgggagagatggagctgagcgcggggctcaggcagggtggcagggcagggccggggccctgggtgggtcaggtgggttcacagccaagtgtgtagagagggcttgggcccagagtgaagcagttgcaagctctcccacaacccattctctctgtctcggcatctgtggcatcccgtgatgggtgggtctgtacacaccccacccctggctgtgccacaatgggggtgtctgtacacccctcacccctggctgtgccacgatgggggggtctgtacaccccccatccctggctgtgccacgatggggggttctgtacatcccccagtgatg

SEQ ID NO: 22 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 23 - DNA, 11252 bp, Homo sapiens
ctagtaaagaggatgtgttaaaggcaagccccatctactcaggcgataaaggcactacacgttgtttggaagtcaccagacacagaatgcctttctatcaaacacttcacatgaataatggctgttcaatgttcttttgaccatacagattgggtttgtttttcacagatgtccaaacagttttgtgatctggttccatcagatgaaaacagcctggtgtgcactaggtggaggaaacatcaaaaaccaatagtaaaaaaagaaaattagtcactcaaaggagctgcacagatggtcttcatccgggcaaaatgcctaccaagagttagaatgaccaatggtgaggaaacaggacccaaatgttgaaaaaggattctgcctgccatctctcagcactcaccattaacaatgactctataaaaacatcctcattgttaatcatgcctttaattcatcatgtcatataaaaagagaattttctccagcagggattccagcacaggatgaagcctgtactcacccccaaagtgttctcatccatcagctcactgttcaggacggccctcaggagcacagccatgcggatacaaacaagcattatgctggagaggaacaatgatgctatcaggttggtaccaagactgatacttcctatttaagaaatcatgttaattatataggtattaagttctggtttttgcaagacaacatcggtaacctcagagatgaacttctagtatgtaatagtattgatatcttcaaattcagggtagatgatctagagcttgataaactgggtctaaatctgagtctcaattaaatcctgattttatcaactccccaaggccttccagatggctctctattgcaaatcttctcctctctaaacatcacgtcttccttttaaatgtctgtttgacattgctccttgtgtgctggcccatttgcaaagtatcagcccctacatttaaaatccaagagaattctctccaacgactcatttattctctctatattaagtgaagatcaagagagtagtcttgcctggtgatgtgcacagcattaatcaaaatggctccaatctttttgaacagccaccatcttttattaactttaagcagaaacaatggcatgatgcctcctcctatactgagacctttgttacttttataaaatggctatagaatataaatttaaaatatattaatctctcaatggtaaccttactacctaagcaaaaattaaaatttatttattttatcttttagcaagccttaattttccaccacactgagacaaaagactcaaaaatactctctccaggacacaaaaggaaaaatttttctttccatcccttacattattctctattgcaagtagaaagaaatactgtttaatggcctaataatttcaaggagccttgaaaatcatttctctaaatcaagaagggcagaaacattttaccactatcatcccttcaaatgcatgtttgtctcctggaaatctttccctgtctccagttgcatacttgcccccacccattgaagctctgaactcacaacatgcagttccccattgcttatggtgatctgtttcaagccctcagagaagcaagattgataaacctggaagacaatcactaacaaaataatcatccctaatttactactcccatttgttctttactatattcatctccagaaaatcatagatgatcaaaagattacctggtgcaaaactcattttataagacaggaaactaagcctcaaagaggtgaggtacccaagggcatgggtagttagtggcagattcacagtgaaaacaaaggtgtcctctcattcagtgttctaggcactctaatgcccagtccagcatgcactccacctcaacggtctcggattgaaacctgagtcagctggctctgcagacaaatgcagaggaagagccctgcccactgtgaacacactcttacacttccagctgctcctccaggagccacaggcttccagctcaagcaaacgtctgggaaatcagccatactcaaggctgcacacacccagctccaaccaccatgtcaaaatgatccttttaacacttctgtgaaaacccattttgtttccccattttattttaaagcatttcaaagcatagcacacaggctgaattacataggcacaggaggcaagactagggaaaacagctagagtcagcttctccccctgcaacttcaatcaaaatagttcacagtaagcacattctcccctccttcttccttgccagaaagcaacaacaaggaatcctccactccagggtgatcctgaataggctatagcctcatcacactaagttcaaaaggatcagaatgcagagccaaggcactagacaactcagtagggttattgagatgcaggtaggttctaagaagaatatagaaaattctgaacttctcaaataacattactcatactgtcaattaatcaaaatgttgaacagctattacttactagacactatgctaagtgctacaaagaccaataagactacttgtcttcaggtagcttgtagtctagcaagaaatgagctacaaagaaaacaagaaagtcagtatgtaaatgaatggcatgcaatacactagaaatatgcatgaacagccctgtatcagagaagaacaagcaacacaaaagcagtctgtgaagacccatgttgtataaagggccacattaggcaccataggagtcacaaagttgaagaacatactagccttgaaggctgacagtaaaatctaaacatggaaataaacacatatcttacccatagcttttaaagccaaatgaaacacattacaaaatgaaagaaaagttactagagaggtaggtgtaagctacatgtgatggatggaactatttatttcaactgaggtaatcaggccagacctgtgagatacatttaaacaggattttgaaggacaagtagcattttgataacctgaggatggggaagggcatttcagaaacaataccttaacaaagggtgggtaagctttgaaagaaggcctggagaaaaatcaggaccccatcatcccaggtcctggagcatggtggggggtaaggctagaaaggaagttgtacgaactcaaatattaattcaaatgctaagaggcttacccttcattctgtatgcaagctaatggcagaagaaaaggcacaattagagccatgctttgagaatcattattgaaagcatgtcgaagatggtctgaaggaagcaattgggaaaagcaagcatagctcatccaagtgggtgagagtgtgagttagaggaagcttggaaattggtgatgtgagagatgctgcagcttctgggattgctgcctggtcgtgtgtagaggaggggcagtagggctcattctgaatcttgtcttgaaaagcacatagatagtgatgccaaaaccaggactacggaaatcacttgaagctgtatcctacctcctcctccatctgtatctgcttcacctatcaaggatatctactattgccactaaaattcaggtgcttatggcctccctcattcatcagccagggtttatctggccaggaaagagaagccccttcaggcatttgcaacagagggagtttaagtcagagaactagtcaccctggtagttgagattgccatcagcaagaagctgttaccatttgaaggctgcagggacaaagggagtgagcagtcctctgggagactgaggaaggaagctcctggcttctccccactttccactttccacttcccacttttactcatctgtcctccaattcccttttggctgagcctagctgaaacccagctgacaggggagtttgagcaagcagcctccagggtcagccctctgagttacaggtagagcaggacagggaggaatggatctcaggacaaacaggttcaggatccggcaaactaattttacactccagccattgagttggaactactggccagcctccccccgagttagcatgtagaatatgggataccagctgagtgcctgagagttatcattcacctcagtgggggtaggggcggagaagggtgacatatagccagccacatctatatccactggcccttccttgtcctagtcctctgtattcctgagaaggaactattctcaaggacctgagtccaagttcatcttacttagagtacagagaaaagaaccgctaactccttctctctttcccccatcatgtctcctctcctttctagttctcatcactctctactcccccctgccccccttttctccatctccatcacctttgtccagtctctatccccattttcaattccttcctcaaaacaccaagttgcttggtagcatgcagggttggaacatgcctttaacagaaaatctcgtgtctcttgaacctagttatttattccttgagcagagtagatattcaacaaaagaattgttaaattcaattaaataggatatatcttattattaaatatttttttcattttttgtttacttatatgatgggaacttgagtagtttccggaattgtctccacaacacctggccaaggaatctgtgaggaaaagaaagatcaaatggaaaatcaaggtacatgacaccagaagacctacatgttacttcaaactttttcttcctcatgaaccattaaaatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaaggagagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacgtgcgatgctcttgctgatttggaccagatagtatttctggaccgtgggcatgaaacgctgggttctgactatggagatccaggaatactgtatatgtaggataggaaatgaaagctttggtaggtatttaagtcattgtgcagcattttcaagaactgatacacagcagtttgaaagataagattaaaactgaaagatagctatattggggctaaaccacacaagaagtgtcacatgatgctgtgcagtaagaaagaaaatttattgaaagtctgtttttctgagtacaaaggatttaatataattctcccacggcatttttctttaaaatgggtcactatccttgagattttgaaagccgtagcagcaacaacctttgtttccattatctcgtaccatattctcagtacattgaaactatgtattctaactaaacataggtataactgtgttttagaataagtggggtttatattttttaaatatttaacttcaagtatcttttttgaaatctgattttattacagaatcaatacatgttaaatttagaacaactggaaaatatacctaagaaaacatgaaggagatcgagtttttagttggatgcctgccagtagcaccaacagcacttctagcatgaatattgataccacatagattttctatagctctttcttccaatgtgaatgtttgacttcacgatgagtttcacagaatatgggactgagaacaatggtgcaggaggatatttctacctagaaaatcaaggttattattccttcccagacctgacaatgatgcatgtgctgataggctaatgacatgccatgacttgacatttttattaaaattattgccaaccaatggataacatgtctttcctaagtcaaaaggagaatgttgaaactagtttttttaaaaaaattttaaagccatggtgttaacattatgttggtcatctacctagatttttctctagctgatctgaaaaatgtagtatagattgtcctggaacattgtgtgttctctatgattagcaatgcatcatcatcacaattaatttgtcaaaaagaaccacatagtaatctaatctccaacctctctctcctttcccattcaattctagtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccagtacgtaaagcttccagataaaaatgctatattcttcatccctcttatgcatcagactgccagttaaatctccctgaggatgattttattcatttagaattaccagtcaaacctggaaggaccactgtgaagagcaattctcaaactttctacagatttctttaaccaagcacaggacagcctccaataatccctatcctgttagatctaattgtcactgacaccaataatcaacccaaattaattataatcattattctaatatttatgagaccccaagtctattctttatttattcaaagaatagacatttatcaaagaggattaatgcttttattatcttaaccagagctgccattgagaagatttattgcaaataattaataattagggttttttacttttattcttttgcttatttttgtttttgaatcccagtggaataagtatcactggggtatttctacccctttgtgtgttaaatagtcttgatctacttcctaacatacctatgcttgctgtatccttagtatacccagtatttagaccccatcaagggttaaataccaaatgtattttgatcatttgacttcatacaaataagtctctgttctgtggagcctacagattggtctgattgtaggatttcttctcttcttcccattactaggaagagtcaaaataaatcaattcaaaaatgcaagcaaatcattcactgatctaaaagagagagggaagagaaggtcatagagacacttaaccttttgtttccagccctttatctcagctctgggctctgtcccacgaatgtgatctcagataaaattttgatgtattccctcttcaaagacagacttcatcaagtcaaataaacagctatcttattctagatggttccaagtctactcttcctttggtcttcttctgtctgtcaaatgtaccctaaaaaagctatcatttgtgtcaaacttaaattttttctgtggcctcagtctatcttattttattcattcttcaaataaattggagaaaaactgatcactgtcttcttttctataacaattcacgtgcttgaaaaaaaaatccaatttgtccccaaagttcttcttcaaactaacatcatttaaagaatttgcaatgcctataatttgtcatcctgtgaacttgcctctcttcatgtattcctgttttatttctttcccactttaccaggaattcactttcctcctgatttttctcccctctgcagccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgcccccatggcccatgtgctgtcttgccctacttctgaaggcctctagatattctcaggccactctgcaggctccctgcttcttgaaagaccttcctcttcactccatcctgctccatccagtgtgctccagcccacccagaggcccatggcttttctaggcttctttccctattccaaatccaggggttggcctctccagccacctcctctcagtcattttgtgctcacatcttgattcctgtgaccatccctgtcaccattttcctacaggaaatccccaggcagcttccagggacagttcctcacagcctttacgctttcaagcttctcactaaaaacctgctgctcgttttcaagctttctctgaacggtgatgatgaaaagaaaaaattgtttttgtttttgttgggagagggagttgtaaaggatgtatatgtctatatgtgtgtgtgtgtgtgtgtgtgtggtcctgaaccattatttcccactagattttgggtggggggtatgcggatgggaagaggcagttgctttccatgtactcctcccctgatacactttgcttttattctctcttcattttccccagttagcactaccatgtacctagtcatccaagctagaaatctatgtggcttccctgaatctttttccttctgcctctccctcattcctaacatccacatatcaccaaatactatattctcacttccaaaagttctccaaggaacccccacctccccaccaataccatactacccaggttcaggactgcatctattctcatttctctcctccatgattgctacaaccttccacctgttcttgctgccattgttcccttctctcaatccatcctccgacctgtacctatgacacaaattcaaatataacaaaaagcgccccccagaaatctggccttgctccctctcctgttctatcccagggtgcctcatccttgacgttttaccctcgagtcgtggtgaagttgttgcggagaccaccatgctattcttggccttgatgcctatgctatgtggtcccttctgcgtgaatgcccttgccaccttccccctgctatccctccagtcacacgctgccctccccactctcccccatttctatggctctactcacatattaaaactttctttaggtctcatcttctctaaggagactcctggacccatcactgcctgttatcctagattaggtccctctctttcttacgggctccaaaagcactgtgcagatcctttatcactgaccttacatactatatcagctttacctatttagccagtttgtggttcctcaagagcagaaacatcagcacctgacatggcaggtagtagacagtcaatactcagctgaactttgaaccaatagtccacagacacctcaaaatcaacagagacaaaactaagctcgctagctttactgccaaactggctcctcctactgcttttttatctactggaatggcatcgtctactcatctgagcaagaaacctagctttctctccatccctcattcccctcctccaatcagtgaccaactcttcatcaatgtctcacatgtcctccccttccttgctaactccaagtttgctattacagttgcattatattatgacactaactggcctctctgccttgcttcttatatttaataattcggtatctctcaaacttgtaagggagtctagtcagagtaggtaattaatagcttctgcaacagacaaagttcaaataaacaaaggtttgtttcttgtgcatgttacaatccaacaggatcattagggagctttgctctacttggccactgagagacccaggctgacacagaggttctgacttctacaacaccatctagcactgcaatgtctaacacagtagccacgaggcatttgtaactattgagcgcttggaatgtggctagtcccagttgagttgtgttataagtgtaaaatgcatactagatttaaaatacttaagtttt

SEQ ID NO: 24 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 25 - DNA, 20160 bp, Homo sapiens
cttccttcagctccagccttagccttgattccatctgtcatcctcaagagaactctttgactctcaagacaactctgtttttgttttttttgttttttttttttttgagacagagtttcgctcttgtcgcccaggctggagtgcaatggcacgatctcggctcaccacaacctctgcctcccgggttccagcaattctcctgactcagcctcccaagtaactgggattacatgcacgtgccaccatgcccagcttattttgtattttcagtagagacgaggttcctccatgttggtcaggctggtctcgaactcccagcctccggtgatctgcctgcctcagcctcccaaagtgctgggattgcagacgtgagccaccgcacccggccttaaggcaacttttattttatctgtgtgtatctgtgatgcgaattgctgctccttggagatgtgtggtgtgatgacccaggttccctcctgtcttcttcaacctcatttcttcaccatccatccccacaccaagagtaagacccatgaccacccgctagggccaggtcctcacgacctttcacttaaactcttactccaccttttaattgttcttcttatttataacctctcttcagtccagtctatcctagatactatggcccggttagttttattaagtcagcatagattgttacatggctcagaaacaaacaatgtctgctcattgctggccttttgatgtttaattcctgcccctccactatctggttctagcctaccattccaagctgcttctctttatatgctctatgctgattcccccagacccttgttattcttcacttacaatccaagatttcttaccacaatacctttgctttttgcttctaatcccaccctcatttaacttaatttcaatgccaaaattcaatccacctttcaaagtttatctcagtgtccacttcccctacaaagactttgtggatctccccaatagaatatgatttttccttttttaaaatcttaaaacatgactctttacttctcttattgtatattctccatcctctcttacaatagagattctcaactcatcctcattgttacctgggatgctttctaaacttgtgtatacctgggccccaccccagagattctgattcagtagctggagtggagctcagtccattctggctgccaaataagatatcatagactgagtggcttataaacaacagaaatacattatttctcacagttctggaggacggaagtcagatcagcctgctaacatggttaggttctggtgagggccctcttctgggctgcagactgccaacttttcattggatcctcacttggtggaaagacagaaagagagttctctggggtctcttttataagggcactaatcccactcatgaggcctcacctccttaaggccccacctcctaataccgtcacactgggagttaggatttcaacatgtaaatttggggttgggggacacaacatttagttcataaaagaagcctacttgtatgtttaatatagattctgatgtagtgccaagactgagaataatcacttttaaaaattttttcctcattgagagaacaaaggagattttaggaagagtcaatgaaaaggtaaatttgaatcatatctctgaatctgttttcttctttaaaatggggataataactcccacttctctgggttgttatgaagcttaaggaaggtaatagatgctgcatatctaagacttagcacagtgattaatccataatgacttgtgagatctgagtgtatacatttatactctgtgcaaaggatgtgagtcacacacatatgttccctggctgaacttgaacactggctactaaaggaagctccatggaagttacttcacttggtggcttaacttagaagcacttctctttctggaaaagaagaaattttcagcatttctaactcattcacagaacggacgcatgaactaatgtggctataatttcaccacattcataatacaaagagaacagcacagtgaagggtttacacagactttttccatgtatgatctcatctaatacccttgatgccctatgagggagatgttaatgcactagtttcacaggtgtggaaattgaacctcagaagaacttagatttgccaaaagttgcataagtgggaagtggcaggactgtaatttgaacccagtaagtttaatttccacccaaatgctctttacctttcaccacaactggccctaacaattgttctgttaacttgatcagggtatatggacatccattctgatcatgagagttcagaaaggcatgaagctgaggacattttgccttggcttatgtgaatcaaatgagccacggcttcactgggtataattctgtgcttagcggacgctgttccttctactggatcacattccccattggcaactcctactcatcattcaagattcaggcacatgtgctcaccccagctggttcaactcatcctttattttataactaaaaaacaaatagactatatctatgcaatctgtgtcatctcctcgggcctagaaagcaaaactgaactataaaaatacacctgctatgttgctgtagaacccacatctatatccttatccttataagaatatccttatccttatgagaataggctgctgccgaaggggcaggagggaggctagtaatagagaggcagaaagagcctacattgccttgtgagttttctgggcaaactaaatttctttcttcttcttaacttgccctgaaactcatgttataatgaacaataccttcatttttaaaactttgttggcttgtttgttttttctagcaagatatctactcaacaggcgaacaactcctgttcgcactgaagtgtaaatgactgatttgatttttgggggagagagcctgagagaactcagtcccagggaccagaagcaggtgaggcacagacaccacggaggtagacagtggtcaggtgtggagaaaggattcagaaggcacaagctggagccctgaaagcaaatgcagtatagccttctccaccactgcaccagggctgggcctgttgggggaatccagacccaaccgttcaattttttaacatagcatttacccaaacaccaaaaactaagtttaccgaagttttagattctcaactaaatcacttgctgttatgttcagaagaaagtacatttataaaccttgtaagcaagccattgcatcaaaataactcaacatagagttagaccaatgttaaggcattttcgtggaggaaaaagcattttttcaccaaagacctaatgtaaagaaagccacattaagccatgaactcaaggaggatgaacatgaccgtgatctttgaacaatttggcaggaagctgtcacatttatattaatacaaagaatgggttgctacatctctgcatgtctacattctacctcttcatgatccggttcaaattccacctccctcatgaaaccttccttgatttcactccctccacaccactgctgtttcctccaaacacccacagcattttatccaaacctctattaatttatctccaatactctagcacccatcacagcatgtggcatgtagtagatgacaggaaatgtttactgaatgaatgtaggaatatacatcattaacattaatctgctgtccagatatgttctaacccaagacacctttctttggatcattttcaaagttcctaagtgacctcaggaaagatcagaactccttatcatagcatctcaaatacactctttacatattaatacatgctatgtatcatacatgtagacacatgcatttatacacaagaatacctgcctttaccaaatatgtatgcatatatgtaaatgagtattcacaaagactgtgtatgataacaaactacatgcatgtggactcagaataagtgggaccaggttacatgatttgaaaagcatgcccaggggcctctgacaaacagtctcctatgtagccatattgtctggaaatctctcagtgtcttactccttgaagatgtacaacatctcattctccctctcccttgtcttctgcctgtgaatataagccaaagtatccagctgttgaaggcctagagctgctgctccactatggcccagccagagactcaagtgggagactaccatgcagagcttatagtagctcccagctcagtctctctaaacataccctctggggttgaatcctggcccctccacaaatgaaacatgtgacttaaacaggtcatctaaatgtctctggatttgatttcatctgtaaaatggagatagtaatagcatctccctcccaaggctatgtgaagatggattggggtaatatgtggaacacagtacaaatgtctgggcaatcataattttttagcaaatgttcattattatcatcatcagtattgttctctttatcttttccatggaaagtctaacttatagactagggattttctaatatgttgataccaaaccacatcctccagaagcagctctaggccccatacaaagcctgctttgtaataacacagtcactcattctctccacatacaatgagggagggtaagtacagagattctctagttgggctccttggtcccaagatatctaccacaaacctcaaaatattcccctgcataagttaattcaacctcaacatcttacatagtcaaaaaatgtatccacataacttcaggattagtcccttgttccttttcctcaaccaattttgtaaaaggaaataactaaaacatccaaaagcaaagagtagaaagttggagttttgaaagactgttgaaagactcccccatttgggggtactaaatcacattataaaattgcaaaactgtcagagtgtaattaacctatgttgcttcatattcctgatgcagcaattgtttaagacatggctttaaagaggactgcaagagagctgtgagtgacacatgatgctgtgaacgtcagggtgctcgccagggaagggccctacccagagggacagaaagaaagccaggaggggtagagtttgaagagaagatcatgttctccctgaagacgcttccatttctgctcttactccatgtgcagatttccaaggcctttcctgtatcttctaaagagaaaaatacaaaaactgttcaggtaaatgaactatcagttaggtgatgtttgatttgctgattattaggaaatagtgtgggttgttttctcttgttaattaaaacagcctgaaagaaattatttgggtgattagtttgagattgaacatctccttttagtgatttggagggttccaagctggtaaggactaggccttttaaaagccggacatgaaaacaagctgaattctttaaaggctgggaaaatactgctgtagagttacccctagttgggaaacttttgtagaagaaatggttttatttataaaatggtatcattttctttctcatttacctaaggtttttccttccaggatatctgcaatcaagttactatatgtctggtgcttccatacaggcattttctttgccgccaaaaattccagatatagaaaataatgtgaggatggtagattacaatgaactagaaatcagtacacagtaaaatcatatttcccagcatcagattgtggactgtttataaactcttggaagcattataaaagcagaaccaatagtttttttccattgtttcacagtctgaaagtgaacccaaatttttaagaaacacaagggaattctgtcacaaatctctcatgcacagttttcttagcattatagtttggagaaagcattattattattaagaggttatctcttaattgtaacccccgggttaatgtaaagtccattgagtgcttattctatgcaaggcaactgctacacatttcacaaatatctcatttaatcctcacaagaaccttattggagcattattattttcatttcagagacaaaaatatcttaaatcagataaataatgtggccaagtgctcacagttaattcatggcagagttaagatttgaacccaggcctgtctgattccaaagcgtgtgatctggtcagtgtcacctactgctgtggttagagtgtgtcccttgaatttatgtgttaaagatttagtagcccatgtgacagtgggtcctttaagaagtacttaggtcattaagaaagattaatgacactcttgtgtgactggattaatttttgctggagtgggttctggctcttgcagaactggattagtaattaatagaatgggtattataaagaggggctgccctttgtgttttgccccttttcacacatgcttgctccccttttgtttctctgccatgttatgacacagcacaaggccatctcagaagccaaccagatgcagccacctgatcttggacttcccagcctccagaattgtgagagaaaatcagccccttttatgtataagttacccagcttcagatattctgttatagcaacagaaaatggactaagacactgacaaggtgccaggtgttcacaggcagcatgaggtctagttaggttacatttcactgtgtatgaagaaacttttaagccaaacttaaaataggtaatttttaggctaaactctttggattttgactaaatttaacacttccaaagagttactttggtgaataaaatgctaatatcaatgatattgctgttcccctaaaagatatttgaaattattttgaaattatcagttctcatttgagaggcaacatggtgagttaaaatctgaatcatcacttggaaaattccttaattctctgtgcttctgtttgttcagcagaataaataagccttttctacagtagtgaagaggataaaaaagaggaactgcactgatgcaacaaatacctcaagagaagtcaattaatgttagtcctaatttttttgtaaaaacaagaagacagtctcctgactgtagagtagcactttgcctgtgtgatgtgatcctcatcccttatgagacacccttttttatgcataaccattaactggagcactgagtggaggaactgttctttctaaaaagagttgttctctcaaagccaacctacaaattaatttgtgttattgttcctgtggctgtcaagttatcagacctctgtagtaacttagaaagggttgcaaaatgaccagagaactggaggtcctttcacagcaagacccctatgtgcttgtctttgtccaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggtgatatctctagggtttcaggatacactcatttatccagtatggactgggctcttatatgacagacactgttcttgggatacactaggaacaagtagataaatatccctgcctttgaagagtgtgagtagtgaatgaatatgagaatgaaaatgttgagataccgtgggttctttccactccaaggcttcccgttttagtcttccttctccaacaatctaaagaggactaccagcaatgttaggctgtctctctaagcacttactttaggacccaacaaaggcttaatcattgctggagagagtcaagtccctcctttcatacatcttaataataataataatatctaatatttgtggaatattattactatttgacaagtaatgaactaaatgctttatatgtatttaatccccacggcagccctatgagagggatactatattagtatcctcattttatgaataggaaactgaggtttaaagacatgtattaacttgttcagaacccacatctgtcctccaccctgagacctgcactccctccttatcctctccagtggtagcacaccatgatgcaactcacttcaaaacagaacaactcatcagttgatggactactgttctaaataacatttaactcttcatacatcctctcattttgaaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagaggtaggtacaaagcagtcatgctcagttttgctttctttggctaacctgaaacttggcaaggatctaatacaaatgttcttatttcagatcacggtgacaattctccatttgatggacccaatggaatccttgctcatgcctttcagccaggccaaggtattggaggagatgctcattttgatgccgaagaaacatggaccaacacctccgcaagtaagttaatcaagattatgtccacacacagcttctgaattaaccttttacagattagaatacacacacacacacacacacaataaaggtaacataatatttcaaactcacaaaaagtgatcatttgaaatttttgctacctcatgatactaagtcaatttgggatttttttaaaaaaagaaagaaaaagacatgctgaatctgattactgaccttacttcttaggtcagagatcagaaaatacagactacggccaaatcctgcctgacccctgtctgtataaataaagttttattgaaacagggccacatttatttgtttctatatcataaatggctgctttcactctacaatggcaagctgagtaattgcaacatggatcatttggcttacaaagcctaaaatatttgctatctggttctttacagatactgctttagaatatctttattttaatattcccaaatttagtgatgtttgaaaagagcctatttttccatttttggaaattcatttagaaattataatttctataattagaaattagcctatttttccattttttggaaattcatttagaaattataatttctataattagaaattagtctattgagaaaagaacagacaccttccctctcatctagttagttcatctccttggagctcttcttcagtggtatgcttctgtacagcaagccactcctttcaggagaaggctcctattaagaaggactgggctgcttgaagggaagaaatgaggctgaggacaggtatacccagaccatgtggtacttcacaaaatatggggtaacaatatgccagagagcacttttagaaaataatcagaaaggcaccttttacctttgcttggcacctgttcctcctgccattttctcaacatgggtatgttacgagggtctattctacctaagtccatatgctaggagctgtgtgtagccaaagataggtaataaccctcagctcctctccattccctgttgccctcctcactagttcggatgatcattacctcttacctgaacgaatgatctagattcctaactggactctccatctccagtatgtgctccctctaatttttcctaccattgcagcagggtttgtttttggaagcccatcttggatcacatcactctcatgcttaaaagtttcaatgtttacacattctgcaccaaataaggtccaaatacttccacttggtattgaaggctcttggtgcttgttcatccttgtctttcttggcttgtcttcttatttcacgtattccattcacatcgggctaacacccacattgtacattcactgtttctaggtcacaccctatgcttttcatcttcctcacatttgcttgaattgcttcctctccctgaaatactcttcccatgccccacgcaccaccttttaaaatctctctcatcttgccatgctatgtatgtcaaaggccaccttctgtcttgccttgtcctggaaatgagaacttcctctgcagaattctgacagcttttgtatctccttctctgtcatcactcactgtgtgctttcaagttgtttgcacacatctctcctcttctagactatgcactccctgtagagcagagaaccacaacaaattgcccacagttcctttcacggtattccaagcattcaaagatatttttctgaaatgcactcatgaatgcataattaaaaggatgtgatgataagaaacaattcttagagcatgggtagaattttagcaggctaattatcaacaggaagttgaacattgcagtaagagcatggatcttgccattatacctgggtttgaccccaatctgtcacttactagttgagggaccaagtaccagctattcaaaatctatgggcatcagtcactttactataaaaatattgttaataaaacttcagaaaaatgttatgaggattgcataacattagattgcctagtatgttgtaggtactctacaattatggctttttttaaaagtaggcatatacaaaaatgcatcagtgtgtttgtgaggcagcatgtagactggctggactgaaggattcatgttaaaaggcaagcactcaaatatctgctggagccaagtggaggaggtcttgaaggccaggcaaaagaggttgggctttatttgataggaagtgagatcctgggaaggttttaagtctccagttgtgaacggcactatgctcatggaagttaaaacatggagaaagagagcaataagggtaggatttgtctctctccagtagagaattcctagagcatcttagaaagctcagaaacagagaagagctggtgctaagctgtgggtgatgagggagccaacatgagccagccctccatctgaggctgctccactgctgggctggcttctcttctaactacttgctcacagggactctactcgtgaggctgagtgtctgggacagtggagatggtgattgtgggaatgcaagagaagaggggttcccaaagtcatgacggctctgccattacttctccaagtctcagatgcacatttgtaaaatgtgagaattggaataaatgcagcttttctgggttctaagagtttataagaagttcatactcacctagtcgccatctatgaccactatcttgattcttctcaatgtccactttctcaggtatctgaaagaaagatgaaaaagacagccatgggaagggctatttccctgagcagttttgacttggaggttgcattccttgagatcatgtttctgccaccctgggatccacactgagctgtttaccacccgtgtttgcatttcagattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggtaaggtcttctcaagagagtcatagtacatggtaggacctgggaataggcgaacactcccagaatctgaatatctcttcctttgcacttctgcagtttccataaaactgaactctgaatttccccagtaataaagcattaacaaagaaggtgtgagggactgagcctctattgctgccctaaaagaaaaaaagtttaagaagaaatgcactacagtgcttcccagttattgtctttggatggtggggatttttttcttcttttttaggctttccaaatttccaattttaaattttttttggggggggggatggagtctcactctgttgcccaggctggagtgcagtgacgcgatcttggctcactgcaacctctgcctcccaagttcaagcaattctcctgcctcagcctctcaagtagctgagattacagatgcccaccaccacacttggctaatttttgtattttcagtagagaggtgatttcaccacaatgaccaggctggtcttgaactcctgacctcgagtgatctgcccacctcagcctcccaaaatgctgggattacaggcgtgagccattgcacctggccccaattttaaatattctaaaaagacatgactattctttttcctacagcaatcctgctcccagatgtatctatcaaagaaattctcatgtagatccataaggggacatgtacaaatcatgccaaagatgttcactgcagtcttgtttgtggtaatggaagttggaagacacctgggagtctattaggagaagactatacagataaaatgtggtagctgcacaccatgaagtgatgtgcagcagtgagaaacatggtactagatgcacatatggcaacagtacagatgggtctttaaaacttggtactttgtgaaaaagaaacagaatgagatatattgcacaatgtattaattaaattacacaatatataagtctatgtaaatttaaaatgtatataaacaaagcaaggcatttttcaagaagaagaaagggaatggtagtggagtatggagagaaaaaaagaaacttttttaaggcatcactgatacatttcaaggataagtataggggccttttgccatattgcctggataagaccattacatctctcttcacgcttgcccttctgttccctagttggctgtgaataaaaacaagaaaccaataagggatttactgctctctaaaaaagtgtggaatggaatggaatagtggattggagtgaaatataattgaatggagtggaacagagtggagtggaattggggaaagcccttttctgtctctggtgggacagcactataaaatcaagcaagcatgccagggtgccacaaccagggaacttttttgtgattgcagatatttcagaaatgttgagcctatcctaactgcctctattccccaatttcttcctcctgcccaccccaacaacacccctttattttgttcaccaatgtttttatcttaactattattatagcctttcattccacagtgagaaaacacatgaaaattcaaatacttatagaaaaaatattgctaaaatttgacttaggtgtatgctatagcgtaaaagactaatatcattctttcctgaaagtggattttggtgaggagagaggcaatagatactgagtgaccaaaaaagcagaaagaggaactgaaaagtcagagaagaattagtttagaatttaaaagggaaggaaggagcatagaattcaaatttctttacttcacagcttgacctggaacttaccctgaaaaccatcattactcttattaaagaaacaaaaactttaaaaggatgaaatatacaattactttaatactttatggtatagtctggtagcacttaccttcacatgaagacccttcacctttctgagaagtctctgatgtacccaagagtcacaccggacttactctcataagaaagaccttttttttttttttttttttttttccaggactttcaagcaaccctatccaacctactggaccaagcacacccaaaccctgtgaccccagtttgacatttgatgctatcaccacactccgtggagaaatacttttctttaaagacaggtataaaattgaaatatttcttcctttgattcctcttactgacataccttaaaactggattcaaattaagtcacacgttagtcaccttttccttgaattctatacttgtgcttctcatcggaattttttataggttctggggcaccactaggattatcatagccccctgagcttaactttatcatcttcgtcttttaaagactatttgtaccatttttgtgtctatactgaatttatgcaattgagtattttctgggtaactcaagacatccaggagaaaaaaaaaacatgaaaacattcacaaacattcagatgcctttggtgatgtaaacagacatgggctacagctgcttgggaaccccatagctgaaacaccaaagcaccatccagttccatccaaaatcagtcaagtaaatggaacctgcgcttagctagctgtgcagcaaatcatcaaactgagcgaaatttttatttgtccattggctattttgtgagtgtgcaatcacaggctgagggtacatgctgttgcttttatcaatatgttcacagcatttactgggagcaataatgaaagatgctacaaagtaagatttttcaatgtccaaggcaggacaatgccaaatgtaatcaaataagatcactcccataaccttccctgaaagctgtgaagtacttgccagatggacaatatcactataaagcttcgccatatgctggtcactgtgctaggaactttgcattcgttcgattgttatattctcccagcaatcctacaaggaccatcactagcctaaaagtcacaggaactgcctacttagactgttatgtataagtgtgtgtgtgtgtttccttatgttccgttaggtacttctggagaaggcatcctcagctacaaagagtcgaaatgaattttatttctctattctggccatcccttccaactggtatacaggctgcttatgaagattttgacagagacctcattttcctatttaaaggtaacttcctaaagttttcatcttatgttagttctgcctgaagggagggggcaggacttctttgtggtccctttcaggttcatggtgagctaatgacagcattaagctggccaggcttttgccagaagactagggttgggccccatcaggaccactaactactgtgtgaacctgagtgggttatttgcttttcagccctcagttttctcaccaacaaaatgagaccacactagactgtcgttagcattctttcatcctcatcccagatacaattctctaagactcctcaagctgtaatttggtttctgcataaaagtcacacaaactaaggcatccaattgtggggccttgaatgtcttgctcagaagtttttattttttataggctctggggaaccattaaggctctcacagcccccctgaacttaactttatcatcttcatcttttaaagactatttgttccattttcatatctatgtgcaagaccaagaagtggtatatatatatatatatatatatatatatatatatatatatataattttgtaaaaatggaaaagtcgtatttaacacttaacctgaatcattgacaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggaggtaacgtaaactattttttcaattataggttgaaatctgtgtttctctaccctgcttacaaaccttcaagcatttcaaggtttaaaacaatctagatagtctctaataggttttaccgagaatatattcttttttacaggatgattgatgttaccatggcttttgaaatttttcatttctataaaattaaaatgtctatgcaatagtattgaggattctgcatcaagaaacaaagaaaaccttaacaaagcactagggtgagatacaaagaaagatttaagctaattctcacttatgagttcaactcagacatatgcaagcaaatgttatccagatagcctgcagaatttggttcccaagttgaacctattatgtatgtttatatatctatatacacacacaagggagttatactatataccaattaagtaaaagccttgaaaatataagcttactttgggtaataacagtgtgtcagccatcaagcctgttttagaattaatgataaccatatgttttataaatgtgtacccttaggtagagaaattaaccattcttaatcctagtaaacagggcattgtgagaactgaatgaaaaaatatgtgtgaggagactttgtaaattatatacaataaaaaaaatttttttcgttatattctgtattggtgttaaatcggataacattttgtttttaccagatatgataaccaaagacaattcatggagccaggttatcccaaaagcatatcaggtgcctttccaggaatagagagtaaagttgatgcagttttccagcaagaacgtaagtaggaaaaaaaaacctaataaatgtgttgtttgtttgtttattatagaggagacagggtcttactatgctgaccaggttagtgtcaaactcctggcctcaagcaatcctcccacctcagcctcccagagttctgggattacaggcattagccaccgcacctggtgtttaaattttttaaatagcatgtttgcatttgaatgttatgttggaacaggggcagggttttctaactgaagacatctgagatattctataagctaattcaataaattatctctctttttttatagatttcttccatgtcttcagtggaccaagatattacgcatttgatcttattgctcagagagttaccagagttgcaagaggcaataaatggcttaactgtagatatggctgaagcaaaatcaaatgtggctgtatccactttcagaatgttgaagggaagttcagcaagcattttcgttacattgtgtcctgcttatacttttctcaatattaagtcattgtttcccatcactgtatccattctacctgtcctccgtgaaaatatgtttggaatattccactatttgcagaggcttattcagttcttacacattccatcttacattagtgattccatcaaagagaaggaaagtaagcctttttgtcacctcaatatttactatttcaatacttacatatctgacttctaggatttattgttatattacttgcctatctgacttcatacatccctcagtttcttaaaatgtcctatgtatatcttctacatgcaatttagaactagattttggttagaagtaaggattataaacaacctagacagtacccttggcctttacagaaaatatggtgctgttttctacccttggaaagaaatgtagatgatatgtttcgtgggttgaattgtgtcccccataaaagatatgttgaagttctaaccccaggtacccatgaatgtgagcttaccagggtctttgcagatgtaattagttaagttaaggtgagatcacactgaattagggtgggctctaaatccattatgactgttgttcttataagaagaagagaggcatagtcacctaggggaggaggccgtatgaagacagaggcagagattggagtgacgcatctccaagccaaggaattccaaggactgtaagccaccagtagaagctttgaagaggcaaggaaggattccctccaatagccttcaagtgtgaccctgctgacacctgcagaattcggacttctatcctccaaaaccgtgagggaataaatttcctttgttttaagccaccaactttgcaatactttgttacagcaaccctagacatgaggtactagacacagtacatctacacatatgaaaatgaatcaacacagaatgcagaagtagaacccttgctaaggactactgggcatcttcccaggacagcagccaaaagagaaccaccacttcctctcctgcctcctccttgctctctcctagagtccaaacccaaatgggccagttggatctgatgttcgtcagttctttacttctatttcctggggtactcaggagggcacacactatagataacttgggttagctgcataaaattcaatgtctcattaagttgcattaaactgagcttagatgtgtaagtttgctaacggatgggtttttttgttaagaactataggatttatgggaccaagtctagcgagtccagatatcaaaatcattataatgttatatttgctgttattagaatataatatagcttattatacaataaatatgtagactgtaaaatatatttctcactagtacctcctattttctttctctgttgaagtttttaaatcccacagataattaaattggcacctttatgcttgttcaaaaattaaaataatctattaaataagttcaaattaaagatttttacttcaaatgactgatttgtgatttgcttttaatttcttaaaatgttaccacataattcatctaaagtataaaaaggaatagcaaattttagatgttgtgcagatatataatgcttgtgtcagaattattataatattgataataaactttagatattgcttcagagtgtgtgaaatattttataaatatttgaggtcctcaaaaatttcctcaagtatgttaatacattattctcaaattattttacaaaataagaaaactgaaacaagtatatattatcattccattcattgatttattcagcaagaattaactacctacctactattaattatgtttcagggactgtgctaagaaaagtgagagagatgtataagcgattcatcatatcaacgtaacaaatgcaaaaggatctataagaattgaatattccatttttaatagtagcatcaatttcagactatcatgtttcctaaaactttctataatgccagttatgaaagttttaggaaaatgcaggcatgaaaatgagtgtcttatcttctcagaacacctgctagttgtgctggctatgatagactcttggctcaaacctcatctatacatctttgagatgaggactgatctcatactattttttaaccacatttgacagagtcatagacagtaaactgtaatatgaagtgtaataaagaattcagagttattccttttaaaaaaattatcttgttgacaacatgtagtattttccagctattacattgatattttgaatgggcacaagagatataaagacctaagcttagaatgcatttatttattttttgtttatttatttatttttgagacagggtcttgctctgtcacccaggctggtgtacagtggtgcaatcacaactcactgcagcctcaaacccctggcctcaagcaatgccccaccttagcctttcaagtatttgggactacaggcacacaccaccacactcagttattttcttttgtaaaggtggggtcccactacatttcccaggcttgtctcaaactcctgggctcaagtgatcctcctgcctcggcctcccaaagcgctgggattacaggcttgagccaccatgccaccctgcctaagctagaatttaaacaatgagattcatgtttatcttaggttgagattcttcaaaagcaaagcctaagtcaagacattggtgcaattatattttcaggacacagtcccaggaagcaagagtgaggtagcaagaagagtgagtcaaagaatgaggaaaagccaatacaattatgtgttatcaaggtttctgctatgggcaattgaagctcaattccaccaggggcctctgagaagcctgcagaccagcttccagtcgttcacatctccagtgagcaggaggctggaacattcatccttcagtttccattccccattggatgaggatgttccctgagagtgttaacttctttacacttccaggctgtactgtcatgctgacctggaaggcttgatggcatctcaatgctctgggaagaaagcaaaaagacacacagtctgtgcttaaagttgtattaggttggtgcaaaagtaaccacaattacttttgcactttgtcagagcaaagtgagttacacaaaccagtccaccccttgtgcaactaaattaatgctgtgggtatgtggcagaggacatcagaagaatctcctacagctttgttccttggagatttaaatgatgggaaattggatgagctttctctggagtaacttaccagcctcagaaaaaaaagaaattacagataccaacactggagaaatctcagtgtggcagggctgctaatgacctatcttaaccttcagtctacacttcttctgcagtgagactccaatttcatttgaggtagcaatgcgtcatattttgtagccttgcttacagctaggcttacagctggatatggccatgtgtttatttctagccaaaatgtgtaagcagaatgttgtgtggggc

SEQ ID NO: 26 - protein, 467 aa, Homo sapiens [sequence omitted]

SEQ ID NO: 27 - DNA, 11892 bp, Homo sapiens
aggcatggggtaactacaccaagaaggctgccattttgcttagcatgagagtagctgggaggcagattctttttttttttttttttttgagacggagtctcgctctgtcgcccaggctggagtgcagtgacgcgatctcggctcactgcaacctccgcctcccaggttcacgccattcttctgcctcagcctcccaagtagctgggactacaggcgccaaccaccacacctagctaaattttttttttttttttttttttttttgagacggagtctcgctctgtcgcccaggctggagtgcagtgacgcgatctcggctcactgcaagctccgcctcccaggttcacgccattctcctgcctcagcctcccgagtagctgggactacaggcacctgccaccacgcccagctaattttttctatttttagtagagacagggtttcaccgtgttagccagggtggtctcaatctcctgacctcgtgatccgtccacctcggcctcccaaagtgttgggattacaggcgtgagccaccgtgcccgacaagggaggcagattcttttttttttttttttttttttttttgtagttgcaagatttaatacagtgaaaacagagctcccatacaaagggaagggacccaaagagggtagccattgccggcttggatgcccgggtttatgtcccgatcattgtccctcccgctgtgctctcaggcgatagatgattggctatttctttacctcccgtttttgcctaattagcattttagtgagctctctttaccacctgattggtcgggtgtgagctaagttgcaagccccgtgtttaaaggtatatgcggtcaccttcccagctaggcttagggattcttagttggtctaggaaatccagctagtcctgtctctcagtccccgctctcaacaggaaaacccaagtgctgttggggaggttggccgatgactgctctaatgggaggcagattctttataagatcttgtaaactgtagaatgcaccctccaaaatctatttgcataagcacacacacacacacacacacacacaccccagcagttcttgcctgcccagattcctctgcagctaaagtgatgaaacttactgggcggagcttcctaaaaagattattagggtctcctgggttggtgtgcctttaaacctttggactttaccacctcctatctctcctatctccttgcaacaaaggttaggagaacaagaatgcagaaaaaacgggtcctggatgacatctgagtgcctgctttgggcttcttgatgagtgagacagaaaataaaatacaaccccctcttttaaaagccatgcttactcaggttttccttcatttgcagctaaatacagaaatgagagaatattttggagcagggatggaagaagagaggtattccccttcccacaaccttctgatttcccagtacatcccccactggaaaaattcatttaaaatcagtataataagcattgattagatgcctactatgcatctgggcttgagggcaaactggactcaggccttttggcctcaagaagctcacagtgtgagagtggcatttgtgtcctcttgaaattcacaggactaaattgtgcccaggctgacattctatccatccataggtgcctgccttctcacttccctctcttcatgggctcttgccttgtaccaaaatccaaacccaaatctcctcacatgtgagtgttggcattcatgtctcagacatgacctatgggcttgggacttttccccgtggaccccagtgacttttcagatgaacaggtatcttcaaaaacttgagaaataggagtcctgtttgttgttcttgttgctttgtcaatataaggacacagggtctttattcaaatgttcatatctatctcttgacagaaatactatgagacatattgatggagaagccgttatctccatatgctaaatgaggacttgcaccagggaacttgcccatggttctctccaaccacttaaattctgaaattttgaaatgagagtggacagtaatttcaaatcaatggggaaagaatcaaatcttcagcaaatggcttgagataattagctacacatttcagaacaaataaagaagtcagatccgggccgggcacagtggctcatgcctgtaatctcagcactctgggaggccaaggcgggcggatcataaggtcaggagatcgagaccatcctggttaacacagtgaaaccccgtctctaataaaaatacaaaaaaaaataaaaaaacttagccgggcgtggtgccaggcgcctgtagtcccagctactcgggacgctgaggcaggagaatggcttgaactcgggaggcagagcttgcagtgagctgagatcatgccactgcactccagcctgggcaacagagcgagactctgtctcaaaaaaaaaaaagaagtcagatcctaacctcaaccctatttaacagattatagatgaaagaaaggtacaaatggcttttacatacctcccttctccctgacattttgtatgtgtgtgtgtgtgtatttacacacacatctcatataaggaaattgaagggaggctgcctgcatccctgagtcactctccctctccttctgaatgcttacctgtgcccagaccacctccttagcctcgcaccctccaggcttacagggcactcttctatgcccatcccaagtatagctgataccttccaagggccagacttggtgctaagtaccaagtacgcaaagattaataaaacaatgtcctgtttcagggagctcaaagctgattcggcagggcatggtgtgtacatgaatgataaccacgtagggttgcaggtttcctagtgaggtaagcacaaggcaagatgggaaacaaaggaaggaggggttcacagcctcacccagagtccagaacccctggcctgcctggtgcccatgctgagtccacttctggaacacccagctcagagagggggttagacctgcaggctaacacagacacagcccagaaaacccaggagccgagggggaaggagaaaggtgcaagaaggggaaacccaggtcctggtccccttctctctgcttcctggcagcagaactcagacagaacccttaagccagtctaagtctggcaggaccagtaagttctgagttagctccatactagtttctagcaggctctttctcacttcctgattcttaggtttctacattgacactccctgaagagttgggaagagacaccacagtcccctgaccctgatccataggtcacacagcagggacatccacagggtgggcgtgggccctctcatccctccctcccactcacttcacgctggctgggccccaaggtgtttgcaccccttgcagtgagtgaccttctctagtgcagcaagctcagaacctgctgccactggagttgtcccattgctgatgcagaaaggtgaagaactagcagaacactggaaatgccctccatctgggtccatggctacttaagctcaatgctccctggcaggcaggaggacaggtgctattgccctgttgggacagatgaaaaacagacacagggaggatgagtgatttgccctgactatagagtggcagggccaaggcagagcccaggcctcctgcacctaggtcagtgttcctcccagttacagtctaaactggaatggcaggcaaagcccctgtggaaggggaaggtgaaggctcaatcaaaggatccccagagactttccagatatctgaagaagtcctgatgtcactgccccggtccttccccaggtagagcaacactcctcgccgcaacccaactggctccccttaccttctacacacacacacacacacacacacacacacacacacacacacacaaatccaagacaacactactaaggcttctttgggaaggggaagtagggataggtaagaggaaagtaagggacctcctatccagcctccatggaatcctgacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaatatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtactgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaatcaactttttttaattgagaagctaaaaaattattctaagagaggtagcccatcctaaaaatagctgtaatgcagaagttcatgttcaaccaatcatttttgcttacgatgcaaaaattgaaaactaagtttattagagaggttagagaaggaggagctctaagcagaaaaaatcctgtgccgggaaaccttgattgtggctttttaatgaatgaagaggcctccctgagcttacaatataaaagggggacagagaggtgaaggtctacacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttgtgagtatgattccttcctgtcctttctctcttcctgggactgcctgaactagacattctcctggaactataagaaccctcctcctgcgcctccacctccatccccaacacctattcccccaaacttaaattcttaagagaatcctagatcaagccatgggtttggtgagttaagctaagccagatgatacagtaaatgtgcaggaaacctgccttataaagtaaatgcgttctctctcgtgctgagaaacttataagatcctgctggcgctctatactttattggctaggagaagtaaagaaatgtctgattcgaggtgaagatgctccccatgccttgcagcagggaaatttaaattgcctctgcttagagcgtttccagacctgaaagaccagtggtttagggaagcactctacatgagggaaacctgcattagaaggagcttcttaatccctgggatctttccaagctaaactggatgtctacagtggggagaaagaaaagcagagaacaggacatgaggggggctcaaggccccgaagggttgacataggtgtcccttaaagccgaatgtagctccgcagaaagaagaccaggactgagtcaagcttctgctttcccttcaaaatcggccagattttttaaataacttgactctgaggaggaggacctgatttaagtgatggtcccatcactgttgaatcctctgtttttaaaactccccttttgatttttttgggccagagccaattttatttaaaaaaaaaaatctctaaatgaaagggcatcaaaaagaccgcatttcagttatttccccaaacctcaagttcattctccttttgttcttcctgcagcaaatgaaggatcagctggacaacttgttgttaaaggagtccttgctggaggactttaaggtgagagcaggggcggggtgctgggggagtgtgcagcatgattaagggaagggagactctgcttcctgattgcagggaattgggtttgtttccttcgctttgaaaaggagaagtgggaagatgttaactcagcacatccagcagccagagggtttacaaagggctcagtcccttcggggaggcttctggtgaaggaggatcgctagaaccaagctgtcctcttaagctagttgcagcagcccctcctcccagccacctccgccaatctctcactcacctttggctcctgcccttagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtgtaagtagcagatcagttttttcccttgcagctgcccccaaaataccatctcctacagaccagcagggacactcacatccacagacacagcaaagacacagactggcagagctagctgtaaatgaggaaagactcctggagtcagatctcttgctcatttctctttgagcaggcgttgggggtggctgctaggcatttacatgtgaaatttgcaaacagctttcctgttatttgtgagtcatttgtgggttattaactactcccctctctcttcataaaaggagcccagagcttcagtcaggcctccactgcctctttgtaactagaccctgggcggggagctaaggttcccaagcagaggaaacatcattcacctcttttaatctcaatgttttgaaagcaaagctctaagaagggcccaattgactgacaggatttcccctggcattttagaagggacaagggggctattcatccccaggctagtgtctatgagtaattcctccaggtaatttatttctccaactgaaatgatgccctcactactaatggtttcccctgttctgtcaccaatattggaaaatcagttggtgtctatttgtaggacaaggctatgtgaagggtttggtcccagtagcttccctcctcagatgcttagaagtgttcctcggtggctgtgactgacggggaggaacaggagagagaggcagaaaaggacaggctgaagaatgcctcgctcagcactgcaggagatactgtagagttctgggggaggaaggaatcccaagacctgggttgtcatccaagccttgcaaacatcttggagtgagtcctggagaaatacatttaactcccagggccatggaagcagggctcagttctctctgggagctgtgaggcaaggcatttggataaatctggcctcctcatgatgccaccagcttgtcccctaagtgtgatggacatggagctggaagccaggatcaccaacactttctcttttcttccacagcatcgatttcttccctgtgaaaacaagagcaaggccgtggagcaggtgaagaatgcctttaataaggtgagcttggatggtggcagagagggtctgcagagcacaacccatgcccactccccaaccccaaagcatggaaggtggtggggactcaataggccccattcttcattggagagagtgtgggaacctgacagatggtatgacctgctcagccagtgaggagctgctgccttgattgtatttgttttctgttaagtgtctttgggggtttctaaatgactgctcgctgcctttgcaggcttgcgggttaggctggccggccagcctgtgaacacagtgagctgcatgctggggagagtgacaaaggaaacagaaagtacagaaagtagcttgttgggaatctaggctgaacccacacgtgcaggaagctggcacataaatgtgcacatacaaatacacctgggggttcagcccagactccccagaactcagaatgagcaggaagctggattctcacttaacctggagttggttcaagcccgctttccatctgcccttcgcacctgcggaggtgcctgagaatgtcagttcccaaacgaaatggggtttcacacttccaactgtgcgtgaactttttcagtctgatttcccagaaaccgtgcggcctatgtcctcctcgtgggctggggacagacactgcacagagtgccaacatcagggggtgtgaatttctcatagtaggtcagggcggcagggcagggcctgctcagtgtgttggtgggagaacacagacatttaaaaggctccctcctctcctctcaccgtcttgctttcgaagcgcttcctctaatgtcttttcatcaaactctgcataatcatcatgtgaatacgtgacctttaaaattgttgaaaaggcatcattttgaagacagtgctttgcaaaatgaatgctccccttgctagggggaggcctggaggagatgaaggtcaatgcacagcctttcccaaggcagctaggcctatcctctggtttacttcccagcgtgagggagaacaagcaacctctgcactcaaggtcatgcccatccatgagcatgagggaggggagcctatttagtccccagaaaggattttaactgtatgtttcttatctctctgcacagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatcatgttctggtgtcagttgactcttgtctgactaaaattgttctctcaaagctcccaagacaaaagagagtgggtgaatgaattctgcatttcagagaaaggaagccagctaccccctacccccaggagagagggaaggctccaaacctgatcagagaaaaagctattctcactgcaaaagccagtagcaaagacatcatggtcttcccacaactcaatcctttaaagatttgctcctaaatccttaaagatcatgctgattttcaaggctaacggggagattctgtaggtaccaaaattagaaaggtaaacaggggaaggttttttggaggttgtttttgtttttgttgttgcttttcgagacggagtctcgccctgtctccaggctggagtgcaatggcatgatctcagctcactgcaacctccgcctcccaggttcaagtgattctcctgcctcagcctcccaagtagctgggactacaggcatgtgccaccacgcccggctaattttttgcatttttagtacagatggggtttcaccatgttggccaggctggttaggaactcctggcctcaagtgatccgcctgcctcagtctcccaaattgctgggattacaggcgtgagccaccacacccggccaggggaagtttttagggtgaatacaaattgcctgggttatcctgaggattaagataatgagctaacagggcttctggcactaggagatgttcgcagtgcgtatagttggttgttattggttatcacaaccaaaaactcccgccaaatcctctttgagctgagatcagaggcaacagggctggacccaacagagttgaaaataagcaaattagcccacttgagaggatagccaaggacataagctgctgctctttctgtcccctctctgagccaccagacaaatgtgagattggcttctgactgcggtctgctgggaggggcacggtggctggggaagccctactcaaagccccgtgacttagaggagagaggaggaaaaaaatgagctgagtaaacactagtccccctcacgctctgcctgggcagccctggtctggggaaggctgggagcggacaggctgtggtcagtttttcagtaagacctgactcacttcggttaaagtaaccataggaaggaagtgggaaacatcaaagcgaaaattctgagatgaaagggtaggagtaagaatggaggttagcagaagataaggagagggggcctgaagctctctactctgaggctgtccatcctgaactgaagaagacatcctgagtctgtgaaaggaaaatcttggggcctcaaaatcaccaagctaaagggaaaagtcaagctgggaactactcagggcaaacctgcctcccattctattcaaagtcatccctctgttcactgagatagatgcatatctgatagcctcctttggaaaagaatcagaaactcaaaagaatgcaaccatttgtctcttacctacctgtgacctggaagccccttccctgctttgagttgtccccatctttctggatggaaccaatgtacttcttacatatattgattgatgtctcatgtctccctaaaatgtataaaaccaaactgtgccccaaccaccttgggcacatgtcatcagaacttcctgaggctgtgtgacaaagctgtgtcacgggcacatgtcctcaaccttggcaaaataaactttctaaattaactgagacctgtcttaaattttcagggttcacaagtcccctggcctggaaggcttaggccattagccttccaggcaatgcatactgactttagtttgagcctggcatttcatctgagcgaagtgggcatctgaaacaaacagaaactcacctgaaaaaaagctgtacagctgaaaagatgttaatgatagctacaatttattaagcaactacagtttattaagctagtcacttattttaagccatttggtatgtattatgtagcaataagatggcatta

SEQ ID NO: 28 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 29 - DNA, 23273 bp, Homo sapiens
gacagcagtctgaagtcaactggctgtgagaactcgagtaaggccagtcccgatctggtcctcagtgatggagaaaagcccctcttaacctccaattcaatgatcctaaaagagcaggtgcttcgggggtgctgaaactgcgcttttggagggggcttttgggaaggccgggctggggactcaggtctggagggtgacagagccgacctcccgtaaaccagggaggaggaaggtgggggcgggtgggcctaggatctgggggcgcctcctcgctgcggggagctggcttggggctagggcgtgactgtctccctgccaccatcaccgcccgccggccgtgactgcaataagagaagtccgaggcggcttcctcctccctgcccagcaggggcggcggtcagaggcgggcagcaccccagttctccccgcacgccggcactcgcggctgctggagccccggctggctcaccccggggccgggcagaattgggctccaggtaagcgacagcgtcgggtggggactgggcaggtcaagcagtgccctccccctcgaggctctggagagaggactgggggtacacgggaagagaagcctgaacctgggggtcgggggacacatgagcaaggtgacagccaaagggaccccagcccgaaaaggcctaaggaggaaaacgggcgacctgaaaagcaaggctgataaacctggaggagagggcggaggggagcacgggggaagccgaccaaagggacccccaaaaaggtctagtgggtaaaatggaggggactgataagagtttaggaagggggctgaggtgggggagaaggattaaggggaatccccaggacggtctgggggagaaactgaagggatcgtgagagtgggactttggggagaagccgacgggtctgatgggtccaggagaggggaaatgggtgggggtgctggagggaacaggagagggagctggcgggaagggggttgaggagaacgacttctggaggacggagaacctggggtgcaattgcgggtccaggaagttcccctcttccgagccggccgaagtcggggtgaagcccacagcccgcagggtaacgttagcggccgcgaccgcggccccgcgaccctctcggcccgcccttgcggtaggttccgggctgcaggggactcctgccgggcgcgcgaggcgtgggtccccgcttcctggggaagtccccgccctcggcagggacaggcctctccgggcgccccctccgcgcccgcggcggtctcggcccgcgctccccgctggatccgggaattgctgccgccccgacggaaatcctgcctttgaccgcgagtgcccgcaggggctgcctccaaggcaacgagagggcgggccgcgctgggccgccgcggggctccgggtgggcgcagccccctttgctctccgccttcgcccccttttggaatcctcggtctggtgggtggggggtgggcttcccgaccgaggtaggaggcgatgccgctgtgttcagggatcctggggtggaggatctgctgtttgagagacctgggttcttagcaagactgggcccttaattgctgtgtgactgtgggcaagttacagggcttctctgggcctcactttcctcatctgaactatggtgagaaatgatccttgtgtccactcttcccccacgagaatcaggtgcagaaagcagtttttgctgagtgtcctgaagggttaagggctatccctgactcagcgagcctccccttcaccttctcaaaaacatgcagaggaggccagcctgggcagcacagggagacccccgtcctgtctctacaaaaaaatatagttagccagaaggctggaagtggtggcacctgctgtagtcccagctactccagaggctgaggccggaggatcgctggagcccacgagttcaaggctgcagtgagagctcctggatgagagagtgagattttgtctgtaaacaacaacaacaacaaaaagcatggagggggaccagactgcccggctctcactgacagggcctccagggaccagcacacagttggcgtctaaatagtttatggctctgatgagagcccatactggggaggtcagcagagttaaaatctgtctcacaccctctctggtgctgcatccagttggccgcccctcgggtgtgtactctcaggggccctgatgtccttggcatccttccgactgtggtgatgatgggagagcaggaccctgttcccacttcacagatgggaagatggaggtttgggagttggcaacctgcagggccaacagtagggaccagaattccaggacatctcgtctctgatgtagcctgagctgaggcctgaatccttacctcatccctaccaagtttcatgtaccctccggggaggagggtgtggggtttaggggcttcatgactacttcctgttccagtgagtaggtgctctcacaccctcctccagtgagggtgtgagtgtttgtatgttttgggctctgagtgaatgagcatgaacccttgagcatatgagtggatgtttgggtgatctctgcagccacctggggctgatgtggcaaaggaagtggccagacctcaggaggctgggtagggaaccccagagctggggaagtaggggccagggctcttcccacgcagagcagactgggctgacctaaggtgggaaaaggcaggatgtccccctcaccgccccactcccctgagggaccagggagggggcttcctgtctggccacactgtgaaatctagactcccatcccttggctgcccctggacccccgccccccgccgccatggcttccatctcctgaaaatcctgagtcccaggccagatggcatctaaagagctgtgttttagaggctggtgggtggttttcagcaacaggtggaaaaccacttttacccacaagaagtggaaaaaactgctaatggcctcgggaccacatggagggtaaaggccacccccgatcctgcacacacctggcctcaccacggtggtggtggagtcagacagggttgggtgggtatgtcttcttcaggaggcagtttcgaggcctcaagaaaggatggtgtgagatgaaagggggttaatgaaggcagggcacggtggctcacgcctgtaatcccagcactttaggaggccgaggtgggtggatcacctgaggtcagaagttcaagaacagcctggccaacatgactaaaccccatctctactaaaaatacaaaaatttagctgggcatggtggcagacgcctgtaatcccagctactcaggaggctgaggcaggagaattgcttgaacccgggaggcagaggttgcagtgagccgagatcgaaccatcgcattccagccttggcgacagaatgagacactgtttcaaaaaaaaaaaaaagaagaagaagaagaagaagaacaaagaaagaaagaaagggagttaatgagcagtgggggcctcagcaaaggcactggcattcagccaggtggagggtctcggtgagaggactgagggtgagggatggaagcccgcccggtctgagggatagggcctagtgagagatgcggcacagtgggatgggagtcacctgcagacctcagccttgctccctgaccctccagccctggcctgcccccggccagcccagagcctggaggagaagccggaactcttgcaggatggtggtttcctgcccctgcccaaagtcccggttcccttttgatgaaatcccccaggcggctgggccagctcagccctctcacctcaccctgggaacttctctttcttctcagccctgcccagttctgtaccctctggtcccacaccgtcactgccacggaggaccttcctcaagggaaaggagggaagtgaaagttcactgggcacttactgtatgtctgatgctttcagtgatgtgaccccatttgatgctgagaagcaaccccaggaggtggacatttgtattcttattttataggcaaagatgctgccgctcagggagatgaactgacttgcccaatgccttaagctagttgtcaggcaggctagaatttgagcctaaatctgcctctagatcccacagatacttggtgagggttgcgggggcaggacatcctgtgtctacatcaaagaactggcatagctttgggaagtgtgggcctcgaaaaaggatgggtggacctggggttcctatgtccagcagtaggggtgagtgggaggcagggtccctccctgctgggtgaccagctgccatatgacaggggtggtgtgttcgaccggaagtatgacattcactagttaggaacagcatgatctcctgctcaggctttggagaacacagcagtgggggagggaacagaactctggacttgaattaatagtcctgactttgaatcccttgtctggctgtgtgatcctgggaaagtaacttccccctatgcaaaagagaaggtggagtggtaaatggtccctgggggccattctggttttggttattggtcacacttggccactgcacatcgggcaagagccacatgtcatgaagccctcacaacaactactcagaaggtgcagatgcaacaactaaggctcagaggcttatgtcaaagatccaaggtcatagagctactgaagaatggagctggggagggccacagggcagatgttgaagtgaggagcactgcggtccagggttggacctcagtttgatacttgtaacctgattttgaccctgatggggatctcggaggcgactcctgtaaaccagatgttcaagagacatatttataaacagaaccaagtgcccagaatgatgctgtggctactctctgagctgccccctttctggtattagcaggcagcgaagttcagtgctgagaaaagagagacctggcttcttcagattcagcgactgcctgagaaaatctgggcagatatggctctctctctctctctcctgcgcccctccctccccccacctgcgccctgcctgctgtatcaaggatttagagcatgaggcacagggctgagaacactaggtgctccttaagagacacacgttattgcaggggtgtccaatcttttggcttccctgggccgcattggaagaagaaaaattgtcttgagccacacataaaatacactaacactaatgatagctgatgagctttaaaaaattgcaaaaaaggccgggtgcagtggctcatgcctgtaatcccagcactttgggaggccgaggcgggcagatcacgaggtcaggagatcgagaccatccgggctaacacggtgaaaccccgtctctactcaaaatacaaaaaattagccaggcgtgttggcagcgcgcctgtagtcccagctactcaggaggctgaggcagaagaatcacttgaacccaggaggcggaggttgcagtgagccaagattgtgccactttactccagcctgggcaacagagtgagaccccgtctcaaaaaaaatcacaaaaaaaatctcataatgttttcagaaagtttactaatatgtgttgggccacattcaaagctgtcctgggctgcatatggcccatggaccatgggatggacaagcttgcattattgcttctactaggattacagaatggactagtggttacagtggtttttggcaacaagttgatcagcattgcattttagcatcctttactcacaatgtaatcttgggcaagttacttaacatctctgtgcctcagtttcttcatctgtaaaataagggtagtcataaacccacctcataggttcaccaaataagaccatgactgggagttacttggcaagatgctagtcactgtgtaactgtttaataaattagctcatttttactgccacctggatttctaggaagtcaaattactctccaaagaattggtggcacatagagatcagagcaagaggaggatgaagagtcctagggctattttcatgcagatgtctgagcagagtgaacccaatctgctctccctccgtctttttccctccttgtctctttagctctctctctctctttctcttcatctctgcctctctctgtttctatctctttctgtctcttgattactctttctccttaattctcatttctgcctctgtctctttgtctctgtttctttctggcagtgtgtgctggagccagctacttactggctcaggggagttcattctgtgtgtctcttccaccccagctttcaggaagtcactttggtagctttaaatcagccattgtgggaatatttacaccatggaaatcagaaaatgtgacaaatcagggctttttcttttcttttcttttctttttttgagagtcagtttaccagcataccacgctgtgtttctctgtctctgtctcttccttctatctctctgtctttctgtctctctggctctttctctttatctccctgtatctctcaaactctgcctctctctctctttctttctgcctctcctttttctgtctttccccatctctttctttcctctgacaaaacagcatgaacatggctacatgcagtgtggcccctccctgccttgggccatgcagatgtcataccctgtgccctacatcccctgacgcctcagcatactggtccccagagagccaggcctggccagggctttggtctgcatcaagtttggaagtgataagacccaagagctgcctacctctgcctgctgacgacacggtcagtcctgccagggctcttcctgagcccacagagcttccttctcagatgctgatagtctctcccttccagccatactgggcatgcagaattcagggatcccctccatctgatatagatatgttgtggcaatatatacgtgggtattattggaagtggcaatattgtatctgtgtgtgaacatgagagattgtataagtctatgttctcctatagaggagagctctatgtgggtccatgtcaccggagagggctgtgtatatctgtgtgtgtgtgtgtgtgtgtgtgtgtgtgtgtgtgtgtgtatccctacatggggggtggggggacagtgtgggggcatccgcatttgtgcatgctagcaggaatggctgcatctgtctgtatctgtgataggcaatggagagaaatgcttgaaagctcagattctggagccaaacaccaggatacatcatgtctgtggccttcttttttgagacagagtttcactatgctgcccaggctggagtacagtggcatgatcatagctcactgcagcctctatctcctggtcttgagtggtcctcctgcctcagcctctcgaggcacgtgccaccatgccaagctaatttttggagtttttttggtttttttttgttttttttttttttgagacagagcttcactgtcactcaggctggagtgcagtgacaggcgtaagccaccgtgcctggccaatttttgtattttttgtagagatgggatttgccatattgcccaggttggtctcgaattcctggacccaagcaatcctcttgccttggcttcccaaagttctgggattacagatatgagccactgtgactggccaaccctgggcaggtttccaaacctctctgtgcttcagttgcctcatcagtaataataatacttaggccaggtgcagtggctcacccctgtaatcccaacactttgggaggccaaggcaggtggatcacttgaggtcaggaattcaagaccagcctggccaacatagtgaaaacccatcgctactaaaaatacagaaattagcctggcatggtgttgggtgcctgtaatcccagctactcaggaggctgaggcaagagaatggcttgaacccaggaggcggaggttgcagtgagctgagatcatgccattgcactccagcctgggtgacaagagtgaaactccctctcaaaataaataaattaataataataatactacttaaatcatagggttgtgtggataaaatgagataatataggtaaagtgcttaatgcagtggccagcgcatatagtaagcgctataaattttgtatttgctattcttttatagaggatgtgtgtgtgtgtgtgtgtgtgtgtgtgtgttcatgtgcatgcacatctatgtgcagacggatgagggctggaggtgtgtgtatatggggtgtgtgtacactagaagatgtttgtgctaggaatctgtgcagaggagcctggagcctgtgcatgaggcagctgaggtaatgtgccaactgagaaggggtctgaagggctgtgtggatctgggtatcagcatggtgcgccatttgaggtgtgtatgtgtgtcttcctgtgagaaggctttattctcacccttggtttttctgttttctttttaaaatagagacagggtcttactctgtctcccaggctggagtgcagcggtgcaatcacagctcctgcagcctcgaactcccaggcttaagcaatcctcccacctcagcctcccgagtagctgggactacaggtgggtgccatcaggactggctaattaaaaaaagatttttttttatagagacggggtgtccctatgttgctcaggctggtctcaagctcctgggctcaagcaatcatcctgcctcggcctaccaaagtcctgggatagcaggtgtgaaccactgtgcccggcctcacccttgtttttgtatcagccccatctctcttttcaccagttcctgaaatccctcccgctgggccctggatggcttccagtcctccacctctattttctgccctggctctaactagccctgtagcatcctggggcgttttagacacagtggtttcatcccagggaggggtcccggggcaaaggtctcaggcagggcccagtgaacaggggctattttagggcaggcttctcaccacagcccgccccacagttcaccacatgggtgtgatgcccccacccccacccaatacacacatgagagatcacttagagcaaagggtgagaggggcaggtggggctagggtggagaccaaagcactgatgtgacggaaccatcagccaggcaactggacctggtggatccaggaagactttctggaagaggtgagtggtgctaggtagaaaggataggacccagagagaagaggaagagaatatctgtaaggatgactggactggggatcgagagagagaagctgggggccctttcttctaggaccttggggcccctctggggcaaatcagggttcacaaggttggccccaccctaaactctccattctcacatcttaggaaaccaagccctctcaccagtcggttcctctctgagtgttgcaatggcaatgtttctggcagggtgtgggggacccttgctcaatgacctcctgccctgttgctcagaggataccgctgccagaaaagggttggctcattgtggggcttcccaaggtactctggtagccccagcttctgacctggtcctttctctggtatggggataggaggagagctccggaggtaggtatccactctcactcagccaccacatggaaccctagggtggctgggagcacagcagggttcagaggaaggactgttttttgtttgtttgtttgtttgtttttgagatggagtcttgctctgtcacccgggctggagtgcagtggtgcgatctcggctcactgcaagctccacctcccaggttcaagtgattctgctgcctcggcctcccaagtagctgggactacaggcgcccacctccacgtctggctaatttttgtatttttagtagagacggggtttcaccatattggccaggctggtctcgaactcctgaccttgtgatccactcacctcggtctcccaaagtgctgggattataggcgtgagccactgcgcctggccggaagaactggtttttaggagatggtgactggggactgtgagggagctgagcatggcttgatagaaatcctgttagagagatgattataatgttcaaaatcatgtgtgtctgagtgtgttcgtctgttaacctggcaggcaccccatgtatatgtgcatgtgtatgtgtgtgtgctattgtgagcttgggcttgttagagcctgtattggcgtgtgatggggttggcacgcacactcatgcaaatatatgctgtgagtgttattgtgtgactgtgctggtgggtcaggtgagtatgagtgtgaaagagagctggtgtgggtggtttgccctatgtgacgggggttgtgtaagtgtgccaggggtgataggaaggaaagtgaaggcagaagtcatgctggggcagagcccaggccttctggcttcctgaagagggcaggagctgggcagctgctgacagaaacattggcagagacttcatcttccttgtccttctgtctcaccctcaggtctctgacccctcccaaggatcatgccgcagccccactgacccaggagtaggggcctaaggggtgagtggggtagactgagggctttcagggtcaggaaacagggtgggggtggccttcctgaaccccacaactcctcacagcctcctcctcctacaaggaccctgttgctaggtaacggatgggggagccagaatgaggcagcttgagaggctgaaggctggacccacgacaggaaatggccttgatccccctctgcagtgactctccaggtgcagacacacagcctcacacacactcacacacaaacatgcgcagatatatagacatacatgcagagatacacacatccagagacaggcacactgctcccacacagagataggtgcacattcatagacacacagacacagagacacccactcacacagacaggcacacacatactcacacagaaacacatgcacacaaacacggtcttacagacatatacacatgcagccaaacacatacacagagacttttatacactctcgtatacacacaaacctgcacacacagacagacatccacaaagagctgcacacacatgcatacccacacaggcaaactcacccatacttagagacacacaaagacgcacatgtacaagcacactgaaagagtcacagaaacacaacatatcaaagcaataggacccaacctgagcaatatagcaagaccttgtctctactaaaaatcaaacaaattagccaggggtactggcacgcacctgtatagtcccagttcttgggaggctgagaccagaggatcacttgagcccaggagatcaaggctgcagcaagctatgattgtgccactgcactccagcctgggcaacagagtgagatcttgtctcaaaacaaaacaaaaaaagcaaagcaataggaggcaaaaatatgcaaataagcatagcaatatcccaatgtagaaagccagccccagagatatagacatgagccaatgggaagagaagcactgaggggggacatactgtgaggcagactgaacggtacagtaggtggcccagttccgcctttatcccttacagggaggaccccaatctaggcccaagagggaaagccacgtgcctgtatgagcgtatgagcatgtgcatgcgcgtgtgtgcacagggtggtgcacctggcaggggtccttgagtgaggcatgccccattctgtagcagggaacctggaatgggctgtgtgttctgcaagaaattggagccggtggccacggccaaggaggatgctggcctggaaggggacttcagaagctacggggcagcagaccactatgggcctgaccccactaaggcccggcctgcatcctcatttgcccacatccccaactacagcaacttctcctctcaggccatcaaccctggcttccttgatagtggcaccatcaggggtgtgtcaggtgagtccaaggggtcggaggcaggagctgcctggatcctgggagaaactgagggaagaagaagagatgcgaacttgcccttaggagcctccaggaggatgtggcagatacaaccccgctttcaagatcacatgggctgagggagtctgtacagccctcccatcagaaaccacagtctgcaggggagggtcaagaagctctactcccagtctgagaggggcaggatcctgtgacagatgcaagtgacaaagagaaacttcttgcccttttaggtgccacttcccagatgggaagtcttcttggtggggaagaggaggagtgggcaacaaggggatcctccatggtgggaggaatgggcttgaagttgtgtgtcctaagctgtggagaccaaatcagaaattccttggaccccaaaggcctttgggaaccagagcactaaaggagtggggaggtgcagcacctggctggggaacaggaatttggggtgcagcccccttggtgcttctgccccatgccctaccctgctgagtagccctgactctgcagggattggggtgaccctgttcattgccctgtatgactatgaggctcgaactgaggatgacctcaccttcaccaagggcgagaagttccacatcctgaacaatacgtaagtgaccaggccacctagtcagaacattgcctgggctgggagcaggacacagacaggaatcccacctggtccctagcctcagaatgctccagcctagttgggaacacatatacataacaataaaaaccctgggtgactgcaactgtgtgctggttgaggggggtggtgttgggccactgcacccggcctggaggagatgatttttaagctgaggctataaaaatgaaatagacggccgggtgcagtggctcatgcctgtaatcccagcactttgggaggccaaggcgggtggatcacctgaggtcaggagttcgagaccagcctggccaacatggtgaaaccctgtctctattaaaaatacaaaaattagcagggcatggtggcgcatgcctgtaatcccagctacttgagaggctgaggcagaagaatcacttgaacccgggaggcagaggttgcagtgagctgagattgcaccactgcactccagcctaggcaacagagggagactccatttcaaaaaaataaataaataattaaaaaataaaaaataaaaatgaatagacagtgaaggaagaggtaagaaaagaggaagagagtgagagagaatgagaataaatgatgacttctggaaaccacaaagtggttcaccttaggtggttcataaaatatgggatgcagaatgggagagaacagaggctagagaggtaggcagaggcagattctgccaggcttttttttttttttttttttgagttggagtctcgctctgtctcccgggctggagtgcagtggtgcaatcttggctcactgtaacccccgcctcctgggttccagtgatcctgggactacaggcaagagccaccaagcctggctaattttttgtattttcagtaaagatagggtttcaccatgttggccaggatggtctcgaactcctgacctcaggagatctgcctgcctgggcctcccaaactgctggaattacaggcatgagccaccacacctggtctctgtcaggctttttaagccacattgagaagtctagattttatccagaaggaaatcagtagccattttctgtggggaagtgacctagtcagctgtcctctgaattcccaatccccagcccaacccagctggggagcccaaggaagaagctaagagccctaagtgcccccgagcttattccttctgcagggacaagccctcccagggaagctgcagtggctggggcagagcggacaaaagccccagtggtggggggtgtccaagatgagggtttggcaggattcatctctgcagacctgtgtggctccacctggcctcagggtgccttgggggctggaggtgctgctgaccatgccctgttctgtgcctacagtgaaggtgactggtgggaggctcggtctctcagctccggaaaaactggctgcattcccagcaactacgtggcccctgttgactcaatccaagctgaagagtaagtagggattggggcaagaccagccctatggacaggaccctggagtccagactccaaggccacctcttggacaagtcattgctccagtccgagcctgtctccttatctaataattttgtaaggtcattgtgagaacaaaagaagattgtactgataataataatagtagataatagagcatgtactatgttctgggcactattcaaagtactgtctgtgtattaacggggttagaaattactactatcctatttcacaaatgaggaagttgaggcacagagaaagtaaatatcctgtgcaagttcacatggctagtgagtggtggagctgatgtatgaacccagatagttgggctgcatttgctaagcattacacatattgcctcccagtaaaaacaacagtgtatgagctttaaaaaattgtaaagtgctgaaaaagtgcaagggagcattactaggaattaattctattaaggaggcaagaatttttctttttctttttctttttgtttttcttttcttttctttctttctttcttttttttttttttttttttttttttttgagacagggtcctactctgttgcctaggctagagtgtagtgtagtggcacaatctcggctcaccgcagctttgacctccctggctcagatgattctttcaccatgcctggctaatatttttttttttaagtagagatggtgtttcgccatattgcccaggctggtcttgaactcctgagctcaagccatccgcccatcttggcctcccaaagttcagggattacagccatgagccaccgagacctggcccaagaggcaggaatttatcattcattgaatacctttatatacttgagcaagtatactacagcaagtatttatctccttggttcacttaattctcacaataacaccgttatattattagttttttaagagacaggggcgtgctctgatgcccaggctggggtacagtgacatgattttagcttactacagcctcaaactcctgagctcaagtgatcctcccacctcagcctcctgagtagctgatactaccggtgtgtgccaccatgcctggctaatttttgtatatttttttatagaagcagaatcttgctatgttgctcaggctggtctcaaacttctgagctcaagcagtcctctcaccttggcttcccaaagtgctgggatttcaggcatgagtcaccatgcctggccttgatattattattttagacagagtcttgctctgttgcccaggttggagggcagtggcgtaatcttggctcactgcaacctccacctcctgagttcaagcaattctcctgcctcagccttccaagtacctgggactacaggtgcacgccaccacacctggctaatttttataattttagtagagacggggtttcacgatgttggccaggctggtctcaaactcctgacctcaagtgatcttcctgcctcagactcccaaagtgctgagattacaggcatgagccaccgtgtccggccccttgatattattattgttcccattttacagatgaggaaactgaggctcaaaaagcttttaaaaatctatcctcacaaatgtcatacatatatgagaaaagaagggattgaagagactttgcagagtgagaatccaggactctgtgactgatttgggttatggggagtgggaggaatcaagggtggctcaaagtctccttaaggagctgggtggatgatggcaccatttgctaaggcgggaaacacaggagcaggtataagttcatgagttctagggggtacttgttggattgtggtttgtggagaggaacatccaggtggacgaagctgccagcaattcgattaggttgtgctttgtatggcaggcaaccagcccatgctaatccatgcccctgaatcagcccagaggaagggacaccttttcttaattgccactaaaactcctcagtttgtttgctgtggcccttgcagagggcacaacgggctagggcagaaatttgggactcatgagagtaaagatgatcattaaggctatataggagggggctgggggcggtgacacatgcatgtaatcccagcactttgggaggccgaggcgggcggatcacatgaggtcaggagttcaagaccagcctggccaatgtggtgaaaccttatctctactaaaaatacaaaaattagccgggtgtagtggcaggcacctgtagttccagctactcagaaggctgaggcaagagaatcacttgaacctgggaggtggaggttgcagtgagctgagatcgagccactgcactccagcctgggtgacagagcaagactccgtctcaaaaaaaattaattaattaaaaatttttaaaaaagctatatagacggttaaggaagagagtggagagtgcaaaaggttggccctaggaccccactttggggaaagctgccctcgaaggagaaggagccattggagaagagaggaaatccagaccaggccaagtcagaacaaccgagagagcggagaagcttcaggaaacaagagagggcgtgtcagaggctaccgctggatttggcagtgggggtgaccttggtgagagatttctctgtgtgagggtgggaggcggaggccagacagcagagcctgggagggagtgggaggtgaggaagtggagacccaagtgtgaagcactttttcaagtgaagggaaggcgagaagatacagcagaatgttgacggcaagatggaacttagaatagtttcctttggggaagggagaaatgtgggcatgtttggaggttgttggaatagagaggctgcatgcaggtggaggctgctggcaggaagtgggtatcactgagcaggagcgggtaggcgaggttcagaggtcaagtgcggtgaggcccaagtctggggaggtggtaggaggcgtgaagaagaggacggacaattatgcagaggacaggaggtttgtggggagcttcatgcttgtgtccacatcttggagccagtgtcaccaagcactgagaggtgctcagtgcagtgttgtggttacgggtagtgtggttaggagcacaggccctaaagcagacagcctgggttcctgttctagcaactgctgccctgactgtctaatggggtttaacaatagtagctatctcacaacattgttaggagaattaagtgaatacatacgtgtattaaggcagaccctaacacgaaatatgtgtgttattattatagtgattattaaggaggtagctaatgtcatgcgtgtgggagcggggagggctttgaaaaaacttgacaatgtgtcctgtctatacagtccctcaacacttcctccgtccccctcctgcctgccctcctggacacaccacttgccaatgccttcccctgtttcctgaactgtgcctagtacccaggggcggactgaccttcccagggtccactgagacttccaccacccttggtttgaaaacacgaggctcctttaattcaacccaagatggccacaggccagtttcatgatcatctgagtcttctatggcacctgtggtcacttcagctcccccattgtacctgcacagttggtttagggctacggttcaatcccagaaagaatccccaagggagaggagtgattgaggcctgaggttgaagtttagacttaggacaacatccaagtctaagagagtgcaggaagagcagtagccggggcgggagcagccagagagaagagaagcaggagactgtgctgctctggaggccaaaggaggaaagagtttggaggtggaggtgggagaaggatctgagtgatctgattactggagtggtgaaaaaagaactagggagagcggcagaggattttaggtggagctcattagccagcctgtggacaggtcactttccctctctgagcctcaacttcctcatctgtaaaatgagtgcacagaagggctacagtgagatgaactgaggctccatggcaccctgatgactgtgattgtagcaattttaattcaagagaaacctaacgagaagagatgctaacctttgcagggcacctactgtgtgccaggcattttacactccccttctctgtaaccctcccagccaggatgtgaagcaggtgtaccagccccattttacacatggggaaaatggagtcttggcttgtgaggtgactgtgcaggaggctgaggtgaggtttgagcagagcgtacctgactcttgcctgcctttcccaacaggtggtactttggaaagattgggagaaaggatgcagagaggcagctgctttcaccaggcaacccccagggggcctttctcattcgggaaagcgagaccaccaaaggtaggggtggtgccacgccccaaggcgactgggaggcccagccattggggtagggctaggagcggtaggctgcttgggttaaggccaagactgggaccaggtcctagggatgctgctgtcgggcctctcccagctcccagactagggcagaggagaacagcagatcaaaagtgatcctctccacaggtgcctactccctgtccatccgggactgggatcagaccagaggcgatcatgtgaagcattacaagatccgcaaactggacatgggcggctactacatcaccacacgggttcagttcaactcggtgcaggagctggtgcagcactacatgggtgagggcaggggcctcagatccctgaaccaaccaactgaagcattgtccagatgggggaactgaggcccagagaagggaagggactaccaagcagtattggccagacggaaaccagaacccaaggatggggtctgccagcccaggatccagctctgtgagcttctggaggaaagcagtccttcaccaagcagcaccccctaatgactgagcaaggcattggccagtttcttgcctcaaggcctcaatttgtggaaacttgatggagtgtttgtgccgcctgaatgccccaccaaggcaccaggactgccctgtgggcagacagggagccatcatcacagggccctgagcagggggtgacagaggctggtctcacttttgtgagaaactctggctgctggtggagcttggatgccaggggccaaggcagagtgaggggtccattagaaggcagtggctgttgtccaggtgagaggtggtggaggccagactaaatcggtggcagtggagtaaagatgaagcagacgattctgaagctgggtaggaggcagaataggcctgacttggtggagaattggctgtgggggtgaagggaaggcaggagtcaatgcccacgtttctgccttgattggttatgccaaggatgaggtccacggaagaccgtgggcctcatgtccacacctttgcctggaatcccaaccccatttccacctgtgagaatcccaccttatccttcaaggcccagctggaggctacctcctatgggaggcccccagtccttacagaaggcttacagggatcatctctccctctgggtgccacccccttccttgggcaccaacatgttcccctatttagtggatcgggttgcctttcttcctggcctgtgacctcacttggggcctagttccttataactgatcttagggtctggcaccaggctgggataggataaggagtggaggggggtgtcctggcccacctgtgactctacttcatgacccctcccctagaggtgaatgacgggctgtgcaacctgctcatcgcgccctgcaccatcatgaagccgcagacgctgggcctggccaaggacgcctgggagatcagccgcagctccatcacgctggagcgccggctgggcaccggctgcttcggggatgtgtggctgggtacggagctcccgggggccgggacgagggcctgggctcgggggagagggtcctgacaagacagcctccgagcaggcacgtggaacggcagcactaaggtggcggtgaagacgctgaagccgggcaccatgtccccgaaggccttcctggaggaggcgcaggtcatgaagctgctgcggcacgacaagctggtgcagctgtacgccgtggtgtcggaggagcccatctacatcgtgaccgagttcatgtgtcacggtcaggaggcggagcctggtcgggcgggattcggggtgaagttaagaggggagttttcaggcgtgggacctgggacgcgatctgtgagggacaagggacaatgggcagagtcccactaagggaccaggtgtgtaaaacgactggagggctgaggtgggagccgggccgagtgagaccactagggagctggggaggggggcggtgcctccggtgttaggcgggtagggcttgggctaacgaaggcagaatcgggaatgagggagggtctggggcggagtctgggtgggtcgtgtccggaacaccaaggaacagaagaaacgagatgtgggcagagtccgtgtctggcagcagggccaggacgagacaagtgaggggttgaggcacccgcggggtcctaagtgaggggcgggtccaggtgggaggggctgaggggcggggtcaagcgagaggaggaggggctggggcccggggtagggctttgccgctgactttctggcttcttcccaggcagcttgctggattttctcaagaacccagagggccaggatttgaggctgccccaattggtggacatggcagcccaggtaactgggccagcagcctttacctcccggacctcccacctattaactgttcacaaattctctgtcccttcaaacgcctgggagggcggccccgccccctgcatcagctgtgcctccagctgtgcctgagaggtactgcctctctttctgggcctcagtctccccctctggaaagtgggtttttcaaatggtccctcacccctcaaacaggccacggtgttgtgagtccacatgagctcccatctctccacactatggtcccccaggtagctgagggcatggcctacatggaacgcatgaactacattcaccgcgacctgagggcagccaacatcctggttggggagcggctggcgtgcaagatcgcagactttggcttggcgcgtctcatcaaggacgatgagtacaacccctgccaaggtgccctgcttcaccccaccttccaagagctccccatgcaacaagggacttccatggggccccacgcactcaggaacccttcctcactccaggtcgcccgagtcgccccatcctgatgtagtatgagaggcaattctgggctcaaatcccaggtcggccacttaccagccatgtggccttgggcaagtcacctaacctctgggagctgccgtttctcttctgtaaagtgacaatattcagataacaggaagtcagcagatgtttaccaggcacctgctatgtgacaggcacagctataattcttgaatgaaagacaatggcgtgtaacagtgggaattctgtagccagaatgcctgagtatgaatcccagccaggtattaactctgtgatctgggcaagttacctaactactcagtgtctccgtttcctcgtctgtaaaatgagtctctatctcatgggggttttgggagggttaaatgagttaatgcatgcatatcacttaaaacagtgtctggcacacaggaaaggctagccaagtattggccgttattaggataagaattattgcgatttttggaaagtgcccatcactatactagacacatagtaggtgttgactagataccatgtcctttctactatgcccagagacccttgtgctcaggatccccgaaatcctcatccctagagtccccattctctctctgtctcttttttttttttttttttttttttgagatggagtctcactgtcacccaggctgaagtgcagtggtgcgatctcaggttattgaagcctcccaggttcaagcaattctcctgccgcagcctccctagtagctgggattacaggcacccgccaccatgcccggctaatttttgtatttttagtagagacagggtttcgccatgttggccaggctggtctcaaactcctgacctcaagtgatccgcctgccttggcctcccaaagtgctgggattacagacgtgagccactgcgcccagaccccattctcttaatccagctgtttccagggaccccctcactaactttccctgctcccccatcttctccaggttccaagttccccatcaagtggacagccccagaagctgccctctttggcagattcaccatcaagtcagacgtgtggtcctttgggatcctgctcactgagctcatcaccaagggccgaatcccctacccaggtttgcctcgccaggggtagggctggggtgggggatggtcacggggaagggcttccacctggctgtccctttgactgacagagacccatccttcaggcatgaataaacgggaagtgttggaacaggtggagcagggctaccacatgccgtgccctccaggctgcccagcatccctgtacgaggccatggaacagacctggcgtctggacccggaggagaggcctaccttcgagtacctgcagtccttcctggaggactacttcacctccgctgaaccacagtaccagcccggggatcagacatagcctgtccgggcatcaaccctctctggcggtggccaccagtccttgccaatccccagagctgttcttccaaagcccccaggctggcttagaaccccatagagtcctagcatcaccgaggacgtggctgctctgacaccacctagggcaacctacttgttttacagatggggcaaaaggaggcccagagctgatctctcatccgctctggccccaagcactatttcttccttttccacttaggcccctacatgcctgtagcctttctcactccatccccacccaaagtgctcagaccttgtctagttatttataaaactgtatgtacctccctcacttctctcctatcactgctttcctactctccttttatctcactctagtccaggtgccaagaatttcccttctaccctctattctcttgtgtctgtaagttacaaagtcaggaaaagtcttggctggacccctttcctgctgggtggatgcagtggtccaggactggggtctgggcccaggtttgagggagaaggttgcagagcacttcccacctctctgaatagtgtgtatgtgttggtttattgattctgtaaataagtaaaatgacaatatgaatcctcaaaccatgaaa

SEQ ID NO: 30 - protein, 529 aa, Homo sapiens
MGCVFCKKLEPVATAKEDAGLEGDFRSYGAADHYGPDPTKARPASSFAHIPNYSNFSSQAINPGFLDSGTIRGVSGIGVTLFIALYDYEARTEDDLTFTKGEKFHILNNTEGDWWEARSLSSGKTGCIPSNYVAPVDSIQAEEWYFGKIGRKDAERQLLSPGNPQGAFLIRESETTKGAYSLSIRDWDQTRGDHVKHYKIRKLDMGGYYITTRVQFNSVQELVQHYMEVNDGLCNLLIAPCTIMKPQTLGLAKDAWEISRSSITLERRLGTGCFGDVWLGTWNGSTKVAVKTLKPGTMSPKAFLEEAQVMKLLRHDKLVQLYAVVSEEPIYIVTEFMCHGSLLDFLKNPEGQDLRLPQLVDMAAQVAEGMAYMERMNYIHRDLRAANILVGERLACKIADFGLARLIKDDEYNPCQGSKFPIKWTAPEAALFGRFTIKSDVWSFGILLTELITKGRIPYPGMNKREVLEQVEQGYHMPCPPGCPASLYEAMEQTWRLDPEERPTFEYLQSFLEDYFTSAEPQYQPGDQT

SEQ ID NO: 31 - DNA, 26335 bp, Homo sapiens
gaccctgaaaaccgtccctctttagtccactcccccatctgtgccccagtcctttcgcttccaagtggatgccgttttgattgctgtttgtatattattaaaatgtttttatattaaaaatgtttggttggtctgaaaattaaaagaacttcatttaggaaaaaatcattaaaaaacatctgaggatgtttttttaacatctttcttgtgaaccacggaagggacgatactgagcagaccctccaatgcaaaggaaatagactgcagcactgactggcggactttgggtaagtggggtgcatataccgggtaaagaatgggattgggttagaggcccaatttaaaggagttagagtctctcctaagacatagtgggttacagccccctcttaataaaaggcaagaacgcctgaccaactgggttagaggcccaacttatgagggttagagtcccttctaagatttagggggttagaggcccctgtcggtaaagtccctcttggtaaagtcccttttggctaagaacgaatttagcactatgggaattaattgctgttctcttggaaataatctgctttgcactctttgctgatggctgtgggtgacagggttaggcatgtacaggatcagggggcatggggagcattttccttcctaaaaaggggaaacttgagagctgatggcactgctggaaaaaattccttcatgatcagcaagcggctgcccgaacttttcagtgtcgctgcaatgggtgtgtctttctctggcctccctgagtgccttcctcaccctgccttaggcaatcctttctcctctctctctctgtgcaaactggttgaatgaatggtaaaaatcactgtttatctcctctgtaaagttttgattaatggaagaattaattcatgaagctagtcttaagctgtagtgaatttggtatgttttgtgtgtctttctgtgttctataatgcagaggagtactttaggatagaacacaagcctaagacacctacctgtaagcctgcttttcaagacagcccagtaaaccagtcagttacaaactttgctgcaggttcctgaaaaaactggatgagatttccttcctgtcttgtatgtccttgggagcttgaccttgtaaccatgtggccatgttctcttttcacaatggcaggctgggttcagggtttgattcctggcttgggtataagtcctctatcttctgtctatgtatttatatgaattatgtgtgtgtaatataaaatagctagattaattggtttaaaaataagtgcttaaatcaaatattttgtcaaaaaagttaaaaagtgtaatgtcttttggttcatatgacttaagtaatctttgggaaataaaaacagttttaaagattattggtaaaataaaaatattttcaaaaatgtaaatatttggtctaaattatgcaggtcagatattaagtttcctaaatgctttaaggtcataaactgcttctttgacttttaaaagttgttcaatttattttggagcattgattctagataaggcctggggacatgtgaaattagccatgccccctagctatgctaagaaagttcttcataaaatgccactgtgattcttaactattcaacttgtctgctttactgctaggtgaggcctcagacacgtggagtgctcttgtttgactgtgtgagtgagctggttattcagtcatcgtctctctgattaacacttctttaccttggtttgagtggagattctgaaactcgctttgtctttgctggctggttccctgctggactctcgataggggcattagagggagattgggaggctggaggaggaagacaggacatgctcactgattcctatttcattctctttcattgtgagtagcctcagtgatgcttttcacgggcaacagtgactggttccagtttgcagttttccccactctcagaagtagtctcattgggcctcctcagagatatcagcaccagggtcacactatcctcagagatctgtgttccagttccatgtgggcccctcctctgagccagaaaagaagatattttatatgcaatctcattgttaaaaaatgttacacatatattctagctaggttagattaaccaatctttattaaggaagaaactgctaatttaccccatagtgaagatcgagacctactgacttggggcagtgacttattgaagacaaaacaaaacagaataatattggtattatttcattaattgtatcaatgttgaaactgtattgatttcataatagctctgaagatgaagtttggcaaatatggaaaggttcatggtacccaaagactaaatcttcctagtgctagaggccaccctatgagcaagcatggaaatgggacatgtgacccagccttagtaaaattaacaggaaccataggtcacaaatgacagtgctcttctatgagaagccttaagaaataatatcttgttgggagcagtagcttgagaatttaaagagaccctaatagttaggcaggaatatcgtcctgcctattcagcctgaagaagttactgatcgggtcattaaactcctcagaaaaataaagtggtaaggattagagaatgctgccatgtgctgcacaaggaagttttggtccatgacagactgcatatatggtggaggtcccataagattataatgtcataataccgtattgttactatgcctcttctatgtttagatacacaaatacttactattgtgttacaaatgcctacagtattgaggactgtaacatactgtgcaggcttgcagcctaggaatatgctttactttgatttatgttcaagtatcattctcccttagattttgtgaggttgaaaggtatttgtctaaactggatgtgaatggagagggggcctccggagagcaaagggccctctgttccccttcctaatattaataggctataccatacagcctaggtgtacagtaggctgtgccatgttaagttcgtgtaagtgcactccatgatgttcctgcaacacaaaatcacctagcaacacatttctcagaatgtatctgtcattaagtgacacgtgactatttgcactgttgacctcaagtacacaaatagatcagacaagtggagaaagacctcattctacttcattctaatttaaatgcaggtgctgtccgtagttaggaaaatgtgaaggagcaatcagcctccagtgatattcaagtctaattaaaaattcttcttggactgtttagttcaaagctccctcttaatcttccaggagcttaatcctccacacaaagtatatgaagacacaaaaatgtagaaagtatgtgaaatagctattatttaagtgtggaatattaattttgttgtaaggaggggatttattcactgaaataaactttattaatttgactcatcgccacagaagtcacgtaggctcctctctgagtcagaaaatgtcaagagctttctgtctatttttcctctttagggactgcttgagtacagcaaaaggaggggccagcagatttcagttcaagcatgtttgaagcctgttttccgacattatttgtagaacaaaaaataaacatagtaaaatctccactacaaatgatctctataaagtgtaataacatgatttaagtttatgtattattttctatattttataaattgctgcattgaacagctttaataaaaaaaattaaagttagccttgaaaatttttttggttgttgtttgttttaatttacagacatgatagtaccttttagctgttcctatacttgagtagcaatcaatttgggtgtaaaatattagggtcataatgttctttatacctcatcccactctctcatggtatttgttactcagagaagaaatctgagtcctgattatttcaattcctttgaagaaaccaagttatttttttgtttatttagcagtaggaatctttttatttaaatttaaaaattttcacctggaaacttcctattctaggtctcactttgtgaattttttccttgcccatgttgaactctcaacctccaattttagatttcttctcagacctggaaagcatattttttaagataaaatttttaatcttgcttgtgatccaccattccattttatgctttaaggacatctctgatttgtagattggaattctgtgatttgtcctcccttaaatttctgagctcttctaaatgtctttattcatttcacatttgggaatatttctgaagtgaatcctctccattattgatttgattttcacactgtcaaatctgtcttttcttgcttctagtacaaaacctgctggattttttcttcttgcttcagcgtcttgaaccaagtttcctttttaaatctaagtctgttcttgttgggtggtggcagtgtcctctcaaattgtttctggggtccctacacccccgtgagctcccaggccatgtctgtggcaatgctgaaggacaaatgcagggtgtggccctcctggtgatgaagaggaggtgcaccccaggtcaccagaagccaagagaacccacggggctgagcagggcaggatgaagcaacatgttctcacaaaaaccctgaagatgagatacgattgtcgcagcctctccctatgtgggcctgactctcttcactacactaaactggatgtgaatggagagggggcctctggagagcaaagggccctctgttccccttcctaatatgaatgccttactttgatttacattcaagtatcattctcccttagattttgtaaggttgaaaggtaattgtcattattaccacagaaaggaaaactggagttcaaagaaacttaactgagaagtgagtcttggtctctgtattattgtgttgatgtcaggagtcagagcaactggtgatgaaacagaacccaaactcagtgactcccaggaatggcctcatatcctgttgtgcaactttccgaagcctctaggtcattgtggtgccttgtagctgtcccgggagccctcagcagcagttggagctggtgcacaggaaggatgaggaagaccaggctctgggggctgctgtggatgctctttgtctcaggtaagacaaggaagaggactgagcaggggaggcgtttggtccaggatctcggggagcccttttctgttgaagcttcagagcacagggccatccaagatagagccaaccagtagttatggcagaccgtgctgctctacccagaattgggagaacatgcagtgtctgcacacaaaagtaaaagaaggagagagtcagggaaaaaagatgtctgtgcatgtcgatgttgttagagacatgcaaagcctagtatgggaataatgatagagtagatctcatcatggcatgatctacaataagtttatttgatgttgaaatttttttcttatattccatcatgatgtgtttgatgttgacaatctgttaaacccaaaagacagaattgatttaacaaatatttcccaggggtctactatgggacagcaaatatactactgctgaggacacagacgtgaccagaagaggctggtgctcattttggtggaggtgatcatcccagctctccatggcacgctggggttggagggcacagttgctgaggctcagcagagccggcgcatgccccagctctttcttgtgtctcctcttacgatccgcacagctcacatctgcacccaaaccagaagcatgcgggctttattcactcctcctcctaatccttatgaataacaggaggaaagtgtcaatatgagttaaaagcgaggcttgccttgggaaggcatgaaaaatcagggagaggtatcctcctgggctctctactttgagcaaagtcatggacaactgccttgaagagcctgtgctcagacattgctctgggtcttcttggagctggggtgataatctagatgggcagaagggagttagctcctcatggacaagtgcatttccttgtgtttgattcatgtttttctgaaggagaactgggagatggcagagggctgaaggttatgtacctgtgtgagagaaaaccagtggtatatatttcattgtgccctgtggggataggttaggggttaacctcaatgctccttgcaactggatacttattcacacttgggccaggacttctctaatccctatcagactgtgttggaccatgtagctcagtgctttaggtggaaccagagtccttgggtttgaatcctggctctagtacttaactatggtgagactttgggcaaatcacttaatctgtctgtgcctcagtttccttatctataaaatggggagaataatggtgtcaactttttactttataaagattaaataagatagtctttaaaaacccttagcacataatacttaataggtagtatttgctacccttttaattaacatattcaaaggaattcccttcttgcaattgtgcacagacagctcagcagccttcatttcttattgtctccttccttctgtgagagacaggcaagtttgcaaactcagtcacctgtaactcccaccttccctggggagctggcaaataaaagagatttatctgtaacattaacttcagaggttagggcttcagtggtggcaacttgaggatggagtctgtgtgtcttccaaagagcccagcacagtgcctatgatagagaagtctcccagccagtctgatgagtgagtgaacaaatgagggaaccaggcttggtttcagacaacactcagcctctggtcaacagtctcttggaggcacattcctgactggagtgtgtgggagttgcccaagaggtctgtttccgaggtttccctagaaactggagtctgctcccagaagaactggctttcaagactgatttcacctttaaactccttgtgagtccctgagttgagtcttcctaaggtcaggcagatctgttggcctatcctggggtgggcatgccagggggctccatgaaagggtaagcacttgtcatttttcaccaggcctgcaaggacctaagtgtgtggtattaataacagcagtactagtgttaccgtgacccacacagtagacccagcagcactgtctagcctgttatttggtgctccatgggggaaggtaccccaggcctgagtgggtaagctacagtctctcagcaaacttagagagccctgtgtgtccagctgaaaacacggaccttggtcttctcacttccgctctgcttggcctcccatcagcaaggctcccttggacaaggccagatgagcatccccaattctagatggttaacccagttcggactccaagaatgtgatggaaactattgtcactgtaacccaggggttctcaaacatggcactattgacatttggggtgggatgattctttgttgtgggggctgtcctgtgcactgtagattgcttagcctggtatccattagatgttagtagcatccacccctctccctggttataacaaccaaaaatgtctagatattgtccagtgtttcagggagagttgtggcggggagcaaaaattgctccccagtggagaaccactgctgtgactccgtgacatcaaaaataggagggggcatttcttggctcaggtaactgggaagtacaaggggtgctcctttggagcacagctgagtccatgactcagataacgtgggtttttttctctctttctttctctcccctccactatccccctcttccttcctcccactcactttctgcctgtttagccattctcgggcagcctattcctacgtggtagaaagtttcacataataaagtttcacatctcattgacctgtgaatcccagcaagaagcatttccattaatggctacagcagtctcagggaggactctgattggtccagctcagccatgtgctccttcctaaaccaatcagcctggcagggagatggggtactttgtgtatgtgtgtgtgtgtgtgtgtgtttttttttttaacagagtcttgccctgtggcccaggctggagtgcagtgttgtgatcttggctcactgtaacctccacctcccgggttcaagtgattctcccacctcagcctcccatgtatctgggactacaggtgtgcaccaccatgcctggctaatttttttgtatttttagtagaggcaaggtttcaccatgttggccaggctggtctcgaactcccgacctcaggtgatctgacctcctcatcctcccaaagtgtgggattacatgcgtgagccactgtgcctggccccgagatggggtacttggattggcaaatcctgtgtcctgtgcccatccttgagaatgtgggctgccccaacagtagagccacacaggatcgttcccttggaaaggggtgctctgctacccaaggaagggaaaaaggagtgccaggcagtcagatatccatgcccatcatgccctccagacggcttcctcactgtttgtccaaaggtcttttgagctgatctccttgccctgacttagatgaagttcctcaaggtgtccttggagccactctctgctagcaccgtgtctttgcgttcactttttatgtcatacgcccttgtaatcgatccctcccaccatgctgcagccactcgtggtcttagtacaatatcccatgttctgtctccaggcaggcagtttcttcctctcaaagtcagtcttcctcttctgatcatttatgaaatttgatcctcatgcaccatatgcagtctcttatagtaaaaaacctactcacagtggaaagatctgaggtctagtgctggcttgcagggcaagttgtattttctctggaggcctcaagaacctcatctgaaatatgggtggttggacaagaaaaacagggggctcctctcctttttccccactcagagagagaaaaataattcctcacagaacccattctctttccctgcttatagaactccgagctgcaactaaattaactgaggaaaagtatgaactgaaagaggggcagaccctggatgtgaaatgtgactacacgctagagaagtttgccagcagccagaaagcttggcagataataagggacggagagatgcccaagaccctggcatgcacagagaggccttcaaagaattcccatccagtccaagtggggaggatcatactagaagactaccatgatcatggtttactgcgcgtccgaatggtcaaccttcaagtggaagattctggactgtatcagtgtgtgatctaccagcctcccaaggagcctcacatgctgttcgatcgcatccgcttggtggtgaccaagggtgagtggcctgggacttggggggaaagaagggccaggtgaggaaaggggcagtggtggtagcatcagcagagctggggttcaggattcccagcagtctgtcttcctcccaggacctctttatctctcatctaaggtccagtagtaagtagaaccttccatgcatgagattctccttggatagcacagcctaattttgggtgctgtggtaggaattggggtacaattaaggtgagtggtagatttgggataatttagtcccattctttgttctccacttagaagtcatgtcctgactccccctcccaaggatgcgtttggtggcatgcctgtgtgcttctgaagcagcccctctcacatctttctgtgggcacagaccacctggggatcttatttcaaatgcacactccaactgaatgggtacaggggtctgcatctctattgatctcccaggtgatgccaatgctgatggtccatggctcccattttgggggagcaagtctccagagctcattttgtgtccccttgtacacaactctactcactggataggagaggggggtttacttatctgtaccctattcatttcagaactgcttgagggaagcacctgtgccttcctgggctctgcacccctagcaaataatgtagtttctgacacaaagtaggccctcaggagatgttgcattaaggaatgaattatggaagacagaggaaagggaagcaggctcaagttcccagcacctggggaattctaagcctgaggaagacaaggtgcacacacaatatattaataaaagataacttttaaagcaatatgccataagccaggtgaacgaaaggcagagtgaatcaagtaagcctggaattattcttggagaaggttggggcacaagaattggtgaagaacagaaggcaagttcatttccaacagtacgaatgacaaaaaattcctttcttcttctctctttctgctctccctcacttcctgccctttttttcttctttccttttttcgatttctgactcaggttgactccacagtatttctttggctttagcaaaggaaaactttgcatttgggccatgctcatttccctcctcagcttcatttaccgtaccacctacccgtccatcaactcatccacattttcatccatacatccatacatccattcacccacccatccacccattcatcatccatctgtccatccatccatccaatgtgtcttcttgcaggtttttcagggacccctggctccaatgagaattctacccagaatgtgtataagattcctcctaccaccactaaggccttgtgcccactctataccagccccagaactgtgacccaagctccacccaagtcaactgccgatgtctccactcctgactctgaaatcaaccttacaaatgtgacagatatcatcaggtatagtttccaggtccctgggcccctggtttggacactgagccctttgtttcccagtctgtgtgctgagaggatgtgaaagtgagggaaaggggagggtggggcaggagaagacttgagtcacattagtctgggtagaaatgtccaggggaagaaggaagtggtgatggagaataggggaggctctcagccaggctgtccttctccccagttcaccttctttgtttccttgcaacctgagtattaaagagagggaaatggcatcttccccaagttccagtggagctcatccaaccccaggggcctgatgggcagtgggaaagcactctgagtgaggggccctggatctagtgttggcctgactaactgaatgtgactttgggtgagtcaggaaccctctctggctttagcttctttgacaattcagtaggcatggtagaaacccaaagctggaagacattgtccacctaataactctcagcaggagctggagctggggctaaatgcagtattggtttttgccttattgttttttaaataacattgttgcatgtgcccaattatagataaatagaatcagaatttttgcagatgaggtccctctcagtatttttaataagatgtttaggagatttctaatgtgtaggcaaacttaagaaccactaacttagcaatttcacacctctttacagttattaaatgttgatttatattagaatgtgtttatcattaaatactgaattatcaggatggaaatacttttccacatcaccactagtctaacatgtcttttttctcatattctcttctagtctttattcatgtttatacatattttaatatggcagtaagtctaattatacatgtttttattcttttccttgctgaaattattctttgatgctgctacctaaatttcataaatataatttcaatgagtgcatatttttctatcaattgggaaagccataacttacataaccattggcctgttcttagacattgatatagtgttgataactctgagataaacatattcttgcagatatatttttcttcttttaaataattaactcgaaatttccatgagataatttaaacactgatatcattttgtatacaaatagcatctaccaatggtcttccaaaagattggaaacaatttgcagtgtgatccattaagcataaataaagcagtgtcatggcaggcttacagacacggatttagtcttttaaatcattaaatagtgtgagttaaatcacattcctgctaaaacaaatgtgaactggtgcctgctctaatttctctatgagtgtagactccacctccatatgggtagtggcagtgcctttttccccattatgttgtttggggaacaaagtgctcattaaacttctgtggaataaatcaaacgaatgatctactaagagtcaatttttttttttcctctactttcattttgcaatacataagttaagagaacataatctttgcccatattcttacttttaccggaaggagggccttaagtgtaagctgtccaggttctgggccttttaatcaaagtattggagaagacgcacaaagtaacaaaggaatgaagcagtgaaagcaggaatttatttaagcgagaaagcgctccccagggtagaagtggccccacgcaagtggctcaagggccaagttacaaagttttctgggttttaagtactccacgtgaagttcctcccagttaccccttatctggatgaagcatttggtttgtggctaactaaaggctaaggtgaattggcgccctctgtgggtaaagggatggtcgtctctgcttggctcagggccaatctaagacactctccctttccagctgagacgtggtggaagggggagggctgtagggagagtagcctttgattctttgctgctggggcatggggagatggggtttttccttttgttttagctttagaaagtttatgttaattggccttaggttccctgaccccagccccaggagttttccttttgatccagctatgggaagtcagcacgaatccatcctagatttcttgcccctagaccttggtgttgctccatgattcagcatgaattggtcttacgttcctgcctccagaccctattctcctgccttgctgctagagactgcatctctcttttatctgttaactttatctatcatctaatatgaactatcctttcagccaaaatccagtctggaaagtggaatccacactagctgtttgaaaagaagaagctttttttctttttcttttctttttttttttgagacacggtctcactctgtcaatcaggctggagtggagtggcacgatcatggctcactgtagcctcaacctcctcccacctttgcctcccaagtagctgggaccacaggtgcataccaccaggacagctaatactttttattatttttgtagagatgggggtctccctgtgttgcccaggctgatctcgaattcttgggatcaagtgatcctcccacctcggcctcccaaagtgctgggattacaagtgtgagccaccacatccagccatgaaacgaaggaatttaatgcagggaattgtttgcccaggtgatggaagagctgagacgctatcctggggatggagaggcactgcagaggtagccactatcaccctatctctaggtagtttccccagaagcaagctgtaaagtcttgttaaaatgttttaggactaggtgttgtggctcacacatataatcccagcactttggcaggctgaggtgggagaatcacttgagcccaggaatttgagattagcttgggcaacatagcaagactccgtctttacaaaaaaataataaaaaaattagcagggcatggtggcatgcacccatggtcccagctactcgggaggctgaggcaggaggatcacttgagcctgggagatcaaggcttcagtgagctgtgatcaagccactgtactccagcctgggcaacagagcaagatcctgctctctttctctctaaaaaaaaaaaaaaaaaaaaaagacaaaaagaaaaatgttttaggaagtgatttctggcaaagccattgaggaagtgggaccaggaagggaaggaagccaagcaagcgtgtcataaccaaggggtatctcacaggataactctggctcagtcttgttaaggagctctggggactcctgggtcacatttggagttgtccctataaggggcagtaactgagcaagggcagctctaaccaaggccaagcacatgaattcctggcccttcagggggttggtagcttgtgtgcagcctcgaaaaaaatgcagatggtggcctgttgggagtggacatgccccaggagttcagcataggaaaatggtaaagcactcagagtggataatgcagagcactggcaggagagacttggggaggaggtgcagtgagcagagcccggaagccaggacattcaggttgcagcactggaatcggggcagatgctgaggatgctgagtgcgccaccatgtgaggggtgagagagatgctggctccctgccccttgctgcctgtctctctgccccatccctgcccccgtctctgccccctgtcccccaccagttgctgaactcgaccagtcaggcttgggaatgtagtcccctgtgctacaagtcagggcccttgaatgggggtggatttcaggcaaataggcagttttctgctttctagctcctttacgccgtttccctctttgctagtttgtgcatgtggttttaagttttgtataggtgggtgtgttgattttaaatcaattgctttgctttattccttaatgtgtttgattctatttcctggtgacttttcttggattgttttttttttattttatttaaatgtttagtccgggggaagatttattctgctacatgctataaactgtagaaaagcctacaccgttaccatgttgctgtccagatactgcaagactatttactaaatgatcctcctgcctctccaagtagctgggaccatgggtgcatgctaccacgtccggcttatccactggacctctgatttccttagacattatgttcctgcatagcactggctctgcatctggcctttgtgttccattgccatctgtctgtccctgtgtcttaccatgccacatccttgtgctctcatctgcttgcatgtctgctgatggaactggccaagtgccctcactcctttgcatctttttattatgatacctattgatgtttacccagaaaccttagaatcatttcgccaaaatctataaaattcatgatgagattttcactacgggtggattcttctcggtttataggtgaggaaaccgagctaaagaggtgagatgaccatcccagtaggcagggtacggtactgagaatagagagcgggtttcctggccagagtcctctctccctaccacatcacagggaggaggatgggctgatcagcccgactgtctccctccagtgcctctcctggacagggacctatactctccactgaattgtttctaatcagtagtttttggcaatgagacagactgaaaagcacagggtcaggcagtgcgggaaaggtggagcaaggatctaagcagaggagagtgggacatgaaggcattttctagagggaacatgtgcgtgtgggagagactttgccagcagtagcgttcagcctcttgtctcccgcagggttccggtgttcaacattgtcattctcctggctggtggattcctgagtaagagcctggtcttctctgtcctgtttgctgtcacgctgaggtcatttgtaccctaggcccacgaacccacgagaatgtcctctgacttccagccacatccatctggcagttgtgccaagggaggagggaggaggtaaaaggcagggagttaataacatgaattaaatctgtaatcaccggctatttctaaagtcagcgtctcaccttcctgcccactgccctcgttcctctaataatcttgggtgggcatttgtgcctcagaaaagaagttacagccccaaacatgcttggtccttcattccaccagccacttggggttggcatgaaatacagacagctcaatgcttttcaccgtaattctcttgtgggggctgtgacatgcagaaggcacacctgatacttctcctgctcagttttgccctggaccatacaattttggcctgacctggacagagctcccactacagaagcatcctgctcgccccatgctgggacttcctctttctagcatcagacacttgggtttcatgcttatgtgtggttctttccaacactcccagaaaagggtgttgaagattgtggaacctggagaaataagacatcgtggtgagaaagtgcatccttctcagagaaaagagttaaactgagtatcttcttctggggaaatactggcaggccgagatgggatccataggagagcaacaacagaccatgtcagacatcctgtgtgcatttatcgctggatcctgaaaatagccccgtgaaggcagaaatgtatgtgactagaacgaggccacatgaataagccactgcccactggcaggagtgaaaactgaagcgctccttacctgaaggaccccaaaaccatatagaatagaataaccaggagttccgcctgtgtctaaatgcctcttttcctgtatcacacaagggtcagggatggtggagtaaaagctctccccctgggaggcttctggaggctgtccccatgtgcttgcctagttccccactctgccctcctcctcttctctcagtctgctcctggaacacctgcctcagtttccatgctctctccagtgccctccccggtgaagcaggtaggtgttcaggccaccacagagacaatctctgtgggagattgtcttgcaatctcccacagatttcaatcaggattttgttatttcctactttgagctttaaagggaaatgggcctcatgggtggggaaaggatggtgggtccttccagcccaatttagtgatgcccagggcagatattatcctcagttcccaagagcaatcatacttttccacacataccgtgtgtctcatgttaggtaaatgtatttttacaatgagcaccacttctgtggaaaaagttccctgcacggggaggtccagcttccagactgctccatcgcataaggacttccccattcccctaaatgctgctctgtcagaacctgcccaggtaatggtaatgaccctagagagatgatttctgaaccgcaattttgagcccattagaaggtgtgtggtgggcatttatttcatcctgatgctctggtgagaatctttgcagacgcactagatccagaagctgttaatcttggtgcatttattttcctacctaaaagaaccaagcagctcagaggcagtgactgtacaggatgcagtgtttataataatgctgagcttgctggtctggaaccccacactttagcaatcccagcattgttcctgtttatgaagttgacaaagtgaccagggcaagggggtattatcattaaatacactctaggagaggcagaacacatgagggcaatgtttttcagaggtctttaggccaccgcatcagattctcctggagcataaagcaaatgctttatgagtccagggcccctgcagacctactgtatactagtatacagctccctcttagtggatctcaagcttgtttccaaaaagtcattacactccttaccaaagcccatgacacattcatacagattcatccagacataacccactgcatggtccagtgcatgcttgtgtgcttaacttattatagatcaagtgttatttaagtccaacatattaaacgtgactgaaatattatatgcactaaatcggaagacaaatggccgtgctggatactctcccatttgcacttacaggccacgccatgctccatcctccccatcctgcctgggaacctgaccaatgtggctaaaccagcaggctccctggctgccagcttctggctggttgagccaatgggaagcatgagaggagagccgagggtggggagacagtcagatcagggtttctgtcctcagggctccctccctggcaggtggtggcaggagtggctgcattcccctctgaaggctccttcaagcctctcagcaaacagctcccatctccaagtccagccacctgttccatcctctcctctgtaggcccagcagtgggaatgacccaccactattgccagcctcagcatactgaagcaccccttactggattccctaaattctatgcacatgtttattaaatgctcctcaattacccagtttcaatgagccagcggtgtcttgctgttatcttgactgatacaatagtgttgtgtggctaacgacacaggtcttggaggaaaaccatgtatgggtttgaatctagctctgcatccttgcagggtgtctgatcctgggtgactcagttaaccttctgggcttcaatattcctccttgccttaatgggcatgatagtgcctaaccatgccagtgatgattttggatgctaacaacagaatccaactctggctatctaaaacagcaagggaattctagggggatatcagtaactcagagttgatgagaggctgggggattgggtttagaaaatgggcagaagccaagagagaatggacagcagaggccacatgagacagtctggacatgttgccatcaatgccactgccctggatactggaggccaccatgctattcctagactctgaatccttaaattgcctctgagtaagtaatctgcaactggccctgcatttgagcctcatccgcagatctggggtcctgggcagaatcatgcatgtggccaggcttaggtcaagtcacgtgcctgcagcacagctgccgggagaagggtgggaggaatagctgcttccatcctcaatagaaggggaagcagaactctgcatctggtgaaactcacacattggggcctgtgcccccaaaagaaaaaggtgtttggtactggagagtcaaaagatgacacacctccattataccatctcccaatgttgacaggatccaaggagacgcctgagtaaagcacctgacacacagtaggacttcaatccatggtggttactgctattactgttataactggggtctccatgcctgtgcctggacaggttagctcagggaagcccatacctggtactcaggcaaagcccctgagtcaccgagacatgctgcagggtccatcttacctatatgcagctcttgcagacatggtcaacttggactccttgcctcagaaccacctaagctgttgctaaaatacagattcctgggtcccttcccagatctgccaagtcagaatatctggtggtggagcctgggtgtgtatgtctgccacacttcctgagtgaagccacgtgcctgtccttgggaaccatggtgcggggtgatattaaagagggaggatgtagcagaaaggcttgaacccggccttgggggatgaacaatgtttgtttgcgcagcaccatttcacagggagactggggtgcaggcttgcgggttgtaggggtcagtggtcagtgatgaggaggttggtggcctgaaggggtgagtgacgcagtggggacatcaggtgggtgggtctagactcaccatggtgcagaaagggaagcaactgagtgttcccatcaggactgtgacatcatgatggcaggaaagaagctgtcacaagggtggccagcctgcctctctttttctgcatcttcctgagctgttccccgagtcacacttcccccaaggtttgtcaattgtaaagtagccacccagcctcgtggggagatggtgaccgcgtatgtggggcaacatcctgcagcaactacaagaggccctgggccagccggcagacagcacatctgggtggcagatagagtggagggggcagggctctgagggtggccctggctgctgctgctgctgctcctgctgctgcaggtaaggagagtgggtaagcaggctggggaagaggggaccagggagagggatgtagacagcccagcagaagtgcccactaatgggagtttcctcttatatctttgggatggactggaatgggatcaggtaatgtggtacaaatgggaaagggaaaggaggcaatgcatgcagacggtgcttgtccagcctgacccaggagggactgggcgatgaaacagaagctggaggtccctctactgcatcttgaagttgagagaattaggtggattcagcagagcaatcccccagcactgtgctgatatcctgggagatgtaaagaaaagcgagacagagtctccccttaaaaaagcattcactcactgtgcagaggaggcgttagtagagcaaaaggtaaataacagcaaaaagacaggcagtaacatgaaaacaaaacttctgtggaagtcactctagcagggcacagaagggaatgtggagaagaagacaagagttctggaatcagacagggctggggcaaatcctgtctcagccacactctatgtatgtgaccttgagtaaggtttctaacctctattaggctcagtttcctcactgtaaatggaaccgactcacaggtatcagagggttgtcttaagaattaaacagtggatgtatggaaagcccttggctcagtatttagggcctaataagtgcttaataaatgttatgatgggagatgatgatgatggaaccaggtgtagggtgatccgtgatatttagggtgaagaggaggtagagactagtgccagctacaactgttcaggttgactgccctctgggaccaaatgggaatatccatcccatttgggagaaaacagagaagaatgggaagagcatctgtcttggggacaccaggagctaatacaaagagctgtgtgaggccaagtgctatggctcgcacctgtaatcccaacactttgggaggctgaggtgagaggatggcttgagcccaggagttcgagaccagtctgggcaacatagtgagatccctgtttctagaaagaacaaaaagttagctggacatggtggcacacgcctgtagtcccagctatgtggtgggggctgagatgggaggatcacttgaacccagaaggtcaagactgcagtgattgcaccactgcactccagcctgtgtgacagagcaagactctgtctcaaaaaacaaagacaaaaacaaaagaactgtgtaccagaaagcaaaagccatgcctgggccaccatatgtggaatagaaaatgggtggtaaatctggtgaagttcttttgtacctttttatgatgaacattttccagtctacgaaatgtagaaagagtagtcacagtcaactcatcacgtagattcaaacttcaaccatgatggatatttgccttatttgctccatatgggtaggaatcttgctaagatatatatatatatatatatatatatatatatatattatataatatcatatatatgtgcaaacagacacatatatgattgcagaactatttaaaaataatcacagaaatacttaatcactcctacatgcttgacagaggagctaggaatactctttgtctgtggcgctgtgtctgtgttgtggtctgaggttggggatgcagaggtgagagaagaaatcatgtttgctcagtgaagtgcttgacttgtcattcccagattccaagctgtaggtgacatggaggagcaaatgtgcctgctagaaggacagaatctgaccatggcctgttcttacaacatcatgaaatatgcctccagcctgaaggcctggcagtggggttggagccagggtcccccagagactctggtgctcatggagactagaaacaaagacctaaactgggcctgggctgggaggtacctgctggaggattatcccacggaagctataatcagggtcgtggtgatagggctctggaggcaggacttgaggcgtattagtgcatgacccacctgtctcctcaggtcccctttgtcctgcaccatcggatttggctggtattggtgcaaaggtgagcagctgcaggattcaggtcatggaagggtctccttaggcacagccaatgagctggaccccatctgcccattcatgaggggctgtttggagggcagcacagagcaatgggaaaagttgcaggcctgaaaccaggggacctgggtcttccattctcttgctttgtgacttttttgagaaagtcaatagcttctctctgggcctctgttttctcacctgtaagacaaggatgttggactaggtaatataaaaaattatcttaccctgtggcagagggccgctagagaaactggttaattgaacatgtgctatgagccaagcatgtaccttgttaatctctttactctgaaataagtgctgctgtcttcactgcagaagagaagactcaaagagattaagttattcactatgtcattctgcagataagtggtgcagctgggacttacaggaacccatctgatttcatcatacctggtgaatcaatgaaatgggccccgtttggtcaatgaaaagctgttcctcttctcaggaatttcaatagcagtagatgaggcacagttattagggaggaaaatgaaggacattttggtgaccttcggttgcttctcattggtggaaacactgccaattccccctcctcctttgttagcaagccttcctgcctacatggggaggtggaactgggatggtggagctcagtgcttttgcctcaagtttttctataaaactcttttcaagctagtgaatttgagaggcctccattaattagcagaggacattgctcttctgctatttgaatatatgctaatctgaatatgtgaatatcaggacaggctggggctgaagtcagacttcatagcatctgcaaataaacagttgaccaagcaaagggacagagccatgggcaggatggttggaatctactgaacccatgtaatagaataaaataacttcccggttcttgagaaacatccaaggatacccggtcactgacaggctgattacaaatcaatgatgcaattggtcaagcaggtccatcacccactgctcctgactcaggagcattttattaatttggtcttattgcctctttgttacataaatactaaaacttcagtcctttgcaaaggtaagggcagccatcacagggcaatagaagattgtggattcttggtccctctctgccttgatcatacttagtgaccttggggaagtgcctcacccttttctaggcctcagtaaacccctggcagtggggcttcccctctgtgacattcagtggctccaaccttttgggtttgttgatctttattgagttcagggaaataaaggcctatatttcacatgtagcaaagttggcccaattcaggcaccagtggatggcttggtccttctggtatgaatccatcattggagttctgcccagttctttaatttaagtcttaaaaaagacaagagaaatggttgagaaagactgagcgcatactgttaaatgagaactgtatcaagtttaataaggatacgcttaaacataattatcttcattgaaccataaataacaggcagtgccattttgatagcagcttcgtattccttctctccaggaactggagaaaacttgaggctaatctagccagagaagcagcatcccgtacagattgttcttgtctaagggaatttctgatgttgcagagggaccataaaggatgccaagtgaaacgtccttgcctatctcctgtatgggctccctgacccaatcatttcccccgcttgaatttgcagggaaaagagcatgacatttggcatcagaaggctggatgtggaatcccatcctctcccaccactgcacactgggcactttcggataagtcagttcttctcccaggctggaactcaatccttgcattcatgttataaaggaaaacctacgccctgaaagatggcaaggtcaactgtaagcaaacaaaaatgcaaacatgctgtgtttttgttttagctttagcttttgtcttatttttttcacaaaagatcttcatgtaaccaaactgagttgacagactccttatttcatggcccactttaaaggaaacactttaccactcatttggtttcatgtgttttcttaatgtgtatttttatgtatagagatggagtcttgctatgttacccaggctggtcttgaactcctgggctcaagtgatcctccctccttggcctcccaaagtgctaagattatagccatgaactaccactcctagcttaatgtgtgtgttttttttaatctttactaccttcatcctgagctgcactgcctacccttccctcataggcatcttcttaataatcatatttcatattttccatgtctatttgcatgcatgtagattcttgaaaatatatagaattgattttgtgtgcttttgttttttacacatttcactctgttgtagaaaaaaatctgcaattaaactgaaacttctgcattatctattgcactctcttctttatgttatctggtaatcattttgtgagagaagtctaactttcacaaaggcaggttattgatagacaagcaaactctgccctgtcttccttcccctgctgagagggaaaagcaaaccccaaactgatatgcttccacgtacaattcatttcaatattcctttacttcatatacatttgtgccttttgacttccatttttaaactggtataacatcttctcaaataaattatgtattatgagttaaataaaatttttagcatgtgtttatttttgtctcatggtttaagctttttttttgaaaattataactctagttatatgttttcagatagcattgtgcttctgagactatccgttgttgctatatgtaaatctaaatcttcaatttttggctgctaggtaggatttcatataatgtaggggcagtattggatttcatttttctgttccccctagtgatgggaacctaagttgcttcaactctttactaccacaaaacaacacaagcatacaaacagaaaaacaagcaaacaaaatacctcaaaataaacacttccattcatgctcttgaaggagccatataagaattttttaaactacatgttaagttggcattaagccaaatagaaaataggaacaaaatagagggtttaggaacatacccacacatagacagtcacttgactgccactaaagggcctctgcaatttagtgtggaaaagatgttgtttctatcaaagggtaatgatcagttggatacccacccagaaaacaaaataggtttttaactctttcacactgcacatgaaattaattgaaaatgcatcatagacctaaatataaaaggtgaaaacaatatagtttctggaagaaaatgtagaaaaaatgaccttggagaagtcaaatattttctaaacaggacgtaaaaaatagtatgcataacagagcataagtcagggatggaacattgagcatgggatagccacatatttatgccacagaggctccagattgttctccagaatgttgggtcatgttgcattccccccagcagggctcaaggatccccctttcctccactctcaccaactcctagtgtcatccaccttccatgtgctcaccattcagatgagttcaaagtgacagttccatgttttaaaattagtatttctctaactaacacgatcaaacatctgtttatgagtttcttgggcatatagttttttactgcaatgtataacctatttctaaccattttctatttggcttccttccttccgcttgttgatttttaatcagcccttggttagtttagatgttgaaattatttgtataaattcacaggatacaaatgcaatttcattacatgcacgaattgcaaagtgtccaagtaagggtttttagggtatccatcacctgaatgatgtacattataccattaagcaatttatcatcatccaccccacccccatcccctcactcttctgagtctctattgtctattattctactctctatgttcatgtgtacaaatattttagcttcagcttatgagtgaaagtatgcactatttgtctttctgtgtctgacttgttccacttaagctaatggcttccagttccatccatgttgctgcaaaagacatgatttcattatttttatggctgaatagtattccattgtgtatatatatcatactttatttatccagccatctgttgatggacactttggttgattccatatctttgctattgtgaagagtgcagataaacacacaagtgcaggtatccttttgatatattgatttatttttcttttgttaggtatacagttgggattgctggatcaaatgatagttctatttttaattctttgagaaatttattttttgttagtagctgttctaacttacattcccacctgcggtgtataagagttccattttatctgcatccttgccaacatctgttcttttctg

SEQ ID NO: 32 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 33 - DNA, 14179 bp, Homo sapiens
tttttaaatagaaatatatacacctaagttgcattacaaaggagttgtcctccacacacttattctttcccaccatcagaggtttatggtccccgcaaaagtcaccatcagttgggctcaactccaatcagtcatggtaggtcagggtccatctcattgtctccatcactcatgcccacccagggtaagtctaatcatgttcaccaatcagaacctctgccacatcagctctgtgggtttcctctcatgccatctgtaccaacaatgagcacagtctgggtttcctgatgttttctatagcatggtgcccctccctgggacacctcagaagccacaatgatattcattaaacttcttcctgcaagtactccctctattgctccttctactcaccaagcttagtgtcagggaatatgctggtggggagtatgaatcttagcttctcactcccagactcggctcccaacctgctgttgccaagtcttggggacatctataaatgccttctaaatacctgtcttggcctatccccagccacagattcctccctagagcaggctacctctgaggcatctatactgagatcttaatcacagagacatttaaaacatctaggctacaaaagaattcctactatagggttcaaatgaatttctttccgtcacttgagacagctttttagcaagtacatgtattaaagattctgatttccccttttttctcctacagtttgtttggcctaaaggtatgacatctaatctgctgcatttacactctaagtcaaaattaccattttgttcttttaatttctatttctaatcatacattcttgtctactcctggttcatctggagacaaagttgaacttagaagatggaggagaaattcttatctgccctctgctagttgacacttcctttccaagaatcatttgcagttgctgggctttcttgctgtgactctatgccctgttgtgttactttttcatttgtgacaaaaacagtggtaatagtttgcttcatttttattatgaagacttgaccaggataggtaggaacataattactgctttttctgtgggcatcatattggtccagatcatgtttaaccagaattgaagatgtaaaatcctagaactcactaatctattttagtaggtcacaggaagcatgcaaaccataaaaaccgcaatgaaccaaaagcatctgtgttgacaagaggcaagaatttctctattttctaaaggcactaactgaacaaatctattgcccattacttaggaaattgctaagggcatcgttaaagtacttcaggctgttgaaaaaccttttctgtcttcttgccccaccaccaattctctctagctatgctttctctcacatgttcttgccatccatggagtggcagagaggttctgagtacaactctaactttcagtttcaatggcctaatccccaactcaactgctgaccttgagtgtaaaaataaacaagatcttaaacatcttagggccagcattctgggtcttcttttgttatttggggagcataaactattctcatatcattggcttgcagaaaaattgagcttctcctcttccttgccccatgtcaccttgaggtgaccacagccctgccttctatgtaatcctgcttggtcagcaggcacatcagagctcagtggcttgtgacatactttccttcaagcctgctcgaagggccatactcatcattgagactgggaaccttagaaaccatgaacccagtgccaatgggtagatacagaattctcaaactcatgaaaaaacatcctttttaactctctccgtgtgcataaagaattctaagatgtactgatcaagattcccaattttctaactatctattaatatatggacgaggtaagaagaaagttgcaagaaaaaaatttgtttgacttgacggttgtgtggttatttgccacactcccccagatccactctgagtcccaggatgctgattttaggggctacattgcctagactgtccattattccctcttcccatctggttcagtcactgggaagcactggtaggaggtcagagtgggagaaggaggttaggatactttctgcccctgctgtcctactgctttgctgaacttctagcagtggctgagccatcaacctgcagctctcaattccagctgtctgtagcactcaagtggtattgtttattcttttgatccttctagctgttaaggaagaaaaaaataattttttcctcaatgctcataagttcttggaatggacccctgtaacaaaagacagattaacacgagaaaagtttattaacgtacatattttatacgtacataggagctatccagggaatgaataattcttaaaaaggtgactttgaattccagctcatatagcatcttcaacaaagttcagtaacattttagagatatgacaagacaaagaaaatgaactttgagcctctagggacagtgacttgtaggaaggcaaaaggaataaatggaggtaaaggctggttagtaatacttgttaatgaagattcctctggtgccatctccaggtccacaaggatttaaatttgtcttcagtggtgaacctttgttcaccctggcagaaggtgtggaggggggtgcagatccgttttgtctttgtaagtctatatcctgcttttagacaaataaagggagggaaaagagctttccttcatctgcttcttaattgccttcagctcaacaatccttatgcaaaagaggtacattttagggtgtcaaattctggtctcccacaaagcatagggatgacagaccacctcaccatccctgttgcttcttttaagcctgctcacccctccaaaaatattttcattctatactgtctcttcaaaatctcagcggagggtgccctctgttatcctgccctctgtgtcagtataagccaaaggtttgaatcctggctctgcaactatctacctctgtgcctctccttgtttatgaaattacagggctggagacaaagatcacaatgtgaagacaaaattggagagcggtcctaatcagccagagcaaaatttctggctcttgctcttccccatcctgggttgaatcataggaacaggtggcaagatgccagggtcaggagattccagaagtggcagcaagctcagtgttaccaggtcagggatgacctgtcttattattgaaatctcagagatatgctccaattccggcccagagacacattgagagacaactggggaacttgctatgttcctgaacaggcaatgagctgtcttccaagaaaaaacctgagacccttcaagtctcaggtcttacttagcacatataccaggtcttacacaggacacatggttacaactgactgaaatctgggctgggtgtaggagctcacacctgtaatcccagcccttcaggaggctgaggcaggcagattgcctgagcccaggagttcgagaccagcccgggcaacatgacaaaaccccatctctacaaaaaatagtcaggcatggtggcatgcacctgtagtctcagctacttgggaggctgagatgagaggattgcttgaggttgagactgcagtgaagcatgatcatgccaccgcactccagcctaggcaacagagcaagatcttgtcgcaaaagaaagcaaaaacacaacataacacaacaacaacaacaacaacaacaacagcaaaaaagccaacttcttgaaatctggaaaggacacctggactgccctgagcatttgattgttgttggctctagcagtggatgcatccttcaacctctggcactctgcagggctcagactgttctgttctgtttgttacctgtggagtgcctgccagaccctgctctagctgctttaggtccatttaccctcatagacccccagtcttgttattcatatttcatatttgggaaatggaaacttagaaacttgccaagtccacagcatgagatcctgcctccggtgtctgctggattccagaaagtgccaggggccaacttagatgacaccatgttctctgcacaatcttaggaatgctcctagtctgatgtccccattgcaaaatttacattatcttttaacaaaacgtctttccaaggaggggcatttaaaataactgaggttcttcttgctaaggacgttcctgacacaagagataatttagcatttccttttcattaaaaagtttgaaatcctgtaatttgtgataatgtggatgaacctagaggatgttaagtgaaataagccacacacagatagacaaataccacgtgatctcactcttatgtggaatttttttttaaataagttgcttagccgggcatgatggcacacacctgtaatcctagctactcaggaggctgaggtgggaggatggcttgaactcagaaggtggaggtagcagtgagctgagactgtgccagtgcactccggtctgggtgacagaatgaaacccaatttaaaaaaaaaaaaaaagttgctatcttagaaaaagacagtagagcagtggttaccagagactggggaggaaagagaggaggtgagaatgggcagcagttgatcaacgggtacaaagttaccatgagataggagaaacaagtgctggtgctctgctccaagtagggtgacggtagttaataatgaattctgtatatataaatagctagaagagagggttttcaatatcattattatttcaaaagaaatgataaatgtttcagaggatggatatgtaattaccctgatttgatcattgcacaatgtatacatgtagcaaaacatcacattgtgtcccataaatatatacaattattatgtgaattaaataaaaaaaaattttaaagtcttatctaaatgaaatttctaaccagattctgaatccatgataccactgaaaccagcacacatgatcgcagtaaaacctcattatacttcctccactatcaccaataccctttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctggtgagttgtaggcattttttccattactttctgattcataggctcaacgcacctcaaagctggaaatgccgggtctgggtacaccctggggaactgcaaagcctgcacacttggggggaatgatcaagatgagaggcaggggtggggatggcatgtgcaccaggagatgttagagaaaccctgaggaagagcagcgtgcagcaggtgatgggggagagtgggcagcaagcgaggccaggacagccactctgctcagtcaccagtccacacacccaggggctcactctgcccctctgagcacccaaggacgttaaagagctggaactgttagtctaaatataggaccatccaagctctgaaccaaaatgtgtcccttgcctcaactcaggagatccacagaggcagaagtaaggaatttattttctgaaagatagatttctatcagttctgggtgacatgttctgacacttgaaatgacacctaggacagcacatttcaggcatcttgctcattgttcactgtagtagaagctacatgctagccagttgtaaaaatgaaattaagtaatgtgtgcacagcatttaacatagcatctgagcttcaggagcactcaattaatgaccacagttgtgattctttaggcagatgcatttttttccaactttgatcagaggtcttatttagcttctccagatttcaagaatctggctcagtgatatgaaatacaagacttgtgaaaagtgtcaattgcaagagaaatggaaggataaagtatacaggtgggtggaaaagaaattcacagtcactgccagaaaaaaaattcttgagaatcaagtcctgatgatgttagggcttatagttcttattataaagagttttatgtactcattcagtgaacatttattggtgcctcctttagccaggtactatcataagagctgaaaatagaagcataatccagtccttgatcttgaggaacatgctgtgtgtagcagataacataataagtgcttatctagatgcatgcagtgttatgtgataagagtaatatgacagaggatacagattaggcttcacagagaagggggatttgagcaggaggtattgaagggtgaatagaagctcaccaatcattttgggcagaggggcaaggacctgcaaaaccactgaagcatgaaggaaatggtgagtttagggaaaatgaagagaagatggctgtgactgaagcacaggatttgggattggagaagggactggaggtgaggctgaaaagaggcaaactcagaaaagatgttgtgctgggcagtctggacattatctttgaagcccaccacatataagtcatagggctactggaggttttaagctaagagtgactattcaatttcaacttaagagaagataggttgagagggaacatggcttgagatgagccatgagcaaaggaaagactacaacaaagccaggagtgaggagtgtgtgaagcaagaaagtgacagttgaaagcagtgcagaggggatgaatctgagaggcatctatgaggtggaactcaaatgacatgataataatacagggcatttctctgtgtcagatgctgtcctaagtccttactccattgatcttcacagcaactcagcatagttaatattttatgcataaagaaatcggcacttgaaggagtaattggccccagattacactgcctataaggattcaaatccaggtttgtttggctccaaaaactggctcctaattttcagaaggagaagcaacccagggcaatgcccaattttgcttcttaggcaatggaggaatccacaatcggaaggagttttcagcagtgccccatttggggtgggttgaatttgaggtccctgcatgatacccactttgctcacttcagtgcctaaaactgagtatggttcatagtaggtgttcaataagtgttgatgcagtgaatacatgcatggggagatatgcatcaggcaatgggaaattcaactctaaggcttaggggaaagctggagcttgaagacagagctttagaaaacagtagcatagaagggagtaggaaccatgagtttagacaatacaattcaggaagaactttgtagcaaggataaagaggcaaaaaattaaagaggtgagagctaagtgtggtgcctggggaatcttaaggtgtgggcacggggaggagatgccagcaaagaacatgaataaaaagcggtagcacagcccctcccatctggaagccaaaaagaattgtaaatggaggaagttagcagaaggatcaaatacttgaagagggtggaattggaataaaaccagggcatttgaaaaattgggttgtcactgcaatcttaacaagagaagttttggcaggatgatggaggcagaaagctgagagaatcatcagttagaacgtttttgacttcagagaacagaaaatgcagttcataatggctttaaaacaggggcttgtttttctcccagcaatttgagaggccaaggcgggtgcatcaggaggtcaagagaccgagaccatcctggccaacatggtgaatccccatctctactaaaaatacaaaaattagcggggcatggtggtgcacgcctatagtcccatctactcaggaggctgaggcaggagaatcacttgaacccaggaggtggaggttgcagtgagctgagatcatggccactgcactatagcctggagacacagcgagactccgtctccaaaaaaaaaaaaaaaagaaggcagaaggtgaatagttcaagggtgggtttaggactcagtgataataggattctgcctggcttctcatggttctctaggtcttccattcatggcaccatgccctcactaggcatgctgccagagcaggaggggcaggtggagggttctcttgtgtctgtcttatcagggaagaagagctttctcagaagcccccagcagactcccttttcatattatggtccagcaatgagtcacagacctatgcaccacctgcaaaggagccagagaaaacaaacgcccagcgcttttagcctgaaaatgagaatctggtttgctggggaagataaagggtgtcggaaaatggctgttgggtaaatcattgatgtctgccactaggaatgaaaggcaaatcaggaactggcacacatgctttcagggagatggctgcaagggagagggcaaagactgggaagttgcttatgtggtgccagactatttggaagatcatggattgcggtgtttgtgttgtgtggtcatcattttgttctttgtttacagaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaagatgcattatctatgatatgctaatatatgtatatgcaatatatataggctcttgcttgatctctccaggaggtagtgattatgagaagggggtggagaatgatgagttccttcaccaggagcaaaggacggggatcgtgtggaaccactgcagaactatttccgaaatcaactaagtggagagagccaggaaggctgcatcagaacccagtaaagcttcttgtctggatctgagctggtttgttttgtgcttgcttttccctgccttgccactcccctcactcttctcttttccccacagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgataaaagatttaaaaacaactggctgtttttttacactgtggtgtggaagattgtgttgtgttcacaacttttcacttcttcccctgtgtgattacacacacctgcccttgtggtgtgacttgcagtgcgccctacaggccacacaccccacgccctccaccactggctctgctgctggaatgtgagcagaagtgacatctgcctcatccaagcggagcctcttgctcagccacaggaaggcccattccagatcacacccgtcagcccgtgcgccctggtgaatgagaagacacagggagctgcagccacatataacatgagcaagaagtctgtgtttgctgtgataagccactgagttttaggggttgtttgttaagaagcacaaaaaccgattaagacatgtggtatatagtgacttcatatatagaatctggaaaactatccatttattttcaatcatggaattcaatatgacaagcatcccggagggtctacctatgccagactgggttggaaacagaaagacagatgttaatgccagtgtcctttacacctccaagtccagggccagctgtggagtgggaggggtagagaaggtcctgtgcacagtcacagtgcgctgtgcagagcaggaacagaggcatctgtgaaaagtgctgagagcctggaggacagagtgactaatgcaatgacagtcttgcatcataggaataacagccacagcaggattttattgctgccaaagaaactgccatttaaaaattgccagccatccgggaggctgaggcaggagaatggcatgaatccaggaggcggagcttgcagtgagccgagatcgggccactgcactccagcctgggcaacagagccagactccatctcaaaaaaaaaaaaaatgccaaccatctctctttctaaagtggaatcctcaagtctcgtttattctggggacatttgagtaggctgttcgagttaagcaggtggaagatgatgatccctcatcatcattctgagggatgagtgtctgaagactttttggtaaggaagggtcttggcatagcccaccccatgccacctcccttgatctgtgatatccccagcagctggggaggttggagcattttgcaaagaggccccgaagcctccagaaagtaaaccttcaagagcccatccttccttcctcttttgtttttgctcattatgaaaacttccctgacaaattgaaaaggcttcaggtgagattagcccacaggaaaaaccaccaagggccacccagaggggccacactgtgacagaaggagtatgggctggtgagctgacctctggaagacagtgcatgaattgaggagaagagattggttccaggcatgggaggaatgaggctggaaatttcttcagacattgccacttcggcactctgtgtgtgtgtgtgtgtgtgtgtgtgtgtgtgtgtgtgtgtgtgtgtaaacagagtcctgtaatgcaaggtccggccttggcagccccagcctggagccacagtgagatgtgagccgagggttatgctgggaaaaacctctccctcccagcacctgaaaggctctgcaggcccagcagctcagcaagcaagggtaagggcatggactaacatcttatttcatactatcccttataacacatcctaatgtaatcagctcacaatatgaaattatttcatttctctccagtcattgtttcaatggggccttagggttgactggattctggagggccctgcctagaggagggggtgcattctgtccctatgtcccctcctgctccatcctccacagcacgtgcctagtggtctaccttgtggggaattcttgtacctccctcttctaggcatggactagcattgagaagtgggagaggagtgttaggaaaaagggcaaatatagacataccttgtcttattgtgctttacagatattgtttttgttgttgttgttgttgtttacaaattgaaggtttgtggcaac

SEQ ID NO: 34 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 35 - DNA, 14488 bp, Homo sapiens
gcggaaaagagcctcgggccaggagcgcaggaaccagaccgtgtcccgcggggctgtcacctccgcctctgctccccgacccggccatgcgcggcctcgggctctggctgctgggcgcgatgatgctgcctggtgagtggttccagggaaccgtcccgggcgcagcgcggggcagggtggctctccccgcaggacgcgacgcggaccccgagcccctggcctgctcgcaccccgcaccctggcccggcttcgggctggtccctgggagcgggccatcaggtgcacagtggtctctgggtggagagccccgggccggggaggccggggaggtcggagggaaccttcaaaggtgaactgtgagagggaagtcccgcctctgagcttgttagtggggtcggggccgggccaagcaggacgcttcacctgctgcgcgaggcgccctgggttcctgggattcacctgtggggctacccacccccactatttagccgaggagggaagggagggcagacaagctgaggcctggagtgcaggggtgtgggctgaggggacccgtctcctgcctgggggaggacatggggctgctgaccctggatcccacccctgctcctggagtgacttgggctggggtgtgctgaggagcttcaggctggaggccgggttgcttcccgccagtttcctgggcagataggggctggctgctcccctgctgcctctccaggttggagatgtggtcatggagacacgtccactattgtgttttaatgggagcaaagggtcctggggttggtggtctccgaaaccctgcccattgcatgctgggaccctgccctctgctctcggataccagggagtttccagttcttgggaggtgaacagcctccgggaggcacttcctgctccccagttagatgcgatcagatgatcagaagaatggaggaggagaatggaaacgggcaggtcggcggcccgagtcctcctccctggggggaaggttcatccttgccctcgggcccctccagagatctgagacagcctggagacctgtggagtcactggcacccgctggcacccacagcgtctctgagcactgtgtgtgggtgctgcaggacacaggcccaccggccatgcctgggggcgctcctggggtcttttaggcacagaaccctcacttgagtctttgtcccagagctatctggtgagttccccaggggagggtcaagcccagcaagagacagaatctgagggcccaggaggcctctgggaaggaaacctgccccccggcccctcatgcctgctgaccctccctcctgcagcgattgcccccagccggccctgggccctcatggagcagtatgaggtcgtgttgccgtggcgtctgccaggcccccgagtccgccgagctctgccctcccacttggtgagtctaagctggcctggggagggtccctggctggcccctgagcctatttttgtgaaaggagctttagcttacagaaagtggtgagaggccagtgggtgtctggggctgccctgtacagctctagccccctggggcccatctccgttcctccctccagcactgcctgagggctcccagcctcctcgttccttcaggagctctatggacccctggaagggagcaggggcaggttgtgctggccccgggcccctccccagtttcacttggcgaggctgataagccaaattcctctttgagaagtgacgctggctcaccgcagagttgacacaccttcctgtgttggggggggggcctggcgggggtttcccagggctagaaactgagaatgcccccctccccgactggggctgcccgagggtgattcctgaagcctgagtgggtgggtgtgcacacatgtgcacggcatatgcatgtgtgcacctgtttccagggagggcagttccggccccagccctgtccttacctcagcatccggggaacctactaccgacctgcccgctgccctgggctcatccagccggtggcacagctaaggctgtcccagagctctgcaggccgagccccagcctgcacatgggggctggagatgcagtggtccagcaggaggctctgcaggcaccaccaggtccggcagctgctggaagggatgggtcctggaggcaggcagctgtcgctggaggggcctgtgcgtgctatccccgccacccacctcctttgctgcctggctgaggctatgcctcccccaccctatgctttctcggggaaggggcattgtcgttacccatgcgtggtcagatggggaaactgaggcctgggaggaccgaggagcaccgtgtccttcctgtctacccaagtgctgggctccgccggtcgccttcagcacagccccgttccctaccctccaccctccaccccccgccccccaccctctgccctccgccccccgcctccaccctccaccctccactctccaccctccaccttccaccccccaccccctgccccctaccctccaccctccaccctctaccctgcctgaggctgtggaccctccaggatggagccttgacctggggctggaggccggcaccccttcccccccagctgagcttctctggcccccaggattgaggtgggcacagcccgggcaggctgctcacctgggacccttccctcctcgctcagggcctgcacccagagagggtgagctacgtccttggggccacagggcacaacttcaccctccacctgcggaagaacaggtgagtgggtctcgggtcggcccccctgcccgggcctggctccagcacactggtgtccccggcgtccagcctctgggccagcctctctgcaccgagggggccgaggcagtcacagcacttcttttccagggacctgctgggctccggctacacagagacctatacggctgccaatggctccgaggtgacggagcagcctcgcgggcaggtatgggtcgggcggagcagctgggcgggcaggtaggggctgggcggccctccccgagcctgtggggccatgggggcacagcctgtggacccctctcctgcttcccccgcaggaccactgcttctaccagggccacgtagaggggtacccggactcagccgccagcctcagcacctgtgccggcctcaggtgggtgtggcggacgaacgccagggctcccgcaggtggagaggaaggcctaagcctgtggtgggggtggggaagggaggcagggagggtgcctcctcctcccactcaggcctttgctgtcttgcatgggggtctgctcaggggacgccaagcccaagaggctcaccctcctgtaccctggggcctgaagtcacctgcggggggctgctggtggcagggtagggggagctgtaggcaacaaactggagcctctggtccttcggggcaggaccaggccactctgaagcccacagcacacccggctcccactcccctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggtgagcacccttgtctcctgtgtttggctggccctgccggtgtccacttgcctggcctgtgagtcctcctggctccccatgtgctgggtgctgcccattgtctgcagcctggagcaggcctggccacgctcccccagcactggacaggggctggacagtggccccgggtccctgagggagggtccaggccccggcgctgggaaggctgactggcttcttgtggctgttccccaggactctctgccatcccgagagacccgctacgtggagctgtatgtggtcgtggacaatgcagaggtgtgttgggggcagggaaggtgggagagcagccggcggcgggggcatggggggtgtgtgggctccagcctgagggtctcgtgtccccagttccagatgctggggagcgaagcagccgtgcgtcatcgggtgctggaggtggtgaatcacgtggacaaggtgggcagcggtcctggctgcagaggtgccagagcccctggcgggaggtggggggggtgttgccacagggttcattccacctgctccagctctgccccgcacctgtgcctggccccacctcgccgtgggacaggtgcatcctccgccacccaccctccctgctgtaccttccgccaccctgcctgacccccaggactccaccacctctccagctatatcagaaactcaacttccgtgtggtcctggtgggcctggagatttggaatagtcaggacaggttccacgtcagccccgaccccagtgtcacactggagaacctcctgacctggcaggcacggcaacggacacggcggcacctgcatgacaacgtacagctcatcacgtgagctggcggagcgggcagggtggggagggctggggagggagggcgggcagggtggggtggaagggagagcagggctggggtgggagggaagtggggagggagccgtgtccagggcatctgtgtcactcccacttccaggggtgtcgacttcaccgggactaccgtggggtttgccagggtgtccgccatgtgctcccacagctcaggggctgtgaaccaggtgagggggctcctgcccgggtttgccaggccgaggccagagctggataggggaggggacgcctcggtccaggtctctggagcaggccacctcccctcaggccaggtttaatgcctgattttatgttttaaggtggtgttggcgggaagctgtgccaagtcctgggccctggtggggtgagggtggcaggcgtggggagttggccacatggttcctgagagcacagaccctcctgcaggaccacagcaagaaccccgtgggcgtggcctgtaccatggcccatgagatgggccacaacctgggcatggaccatgatgagaacgtccagggctgccgctgccaggaacgcttcgaggccggccgctgcatcatggcgggcagcattgggtgaggcggccaggccctgcagagccagggtatgctggaggtacgaggtgggtggggaaggggccaggccgcctcccccaatgctgccccgcctccccagctccagtttccccaggatgttcagtgactgcagccaggcctacctggagagctttttggagcggccgcagtcggtgtgcctcgccaacgcccctgacctcagccacctggtgggcggccccgtgtgtgggaacctgtttgtggagcgtggggagcagtgcgactgcggcccccccgaggtgcgtccatgccccatacctgctcccgtcagcactgggtgccacaacctctgtcccttcaggccagggtgaccactgcacatccacacgtggaacttggggctctgtgggtgtccagagcggtcacagggagggagtggctgcagggcaccccagtggtcaggggaggcggccaggtccgccctggagcccagcccggccccagtgcagcctgccaaggcctttgccgtcgccccaggactgccggaaccgctgctgcaactctaccacctgccagctggctgagggggcccagtgtgcgcacggtacctgctgccaggagtgcaaggtgagtgcagcctcccaccagtgacccccagagcccatggctggcaggtgggaagggaccctggggcaccagggaccaggcagcttgcgtggacctgtccccatggtgagctgttctctgctgggccaggtgaagccggctggtgagctgtgccgtcccaagaaggacatgtgtgacctcgaggagttctgtgacggccggcaccctgagtgcccggaagacgccttccaggagaacggcacgccctgctccgggggctactgctacaacggggcctgtcccacactggcccagcagtgccaggccttctgggggccaggtgaagtgggcacagagctggcctggcagggtggctgcaggccttgccagggctaacccgcttctggtcccacaggtgggcaggctgccgaggagtcctgcttctcctatgacatcctaccaggctgcaaggccagccggtacaggtgagctgagaccgccctgggcccaggacatgtgctgttcccacagcggggtcagccccagaagggcctctgcaaagggtcctgctcccatgagggtctgctctggacccatggaccccaggctcaagcgcaccgtgttgtgggagggaggtgggtcactgtggctcctgggctccccagcatgctcccaggcaagctggcccacgacctgccgtccatccacagggctgacatgtgtggcgttctgcagtgcaagggtgggcagcagcccctggggcgtgccatctgcatcgtggatgtgtgccacgcgctcaccacagaggatggcactgcgtatgaaccagtgcccgagggcacccggtgtggaccagagaaggtgagcccctggctcagaggccgcgccaccccccaggcatcccgggcatgtgccccacgtgggtgcagatgcgtttctgagcttggcagctgctcgaggccatgggaacagagttctctccgcgcttctagtagagactgaaaggtttccgggggtggggagagacatggttctcagttcatctgatggccgtcgccggcggccacgtgccccaggagggccacttcccaggggctttgggggtaggcagccggcagatgggctgaagcacagccaccttggacgtggtgaccacagcccacccccagcttaccatgcagtttcaagggacgctggcggggcagaggtccagagcggctgcctgagagacgcctcctacccccaggtttgctggaaaggacgttgccaggacttacacgtttacagatccagcaactgctctgcccagtgccacaaccatggggtatgtgtggcctctcgggacagggcccctgcacagaggctgccgctctgagggagccggtgctggggacagctctggacacgggagcctgaggctgggggagcctggcgtgtcctgtccatgggggtctctgtggagcccctgtcggctgaggcccggcctgggtggcctccgtggatggaacctgcatggtcaccgggcagcgggcaggggctacatggagaggcctcgccgtcggcgcatgtgggcagggaccgaggggccctcgggggtggggctggcccggggccctcatgctggaggcacggcccccacggccccttggggtccacaacagggttggccctgagcccagccgtccccgcaggtgtgcaaccacaagcaggagtgccactgccacgcgggctgggccccgccccactgcgcgaagctgctgactgaggtgcacgcaggtaggcctccctggccagcgcaggtggggctggggtccaagctcagagggcccgaacagagcccaggcagaagccgcgggcccgggaagctggtggtgttcacggtgccagtttgctgccacaggcatcgacacacaggtataggcagagcctgtctgtccccaggctgatgtgaggaggggagagccctgtcccagagggaggcagtggccgagggcagggcacggccgtgagctgagactccagtgtcctcagcctgtgtcttccagcccctcctcggcccggggccctgaggccacccagcatttcctccccggcggccactgagcatgtggtcgtccagagcatcaggcctcaaaggcacagccgtgcccacagcactgtggcccagggccaccctgccaagctctccaacctgtcactggcaagctgggagtgccagtgtaaggtaccaggtcctccctgaggggtccacagggcacagcctgggggtgctgagaaagaccgtgtctctgggaacctccctggctcctgcctgaggtcgcggcctgcctgcctgatgtggggccgtgtgtcccacagcgtccgggagcctccccgtcttcgtggtggtggttctggtgctcctggcagttgtgctggtcaccctggcaggcatcatcgtctaccgcaaagcccggagccgcatcctgagcaggtgagggtggtgggggctgtgacggagggtggcgagtgcttcccgtgctcagctgccagaggaaggagtgcccagcacgggcccatggtggacggcacggacacccaggctcctctgtgccccgacgtcatgacgccccccggcttgggctgagtttgaagggccttcctggctccactcggccagtgccactctggactccaccttcccctgaggctggggcggggcatggttttggtggccggttgggcttggggcaaatccagagaggcccggtggggtcccccataccctgacccagccccgtgagcaccctgccagtttcctgctgagccctgctctagggacctggacccccacacagcaggagacccccacgcagcaggacacactggccccacagcctccaagcatctcccgcccacctgactggggcccccaggacatggagggggtgtccctgcctcttcagggtccgccccacctcctgggcctgtctgtctctcatggccacaacctctcgccctgaagcctctcgggtcggctggagctgggagggagagactcggggtgtggtgctggggccccatggccggacactgaccatgcgccctgtgcttcctccccaggaacgtggctcccaagaccacaatggggcgctccaaccccctgttccaccaggctgccagccgcgtgccggccaagggcggggctccagccccatccaggggcccccaagagctggtccccaccacccacccgggccagcccgcccgacacccggcctcctcggtggctctgaagaggccgccccctgctgtaagcaccacccctggcgggttcatgaggccgaaagcagtgcccaggccccatcctgcgaccccaagtgcagcccgaggccagcagagccctttcattaaattcacgcacaggccagcggtggccccgaggcccagagcagctgctgtggtcgccctgctgggaacactgaggctttgggagtgggcacttcccagacccctttgagccatgatagaatctgagcttgaacttaaacttgggggctcccagcccagggaagcttcctggcctccacagagcccatctgaggacagtgtcgagacacaaatgctctccggcccccagctcgggatcacacggctgaggggaggaatggggcggggcctctcatctggggtggggccctgtgggccgcgcagacgcctccagcacacacaagaggcaggaacctcggatcctggagctgggggtgctggaggggcacgggggtcccagctctgacccaggctctcggccctgtctcctccagcctccggtcactgtgtccagcccacccttcccagttcctgtctacacccggcaggcaccaaagcaggtgagcagtggtgattgggcggccaagccttctgagggtgtccaggccttcctgatgggattggggtctgtgcactgcaccgggggccagggatccccaggtggcccctgctgtacacagaaggctggttagcccgacctcgtgaggacatcagacagaaaggcaggtcagcgaggccacaggaaggcaggagctcagtcctgccctgcgttggagctggtttgggggtcctggctccttctccttccactcagacctgctccgcccaccaagcccctcccagagctgaagctctggcagagaagaggactccctggggccctgcaggacagatgggtgggtgggctcagggcagggacacgttccgcgcttcccttgggtttgggggctttgggggttttggaaagatctctcaaaacccgaatcactggagccctggcatgctgagggttaactcccaggcctccgtgtccccccagggttggggtcctgtccctgcgccctggctcccacctctgggctaaccttggagccctttttggaccccgtgggggcagctcattggcctcagccctgggaaaccgctctggcagtgggtggagaaggacccttgagctcccttcctggggccactgactcagctagggcagcagaaagggccaggctttctctctggggcctggcacggggccccagtggtaccatcgaggtccacttggcattgcgggacattgtgagcagcctctgtcctcagagagggcaggacggtggcgggcaggctggggctggggaggtgcctgtgtgaagaggtcccacggtgcatctccggggaccccctccacagaggttcgtggtcagccctaggactgggctgtgcatggaagacctggggcaggagagcccctgccctctgggttgaggctttttttttgttaacatttaattttgaacaaattttagacttacagaaaagttgcaaaaatagcctagagaatcctgcatgcccttcgcctggcctgctcccgaatggccacagtgtgggcgcgcagccgaggctccacgtcaccaatgcgacccagtcagtaccgcctgctccctctggacgcccttctccagccatggtccccccaggcactcagcccccggcccctctgcctttcctcagcttgccctttggaagtgtggccagcagtgttttcagagtccctcagcctgggtttgtcccgtctttccttgtgtggatgagggccctgcactgttggcgggggcagcactggacacacccttccccaggcacagcccaggcctggcccacatgcagcttcggtcacgggggatgttgacacagcgcctagttggagcagcgtccacagatttttccttagaaagctcctgtcttctttttatagtgagcagatgcctcagggagataccttgagactctttgaaccttaagcttggacccactagttttggcatccattggtgaatattggctgcaatagttgttctgtgctgtttgcctaatagtgattttctctcttttttttttttgagatggagtctcgttctgtcgcccaggctggagtgcagtggcgcgatctcagctcactgcaatctccgcctctcaggttcatgccattctcctgcctcagcctcccaatgtatctgagactacaggtgcccgcgaccatgcctggctaaatttttgtattttttttttcagtagagacggggtttcactgtgttagccaagatggtcttgatctcttgacctcgtaatctgcctgccttggcctcccaaagtgctgggattacagacgtgagccaccatgcccggccctaatagtgattttctatttctctctttcctttttaactttttatttgaactaacttcagacttgtacgtgagttgcagaaatagtcctagaaggggtctggacaggcaaaatgggggcctgaagggggcagagagatttctggccaaggaagttatggttaccaggcatagcagtatatggggcagaggctgcagcaggagccgggcgtggtggtagatggggcagaggctgcagcaggagccgggcgtggcggtagatggggcagaggctgcagcaggagctgggcgtggaggtagatggggcagaggctgcagcaggagctgggcgtggaggtagatggggcagaggctgcagcaggagctgggcatggaggtagatggggcagaggctgcagcaggaacaggcgtggaggtagatggggcagaggctgcagcaggagctgggcgtggaggtagatggggcagaggctgcagcaggagccgggcgtggaggtagatggggcagaggctgcagcaggaacaggcgtggaggtagatggggcagaggctgcagcaggagctgcgcgtggaggtagatggggcagaggctgcagcaggagctgggcatggaggtagatggggcagaggctgcagcaggaacaggcgtggaggtagatggggcagaggctgcagcaggagccgggcgtggaggtaggtggggcagaggctgcagcaggaaccaggcatggaggtagatgcggcagaggctgcagcaagagccgggcgtggaggtagatggggcagaggctgcagcaggaacaggcgtggcagtagatggggctgaggctgcagcaggaacaggcgtggcagtagatggggcagaggctgcagcagaactgtgcaggagggaagtcactcactcttcactctcacgggcttcccctggctgctcagtagggtcctttggaagctgctgaaaactgcaaatgcttagcccacccaggctccgtgtgacacagccaggagtgagctccacagctctgtgtggtggacatccagcctccctaccctgggaaagctgaaatgcaaagaaacacgtgttttagtagctaattattggcctttgagcttccaaaaccccacattccggcaatctgtagagctcttcaggccaggcgcattggctcatgcctgtaatcccagcactttgggaggctggggcgggaggatcacttgagcccaggagttcaagaccagcctgggcaacatagggagaccccgtctctacaaaaaataaaaaattagctgggtatggtggcttgtgcctgtcgtcccagctactcaggaggctgaggcgggaggatcgcttgaacccaggaagaggaagctgcagtgagccaacatcacgccactgcactccagtctgggtgacagagggagaccctgactcaaaaaaaaaaaaaaaaaaaaaggaaaggaaaaaagctcttggagggtaatatgggggttggttaggggatgtggatggtgtactgggcaggtgggcccaggatggcgttctgccactgatgggctttattcctgggcagtggcagccaggagggcttttctgtagcgagctcagtgggggcggagcctgcaggctccctggggggcagagcgaggtctgtggggagcttcgttccaggtccagagaaggtgatgtccaggctagagccgggagtgcctgggggacccatgtcagctgtgtctgtttcctcccaggtcatcaagccaacgttcgcacccccagtgcccccagtcaaacccggggctggtgcggccaaccctggtccagctgaggtgaggccccagcacacaccaggcagagtccactgtgcaatggacggcacggcctccccacggggctgacggtggcactgccctggagggtggggtttttttctgattatctgtggataaacttgggtgactgaaagaaaagggaaaggcattggcagtggctgtactgtggtatctgtgctgtgatcaggggacttctcagcagatgccgaccccggctgtcagtggtgcctgaccttcccccagcaactgcagggaagaaagtctgtcctctgtgagctgtatatattgtgactgccacacctgccattgtgtgcatgggccatgttgttggtggatgaaccaagaatccacattttaagagatgttaaaggacgtaaaatttcgttgtaccctttatataccttttatagcaggtagtttttcaacatcaattatcttttcctcctcccagggtgctgttggcccaaaggttgccctgaagccccccatccagaggaagcaaggagccggagctcccacagcaccctaggggggcacctgcgcctgtgtggaaatttggagaagttgcggcagagaagccatgcgttccagcattccacggtccagctagtgccgctcagccctagaccctgactttgcaggctcagctgctgttctaacctcaggaatgcatctacctgagaggctcctgctgtccacgccctcagccaattccttctccccgccttggccacgtgtagccccagctgtctgcaggcaccaggctgggatgagctgtgtgcttgcgggtgcgtgtgtgtgtacgtgtctccaggtggccgctggtctcccgctgtgttcaggaggccacatatacagcccctcccagccacacctgcccctgctctggggcctgctgagccggctgccctgggcacccggttccaggcagcacagacgtggggcatccccagaaagactccatcccaggaccaggttcccctgcgtgctcttcgagagggtgtcagtgagcagactgcaccccaagctcccgactccaggtcccctgatcttggggcctgtttcccatgggattcaagagggacagccccagctttgtgtgtgtttaagcttaggaatcgcctttatggaaagggctatgtgggagagtcagctatcttgtctggttttcttgagacctcagatgtgtgttcagcagggctgaaagcttttattctttaataatgagaaatgtatattttactaataaattattgaccgagttctgta

SEQ ID NO: 36 - protein, 824 aa, Homo sapiens
MRGLGLWLLGAMMLPAIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 37 - DNA, 14020 bp, Homo sapiens
gtagaatacagcaacgacagacattttgggagagaagcattttatcatagcttttagaagagaagtatttttcagcatcataagcacacaattccaaggacagataccttcaagggattgcttttgacagttatgacaaagtcttaaagaagaataaaaggacaaaggaaatcctccagcaacaaagctgccacttatagatgagaaagtgaatgggaataaggaagaaactcagaaaagggaagagagatcactaaaaaccctgatttggaaagtcccagtactaccctgagaggagaaagaaacaaattcacacagcacgtcaccgccagaagaagaaaggagggaagacaagggaacagagaggatctcaatcctaaaaggacaatgtggaaacatttaggggacagaggtgaatctgccaggccaatgtagtttagaatgtcactcaatcactgagaatgagaaaggagtctgccgatgggcaccatgtggacaggagatgaggcaataaacatatgtaacaattaaaagtgagaaataaagatctggttttggttcagttaacccatatttgagtcgtagttccagcatttactgtttgtgcggtcttaaataatttattgtctcaacctcagtttctgcataggtcaaatggttcaatattatctactttaaaggttattatgaagagttaataagataatgagggaaaaaaaggtacctggcacttagtaggtgctcaataaaggacggctttttttttttaagtattgcttctaaatttgtatgtaagaaaaaatgatataatacaatgatataagacaggggaccgcaggacaagtccagccacacccattcatttatgtattatctgactgcttttctgaaatgatgcaaggttgagtagttgtacctgaaacatttattatctggccctttacagaaaatgtttccagaccctggataagtggtaccagagccccctctgtttgtggtcccctctcttatacccactaggtgtgagaaaagacatagagtaggagagccctgccatccatcttacccacccaggggctttttctgatggatccaaaggaaggacaaggtcttattggtctcccagaactgacataacaactccgacatcagggaaaagccattggagactacatagctcgccagccccagccacctgctcatatatctaagccctccttgttctagaccagggaggagaatggaatgtcccttggactctgcatgtccccaatctgagaacctggatccaagagggagaagaagcccattggagatgatgccataaaggaagtggaagcgatatgataaaaatcatagtgcccattcccaaataatcccagaagcagaagggaaaggagagaaatatccacaaagacaggtgtgggtacacacaacatttttcatactttaagatcccagagggactcatggaaatgatacaagaaaatgactcataagaacaaatattaggaagccagtgccaagaatgagatgggaaattggggaaaatgttgggggcagattgcttagttctgttctaagcaagagggtgaacaaggaaggaacagctcactacaaagaacagacatcactgcatgtacacacaataatataagaactaacccatgattattttgcttgtcttcttgttcaaaatgattgaagaccaatgagatgagatcaaccttgataactggctgcgaagcccatgattagacacaagatggtatcagggcacttgctgctttgaataaatgtcagtctcctgtcttggaagaatgacctgacagggtaaagaggaacttgcagctgagaaaggctttagtgactcaagagctgaataattccccaaaagctggagcatcctggcatttccagctccccatctctgcttgttccacttccttggggctacatcaccatctacatcatcatcactcttccactccctcccttagtgccaactatgtttatagcgagatattttctgctcattggggatcggaaggaagtgctgtggcctgagcggtctccttgggaagacaggatctgatacatacgttgcacaacctatttgacataagaggtttcacttcctgagatggatgggatggtagcagatttgggtccaggttacagggccaggatgagacatggcagaactgtggagactgttacgtcagggggcattgccccatggctccaaaatttccctcgagcgaaagcatcaggggctcatgcaacctggatactagtgctgcttcaaccacactgtgctattggatgagtcacttccaccctcctagccttgatttcttcgtctgctgttcacattcaaatagctattcatgtcttcatctctgtggtcccaccatatcccaccagacaatcattagggctcctcttagctggcagattctgaggtcctggatgctacaattggaagatggagaagtagaagctcaaggtttctgacctgtatcccaagtcccagaagcagaatggactaactcagagctgatgctcgggtcccttgcatatctcccttcctgtcactggctttgatcctccttcgttcagcttgtaatcacatcaacagaccaaagacatctctgtgttctgtcaggagagttcacagagccaccaaccctccagaccctgctggttgccgcataaagactctgaggaagggtttgaggctgctgtgatcatgcaatgaatgcatgattgtaccactgcactccagcctgggggataaaggtagatcctgtctaggagagagagagagagaaagagaaagagagagagaagggagggagagacaaagaaaaagagagagagggagggagaaagaaagagagaaagaaaagagaaaagaaagaaaaagaaagaaagagagagagggagggagggagagagaaagaaagaaagaaagagaaagagagaaagagagaaagagaaagaaaggaagaaagaaagaaagaaagaaagaaagaaagaaagaaagaaagaaaagaaaagaaagaaagagagagagaaagaaaaagaaagaggaaggaaggaaggaaggaagaaagacaggctctgaggaaggtggcagttcctacaacgggagaaccagtggttaatttgcaaagtggatcctgtggaggcaaaacagaggagtcccctaggccacccagacagggcttttagctatctgcaggaccagacaccaaatttcaggagggctcagtgttaggaatggattatggcttatcaaattcacaggaaactaacatgttgaacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttggataatgcagagcgagcacgatacctggcacatactaatttgaataaaaatgctgtcaaattcccattcacccattcaagcagcaaactctaccacctgaatgtacatgccaggcactgtgctagacttggctcaaaaagatttcagtttcctggaggaaccaggaggagcaaggtttcaactcagtgctataagaagtgttacaggctggacacggtggctcacgcctgtaatcccaacactttgggaggccgaggcgggcagatcacaaggtcaggagatcgagaccatcctggctaacatggtgaaaccctgtctctactaaaaatacaaaaaattagccgggcgtggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaagtgttatgatgcagacctgtcaaagaggcaaaggagggtgttcctacactccaggcactgttcataacctggactctcattcattctacaaatggagggctcccctgggcagtaccctggagcaggcactttgctggtgtctcggttaaagagaaactgataactcttggttggtattaccaagagatagagtctcagatggatattcttacagaaacaatattccacttttcagagttcaccaaaaaatcattttaggcagagctcatctggcattgatctggttcatccatgagattggctagggtaacagcacctggtcttgcagggttgtgtgagcttatctccagggttgccccaactccgtcaggagcctgaaccctgcataccgtatgttctctgccccagccaagaaaggtcaattttctcctcagaggctcctgcaattgacagagagctcctgaggcagagaacagcacccaaggtagagacccacaccctcaatacagacagggagggctattggcccttcattgtacccatttatccatctgtaagtgggaagattcctaaacttaagtacaaagaagtgaatgaagaaaagtatgtgcatgtataaatctgtgtgtcttccactttgtcccacatatactaaatttaaacattcttctaacgtgggaaaatccagtattttaatgtggacatcaactgcacaacgattgtcaggaaaacaatgcatatttgcatggtgatacatttgcaaaatgtgtcatagtttgctactccttgcccttccatgaaccagagaattatctcagtttattagtcccctcccctaagaagcttccaccaatactcttttcccctttcctttaacttgattgtgaaatcaggtattcaacagagaaatttctcagcctcctacttctgcttttgaaagccataaaaacagcgagggagaaactggcagataccaaacctcttcgaggcacaaggcacaacaggctgctctgggattctcttcagccaatcttcattgctcaagtatgactttaatcttccttacaactaggtgctaagggagtctctctgtctctctgcctctttgtgtgtatgcatattctctctctctctctctttctttctctgtctctccctctccttccctctctgcctccctctctcagctttttgcaaaaatgccaggtgtaatataatgcttatgactcgggaaatattctgggaatggatactgcttatctaacagctgacaccctaaaggttagtgtcaaagcctctgctccagctctcctagccaatacattgctagttggggtttggtttagcaaatgcttttctctagacccaaaggacttctctttcacacattcattcatttactcagagatcatttctttgcatgactgccatgcactggatgctgagagaaatcacacatgaacgtagccgtcatggggaagtcactcattttctcctttttacacaggtgtctgaagcagccatggcagaagtacctgagctcgccagtgaaatgatggcttattacaggtcagtggagacgctgagaccagtaacatgagcaggtctcctctttcaagagtagagtgttatctgtgcttggagaccagatttttcccctaaattgcctctttcagtggcaaacagggtgccaagtaaatctgatttaaagactactttcccattacaagtccctccagccttgggacctggaggctatccagatgtgttgttgcaagggcttcctgcagaggcaaatggggagaaaagactccaagcccacaatacaaggaatccctttgcaaagtgtggcttggagggagagggagagctcagattttagctgactctgctgggctagaggttaggcctcaagatccaacagggagcacccagggtgcccacctgccaggcctagaatctgccttctggactgttctgcgcatatcactgtgaaacttgccaggtgtttcaggcagctttgagaggcaggctgtttgcagtttcttatgaacagtcaagtcttgtacacagggaaggaaaaataaacctgtttagaagacataattgagacatgtccctgtttttattacagtggcaatgaggatgacttgttctttgaagctgatggccctaaacagatgaaggtaagactatgggtttaactcccaacccaaggaagggctctaacacagggaaagctcaaagaagggagttctgggccactttgatgccatggtattttgttttagaaagactttaacctcttccagtgagacacaggctgcaccacttgctgacctggccacttggtcatcatatcaccacagtcactcactaacgttggtggtggtggccacacttggtggtgacaggggaggagtagtgataatgtttcccatttcatagtaggaagacaaccaagtcttcaacataaatttgattatccttttaagagatggattcagcctatgccaatcacttgagttaaactctgaaaccaagagatgatcttgagaactaacatatgtctaccccttttgagtagaatagttttttgctacctggggtgaagcttataacaacaagacatagatgatataaacaaaaagatgaattgagacttgaaagaaaaccattcacttgctgtttgaccttgacaagtcattttacccgctttggacctcatctgaaaaataaagggctgagctggatgatctctgagattccagcatcctgcaacctccagttctgaaatattttcagttgtagctaagggcatttgggcagcaaatggtcatttttcagactcatccttacaaagagccatgttatattcctgctgtcccttctgttttatatgatgctcagtagccttcctaggtggcccagccatcagcctagctaggtcagttgtgcaggttgggaggcagccacttttctctggctttattttattccagtttgtgatagcctcccctagcctcataatccagtcctcaatcttgttaaaaacatatttctttagaagttttaagactggcataacttgttggctgcagctgtgggaggagcccattggcttgtctgcctggcctttgcccccattgcctcttccagcagcttggctctgctccaggcaggaaattctctcctgctcaactttcttttgtgcacttacaggtctctttaactgtctttcaagcctttgaaccattatcatgccttaaggcaacctcagtgaagccttaatacggagcttctctgaataagaggaaagtggtaacatttcacaaaaagtactctcacaggatttgcagaatgcctatgagacagtgttatgaaaaaggaaaaaaaagaacagtgtagaaaaattgaatacttgctgagtgagcataggtgaatggaaaatgttatggtcatctgcatgaaaaagcaaatcatagtgtgacagcattagggatacaaaaagatatagagaaggtatacatgtatggtgtaggtggggcatgtacaaaaaagatgaacaaagtagaaatgggatttattctaaaagaatagcctgtaaggtgtcagaaagcccacattctagtcttgagtctgcctctaacctgctgtgtgcccttgagtacacacttaacctccttgagcttcagagagggataatctttttattttattttattttattttgttttgttttgttttgttttgttttatgagacagagtctcactctgttgcccaggctggagtgcagtggtacaatcttggcttactgcatcctccacctcctgagttcaagcgattctccttcctcagtctcctgaatagctaggattacaggtgcaccccaccacacccagctaatttttgtatttttagtagagaaggggtttcgccatgttggccaggctggttttgaagtcctgacctaaatgattcatccacctcggcttcccaaagtgctgggattacaggcatgagccaccacgcctggcccagagagggatgatctttagaagctcgggattctttcaagccctttcctcctctctgagctttctactctctgatgtcaaagcatggttcctggcaggaccacctcaccaggctccctccctcgctctctccgcagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaaggtagttagccaagagcaggcagtagatctccacttgtgtcctcttggaagtcatcaagccccagccaactcaattcccccagagccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccgcctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccacccaagtctcttgggcatagtgcaatgtcaattcttccacaatatggggtcatttgatggacatggcctaactgcctgtgggttctctcttcctgttgttgaggctgaaacaagagtgctggagcgataatgtgtccatccccctccccagtcttccccccttgccccaacatccgtcccacccaatgccaggtggttccttgtagggaaattttaccgcccagcaggaacttatatctctccgctgtaacgggcaaaagtttcaagtgcggtgaacccatcattagctgtggtgatctgcctggcatcgtgccacagtagccaaagcctctgcacaggagtgtgggcaactaaggctgctgactttgaaggacagcctcactcagggggaagctatttgctctcagccaggccaagaaaatcctgtttctttggaatcgggtagtaagagtgatcccagggcctccaattgacactgctgtgactgaggaagatcaaaatgagtgtctctctttggagccactttcccagctcagcctctcctctcccagtttcttcccatgggctactctctgttcctgaaacagttctggtgcctgatttctggcagaagtacagcttcacctctttcctttccttccacattgatcaagttgttccgctcctgtggatgggcacattgccagccagtgacacaatggcttccttccttccttccttcagcatttaaaatgtagaccctctttcattctccgttcctactgctatgaggctctgagaaaccctcaggcctttgaggggaaaccctaaatcaacaaaatgaccctgctattgtctgtgagaagtcaagttatcctgtgtcttaggccaaggaacctcactgtgggttcccacagaggctaccaaattacatgtatcctactcatggggcctaggggttggggtgaccctgcactgctgtgtccctaaccacaagacccccttctttcttcagtggtgttctccatgtcctttgtacaaggagaagaaagtaatgacaaaatacctgtggccttgggcctcaaggaaaagaatctgtacctgtcctgcgtgttgaaagatgataagcccactctacagctggaggtaagtgaatgctatggaatgaagcccttctcagcctcctgctaccacttattcccagacaaccaccttctccccgcccccatccctaggaaaagctgggaacaggtctatttgacaattttgcattaatgtaaataaatttaacataatttttaactgcgtgcaaccttcaatcctgctgcagaaaattaaatcattttgccgatgttattatgtcctaccatagttacaaccccaacagattatatattgttagggctgctctcatttgatagacaccttgggaaatagatgacttaaagggtcccattatcatgtccactccactcccaaaattaccaccactatcacctccagctttctcagcaaaagcttcatttccaagttgatgtcattctaggaccataaggaaaaatacaataaaaagcccctggaaactaggtacttcaagaagctctagcttaattttcacccccccaaaaaaaaaaaattctcacctacattatgctcctcagcatttggcactaagttttagaaaagaagaagggctcttttaataatcacacggaaagttgggggcccagttacaactcaggagtctggctcctgatcatgtgacctgctcgtcagtttcctttctggccaacccaaagaacatctttcccatagcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaagaaaaaaaaagggtctttcctgatcattgacttgtcttggatttgacactgaacagtaaagacaaacagggctgtgagagttcttgggggactaaagcccactcctcattgctgagtgctgcaaagtacctagaaatatccttggccaccgaagactatcctcctcacccatcccctttatttctgttgttcaacagaaggatattcagtgcacatttggaacaggatcagctgaagcactgcagggagtcaggactggtagtaacagctaccagtgatttatctatcaatgcaccaaacatctgttgagcaagcgctatgtactaggagctgggagtacagagatgagaacagtcacaagtccctcctcagataggagaggcagctagttataagcagaaacaaggtaacatgacaagtagagtaagataaagaacaagaggagtagccaggaaggagggaggagaacgacataagaatcaagcctaaagggataaacagaagatttccacacatgggctgggcatggtggcttacgcctgtaatcccagcactttgggtggcaggggcagaaagatcgcttgagcccaggagttcaagaccagcctgggcaacatagtgagactcccatctctacaaaaaataaataaataaataaaacaatcagccaggcatgctggcatgcacctgtagtcctagctacttgggaagctgacactggaggattgcttgagcccagaagttcaagactgcagtgagctgtgatcgcaccattgcactccagcctgggtgacagagtgagaccctgtctctaaaaaatgttcccagatagaaaagaaaagaaaaagccctcaggtagaggaaccagtgtgaacaagagcatgggtgtataagaatgaatgatgcaaaaagtacactttcagaattgcgggaatcaccagaagcaaagtcaaagcgcaaaacaagccagaggaaaagtagttgtaactcacaccccagataaagagataacttgtttcacatgtaaagaactgcctcaaaacatttgaaaaaaaagaacaaaaatccagcaaaacaagaggcaagggatcttaacagtctgttcacagaaaagaaaatactatttgatcttgggcagagtaaaatgatgcttaacattgtaataagaaagtcaaattaaaagcactttgagatactagtattttcccatcagattgacaaaaatcaaaagtttaacaacagaccttgttggtcaaactgtcgggaaatccccactgttaaatattacgcgtgggactataaattgatatgacccttatagaacaaaatttgctaactatgaaaatcacaagtgcacttcccctttgatccagcaatttcactcctggagatttatcccacagatggacataacccatgtgaaatggaaaatgatcaaaattattcattgcacatcatttgtaataggaaaaattggaagtaacccaagtgtctatcaacaagagactgcctaaatgaagtaaaggacatagaatactaggcagctatagaaaagaatgagaaagcactctggtattgtttggttctgtgtcccagcccaaatctcatgtcaaattgtaatccccgatgttggaggtggggcctggtgtggggtgattggatcatgggggtggagttcttatgaatggtttagcactatccctttggtgctgttctcgtgacagagttcccacaagatctggttgtttaaaagtatgtggcatcctttctctctctctctctctctctcagtcctgctcctgccatataagacatccactcccgctttgtcttctgcatgagtaaaagcttcctaaggcctccccagaagcagatgctgccatgcttcctgtggaacagcctgcgaagctgtgagccaattaaacctcttttctttataaattatgcagtctcaggtatttccttatagcaatgcaaggactgactaatacatgctgtctg

SEQ ID NO: 38 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 39 - DNA, 2765 bp, Mus musculus
ccatgagtccctggcagcccctgctcctggctctcttggctttcggctgcagctctgctgccccttaccagcgccagccgacttttgtggtcttccccaaagacctgaaaacctccaacctcacggacacccagctggcagaggcatacttgtaccgctatggttacacccgggccgcccagatgatgggagagaagcagtctctacggccggctttgctgatgcttcagaagcagctctccctgccccagactggtgagctggacagccagacactaaaggccattcgaacaccacgctgtggtgtcccagacgtgggtcgattccaaaccttcaaaggcctcaagtgggaccatcataacatcacatactggatccaaaactactctgaagacttgccgcgagacatgatcgatgacgccttcgcgcgcgccttcgcggtgtggggcgaggtggcaccgctcaccttcacccgcgtgtacgggcccgaagcggacattgtcatccagtttggtgtcgcggagcacggagacgggtatcccttcgacggcaaggacggccttctggcacacgcctttccccctggcgccggcgttcagggagatgcccatttcgacgacgacgagttgtggtcgctgggcaaaggcgtcgtgatccccacttactatggaaactcaaatggtgccccatgtcactttcccttcaccttcgagggacgctcctattcggcctgcaccacagacggccgcaacgacggcacgccttggtgtagcacaacagctgactacgataaggacggcaaatttggtttctgccctagtgagagactctacacggagcacggcaacggagaaggcaaaccctgtgtgttcccgttcatctttgagggccgctcctactctgcctgcaccactaaaggccgctcggatggttaccgctggtgcgccaccacagccaactatgaccaggataaactgtatggcttctgccctacccgagtggacgcgaccgtagttgggggcaactcggcaggagagctgtgcgtcttccccttcgtcttcctgggcaagcagtacccttcctgtaccagcgacggccgcagtgatgggcgcctctggtgtgcgaccacatcgaacttcgacactgacaagaagtggggtttctgtccagaccaagggtacagcctgttcctggtggcagcgcacgagttcggccatgcactgggcttagatcattccagcgtgccggaagcgctcatgtacccgctgtatagctacctcgagggcttccctctgaataaagacgacatagacggcatccagtatctgtatggtcgtggctctaagcctgacccaaggcctccagccaccaccacaactgaaccacagccgacagcacctcccactatgtgtcccactatacctcccacggcctatcccacagtgggccccacggttggccctacaggcgccccctcacctggccccacaagcagcccctcacctggccctacaggcgccccctcacctggccctacagcggcccctactgcgggctcttctgaggcctctacagagtctttgagtccggcagacaatccttgcaatgtggatgtttttgatgctattgctgagatccagggcgctctgcatttcttcaaggacggttggtactggaagttcctgaatcatagaggaagcccattacagggccccttccttactgcccgcacgtggccagccctgcctgcaacgctggactcagcctttgaggatccgcagaccaagagggttttcttcttctctggacgtcaaatgtgggtgtacacaggcaagaccgtgctgggccccaggagtctggataagttgggtctaggcccagaggtaacccatgtcagcgggcttctcccgcgtcgtcctgggaaggctctgctgttcagcaaggggcgtgtctggagattcgacttgaagtctcagaaggtggatccccagagcgtcattcgcgtggataaggagttctctggtgtgccctggaactcacacgacatcttccagtaccaagacaaagcctatttctgccatggcaaattcttctggcgtgtgagtttccaaaatgaggtgaacaaggtggaccatgaggtgaaccaggtggacgacgtgggctacgtgacctacgacctcctgcagtgcccttgaactagggctccttctttgcttcaaccgtgcagcgcaagtctctagagaccaccaccaccaccaccaccaccacacacaaaccccatccgagggaaaggtgctagctggccagatacagactggtgatctcttctagagactgggaaggagtggaggcaggcagggctctctctgcccaccgtcctttcttgttggactgtttctaataaacacggatccccaaccttttccagctactttagtcaatcagcttatctgtagttgcagatgcatccgagcaagaagacaactttgtagggtggattctgaccttttatttttgtgtggcgtctgagaattgaatcagctggcttttgtgacaggcacttcaccggctaaaccacctctcccgactccagcccttttatttattatgtatgtggttatgttcacatgcatgtatttaacccacagaatgcttactgtgtgtcgggcgcggctccaaccgctgcataaatattaaggtattcagttgcccctactggaaggtattatgtaactatttctctcttacattggagaacaccaccg

SEQ ID NO: 40 - protein, 730 aa, Mus musculus
MSPWQPLLLALLAFGCSSAAPYQRQPTFVVFPKDLKTSNLTDTQLAEAYLYRYGYTRAAQMMGEKQSLRPALLMLQKQLSLPQTGELDSQTLKAIRTPRCGVPDVGRFQTFKGLKWDHHNITYWIQNYSEDLPRDMIDDAFARAFAVWGEVAPLTFTRVYGPEADIVIQFGVAEHGDGYPFDGKDGLLAHAFPPGAGVQGDAHFDDDELWSLGKGVVIPTYYGNSNGAPCHFPFTFEGRSYSACTTDGRNDGTPWCSTTADYDKDGKFGFCPSERLYTEHGNGEGKPCVFPFIFEGRSYSACTTKGRSDGYRWCATTANYDQDKLYGFCPTRVDATVVGGNSAGELCVFPFVFLGKQYPSCTSDGRSDGRLWCATTSNFDTDKKWGFCPDQGYSLFLVAAHEFGHALGLDHSSVPEALMYPLYSYLEGFPLNKDDIDGIQYLYGRGSKPDPRPPATTTTEPQPTAPPTMCPTIPPTAYPTVGPTVGPTGAPSPGPTSSPSPGPTGAPSPGPTAAPTAGSSEASTESLSPADNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRPGKALLFSKGRVWRFDLKSQKVDPQSVIRVDKEFSGVPWNSHDIFQYQDKAYFCHGKFFWRVSFQNEVNKVDHEVNQVDDVGYVTYDLLQCP

SEQ ID NO: 41 - DNA, 1665 bp, Mus musculus
cgcgggctcgccagcaccggcaccggctgcgccaggccggaacggggcgcgcttgcggccgtcgggcgccttctttttggacctcggggccctccacaccgtcccctcccccgcccgcctccctccccgcctccagcgccctccccgcggaggtccccccacccccatcccgcctccctgcgtcctccgggccgcaccgcccgggccggcgccgagcgcggggaagctggcgggctagggcgccccgctcctctgctctaccctcccccgtcccggctccgcgaggccgcacgtcgccgcccgcgagatgatgcaggacgtgtccagctcgccagtctctccggccgacgacagcctgagcaacagcgaggaggagccggaccggcagcagccggcgagcggcaagcgcggggctcgcaagagacgcagcagtcggcgcagcgcgggcggcagcgcggggcccggcggggccacgggcgggggcatcggaggcggcgacgagccaggcagcccggcccagggcaagcgcggcaagaaatctgcgggcggaggcggcggcggcggcgcgggcggaggtggtggcggcggcggcggcagcagcagcgggggcgggagcccgcagtcgtacgaggagctgcagacccagcgggtcatggctaacgtgcgggagcgccagcgcacgcagtcgctgaacgaggcgttcgccgccctgcgcaagatcatccccacgctgccctcggacaagctgagcaagattcagaccctcaaactggcggccaggtacatcgacttcctgtaccaggtcctgcagagcgacgagctggactccaagatggcaagctgcagctatgtggcccacgagcggctcagctacgccttctccgtctggaggatggagggggcctggtccatgtccgcgtcccactagcagcggagctccccaccccctctgcaggccggagacctagatgtcattgtttccagagaaggagaaaatggacagtctagagactctggagctggataactaaaaataaatctatatgacaaagattttcatggaaattagaagagcagagaccaaattcacaagaatcagggcgtggggcacacttttaaaagagaaagcgagacaggcccgtggacagagattcccagaggggcagcagcagaccccccacacctctgcattctgatagaagtctgaacactcgtttgtgtccccccaccccactttttgacgaagaatgttttatttttatttttcatgcatgcattctcaagaggtcttgccaatcagccactgacaggaaaggcatccctgtggactggctccattttaaaatggtaacaatcagaggaattttaacacctttagaaataaaaacgctgggatcaaactggcctgcaaaatcatagtcagtgaattccttaaaaaccatacttcctctgaaggaaaagaaaaacataaattacaaaagacaacattctatttatttattgatgacccatggtaaaatgcaaatagatccggtgtctaaatgcattcatatttttatgactgttttgtaaatatcttttgtatattatttttctgcaataaataaatatgattgaaaaattttaagaaaaaaaaaaaaaaaaaaaaaaaaaaaaaaaaaaaaa

SEQ ID NO: 42 - protein, 206 aa, Mus musculus
MMQDVSSSPVSPADDSLSNSEEEPDRQQPASGKRGARKRRSSRRSAGGSAGPGGATGGGIGGGDEPGSPAQGKRGKKSAGGGGGGGAGGGGGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 43 - DNA, 3796 bp, Mus musculus
ttttctctgcccggctttgatctctgcttaacaacagtaacgtcacacggactacaggggagttttgttgaagttgcaaagtcctgcagcctccagagggctgtcggcgcagtagcagagagctacagactccgcgcgctccggagaccggcagtacagcgcgaggcagcgcgcgtcagcagccgccaccggagcccaaccgagaccacagccctccccagacggccgcgccatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctgagggccaccgggcaggcgggagccaccaagtagtggcacccgcaaagaggaaggagccagcccgggtgctcctgacgacgtcccccttggggacatgttgttaccagaagaggaagttttgttctctttgttggttgtttttccttaatctttctcctttctatctgatttaagcaaaagagaaaaaaatatctgaaagctgtcttaaagagagagagagagagatagaatctgcatcaccctgagagtagggagccagggggtgctacaaaaatagaattctgtaccccagtaatcaactagttttctattaatgtgcttgtctgttctaagagtaggattaacacaggggaagtcttgagaaggagttttgattcttttatatgtttttaaaaaaaagcttaagaaacattgctttaaaaaggaaggaaaaaaaatacagcaaaccattgttaaagtagaagagtttttaggttgagaaatgtactctgctttgctgaaaagccacagcttaggccctcagcctcactccctggcttgctcagtgcctacagccctgttacctgatacctgtgctttatcccaggggtgggcagacctcttaaccttatagatggtcagtgcgacctctagtggtctcatggcgtgtggcacaacccccctccccagggctcagcttaatgtgccctctccccccaacaacctgcaggttcacagcaccagccacacagcggtagggatgaaatagtgacataatatattctatttttgtaaccttcctattttgtagctctgtttagagagatgctggtttttgcctgaaggccctgcagcctgcccacatcaggttaaacccacagcttttgtgtgtggtttgttttgttgtgttttctttctctatgttccaaaaccattccatttcaaagcacttttggtcagctagctggaggcagtgttgctggtgtgtgttggggggaggggttctaatggaatggatggggatgtccacacacgcattcagatggctgtacaacaggttgtagggctggtagtatgaggtgcttgggaagttttgttgggtcaagaagagagaactctgttctcgcaccaccgggatctgtcctgcaaagttgaagggatcctttggtgccagctggtgtttggaagtaggaaccatgatggcattacctggacaaggagattggggacaactcttaagtctcacacaggaggcttttaaacactaaaatgtctaatttatacttaaggctacagaagagtatttatgggaaaggctgcccatgaccagtgtgactcaaagcaatgtgatctcccttgattcaaacgcacacctctgccctgctggagaaggtttagggccatgtctgagagattggtctttcattgggcaacggggggggggggggggtccttaaaaaaaaaaaaccacaaagacagagatttggtctgcttgactttcccaacccaattggccccattggagagccatccaaactgaggaaaattaggggactccaaaagagtttgattctggcacattcttgccgctgcccccaagttaacaacagtaggtaatttgcacacctctggctctgtgcctttctattaggactttttggcagaaggtggagagcgggaggcttaagaggggatgtgagggaagaggtgaaggtgggaccacatgggacaggccacggctcctctcatggcgctgctaccgatgactcccaggatcccagacgttcagaaccagattctcattgctttgtatctttcacgttgttttcgctgctattggagggtcagttttgttttgttttgttttacaatgtcagactgccatgttcaagttttaatttcctcatagagtgtatttacagatgcccttttttgtacttttttttttaattgtgatctattttggcttaatgtgattaccgctgtattccaaaaaaaaaaaaaaaacaggttcctgttcacaatacctcatgtatcatctagccatgcacgagcctggcaggcaggtgggcggtctgcctccagggatcctgggaccctgatggcgatcgtcctgtcatgctgggcccttcatttgatctgggacatagcatcacagcagtcagggcacctggattgttctgttatcgatattgtttcttgtagcggcctgttgtgcatgccaccatgctgctggcccggggggatttgctctgagtctccggtgcatcatttaatctgttaggttctagtgttccgtcttgttttgtgttaattacagcattgtgctaatgtaaagactctgcctttgcgaagccagctgcagtgctgtaggcccccaagttccctagcaagctgccaaaccaaaacgggcaccaccagctcagctgaggcatcccagccaggcaggacccttgagggccgctgtatccatggtgatggggtgaggttttggccaaaaggccaaagactggtggtgggtccacggaatctgccctgtgacatgaaaggctttgaggggctctggctggtggccaggttggctttttgtatttctggttgacacaccatggcgcttcccagcacagacatgtgaccagcatggtccaggaaaaaaaaaaagacaaaaaatctagaaaataaaattggtaaaatctca

SEQ ID NO: 44 - protein, 295 aa, Mus musculus
MEHQLLCCEVETIRRAYPDTNLLNDRVLRAMLKTEETCAPSVSYFKCVQKEIVPSMRKIVATWMLEVCEEQKCEEEVFPLAMNYLDRFLSLEPLKKSRLQLLGATCMFVASKMKETIPLTAEKLCIYTDNSIRPEELLQMELLLVNKLKWNLAAMTPHDFIEHFLSKMPEADENKQTIRKHAQTFVALCATDVKFISNPPSMVAAGSVVAAMQGLNLGSPNNFLSCYRTTHFLSRVIKCDPDCLRACQEQIEALLESSLRQAQQNVDPKATEEEGEVEEEAGLACTPTDVRDVDI

SEQ ID NO: 45 - DNA, 1171 bp, Mus musculus
atccacctcacacgaggcacaagtgcacccagcaccagctgatcaggacgcgcaaacatgagtccagggagagcttcatctgtgtctctgatgctgttgctgctgctgagcctggcggctacagtgaaggcagcagcgatcatccctcaaagctcagcgtgtccaaacactgaggccaaggacttcctccagaatgtgaaggtcaacctcaaagtctttaactcccttggcgcaaaagtgagctccagaaggccctcagactacctcaaccgttccacgtcaccctggactctccaccgcaatgaagaccctgatagatatccctctgtgatctgggaagctcagtgccgccaccagcgctgtgtcaatgcggagggaaagctggaccaccacatgaattctgttctcatccagcaagagatcctggtcctgaagagggagcctgagagctgccccttcactttcagggtcgagaagatgctggtgggtgtgggctgcacctgcgtggcctcgattgtccgccaggcagcctaaacagagacccgcggctgacccctaagaaacccccacgtttctcagcaaacttacttgcatttttaaaacagttcgtgctattgattttcagcaaggaatgtggattcagaggcagattcagaattgtctgccctccacaatgaaaagaaggtgtaaaggggtcccaaactgcttcgtgtttgtttttctgtggactttaaattatttgtgtatttacaatatcccaagatagctttgaagcgtaacttattttaatgaagtatctacattattattatgtttctttctgaagaagacaaaattcaagactcagaaattttattatttaaaaggtaaagcctatatttatatgagctatttatgaatctatttatttttcttcagtatttgaagtattaagaacatgattttcagatctacctagggaagtcctaagtaagattaaatattaatggaaatttcagctttactatttgtttatttaaggttctctcctctgaatggggtgaaaaccaaacttagttttatgtttaataactttttaaattattgaagattcaaaaaattggataatttagctccctactctgttttaaaaaaaaattgtaacaatatcactgtaataataaagttttgg

SEQ ID NO: 46 - protein, 158 aa, Mus musculus
MSPGRASSVSLMLLLLLSLAATVKAAAIIPQSSACPNTEAKDFLQNVKVNLKVFNSLGAKVSSRRPSDYLNRSTSPWTLHRNEDPDRYPSVIWEAQCRHQRCVNAEGKLDHHMNSVLIQQEILVLKREPESCPFTFRVEKMLVGVGCTCVASIVRQAA

SEQ ID NO: 47 - DNA, 2453 bp, Mus musculus
ataagggaccagacaagagcagtgattggcaggggatgctgtgagcttgagggcacttgccagaggggcagaggaaggcaggagaggttgtcttggaagataagattatgtttcgcctgaagacacttccattactgatcttcctccacacacagcttgccaatgccttcccagtacctgaacacctggaagagaaaaatataaaaactgctgagaattacctacgaaaattctacaacttaccaagcaatcaattccggtcttcgaggaatgccacgatggttgcagagaagcttaaagagatgcagcgcttcttcagcttagcagagactgggaagttagatgcagctacaatggggattatggaaatgcctcgatgtggagtgcctgactctggtgatttcttgctaaccccaggaagtcccaagtggacacacactaacctgacctacaggattataaaccatacgccacagctgtcaagggctgaagtgaaaacagcaattgagaaagcttttcacgtctggagtgtagcatcgcccctgaccttcaccgagatcttacagggagaagcagacatcaacattgctttcgtctcaagagaccatggtgacaattctccatttgatggacccaatggaatccttgcccatgcctttcaaccaggccaaggtattggaggagatgctcattttgattcagaagaaacgtggactcaagattccaaaaattacaacctatttctcgtggctgctcatgaatttggacattctttgggactctctcactccactgatcctggtgccttgatgtacccaaactatgcttacagggaacccagcacctattcactacctcaagatgatatcaatggcattcagacaatctatggaccttcagacaaccccatccaacctactgggcccagcacacccaaagcctgtgacccccacctgagatttgatgctactaccacactccgtggggagatttacttctttaaagacaagtacttctggagacggcatcctcagctgagaacagttgacctcaatttcatatctctgttctggcccttcctacccaacggtcttcaggctgcttatgaagattttgacagagacctagttttcctatttaaaggcagacagtactgggctctaagtggctatgacctgcagcaaggttaccccagagatatatccaactatggattcccaaggagtgtccaagccattgatgcagctgtttcctataacgggaagacatacttcttcataaacaaccagtgctggagatatgacaatcaaagaagatccatggacccaggttaccccaaaagcataccaagcatgttcccaggagtaaactgtagagtcgatgcagttttcctgcaggactccttcttcctcttcttcagtggaccacaatattttgcatttaattttgtcagtcacagagtcactagagttgcaagaagcaatttatggcttaactgttcatagagttgagcaatgtcaaatgtccttgcacgcaccctatgaggacaaaaagggaagctcagtctgtatactcatcccacgtgtcctgcccagactttcctcagtggtaactaactctgcagccctcttcgaagaaccagcgtttgtaatctggcattcccagccatggctaactccttctgcatattccgtccctggttcgtgaatgcatcacaaagagagcaagccgtttactaccttactcttcactatttcattatctgctcttctgccttctaaaaccttttgttttattacttgcttatctagcttcaaatgttcctaaatctcttcaaacagggaatctagcattggattttcattacaaatgaagattataaacacatagatattatccccttatctgtacagaaaaaaagtgctgttatttcctccgttggaaaaatatgaaaaatatgtaatgtgtgttgaatcatgcccctctgcatgccagaaaaaaaaaaaaaaaaaacattgaaatcttaaccccagctgtttatctagctgttaaccagagtctttgaagatataatgaattatgtgtaaatgacaagacagccttaaacagggttaggatgagccataagtgcatacatatgttcttataaaacggagtgagaggtgtggatgtcactcagagattaaagttttttcctaacatgttttattctgaggagatttgggaagaatcagcagaggtatgggcacatgatagaagagaccaagcaaagagaaccagacactggagggctgtatctataaatctatttgccaataagttcccaggcagaggcaggtaggaggggtttaaaaaaaaggaccattcctttctcaagcacattccagaattgaggcacttgttgatatgggttttctggcttttgtgaaaaaataaagttgttattctgctct

SEQ ID NO: 48 - protein, 465 aa, Mus musculus
MFRLKTLPLLIFLHTQLANAFPVPEHLEEKNIKTAENYLRKFYNLPSNQFRSSRNATMVAEKLKEMQRFFSLAETGKLDAATMGIMEMPRCGVPDSGDFLLTPGSPKWTHTNLTYRIINHTPQLSRAEVKTAIEKAFHVWSVASPLTFTEILQGEADINIAFVSRDHGDNSPFDGPNGILAHAFQPGQGIGGDAHFDSEETWTQDSKNYNLFLVAAHEFGHSLGLSHSTDPGALMYPNYAYREPSTYSLPQDDINGIQTIYGPSDNPIQPTGPSTPKACDPHLRFDATTTLRGEIYFFKDKYFWRRHPQLRTVDLNFISLFWPFLPNGLQAAYEDFDRDLVFLFKGRQYWALSGYDLQQGYPRDISNYGFPRSVQAIDAAVSYNGKTYFFINNQCWRYDNQRRSMDPGYPKSIPSMFPGVNCRVDAVFLQDSFFLFFSGPQYFAFNFVSHRVTRVARSNLWLNCS

SEQ ID NO: 49 - DNA, 7207 bp, Mus musculus
cagtcaggagagagggcagtgagggtccatgctagctgggtcttgagcctcttctggggttcagtctctgatctacagcagtgtgtccacacctaaaacatcagctcagagaggcagttgcttctgctgttggaaacggacatcccaaaaaaaaacaaaaaacagaaatcaaaagggaaggagaaagtgaaagggatggaggcagcttgtccccttccctgtgcttgctgctggtagaaaactcagcctggaactgaccggagcagcagttcttgagtcaattccattccaacttctagaagattcttttcccgtcgaagagtgtcaggaggagaggccagacccccttgatcctgatctgccagccactgcatcagataagacgagataaccccgagttcctgttctaccagccctggtgtggtaaccctctccaatggggcaggcttggaaccctgtgccaacgaagatcctcccccgtactgatgcaggaaggacagcccgggagtgtaccctctacatgggtctacttttatttaagcaaacattccctggtcaacaggacgtgtagcattgcccccccccttgggtcacacagaaaacaggtaccaggaggacaagtagttgcttgcccagggtacagaatgaaaggcaataggggactctaggcgaatgttcttcccacccaaactgaggtagtaggagaagtccctactgaagggaaggtccagacataatcaaaggactaccagagatctcccaggtatctgtagaagtactaacatctccatccttcaacagctacaggttacacgtctccaaggctgggacattgtaaaacagggccatggtaaggtctacccgacagcacagagcaagcctcccagaagtctgagttccttctcctaacttctcatgctgggatctgagcttcttcgtgaaacacggggcagaggaggcaccagaactctcctctgaccaactgccccacagcacacatatcctcaaaggatagtcttgaatacgtgatggaagaattaaagagagtgaggtctgaagaaaatcagccctctcggggtttcctttgggtaactgagtgctaaggtgacttccgagtcagcaagaaatatcggacgttcaacccaggttgagtggaggaaacaattatttctcaatcctaatatgttctggaatagcccatttatccacgtcattatgacctgggagtgcgtgaatggaatccacagatgagggcctctgtacatagaacagctgtctgcctcaggaaatacaacttttagtattgagaagctaaaaagaaaaaaaaattaaaagagaggtagcccatactaaaaatagctgtaatgcagaagttcattccgaccagttctttagcgcttacaatgcaaaaaaaagggaaaggaaaaaaaaaaagaaagaaattaaactcaaaaattgcatggtttagaagagggaggaggagcctgaataacaaaaacctttgccaggaaggccccactgagccttcagtataaaagggggaccaagaacaggaggtctacatttagagacttgctcttgcactaccaaagccacaaggcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttgtaagtatgagctcgcctagcctttcttcctgccatcacctgaaatatgcattctgatggaactgcaaaaatagctctccttctcctcctccttctcctccttctctttctcctcctctgctttctcctccttctcttcctcctccttctcttcttcctcctctccctcttcctcctccagctcttacctcatgctgctcctcccctatttccagcacctattacccttaaacttaaatctagagagtcctagggaaagccatgagttaaactaaacccaggcacatccgaaaagctaactaggaggtgaatgcattgtctctcccatgctcaagaactttctgttaagtttccaataaggctccatgttttgctggctaggacaaaagtctgtggtctctgcgtagtctctagatctgggggacagaggtttgggggtttgaagcagcaccagcatagagagcttgcattacaaaagtattcccctttcagagctcctggaactggtatggaggtccaaagaaggcagagagctatgcaaaacaggctttctataagggactccagaaaggaagggttgacacaggtgctcttgaaacctgatctgactcatctgaaagaagaccgggcctgggtctaacttctcccagtcagttaaattcagtcagaagtttttaaaaacaactggatcccaagaggaaggactttgatttaactccgggctctagcactgtcaaatcctgttcttaaaactctcctttccacagttgccagggcatttgaattgattacaaattcaaaactctacaaatggaaaagtattcaaatgacatgcacattgggtattttccccagtccttgcattcacgttctttatgttcttccagcaaacaaaggaccagctggacaacatactgctaaccgactccttaatgcaggactttaaggtaagagtccggtgggtgctggggagcgaccaagcgattagggacgggacgggaggtgctgcttgtgacagcagagctgggtctgatgccttgactttcaaaaagagaagtgagaagaccttgactcagcatatcgccagcagtcaagggttgtgaagagctcattctgtggtaaagggagaaaatgtaactagaaggggcggggatggggggggggtccctaggaacaggacagtccatcaaggttagcagcaacagatcaccattccagtaagtcacacccaacctctgatccctgcctctagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtgtgagtagcagatgcgttcttccccacccccaatccccttagagccacccaacaaatactgtctcctacagcccagtcaggccacatgcatccagagacacacacagactagacaggagactaggtaaatctagagagacgcctgtccggtgtcgggtctcttgctcatctgtctctgagcgagtgtgggagtgactttgaggcactcacacgtgaagatttgcgcatagccttcctgttatttgtgagtcattgtgggttattagctactcccctctctctccatgggaaggctggggcttcagtcatggctccctcacctctgggctgccagctgaggctcccaagcacaggaaaacattcatctctttatctcatcttttgggaagcaaattcagtggcacagagctgggctgacaggacgctggtgtttcagaagggccagaagactgctatccctaagccagtatctatgaataatacagtcggggcatctatgtctctgaagcgatgccttggctgctcttgtttttcttacttctctcactgatactgggacatcagctggagtctacttgtgcaacggttctggcctgacgatttggccccagtagctgagactttcgctcctctctcagacacctagaaatagttcatggtggttgagattggagatagacaagaagagacactaaaaacagaacaggtgccttggagctctgagagacaaaacccgagacctgagttcacacccacacttagcaagccatcttagtgattgcaaagacatacattgcatctttctaggctgtcctctgctgtccccctcagtccctaccgaaatagataaggaccaatcacggctcagttctccctgggagctaagaagtgggcattgaagatgagcaagggtgtctccttcctcacacaacaagatagtttcccccaagtaccatggatgcagaattcaagcaagaattaccaatgctctcttttttcttcttcagcatcgatttctcccctgtgaaaataagagcaaggcagtggagcaggtgaagagtgattttaataaggtaagtggcaaagggggcgagtgtaacaagacctctgtctactcaccaaagcgcagaaggagggcgggagctattctgcacctggagtgtgggaacccagcaaatggtgtgacctcctctgccagttagaaagccaccacctcagttacatttgttttctgcaaagcgtctctggcagtttctaaatgactgctccacttttgcaggcttttggcttagactgaccagacagcctatgagcacagggcactaggtgttgaggagagtgacataggaaacagaaagtacagaaagtaccttgttgggaaacaggctgaacccacaagtacagaaagcagacatgaattaatttactcaggtacatcattggggctcagcctggatgcccctcccccaaatcagaacgagcagaaagcagaattcttacttgtcccggggcactttccacctggcaaacaaaatgaggtttcacacttccaactgcctgtgaacctattcaaccctagttcccagaagccatgtggcctacatcatcatctttgtgggctaggcagagacacctggcagggctctaacattagtgggcatgaattccatgacagcaggtcagagctgcagggtgaggactgcctacacactcaaaacataaaaaaaaaaaaaaaaaaacaccaaccatttccctgctatctctatcactgccctgcttacaaactgctccctctggcatctttgcataacattctgcataagcattatatgagcactggcctcttaaaaaaaattagttgaaaaggtgccaccctgaagacagtgctttggggactgaatgcttcccttgctgactcctggcagtgcttaaagctgggagaaggttggtcccaccacatcctgactactacagtatagttgtctctcttctttcatttccctgtccccatttctttattgtacataaagtaactggtgtatgtgcacacacatagtgctctctctttcttttccttttttaaactaaatggccgatgttctgttctggttggcatcagatggagatggtctgggggaaagtactgggtttgtgaaaatacccccttctccattagtggcatgctctttcagctcttatctttatattccagtaagttattttgctctcattgttttaacaaaagaacccaacaacaccaaatctttgcataccttgttcgattggagaattttaatgtttttcatttatcattgtaaaaccgaggacaattttataacttttttgtacgtggctgttacatgtagggcaatctgtctttaagtagggataaattactcttgaataaaatgatcctagatagttttcccttcaagtcaagcgtcttgttgtttaaataaacttcttgtttaaaatgaaaaaaaaaaaaacaaacaaacaaaaaaaagctgggagaaggtataggagaaacaggggaaggcacggccccttcccaaagcagacggacaaatctttgggtccttcagctcccaacacaagaaagcagagatctttctatgctcagcatccttcccagggcagggagcccctcagcccaccaatgggtactaaccagatgcttctctccccacacagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagactctaattttgaataaatggatcttattcgaattacagtgtggtgtctattgagttctgtctgatttaaaagaaaaatcccttaaaattccagagggcagaagcagtgaatcttagactccacaaaaagaatgccagcaacctctccaggaaggggttgagctcggatcagatcagaggaaagtcttgcctggctttcaaagcatcagcataaactactgatatcttcaagctcagtcctttaaaatgtgtcctgaaaccttaaaaggcattctaccttccaaagctagggaaggtgattctaagtaaccatcggaaggaagtgggaagcatcaaagctgaaactctgagacgaaatgttggggttaaaaatggaagctaggggcaggagggtggggagatggctcagcagttagagtgtgtgccggcaccagtgagacaggagttcatatccttggaatccaaccaacacacaaccaacactaggcaagagatgcctaatgtgtcagtggttgcttcaagctccagaggtcctggggtgaacttagatcc

SEQ ID NO: 50 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 51 - DNA, 3353 bp, Mus musculus
agtcaccaaaataagagaagtccgagacggcttcctcctccctccccagagtaggtagtaggggcagaggcgggtagcacctcactttaccccccacatcgcctctcctggttgctggaagttagggttggctcgccccagatcgagtcacccgcagctggctcccttgagaccaggtttccggcacctcccagggatcatgcatcagtcctattgacccaggagtaggggtctgtgggggcatctggaatgggctgtgtgttctgcaagaagttggagcctgcatccaaggaggatgtgggcctggaaggggacttccggagccaaacggctgaagaacgctatttccctgaccccactcaaggccggacttcgtccgtctttcctcagcccaccagccctgctttcctcaacactggcaacatgagaagcatctcagggaccggagtgaccatattcgtcgccctgtacgactatgaggccaggacaggggatgacctcaccttcaccaaaggcgagaagttccacatcctgaacaatacggagtatgactggtgggaggctcgctccctgagctccggacacagaggctatgttcccagcaactatgttgctcctgtggattccatccaggctgaagagtggtacttcggaaagatcagtagaaaggatgcagagaggcagcttctgtcctctggtaacccccagggggcctttctcattcgggaaagcgagaccaccaaaggggcctactccctgtccatccgtgactgggaccagaacagaggcgatcacataaagcattataagatccgaaagctggacacgggcggctactacatcaccacacgggcccagtttgactccatacaggacctagtgcggcactacatggaagtgaatgatggtctgtgctacttgcttacggcgccttgtaccaccactaagccccagactctaggcctggccaaggatgcctgggagatcgaccggaactccatagcactggaacgcaggctgggcaccggctgctttggagatgtgtggctgggcacatggaactgcagcacaaaggtggcagtgaagacgctgaagccgggcaccatgtccccgaaggcattcctggaggaggcacagatcatgaagctgctgaggcacgacaagctggtgcagctgtatgcggtggtgtcggaggaacccatttatattgtgacagagttcatgtgctatggtagcttgctggatttcctaaaggatcgagaaggtcagaacttgatgctgccccatctagtggacatggctgcccaggtagccgagggcatggcctacatggaacgcatgaactatatccaccgagacttgagggcagccaacatcctggtgggggaatacctaatatgcaagatcgctgacttcgggctggcacgcctcatagaggacaatgagtataacccccaacaaggaaccaagttccccatcaagtggacagccccagaggccgccctctttggcagattcactgtcaaatcagacgtgtggtcctttgggattctgctcactgaactgatcaccaagggcagagttccctacccaggtatgaacaaccgggaagtgttggaacaggtggagcatggctaccacatgccgtgccctccaggatgtcctgcatccctgtatgaggtcatggagcaggcgtggcgcctggatccagaggagaggcccacctttgagtacctgcagtctttcctggaagactatttcacctccacagaaccacagtaccagcctggagaccagacatagcctgactgggcatcactgaccctctgggagtatgtacttgctcttgccaatcccccggggcactctggtcccagtgcccaagacttggaacccctgtggaatcctagcacaggatcagagaaggtccgacgctctgaagccattttatagagggtgcaaatggagacgagaaactcatctcttacctcctctgaccccaagtgttcatttttcccttcccacccatgccccatccaaaaatctagggcatcccactcatttcacagatgaagtaagtggatgctcagagctcctctctcgaacactctgaccccagtaccacttttcctgtcccattaggctccgacataccgttagtctctctccccatctcctaaaaatactcatcctttgtctagttatttataaaactgtatttctcttcttccacttatcccttattcctgctttcctacctcctatcagcctgctccagcccagaccctaagaatttaattcttagcatctcttgggtttctctacattacaaaagccttcagactggatgctccggtccgggcccaggtctgagggagagagctgttgaatgctcaccacccctctgagggacgtgtatgtgtgtattcattatgtaagaacaacgatgatatgcacaccaggctagaacaatttcctgagccatctatggagaggggtggtagtctgcaggaggcgaacaggatagctttggctatttgtggggagggcggagtggggtggttagcagttccctcttacctactaagtgccagatacccagcacgtggggcttcagggcacccctatgtcccactgttcacccacatgtcagttcaccatgagttgagagacccagaaagcaggtgattgcaaataagacaaatgtggttttgaatcttcgtacagctaattctttacctgatttcagcctacttcccaaccatcacaggcacactggcacagtcttaggcctcccatggcagaggcatggagaagaatacaacaaactctctacagctctcaggctccccagaacacagcaagaggaggatgaaggcctagctcataccacctcactggggagtagggtgggctcttccccaccccagggagcttttggagacctttggagatatttttattatagtgactggaagcacctactggctgggatcggagatggtagaccatctttgatgtacttcttggatcctctgtggagaaactggtagctgacatgcctctttggaaaccaggagaacaggatatatggagtctcacctctcctgtcaggaagtgtttattggccctctggcttattgctgggagagaggaaaggagcctgggagtttggagtcactaatgcactaggcaattactaagaattgactctgatgggcccttcactgagcaggaccctgagcaaataaaaatgagtaagagctattcctatcctggaggctttcccttgggtggagaaataatgaatgaataaataaacatatacttaccttttaagaaaaaaaaaaaaa

SEQ ID NO: 52 - protein, 517 aa, Mus musculus
MGCVFCKKLEPASKEDVGLEGDFRSQTAEERYFPDPTQGRTSSVFPQPTSPAFLNTGNMRSISGTGVTIFVALYDYEARTGDDLTFTKGEKFHILNNTEYDWWEARSLSSGHRGYVPSNYVAPVDSIQAEEWYFGKISRKDAERQLLSSGNPQGAFLIRESETTKGAYSLSIRDWDQNRGDHIKHYKIRKLDTGGYYITTRAQFDSIQDLVRHYMEVNDGLCYLLTAPCTTTKPQTLGLAKDAWEIDRNSIALERRLGTGCFGDVWLGTWNCSTKVAVKTLKPGTMSPKAFLEEAQIMKLLRHDKLVQLYAVVSEEPIYIVTEFMCYGSLLDFLKDREGQNLMLPHLVDMAAQVAEGMAYMERMNYIHRDLRAANILVGEYLICKIADFGLARLIEDNEYNPQQGTKFPIKWTAPEAALFGRFTVKSDVWSFGILLTELITKGRVPYPGMNNREVLEQVEHGYHMPCPPGCPASLYEVMEQAWRLDPEERPTFEYLQSFLEDYFTSTEPQYQPGDQT

SEQ ID NO: 53 - DNA, 3025 bp, Mus musculus
gtcttctagctgtcccagcagcccacagaggcagtcgttggagctgagcttgaaggatgaggaaggctgggctctggggactgctgtgcgtgttctttgtctcagaagtcaaagctgccattgttctagaggaagaaaggtatgacctagtggagggccagactttgacagtgaagtgtcccttcaacatcatgaagtatgccaacagccagaaggcttggcagagactaccagacgggaaggaacccttgaccctggtggtcacacagaggccctttacaagacccagtgaagtccacatggggaagttcaccctgaaacatgaccctagtgaggccatgctacaagttcaaatgactgaccttcaagtgacagactctggattgtatcgttgtgtgatttaccatcctccgaatgaccctgttgtgctcttccatcctgtccgcctggtggtgaccaagggttcttcagatgtgttcactcctgtcatcattcctattacaaggctgacagagcgtcccatccttattaccacaaaatactcacccagtgacacaactacaacccgatccctacccaagcccactgcggttgtttcctctcctggtcttggagtcactatcataaatgggacagatgctgacagtgtctccacatccagtgttactatttcagtcatctgtggacttctcagcaagagcctggttttcatcatcttattcattgtcacaaagaggacatttggatgacagaacttgaagctatacaatagtgaccttcagcggtgtctatttcacaggaggagctgaggtggtggggctgaggaggagctatgacatgaattgaacctgtaatcaccggtgacgtctaaggctcaggatatcctcagctgaccctgtccactctcctcattttatccatcatcttggggatgtgctctgcacccttagaaaaggggaaaccattcccagaacactctggccattccccctaaatagttgggttggcctgaaataaagagaaactccagagcttccccctgtctcttgttgtgggagtcaagaagattacatggtcaccactgatatccccacctcttgtccttctcctggatggtacaattctagtcattttagcagggcccttgctcatgaagcagcctgtcctcctacctctgggccttcctttctgggagtcagacactcagatttctgtcttcatctgtagtcctctgaacactcccaaccatggaccaagaagacatgatcctaccttgacaataatgcaactgggagagagaaatcaagttaagctggatgctgttgcctgggaaacacctggaagagatagtatccgtagcattgaatcagtacatggatggcatcaacatgctgtgtgcattttattattgacttctcagtgcatgagggttaaagtgtatgtgatcagagtaaggccacaggggtagcaggccatcacctcctggcaggaatgaaatctagagagttcccttactcaagagctccagaggcagggagatggggagccagactctgctgctagctgaatgcctttttgcctggactgagacagggtctagcatggcggggggggggggggcaggatcctatctcagactgccccctgtgtgagtctacctcccactcaccttccttcacctctcagtctcagcctgggtaccctgtggctcctgtgctttctctaggacacactggcatttgcttgtttccacctctttgggtgggaaaaggatggtagatccattccttctacttcagcagtggctggaatcaacttcatcgaaaggttcccaagatcaaaccgcatcaccacaaagcatccagctcacttgatcgcacccagaaggccgaggagtggctgaaccacgtatcttatatcaggaaagcactttcctgtcagggagactttcccttccacctcctgtgaagatcccaggtcttaaggtgtgcttctgtcacataaggacggttcactgtgaacgtggctctctgtgtaactccaccctggggtggcatcgctgagcacagggtattcacagctgcataatctccagtctgctgggagaggccgcagtggaatccactttgggttggtgctctgggacacgtctttggagggctggtgcagatgctgttcatctcagtaggtttgtttcctgactgaagaatcaagcaggggtttatagtcaggggctaaaatgcttcccaagaaagcctggggaccagagttcaagccattagagcaaaatgtaaaaaagctgcttagggcgggccatacctataatctcggcactgggaaagccaagacagccagttctctggggcttagtgactagttagagaccctcttgtggtcagtgagagaccttgtttcagatacaacaacaacaacaaaaaaacaataacaacacacacacacacgttgtatgataactgtcatatttcttaggttattcaatttgcagtagtccagaaaccaccctggaggagagcctcagttgatgaattgtcccctgcaggcatgcctgtgctgtattgccttcgctgttaattgaggtgggagtatccaccctgtatatggctggtgccacttcatacactgggtcccaactgtgtataaaaataaggaagcctagttgatcagaagcaggcagaccccaagggtccagtttatcctctgctcttgactatggatatgatgtgactagtgcttaagttcctgcctcgacttctccacaataagggactgtgacctgtgttgttgttgttgttgttgttgttgttgttgttgttgtatttggggggtggggtcagggtgccttcccagggcatcagagtaaaactggaacaatgacacatgtctgtgatcccagcacttgggggactaaggcaaggggcctgaggattcaaggacagtttggactttgttgtgaaatcccatcccaaactgaaaaataaataaaataaaagcagttgagattctg

SEQ ID NO: 54 - protein, 230 aa, Mus musculus
MRKAGLWGLLCVFFVSEVKAAIVLEEERYDLVEGQTLTVKCPFNIMKYANSQKAWQRLPDGKEPLTLVVTQRPFTRPSEVHMGKFTLKHDPSEAMLQVQMTDLQVTDSGLYRCVIYHPPNDPVVLFHPVRLVVTKGSSDVFTPVIIPITRLTERPILITTKYSPSDTTTTRSLPKPTAVVSSPGLGVTIINGTDADSVSTSSVTISVICGLLSKSLVFIILFIVTKRTFG

SEQ ID NO: 55 - DNA, 3589 bp, Mus musculus
tgccatcataaaggagccatacctgtaaatgccatgcaagttcagctgcctgcaaagaccagaagagggcattggattcaccacatgtgctaagaattgaacttgaatcatctgcaaaaacaaatcaaaggaaatggaagacaataatatgttacctcagttcatccacggcatactatcaacatctcattctctatttacacgaagtatccaagagcttgatgaaggggccaccacaccgtatgactacgatgatggtgagccttgtcataaaaccagtgtgaagcaaattggagcttggatcctgcctccactctactccctggtattcatctttggttttgtgggcaacatgttggtcattataattctgataggctgtaaaaagctgaagagcatgactgatatctatctgctcaacttggccatctctgacctgctcttcctgctcacattaccattctgggctcactatgctgcaaatgagtgggtctttgggaatataatgtgtaaagtattcacagggctctatcacattggttattttggtggaatctttttcattatcctcctgacaattgataggtacttggctattgttcatgctgtgtttgctttaaaagccaggacagttacctttggggtgataacaagtgtagtcacttgggtggtggctgtgtttgcctctctaccaggaatcatatttactaaatccaaacaagatgatcaccattacacctgtggcccttattttacacaactatggaagaatttccaaacaataatgagaaatatcttgagcctgatcctgcctctacttgtcatggtcatctgctactcaggaattctccacaccctgtttcgctgtaggaatgagaagaagaggcacagggctgtgaggctcatctttgccatcatgattgtctactttctcttctggactccatacaatattgttctcttcttgaccaccttccaggaatccttgggaatgagtaactgtgtgattgacaagcacttagaccaggccatgcaggtgacagagactcttggaatgacacactgctgcattaatcctgtcatttatgcctttgttggagagaagttccgaaggtatctctccatatttttcagaaagcacattgctaaacgtctctgcaaacagtgcccagttttctatagggagacagcagatcgagtgagctctacattcactccttccactggggagcaagaggtctcggttgggttgtaaagtaagtgacagtttgcctttttttaagcagggaaggagggagcagtttgtacatagcaacaagcctcaaagacagtggttcttactttgggtcatgatccctatgtgggggtcaaatgaccctttcacagaggtcactaaaaccattagaaaacataggtatttacattataattcataacagtagcaaaattacagctatgaagtagcagcaaaaataattttattgttgtagatcaccacaacatgaggaactgtattaaagggtcacaggattaggaaggtttcaaaccactgctcatggatatattgaacaatagagacctttaaatcaggtgcccagaacgttataattatgccgtgactgaaccatatctcttcttgtatatataatacatgtttgagaaatattttagaataataattggtaaattcttcagctttccagggaactctatccctatgaggagcctctttgccttgtggcaatattattttaccaatcttcaccatgtttttccttacttataacatgaaagacatggaatttaagaaaggagggcatgaggctgccaggactgagtgagaaggaggagatatgcactgttgctcctgagtgaaaatggaaatgaaagtgagcagggctctcaaatttctttctcatttgggaagaaatgtcttgctctttggttccttctcctatctgtgtttaattttggaggctttatcaggttgtggaaaatctagaaactgtaataggttgttggaaacctcatttgagtaccatagtccttttatacataattgcacaacacatttttgctttgttatttttttttatctttgacaacatggacaatttttttcaatatcacactccactgttgatatgtttctcaagccacagttccttacatgatatttataaaattattgtttataaaatatgcattattagtgattagactaaaaataataagggctcttgtttgatctttccttctgctttccagggagagcagaaggctaaaagtagtgaatgaccaagaataaggagaaaagccaactccttcatcaggcacagagagctgcagcaaaaaggcagagatctgtgggacagaggaagtggtgtgcttagaactacatagaatcagaagcaaaatggatgccttagcactgaggaaaggtttctgtcaggttataaactggttttatattattgctgctattttctgcctcctcagtacctttgcaactgcctctttcctcatagcatttatctctcaacctcttcctgggaaaggtgtatatggaatagccaaacatgaaaacctggatagttctatgagaaaaaaaaaaaacactgttatattatgtttttgtgggtaaaacaaaactggatgaagcagggacagaagtcttgctttgaaaataatacttgggaacatatcttaattacaggcaaaataaacctggataaaaatgattataaatggagtgggaagaagtatgtcacagattatcttccaatgccttatttttggatataaacataattcaaatatttaaaaattgaaaatagaaaaaaaaataaaatcaagactggaaacttcaacttctaaatgtgatgaaatacctttgtaagagctcttatcttgccaaggttgaaaatagttcattagttataaaagaaattctgactttgagcttatgaactaacatagacagctcaggattaacagggacttgtggtttgtggtctgtgggcttatccaagcatggtgatttagactctaaggtccgtctggatcctagcatatgctacatagatgtcatgtgtgcatgcaagtggtgtacttgctctttcaccaaactcctttggatcttttgtcctaaatgtgatcttggtgaaagattgtcctttgagtgagtgggaactcctaagtcaaatcaacttcttatcaaggatttaaaagtaaatatgcaaaggagagaagttagagaaactcctctcataagaaaaatgtcttccctgaattgagccaaaaatgttcttccctgtccactaatgcgtttcttatctaacagaagcaagaaacaaaggaacagacaacttgagagttaaaagtcattgtggtctggctttgctaccacatgatgagaagaaatttctgaaagaagtaggggatgagagtatcaccttcagatagcctaacagtttcattttaatgataatgagatgcaaatacctgtaaaactatgatttagaaaataaaccaatgctctatctatattgctagaaatattaaaaagaaacaagcatatgctttgtta

SEQ ID NO: 56 - protein, 373 aa, Mus musculus
MEDNNMLPQFIHGILSTSHSLFTRSIQELDEGATTPYDYDDGEPCHKTSVKQIGAWILPPLYSLVFIFGFVGNMLVIIILIGCKKLKSMTDIYLLNLAISDLLFLLTLPFWAHYAANEWVFGNIMCKVFTGLYHIGYFGGIFFIILLTIDRYLAIVHAVFALKARTVTFGVITSVVTWVVAVFASLPGIIFTKSKQDDHHYTCGPYFTQLWKNFQTIMRNILSLILPLLVMVICYSGILHTLFRCRNEKKRHRAVRLIFAIMIVYFLFWTPYNIVLFLTTFQESLGMSNCVIDKHLDQAMQVTETLGMTHCCINPVIYAFVGEKFRRYLSIFFRKHIAKRLCKQCPVFYRETADRVSSTFTPSTGEQEVSVGL

SEQ ID NO: 57 - DNA, 3117 bp, Mus musculus
agtgggaagggcactcagaaagaagtaccaggccagtgatgaagagagtactttgtggaacctctagggttagtgtgctccagatcccatcatgcttggcctctggctgctcagcgtcttatggacaccagcagtagcccctggacctcctttgccccatgtgaaacagtatgaagtggtttggcctcggcgcctagctgcatcccgctcccgcagagccctgccctcccactggggccagtacccagagagtctgagctatgctcttgggaccagcgggcacgttttcaccctgcaccttcgaaagaacagggacctgctgggctcaagctacacagagacctactcagctgccaatggctctgaggtgacagagcaactgcaggagcaggaccattgcctctaccaaggccatgtggaagggtacgagggctcagctgccagtattagcacctgtgctggcctcaggggctttttccgagttgggtccactgtccacttgattgagcctctggatgctgatgaagaggggcaacatgcgatgtatcaggcaaagcatctgcaacagaaggctgggacctgtggggtcaaagataccaacctgaatgacctagggcctcgggcattagaaatatacagggctcagccacggaactggctgatacccagagaaacccgctatgtggagttgtatgtggttgcagacagccaagagttccagaagttggggagcagagaggccgtgcgccagcgagtgctggaggttgtaaaccacgtggacaagctttatcaggaactcagttttcgagttgtcctggtgggcctggagatctggaacaaggacaaattctacatcagccgctatgccaacgtgacactggagaacttcttgtcctggagggaacagaacttgcaagggcagcacccacatgacaacgtgcaacttatcacgggggttgatttcattgggagcactgttggactggctaaggtgtctgccctgtgttcccgtcactccggagctgtgaatcaggaccactccaagaactccattggtgtagcctccaccatggcccatgagctgggccacaacctgggcatgagccatgatgaggacattccaggatgctactgtcctgaaccacgggagggtggtggctgcatcatgaccgaaagcatcggctccaagttccccaggatattcagcaggtgtagcaagattgacctagagtcattcgtgacaaaaccccagacaggctgcctgaccaatgttccagatgtcaaccggttcgtgggtggccctgtgtgtggaaacctgtttgtggagcatggagagcagtgtgactgtggcacacctcaggactgtcaaaacccctgctgcaatgccaccacttgccagctggtcaagggggcagagtgtgccagtggtacctgttgtcatgaatgcaaggtgaagccagctggagaggtgtgtcgtctcagtaaggacaaatgtgacctggaggagttctgtgatggccggaagccaacatgtcccgaagatgccttccaacagaatggcactccctgcccagggggctactgctttgatgggagctgtcccaccctggcacagcagtgccgggatctgtgggggccaggtgctcgggtagcagccgactcctgctatacctttagcatccctccgggctgcaatgggaggatgtactctggcaggatcaaccggtgtggagcgctgtactgtgagggaggccagaagccccttgaacgctccttctgcactttctcctccaaccatggagtctgccatgctcttggcacaggcagcaacattgacacctttgagctggtattgcagggcaccaagtgcgaggagggaaaggtttgcatggatggaagctgccaggacctccgtgtatacagatctgaaaactgctctgctaaatgcaacaaccatggggtatgcaaccacaagagggagtgccactgtcacaagggctgggcaccacccaactgtgtacagcggctggcagatgtatcagatgaacaagcagcgtctacgagcctcccagtcagtgtggttgtggtcttggtgatcctggtggctgcgatggtcatcgtggcaggcatcgtcatctaccgaaaggctccgagacaaatccagaggaggagtgtggcacccaagcctatctcggggctctccaaccccctattctacacaagggacagcagcctgccagctaagaacaggcctccagacccttctgagacagtttctaccaaccagcccccaagacccatagtgaaaccaaagaggcctccccctgcacctccaggtgctgtgtccagttcaccactcccagttcctgtttatgccccaaagataccaaatcagtttagacctgatcctcccaccaagcccctcccagagctgaaacccaagcaggtcaagccaacctttgcacccccgacaccaccagtcaagcccgggactggagggacggtgcctggagcaactcagggagctggtgagccaaaggttgctctgaaggtccccatccagaagaggtgaccagctagggcaccccagggccatcgtttgtggacgtttggagataccactgctcctataaatgtgttccttcagcaacaccacaaccaccacccaggctggacctgcacacgctgttcctacctcaggaataatctttctgtggctcttgctgtccagatgccctcatgaaccagtggcctctcactggccttggccagcttagaccatacatctgcgtatgctagttgctaggcaggggcggtgtaccctgcagtgggcgtgatcaagtctgtctgtctctgggtggacagaatgccaggagtcccctcccagcaacttctgcactggccctcacacagtgacctctctccaaggcacagttcctgtatctacatcccactcgtgtatgagacttaggaaaggaaaagacgtcgttcttggggtgttgggacaagaagttgccgtcatctccggattgaaacagattagttgttgaaattatgactaaacactattcactaataaagattctattttatcaatagattatagatcacattcta

SEQ ID NO: 58 - protein, 826 aa, Mus musculus
MLGLWLLSVLWTPAVAPGPPLPHVKQYEVVWPRRLAASRSRRALPSHWGQYPESLSYALGTSGHVFTLHLRKNRDLLGSSYTETYSAANGSEVTEQLQEQDHCLYQGHVEGYEGSAASISTCAGLRGFFRVGSTVHLIEPLDADEEGQHAMYQAKHLQQKAGTCGVKDTNLNDLGPRALEIYRAQPRNWLIPRETRYVELYVVADSQEFQKLGSREAVRQRVLEVVNHVDKLYQELSFRVVLVGLEIWNKDKFYISRYANVTLENFLSWREQNLQGQHPHDNVQLITGVDFIGSTVGLAKVSALCSRHSGAVNQDHSKNSIGVASTMAHELGHNLGMSHDEDIPGCYCPEPREGGGCIMTESIGSKFPRIFSRCSKIDLESFVTKPQTGCLTNVPDVNRFVGGPVCGNLFVEHGEQCDCGTPQDCQNPCCNATTCQLVKGAECASGTCCHECKVKPAGEVCRLSKDKCDLEEFCDGRKPTCPEDAFQQNGTPCPGGYCFDGSCPTLAQQCRDLWGPGARVAADSCYTFSIPPGCNGRMYSGRINRCGALYCEGGQKPLERSFCTFSSNHGVCHALGTGSNIDTFELVLQGTKCEEGKVCMDGSCQDLRVYRSENCSAKCNNHGVCNHKRECHCHKGWAPPNCVQRLADVSDEQAASTSLPVSVVVVLVILVAAMVIVAGIVIYRKAPRQIQRRSVAPKPISGLSNPLFYTRDSSLPAKNRPPDPSETVSTNQPPRPIVKPKRPPPAPPGAVSSSPLPVPVYAPKIPNQFRPDPPTKPLPELKPKQVKPTFAPPTPPVKPGTGGTVPGATQGAGEPKVALKVPIQKR

SEQ ID NO: 59 - DNA, 1348 bp, Mus musculus
aacaaaccctgcagtggttcgaggcctaataggctcatctgggatcctctccagccaagcttccttgtgcaagtgtctgaagcagctatggcaactgttcctgaactcaactgtgaaatgccaccttttgacagtgatgagaatgacctgttctttgaagttgacggaccccaaaagatgaagggctgcttccaaacctttgacctgggctgtcctgatgagagcatccagcttcaaatctcgcagcagcacatcaacaagagcttcaggcaggcagtatcactcattgtggctgtggagaagctgtggcagctacctgtgtctttcccgtggaccttccaggatgaggacatgagcaccttcttttccttcatctttgaagaagagcccatcctctgtgactcatgggatgatgatgataacctgctggtgtgtgacgttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaaagtatgggctggactgtttctaatgccttccccagggcatgttaaggagctcccttttcgtgaatgagcagacagctcaatctccaggggactccttagtcctcggccaagacaggtcgctcagggtcacaagaaaccatggcacattctgttcaaagagagcctgtgttttcctccttgcctctgatgggcaaccacttacctatttatttatgtatttattgattggttgatctatttaagttgattcaaggggacattaggcagcactctctagaacagaacctagctgtcaacgtgtgggggatgaattggtcatagcccgcactgaggtctttcattgaagctgagaataaataggttcctataatatggatgagactttttatgaatgaagcaccagcacattgctttgatgagtatgaaataaatttcattaaaacaaacaaac

SEQ ID NO: 60 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 61 - DNA, 4000 bp, Homo sapiens
acaatcatccaagccagaccacactgagacgggaatgattcctggcggattagtgggctgggctgggccgggccgggagatgcacccccaagaccaagcagagaaggaggatctcttccccaaggtctttagccaacctcatctggaccaaaggatcttcccagggattcaggtctgagttacagcccagagagcaatggaggcccatacatacctcaggaaaccatggagatgggggaggcggtgacaagacagacagtcccagagaaaagccgctggggtgaagccagaaaggcaaggaggaaaagagaaagagagaccaaaagaacaaaacagatggaggagaccataagatagactgagggggaaagagagacaggaaaaatgtttgtgtgtgagagagagacagagagaaagagggtgaaagagggtgaggagagacagggatagaaagagggggtcaagggctaaaggaaaactttctggaacaagtctggcccagcccacacactcagaagccctcacaccctcccagcagcaactggaggtcttaggagttgattaagcctgcatgggggtgactttttctagacaagggggtaagcaaagggaaacaagaggaggggacgcggctctcccccatccccagccacagttcccaggtgaacccccctttcccctgctggatgtgaaatcgccatttcaccccaccacactccttaattgttgcaaaaatcatttgtgaaattggtctccaacaggcaagaacggccacgctcattccaatagacaaccccacctcaacccttccctctgcaagggcccatggaatcccggagctgcagaaaccctgggggcctgtgtcgctactgcacccactactgtgagtcttttgcagggagggtgagggaccacactgtgggcaaggtggccccagtgcaggactcctgaggttctgagggtccctggcagcagaagtgtccagctatgcactcgccccactcagctcaagatgctacatgagcactggccagaagagagggagcctcccacccttggcacaccctggaggtcttttctcacttgagaattgacctaaacacacactcacacattcctgacgcgcgcttagagccataccaaggcaacctggaggtgacctcaaagcagaaatacagacatggaccctcacttagaaacacacatttactcaaactgaccccaaacagacctacacacaaaacccacctgcaccttgacacacagacccgggcacaggcctatattcacacgcaagcacactggcccattccacaaatacagggaagaagtgccttgaaaccttccaggcagaggacagctagtgacgaatgaatggatgaatatgcaatatacctaatatatatgtgtgtgtgttatacgtgtgtatgcataagtgtatatgatacaaatacatgtatatatacacacagagaatatatatccacctatggaggtcaaagcaggccaggtccccagcaaggctggtgatgtaagggggttgctgtcttccctgggcacacatgctatggcttaaagcttgatctcccaatctctcccgggggagggccccataacttgggccaggcaaagtaccccggtgtcccccttccaattcgaaagctacttggcatctgtcttctcagttagagagagtatgagctcaacaagtttttcatcagggcccccattcccagaaggcccccccaaaatctgatgagccccttgtgctaggagagtggggagaggagggagatctgtcacatgctgcaaggggcaaaggtcacaggtgaggcgaaggggagggatggggacaccctggggccagccagcggtgagcgccccaggctaagacaaggctgaccccactgccgagagagggcgggcagggggtgagattgagggctgggttcattttattgtattttgcctccagcgtggggccagagacgctctttcccccgggagacaaaaggcctgcagttgtggcgtcctgccccgctgggagggcgtttcgcccccaggactcggtgtcccgaggttgaggctactggtcaccctggcttcagaaagcgaagaattcaggaacctctgatcaccccaccctgactcctgatcattcagttggggcgtcccctcaaacccaccagcaaggagctcagagttggtgagttcagagctgtatcaagtcttgacgggctccctccctcccatttcccacctctttgggcccccagatcctcacaccagcaccaactccctgtaaacccaactacctgctggaaaggagttataggcccggcctgggggcctgaggaggacggcggcggctggagacgtctgcacggggcggaactggaaggggaggaagaaacctgtgcctagcacttggcagacccactttgccaagcctcggaaccagatacggttactgggggcttctcagagcggcaggaaggtagagtaagaaccggacaccggggctctgacattacacagaaaaggtcagaattcctcccttcaagtcaccctaggccggttcccggggagcctcactggaggaatttcagagggtgtaagacgagggagaagatgagaaatagggcggactctggccctcggaggccagagaaaaggcgagactccagagaggcgccggacccgaggcagacattcccatacagccgggactccgagagcagccgtgggcacctgggggccggggactcggcggatccgccccattcatcaaaggaaggcactgggacaaagttggccccctccccttctcccccagcccccaactcgccgggatcgtccctctcttttcacggcggttcaaattctcccccaaaacgaggtcgagccggggattagaaaagagggaaaaaaatattcgttggcgatttcttttcgttattggtcctccgctcccttgcgccctggcccccttccctcctcatgaataaaagaaaagtccgaacctatcagatcgcgctcactgcctcctcctcctcctcctcctcctcctcctcgtacttcggtgaacacttttgcacaacttacccagctgatcactcgcgccccctcgcttttccatttctctttccccaaccccgtcaccccctgtctcctccgtccagccctgaaacccgagcaggctccttcttccgtctgtccccgggtctcctaggggacggggctgtgaaagctggtgtggagggagaagcgagtgtggtccggagaaagaaggcgtggagaagagggagggagcgagaagcgagaataaatatataaataaatacgagaacgaaatccactccgcagtctccgggctcggaaactttggccccgacggccagacgccagagcgagagcgcggcgctcgccactctgaggctgcggcctcgattccgcgcgttcccccggcccccctccgtccgcggggcctggtctccgggttctgccaggcgcatcagcccgcacaacttctggccgaggcagccggcagaggcggacttggggttggagtgtttgtttgtttgaacttcctcgtcgtcgccaccttccctccccccaacctccaacccacctcacccccctccccagcttctggacgcgtttgactgcagccaggggtggggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtaccgagaatgatgcggccggctccggggcagaactacccccgcacgggattccctttggaaggtaagaacgcccaggctggcctcgccgcgactccgccgcccggaactcggggtccttggagaggctgcggtctccaggg